(19) United States
(12) Patent Application Publication
(10) Pub. No.: US 2003/0086872 A1
(43) Pub. Date: May 8, 2003

(54) SUT-2 and SUT-3 genes, proteins and assays for inhibitors of lymphocyte adhesion

(76) Inventors: Jean-Philippe Girard, Rebigue (FR); Jean-Baptiste Vincourt, Toulouse (FR); Francois Amalric, Toulouse (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/222,009

(22) Filed: Aug. 14, 2002

Related U.S. Application Data
(60) Provisional application No. 60/312,442, filed on Aug. 15, 2001.
(60) Provisional application No. 60/333,673, filed on Nov. 26, 2001.
(60) Provisional application No. 60/323,656, filed on Sep. 19, 2001.

(51) Int. Cl. A61K 49/00; G01N 33/53; C07H 21/04; C12N 9/64; C12N 9/00; C12P 21/02; C12N 5/06

(52) U.S. Cl. 424/9.2; 435/69.1; 435/183; 435/320.1; 435/325; 435/226; 536/23.2; 435/7.1

(57) ABSTRACT

The present invention is directed to the SUT-2 and SUT-3 sulfate/anion exchanger polypeptides expressed in high endothelial venules endothelial cells (HEVECs). The invention also relates to drug screening assays for identifying compounds capable of inhibiting sulfate/anion transport and L-selectin mediated lymphocyte adhesion to high endothelial venules. Such compounds may be useful in the treatment of inflammatory conditions.

### RELATED APPLICATIONS

This application claims priority to U.S. Provisional Patent Application Serial No. 60/312,442, entitled SUT-2 GENE, PROTEIN, ASSAYS FOR INHIBITORS OF LYMPHOCYTE ADHESION, and filed Aug. 15, 2001, U.S. Provisional Patent Application Serial No. 60/333,673, entitled SUT-2 GENE, PROTEIN, ASSAYS FOR INHIBITORS OF LYMPHOCYTE ADHESION, and filed Nov. 26, 2001, and U.S. Provisional Patent Application Serial No. 60/323,656, entitled SUT-3 GENE, PROTEIN, ASSAYS FOR INHIBITORS OF LYMPHOCYTE ADHESION, and filed Sep. 19, 2001. The disclosures of each of the foregoing U.S. Provisional Patent Application are incorporated herein by reference in their entireties.

### FIELD OF THE INVENTION

The present invention is directed to polynucleotides encoding SUT-2 and SUT-3 sulfate/anion exchanger polypeptides expressed in high endothelial venules endothelial cells (HEVECs). The invention also concerns polypeptides encoded by the SUT-2 and SUT-3 genes. The invention also relates to drug screening assays for identifying compounds capable of inhibiting sulfate/anion transport, which compounds may be used in inhibiting L-selectin mediated lymphocyte adhesion to high endothelial venules.

### BACKGROUND

Patrolling the body in search for foreign antigens, lymphocytes continuously recirculate from blood, through lymphoid and other tissues, and back through the efferent lymphatics to the blood. The first critical step in lymphocyte migration from circulation into lymphoid tissues is the adhesion of lymphocytes to specialized postcapillary vascular sites called high endothelial venules (HEV) (Girard and Springer (1995) Imm. Today 16: 449-457). In contrast to the endothelial cells from other vessels, the endothelial cells of HEV have a plump, almost cuboidal, appearance, express specialized ligands for the lymphocyte homing receptor L-selectin, and are able to support high levels of lymphocyte extravasion from the blood (Marchesi et al, (1964) Proc. R. Soc. London B. 159: 283-290; Girard and Springer, supra; and Anderson and Anderson, (1976) Immunology 31: 731-748).

Extravasation of lymphocytes through HEVs occurs as a cascade of events, the first of which involves tethering and rolling of the lymphocyte along the HEV. This is followed by chemokine mediated activation of beta-2 integrins on the lymphocytes, leading to their arrest and subsequent transmigration into the lymph node parenchyma. L-selectin, the peripheral lymph node homing receptor, is a lectin-like cell adhesion molecule and has been shown to mediate tethering and rolling of lymphocytes along HEVs. L-selectin interacts with HEV-associated ligands collectively known as the peripheral node vascular addressin (PNAd), initially identified using the monoclonal antibody MECA-79 (Streeter et al, (1998) J Cell Biol 107: 1853-1862). MECA-79 binds to HEVs and blocks the adhesion of lymphocytes to HEVs in peripheral nodes, but not in Peyer's patches. MECA-79 antigens are also induced in a number of human inflammatory diseases, including models of rheumatoid arthritis, various types of skin inflammation (Michie et al, (1993) Am. J. Pathol. 143: 1688-1698), cardiac allograft rejection (Toppila et al, (1999) Am. J. Pathol. 155: 1303-1310), and bronchial asthma (Toppila et al, (2000) Am. J. Respir. Cell. Mol. Biol. 23: 492-498). Furthermore, several experimental inflammation models have been established, including a transgenic mouse model of pancreatic inflammation (Onrust et al, (1996) J. Clin. Invest. 97: 54-64) and thymic hyperplasia in the AKR/J mouse, a model of aberrant lymphocyte migration (Michie et al, (1995) Am. J. Pathol. 147: 412-421). The occurrence of the MECA-79 epitope is currently accepted as a predictor for L-selectin ligand activity. Studies have shown that the MECA-79 epitope represents a carbohydrate-based post-translational modification that is presented on HEVs by several endothelial glycoproteins.

A unique characteristic of the HEV endothelium that has intrigued many investigators over the past 20 years is the capacity of HEV endothelial cells (HEVEC), both in humans and in rodents, to incorporate large amounts of inorganic sulfate (Andrews et al, (1982) J. Cell Sci. 57: 277-292). Interestingly, HEV-like vessels that develop in chronically inflamed nonlymphoid tissues exhibit a similar ability to incorporate high levels of radioactive sulfate, and this property of the HEV endothelium has been shown to be closely related to the concentration of lymphocytes in the perivascular infiltrates (Freemont, (1988) J. Path. 155: 225-230).

More recently, the functional implication for this major biosynthetic activity of HEV and HEV-like vessels has been found when it was discovered that large amounts of sulfate are incorporated into sialomucin-type L-selectin counter-receptors (Lasky et al, (1992) Cell 69: 927-938; Baumhueter et al, (1993) Science 262:436-438; and Imai et al, (1993) Nature 361: 555-557). Sulfate residues were shown to be essential for recognition of HEV sialomucins GlyCAM1 and CD34 by L-selectin and HEV-specific monoclonal antibody MECA-79 (Imai et al, supra; Hemmerich et al, (1994) J. Exp. Med. 180: 2219-2226; Shailubhai et al, (1997) Glycobiol. 7: 305-314), an antibody that blocks L-selectin-dependent lymphocyte adhesion to HEV in vitro and in vivo (Streeter et al, supra).

In view of the important role of sulfation in lymphocyte adhesion, genes involved in the sulfation of L-selectin ligands in HEV have been of great interest as biological targets for the development of therapeutic molecules. Efforts to date have concentrated on the genes encoding PAPS synthetase, a bifunctional enzyme catalyzing synthesis of PAPS (3′-phosphoadenosine-5′-phosphosulfate), the activated sulfate donor used by all sulfotransferases (Girard et al. (1998) Faseb J., 12:603-612, the disclosure of which is incorporated herein by reference in its entirety), and sulfotransferases of two specificities, N-acetylglucosamine-6-O-sulfotransferase(s), and galactose-6-O-sulfotransferase(s) (Kimura et al, (1999) PNAS USA 96: 4530-4535; Bistrup et al, (1999) J. Cell Biol 145: 899-910; and Hiraoka et al, (1999) Immunity 11: 79-89).

However, although the transfer of sulfate from PAPS to HEV sialomucins by sulfotransferases appears to be the most specific step in the pathway, sulfation of HEV sialomucins may also be controlled at earlier steps. For example, the observation that sulfate incorporation is the rate-limiting step for sulfation in cartilage (Hastabacka et al, (1994) Cell 78: 1073-1087; and Superti-Furga et al, (1996) Nat. Genet. 12: 100-102) suggests that sulfate transport could play a major role in the control of HEV sialomucins sulfation in vivo.

Two families of vertebrate sulfate transporters, which play a role in sulfate incorporation in other tissues, have recently been defined. The prototype members of these families, SAT-1 and NaSi-1, were initially described in rats (Markovitch et al, (1993) PNAS USA 90: 8073-8077; and Bissig et al, (1994) J. Biol. Chem. 269: 3017-3021). SAT-1 is a member of the superfamily of anion exchangers functioning as a Na+-independent sulfate transporter in rat hepatocytes and being sensitive to the anion-exchanger inhibitor 4,4′-diisothiocyanostilbene-2,2′-disulfonic acid (DIDS). NaSi-1 is a member of the superfamily of Na+-coupled transporters functioning as a DIDS-resistant Na+-dependent sulfate transporter in rat kidney and small intestine. Another DIDS-resistant, Na+ coupled sulfate transporter/anion exchange protein expressed in HEVs, SUT-1, has recently been characterized (Girard et al, (1999) PNAS USA 92(22): 12772-12777). In contrast, two members of the superfamily of anion exchangers, human DTD and DRA, have been described (Hastabacka et al, supra; and Silberg et al, (1995) J. Biol. Chem. 270: 11897-11902), that contain 12 membrane-spanning domains and are sensitive to DIDS (Satoh et al, (1998) J. Biol. Chem. 273: 12307-12315). Both DTD and DRA have been implicated in the pathogenesis of human diseases. While DTD is mutated in three different chondrodysplasias (diastrophic dysplasia, atelosteogenesis type II, and achondrogenesis type IB), DRA is mutated in congenital chloride diarrhea (Hoglund et al, 1996 Nat Genet 14:316-319, the disclosure of which is incorporated herein by reference in its entirety). DTD mutations result in undersulfation of cartilage proteoglycans and thus lead to severe developmental defects of the skeleton (Hastabacka et al, supra, Superti-Furga et al, supra). It is unknown whether DTD and DRA are expressed in HEV.

There is therefore a need for the identification of biological targets for the development of therapeutic molecules for the treatment of inflammation, particularly for inhibiting lymphocyte extravasation from HEV and HEV-like vessels, and more specifically for the identification of sulfate transporters involved in the sulfation of L-selectin ligands in HEVs and HEV-like vessels.

### SUMMARY OF THE INVENTION

The present invention relates to genes and proteins involved in sulfation of L-selectin ligands as targets for the development of therapeutic molecules and provides molecular mechanisms controlling lymphocyte extravasation through HEV. The genes and proteins of the present invention are involved in anion transport, particularly sulfate transport. Provided herein are the molecular cloning and functional characterization of SUT-2 and SUT-3, two sulfate transporters expressed in human HEVECs. In order to characterize the function of the SUT-2 and SUT-3 proteins and develop screening assays that can be used in drug screening, functional assays for SUT-2 and SUT-3 activity were developed, allowing sulfate transport and anion exchange function to be examined. SUT-2 and SUT-3 therefore provide valuable biological targets for the inhibition of sulfation of L-selectin ligands, thereby inhibiting the adhesion of lymphocytes to HEVs and ameliorating or preventing inflammation, particularly chronic inflammation. The invention is thus directed to methods for the screening of substances or molecules that inhibit the expression of the SUT-2 and SUT-3 genes, as well as with methods for the screening of substances or molecules that interact with and/or inhibit the activity of a SUT-2 and SUT-3 polypeptides.

In one aspect the invention encompasses a method of identifying a candidate compound for the treatment of an inflammatory condition as well as a method of selecting a SUT-2 or SUT-3 inhibitor, said method comprising: (a) providing a compound capable of inhibiting the activity of a SUT-2 or SUT-3 protein; and (b) determining whether said compound is capable of, or likely to be capable of, inhibiting leukocyte adhesion or migration.

In another aspect, the invention relates to a method of identifying a candidate compound for the treatment of an inflammatory condition, or a method of selecting a SUT-2 or SUT-3 inhibitor, said method comprising: (a) providing a compound capable of inhibiting the activity of a SUT-2 or SUT-3 protein; and (b) performing an assay for SUT-2 or SUT-3 activity, said assay measuring a property indicative of leukocyte adhesion or migration.

In preferred aspects, the invention comprises a method of identifying a candidate compound for the treatment of an inflammatory condition, or a method of selecting a SUT-2 or SUT-3 inhibitor, said method comprising: (a) providing a test compound; (b) determining whether said test compound is capable of inhibiting the activity of a SUT-2 or SUT-3 protein; and (c) for a compound capable of inhibiting SUT-2 or SUT-3 activity, determining whether said test compound is capable of, or likely to be capable of, inhibiting leukocyte adhesion or migration. Preferably, the assay of step (b) is distinct from the assay of step (c), preferably assessing a different endpoint.

In the methods of the invention, said step of determining whether said test compound is capable of inhibiting the activity of a SUT-2 or SUT-3 protein may comprise determining whether said test compound inhibits sulfate transport activity.

Determining whether said test compound is capable of, or likely to be capable of, inhibiting leukocyte adhesion or migration preferably comprises performing an assay designed to measure a property indicative of leukocyte adhesion or migration.

Preferably, the step of determining whether said test compound is capable of inhibiting the activity of a SUT-2 or SUT-3 protein comprises: (i) contacting a SUT-2 or SUT-3 polypeptide or a fragment thereof with a test compound; and (ii) determining whether said compound selectively inhibits SUT-2 or SUT-3 activity.

In futher preferred embodiments, determining whether said test compound is capable of inhibiting the activity of a SUT-2 or SUT-3 protein comprises: (i) providing a cell comprising a SUT-2 or SUT-3 polypeptide or a fragment comprising at least 6 consecutive amino acids thereof; (ii) contacting said cell with a test compound; and (iii) determining whether said compound selectively inhibits SUT-2 or SUT-3 activity.

Determining whether said compound selectively inhibits SUT-2 or SUT-3 activity preferably comprises assessing sulfate uptake activity.

As discussed below, a determination that said compound is capable of, or likely to be capable of, inhibiting leukocyte adhesion or migration indicates that said compound is a candidate compound for the treatment of an inflammatory condition.

Preferably, the property measured in determining whether a compound is capable of, or likely to be capable of, inhibiting leukocyte adhesion or migration is indicative of migration from endothelial vessels, most preferably from high endothelial venules. In other embodiments, said property measured is indicative of adhesion to an endothelial cell or lymphatic tissue, or most preferably to a high endothelial venule endothelial cell (HEVEC).

Determining whether said test compound is capable of, or likely to be capable of, inhibiting leukocyte adhesion or migration can be carried out according to any suitable method. In one example the sulfation of an L-selectin ligand, preferably a sialomucin type L-selectin counter-receptor, is measured. In another embodiment, L-selectin dependent leukocyte adhesion to cells comprising an L-selectin ligand is measured. In another aspect, an interaction between L-selectin ligands is measured; the interaction is preferably an interaction measured between a sialomucin-type L-selectin counterreceptor and L-selectin. In yet further examples, adhesion of leukocytes to HEVs or HEVECs is measured or assessed, preferably by observing the ‘rolling phenotype’ in vivo in mouse lymph node HEVs.

In further aspects, the invention includes a method of identifying a candidate compound for the treatment of an inflammatory condition or a method for selecting a SUT-2 or SUT-3 inhibitor, said method comprising: (a) providing a compound capable of inhibiting the activity of a SUT-2 or SUT-3 protein; and (b) administering said compound to an animal model of an inflammatory disorder, and assessing the ability of said compound to ameliorate said disorder. A determination that said compound is capable of ameliorating said condition indicates that said compound is a candidate compound for the treatment of an inflammatory condition. Preferably, ameliorating said disorder comprises ameliorating a symptom of said disorder.

In another aspect, the invention relates to a method of identifying a candidate SUT-2 or SUT-3 inhibitor, said method comprising: a) providing an insect cell transfected with a baculovirus expression vector comprising a nucleic acid encoding a SUT-2 or SUT-3 polypeptide; b) contacting said cell with a test compound; and c) determining whether said compound selectively inhibits sulfate uptake activity; wherein a determination that said compound selectively inhibits sulfate uptake activity indicates that said compound is a candidate inhibitor of said SUT-2 or SUT-3 polypeptide.

The invention also provides a method of identifying a candidate SUT-2 inhibitor, said method comprising: a) providing a cell comprising a nucleic acid encoding a SUT-2 polypeptide; b) contacting said cell with a test compound; and c) determining whether said compound selectively inhibits sulfate uptake activity; wherein a determination that said compound selectively inhibits sulfate uptake activity indicates that said compound is a candidate inhibitor of said SUT-2 polypeptide.

Also disclosed is a method of identifying a candidate SUT-2 or SUT-3 inhibitor, said method comprising: a) providing a Xenopus oocyte comprising a nucleic acid encoding a SUT-2 or SUT-3 polypeptide; b) contacting said cell with a test compound; and c) determining whether said compound selectively inhibits sulfate uptake activity, wherein a determination that said compound selectively inhibits sulfate uptake activity indicates that said compound is a candidate inhibitor of said SUT-2 or SUT-3 polypeptide.

It will be appreciated that in any of the methods of identifying or selecting a candidate compound, the methods of the invention may further comprise administering a candidate compound to an animal model of an inflammatory disorder and assessing the ability of the compound to ameliorate said disorder.

Preferably, in the methods of the invention, a determination that said compound is capable of, or likely to be capable of, inhibiting leukocyte adhesion or migration indicates that said compound is a candidate compound for the treatment of an inflammatory disorder.

Preferably, an inhibitor is a selective inhibitor of said SUT-2 or SUT-3 polypeptide.

In one aspect, the invention provides a nucleic acid encoding a SUT-3 or SUT-2 polypeptide having sulfate transporter activity selected from the group consisting of:

(i) a nucleic acid molecule encoding a polypeptide comprising an amino acid sequence selected from the group of sequences consisting of SEQ ID NOs: 4 to 6, 10, 13, 14 and 27;

(ii) a nucleic acid molecule comprising a nucleic acid sequence selected from the group of sequences consisting of SEQ ID NOs: 1 to 3, 9, 11 and 12, or a sequence complementary thereto;

(iii) a nucleic acid molecule the complementary strand of which hybridizes under stringent conditions to a nucleic acid as defined in (i) and (ii); and

(iv) a nucleic acid the sequence of which is degenerate as a result of the genetic code to a sequence of a nucleic acid as defined in (i), (ii) and (iii).

In another aspect, the invention provides an isolated nucleic acid, said nucleic acid comprising a nucleotide sequence encoding: i) a polypeptide comprising an amino acid sequence having at least about 80% identity to a sequence selected from the group consisting of the polypeptides of SEQ ID NOs: 4 to 6, 10, 13, 14 and 27, and the polypeptides encoded by the nucleic acid of SEQ ID NOs: 1 to 3, 9, 11 and 12; or ii) a fragment of said polypeptide which possesses sulfate transport activity. With respect to said nucleic acids of the invention, polypeptide identity can be determined using an algorithm selected from the group consisting of XBLAST with the parameters score=50 and wordlength=3, Gapped BLAST with the default parameters of XBLAST, and BLAST with the default parameters of XBLAST. Most preferably, said polypeptide comprises an amino acid sequence selected from the group consisting of the sequences shown as SEQ ID NOs: 4 to 6, 10, 13, 14 and 27 and the polypeptides encoded by the nucleic acid of SEQ ID NOs: 1 to 3, 9, 11 and 12.

In preferred aspects, a nucleic acid of the invention is operably linked to a promoter. Also envisioned is an expression cassette comprising the nucleic acid as well as a host cell comprising said expression cassette.

The present disclosure also provides a method of making a SUT-3 or SUT-2 polypeptide, said method comprising: a) providing a population of host cells comprising a nucleic acid encoding said SUT-3 or SUT-2 protein of the invention; and b) culturing said population of host cells under conditions conducive to the expression of said recombinant nucleic acid, whereby said polypeptide is produced within said population of host cells. Optionally the method further comprises purifying said polypeptide from said population of cells.

In further aspects, the invention provides an isolated nucleic acid, said nucleic acid comprising a nucleotide sequence having at least about 80% identity over at least about 100 nucleotides to a sequence selected from the group consisting of the sequences shown as SEQ ID NOs: 1 to 3, 9, 11 and 12, and sequences complementary thereto. In further preferred embodiments, said nucleic acid hybridizes under stringent conditions to a nucleic acid comprising SEQ ID NO: 1 to 3, 9, 11 or 12, or the sequence complementary thereto. Preferably, identity is determined using an algorithm selected from the group consisting of NBLAST with the parameters score=100 and wordlength=12, Gapped BLAST with the default parameters of NBLAST, and BLAST with the default parameters of NBLAST.

The invention also provides a polypeptide encoded by the nucleic acid of the invention, wherein said polypeptide has sulfate transport activity. Most preferably, the invention provides an isolated polypeptide or fragment thereof possessing sulfate transport activity, said polypeptide comprising an amino acid sequence having at least about 80% amino acid sequence identity to a sequence selected from the group consisting of SEQ ID NOs: 4 to 6, 10, 13, 14 and 27. Preferably, identity is determined using an algorithm selected from the group consisting of XBLAST with the parameters score=50 and wordlength=3, Gapped BLAST with the default parameters of XBLAST, and BLAST with the default parameters of XBLAST. Preferably, said polypeptide is selectively bound by an antibody raised against an antigenic polypeptide, or antigenic fragment thereof, said antigenic polypeptide comprising a polypeptide selected from the group consisting of SEQ ID NOs: 4 to 6, 10, 13, 14 and 27. Most preferably, said polypeptide comprises a polypeptide selected from the group consisting of SEQ ID NOs: 4 to 6, 10, 13, 14 and 27.

In further aspects, encompassed by the invention is a nucleic acid comprising a contiguous span of at least 20 nucleotides of a sequence selected from the group consisting of SEQ ID NOs: 1 to 3, 9, 11 and 12, or the sequence complementary thereto. Encompassed is also a polypeptide comprising a contigous span of at least 6 amino acids of a sequence selected from the group consisting of SEQ ID NOs: 4 to 6, 10, 13, 14 and 27.

In other aspects, the invention encompasses an antibody that selectively binds to a polypeptide of the invention.

Futhermore, the invention provides a method of determining whether a SUT-3 or SUT-2 is expressed within a biological sample, said method comprising the steps of:

a) contacting said biological sample with:

(i) a polynucleotide that hybridizes under stringent conditions to a nucleic acid of the invention; or

(ii) a detectable polypeptide that selectively binds to the polypeptide of the invention; and

b) detecting the presence or absence of hybridization between said polynucleotide and an RNA species within said sample, or the presence or absence of binding of said detectable polypeptide to a polypeptide within said sample;

wherein a detection of said hybridization or of said binding indicates that said SUT-3 or SUT-2 is expressed within said sample. Said polynucleotide is preferably a primer, and said hybridization is preferably detected by detecting the presence of an amplification product comprising said primer sequence. Said detectable polypeptide is preferably an antibody.

Another embodiment relates to a method of determining whether a mammal has an elevated or reduced level of SUT-3 or SUT-2 expression, said method comprising the steps of: a) providing a biological sample from said mammal; and b) comparing the amount of a SUT-3 or SUT-2 polypeptide of the invention or of a SUT-3 or SUT-2 RNA species encoding a polypeptide of the invention within said biological sample with a level detected in or expected from a control sample, wherein an increased amount of said SUT-3 or SUT-2 polypeptide or said SUT-3 or SUT-2 RNA species within said biological sample compared to said level detected in or expected from said control sample indicates that said mammal has an elevated level of SUT-3 or SUT-2 expression, and wherein a decreased amount of said SUT-3 or SUT-2 polypeptide or said SUT-3 or SUT-2 RNA species within said biological sample compared to said level detected in or expected from said control sample indicates that said mammal has a reduced level of SUT-3 or SUT-2 expression.

A further embodiment relates to a method of identifying a candidate inhibitor of a SUT-3 or SUT-2 polypeptide, said method comprising: a) contacting a SUT-3 or SUT-2 polypeptide according to the invention or a fragment comprising a a contiguous span of at least 6 contiguous amino acids of a polypeptide according to the invention with a test compound; and b) determining whether said compound selectively binds to said polypeptide, wherein a determination that said compound selectively binds to said polypeptide indicates that said compound is a candidate inhibitor of said polypeptide.

Also encompassed is a method of identifying a candidate inhibitor of a SUT-3 or SUT-2 polypeptide of the invention or a fragment comprising a a contiguous span of at least 6 contiguous amino acids of a polypeptide according to the invention, said method comprising: a) contacting said polypeptide with a test compound; and b) determining whether said compound selectively inhibits the activity of said polypeptide, wherein a determination that said compound selectively inhibits the activity of said polypeptide indicates that said compound is a candidate inhibitor of said polypeptide.

Further aspects include a method of identifying a candidate SUT-3 or SUT-2 inhibitor, said method comprising: a) providing a cell comprising a SUT-3 or SUT-2 polypeptide or a fragment comprising at least 6 consecutive amino acids thereof; b) contacting said cell with a test compound; and c) determining whether said compound selectively inhibits SUT-3 or SUT-2 activity;

wherein a determination that said compound selectively inhibits the activity of said polypeptide indicates that said compound is a candidate inhibitor of said polypeptide. Preferably said cell is a Sf9 cell or a Xenopus laevis oocyte. Preferably said step a) comprises introducing a nucleic acid comprising the nucleotide sequence encoding said SUT-3 or SUT-2 polypeptide according to the invention into said cell. More preferably, step a) comprises introducing a baculovirus vector comprising a nucleic acid encoding a SUT-3 or SUT-2 polypeptide into said cell, or introducing a SUT-3 or SUT-2 cRNA into said cell. Preferably, step d) comprises detecting sulfate uptake by said cell.

In other aspects, the invention encompasses a polynucleotide according to the invention attached to a solid support, as well as a polynucleotide according to the invention further comprising a label. Also encompassed is an array comprising a polynucleotide of the invention, preferably an addressable array, of polynucleotides comprising at least one polynucleotide according to the invention.

Disclosed herein is also a method of modulating the extent of sulfation in a cell comprising modulating the activity of the SUT-3 or SUT-2 protein, as well as a method of modulating extravasion of lymphocytes in an individual comprising modulating the activity of the SUT-3 or SUT-2 protein in said individual. In another aspect, provided is a method of reducing inflammation in an individual comprising inhibiting the activity of the SUT-3 or SUT-2 protein in said individual

In other embodiments, disclosed is a method of increasing extravasion of lymphocytes in an individual comprising increasing the activity of the SUT-3 or SUT-2 protein in said individual.

In another aspect, the invention provides a method of identifying a candidate activator of a SUT-3 or SUT-2 polypeptide, said method comprising:

(a) contacting a SUT-3 or SUT-2 polypeptide according to the invention or a fragment comprising a a contiguous span of at least 6 contiguous amino acids of a polypeptide according to the invention with a test compound; and

(b) determining whether said compound selectively binds to said polypeptide; wherein a determination that said compound selectively binds to said polypeptide indicates that said compound is a candidate activator of said polypeptide.

In yet another aspect, disclosed is a method of identifying a candidate activator of a SUT-3 or SUT-2 polypeptide of the invention or a fragment comprising a contiguous span of at least 6 contiguous amino acids of a polypeptide according to the invention, said method comprising: a) contacting said polypeptide with a test compound; and b) determining whether said compound selectively increases the activity of said polypeptide, wherein a determination that said compound selectively increases the activity of said polypeptide indicates that said compound is a candidate inhibitor of said polypeptide.

Further provided is a method of identifying a candidate SUT-3 or SUT-2 inhibitor, said method comprising: a) providing a cell comprising a SUT-3 or SUT-2 polypeptide or a fragment comprising at least 6 consecutive amino acids thereof; b) contacting said cell with a test compound; and c) determining whether said compound selectively inhibits SUT-3 or SUT-2 activity, wherein a determination that said compound selectively inhibits the activity of said polypeptide indicates that said compound is a candidate inhibitor of said polypeptide. Said cell is preferably a Sf9 cell or a Xenopus laevis oocyte. Preferably, step a) comprises introducing a baculovirus vector comprising a nucleic acid encoding a SUT-3 or SUT-2 polypeptide into said cell, or introducing a SUT-3 or SUT-2 cRNA into said cell. Preferably, step d) comprises detecting sulfate uptake by said cell.

Additional embodiments of the present invention are summarized in the following numbered paragraphs:

1. A method of identifying a candidate compound for the amelioration of an inflammatory condition, said method comprising:

(a) identifying a compound which inhibits the activity of a SUT-2 or SUT-3 protein; and

(b) determining whether said compound is capable of, or likely to be capable of, inhibiting leukocyte adhesion or migration through blood vessels.

2. The method of Paragraph 1, wherein said SUT-2 or SUT-3 protein comprises an amino acid sequence selected from the group consisting of SEQ ID NOs: 4, 5, 6, 10, 13, 14 and 27.

3. The method of Paragraph 1, wherein said SUT-2 or SUT-3 protein comprises an amino acid selected from the group consisting of an amino acid sequence having at least about 60% amino acid identity to one of SEQ ID NOs: 4, 5, 6, 10, 13, 14, or 27, an amino acid sequence having at least about 65% amino acid identity to one of SEQ ID NOs: 4, 5, 6, 10, 13,14 or 27, an amino acid sequence having at least about 70% amino acid identity to one of SEQ ID NOs: 4, 5, 6, 10, 13, 14 or 27, an amino acid sequence having at least about 75% amino acid identity to one of SEQ ID NOs: 4, 5, 6, 10, 13,14 or 27, an amino acid sequence having at least about 80% amino acid identity to one of SEQ ID NOs: 4, 5, 6, 10, 13, 14 or 27, an amino acid sequence having at least about 85% amino acid identity to one of SEQ ID NOs: 4, 5, 6, 10, 13, 14 or 27, an amino acid sequence having at least about 90% amino acid identity to one of SEQ ID NOs: 4, 5, 6, 10, 13, 14 or 27, an amino acid sequence having at least about 92% amino acid identity to one of SEQ ID NOs: 4, 5, 6, 10, 13, 14 or 27, an amino acid sequence having at least about 95% amino acid identity to one of SEQ ID NOs: 4, 5, 6, 10, 13, 14 or 27, an amino acid sequence having at least about 97% amino acid identity to one of SEQ ID NOs: 4, 5, 6, 10, 13, 14 or 27, an amino acid sequence having at least about 98% amino acid identity to one of SEQ ID NOs: 4, 5, 6, 10, 13, 14 or 27, an amino acid sequence having at least about 98% amino acid identity to one of SEQ ID NOs: 4, 5, 6, 10, 13, 14 or 27, an amino acid sequence having at least about 99% amino acid identity to one of SEQ ID NOs: 4, 5, 6, 10, 13, 14 or 27, and an amino acid sequence having at least about 99.8% amino acid identity to one of SEQ ID NOs: 4, 5, 6, 10, 13, 14 or 27.

4. The method of Paragraph 1, wherein said SUT-2 or SUT-3 protein comprises an amino acid sequence selected from the group consisting of residues 164-471 of SEQ ID NO: 13, residues 137-512 of SEQ ID NO: 5, residues 137-468 of SEQ ID NOs: 4,6 and 10, and residues 77-98 of SEQ ID NOs: 4, 5, 6 and 10.

5. The method of Paragraph 1, wherein said SUT-2 or SUT-3 protein comprises an amino acid seequence selected from the group consisting of an amino acid sequence in which at least about 50% -80% of the residues are identical or similar to residues 164-471 of SEQ ID NO: 13, residues 137-512 of SEQ ID NO: 5, residues 137-468 of SEQ ID NOs: 4, 6 and 10 or residues 77-98 of SEQ ID NOs: 4, 5, 6 and 10, an amino acid sequence in which at least about 60-70% are identical or similar to residues 164-471 of SEQ ID NO: 13, residues 137-512 of SEQ ID NO: 5, residues 137-468 of SEQ ID NOs: 4, 6 and 10 or residues 77-98 of SEQ ID NOs: 4, 5, 6 and 10, and an amino acid sequence in which at least about 65% of the amino acid residues are identical or similar amino acids-to residues 164-471 of SEQ ID NO: 13, residues 137-512 of SEQ ID NO: 5, residues 137-468 of SEQ ID NOs: 4, 6 and 10 or residues 77-98 of SEQ ID NOs: 4, 5, 6 and 10.

6. The method of Paragraph 1, wherein said SUT-2 or SUT-3 protein comprises an amino acid sequence selected from the group consisting of residues 481-656 of SEQ ID NOs: 13, residues 514-620 of SEQ ID NO: 5, and residues 470-576 of SEQ ID NOs: 4, 6 and 10.

7. The method of Paragraph 1, wherein said SUT-2 or SUT-3 protein comprises an amino acid seequence selected from the group consisting of an amino acid sequence in which at least about 50% -80% of the residues are identical or similar to residues 481-656 of SEQ ID NOs: 13, residues 514-620 of SEQ ID NO: 5, and residues 470-576 of SEQ ID NOs: 4, 6 and 10, an amino acid sequence in which at least about 60-70% are identical or similar to residues 481-656 of SEQ ID NOs: 13, residues 514-620 of SEQ ID NO: 5, or residues 470-576 of SEQ ID NOs: 4, 6 and 10 , and an amino acid sequence in which at least about 65% of the amino acid residues are identical or similar amino acids-to residues 481-656 of SEQ ID NOs: 13, residues 514-620 of SEQ ID NO: 5, and residues 470-576 of SEQ ID NOs: 4, 6 and 10.

8. The method of Paragraph 1, wherein said compound inhibits a SUT-2 or SUT-3 activity selected from the group consisting of anion exchange activity, sulfate ion transport activity, and sulfation of L-selectin ligands.

9. The method according to paragraph 1, wherein the method further comprises administering a compound determined to be capable of, or likely to be capable of, inhibiting leukocyte adhesion or migration through blood vessels to an animal model of an inflammatory disorder and assessing the ability of the compound to ameliorate said disorder.

10. The method according to paragraph 1, wherein said compound is a candidate compound for the treatment of an inflammatory disorder.

11. The method according to paragraph 1, wherein a determination that said compound is capable of, or likely to be capable of, inhibiting leukocyte adhesion or migration through blood vessels indicates that said compound is a candidate compound for the treatment of an inflammatory disorder.

12. The method according to paragraph 1, wherein said inhibitor is a selective inhibitor of said SUT-2 or SUT-3 polypeptide.

13. The method according to paragraph 1, wherein said SUT-2 polypeptide is human SUT-2 polypeptide.

14. The method according to paragraph 1, wherein said SUT-3 polypeptide is human SUT-3 polypeptide.

15. A method of identifying a candidate compound for the amelioration of an inflammatory condition, said method comprising:

(a) providing a test compound;

(b) determining whether said test compound is capable of inhibiting the activity of a SUT-2 or SUT-3 protein; and

(c) for a compound capable of inhibiting SUT-2 or SUT-3 activity, determining whether said test compound is capable of, or likely to be capable of, inhibiting leukocyte adhesion or migration through blood vessels.

16. The method of paragraph 15, wherein determining whether said test compound is capable of inhibiting the activity of a SUT-2 or SUT-3 protein comprises determining whether said test compound inhibits sulfate transport activity.

17. The method of paragraph 15, wherein determining whether said test compound is capable of inhibiting the activity of a SUT-2 or SUT-3 protein comprises:

(i) contacting a SUT-2 or SUT-3 polypeptide or a fragment thereof with a test compound; and

(ii) determining whether said compound selectively inhibits SUT-2 or SUT-3 activity.

18. The method of paragraph 15, wherein determining whether said test compound is capable of inhibiting the activity of a SUT-2 or SUT-3 protein comprises:

(i) providing a cell comprising a SUT-2 or SUT-3 polypeptide or a fragment comprising at least 6 consecutive amino acids thereof;

(ii) contacting said cell with a test compound; and

(iii) determining whether said compound selectively inhibits SUT-2 or SUT-3 activity.

19. The method of paragraph 18, wherein said step of determining whether said compound selectively inhibits SUT-2 or SUT-3 activity comprises assessing sulfate uptake activity.

20. The method of paragraph 18, wherein said cell is an insect cell transfected with a baculovirus vector comprising a nucleic acid sequence encoding a SUT-2 or SUT-3 polypeptide.

21. The method of paragraph 15, comprising performing a first assay to determine whether said test compound is capable of inhibiting the activity of a SUT-2 or SUT-3 protein, and performing a second assay to determine whether said test compound is capable of, or likely to be capable of, inhibiting leukocyte adhesion or migration through blood vessels, said first assay having a protocol different from said second assay.

22. The method of paragraph 15, wherein determining whether said test compound is capable of, or likely to be capable of, inhibiting leukocyte adhesion or migration through blood vessels comprises performing an assay designed to measure a property indicative of leukocyte adhesion or migration through blood vessels.

23. The method according to paragraph 15, wherein a determination that said compound is capable of, or likely to be capable of, inhibiting leukocyte adhesion or migration through blood vessels indicates that said compound is a candidate compound for the treatment of an inflammatory condition.

24. The method according to paragraph 15, wherein said leukocyte is a lymphocyte, neutrophil or monocyte.

25. The method according to paragraph 22, wherein the property measured is indicative of migration through endothelial vessels.

26. The method of paragraph 25, wherein said property measured is indicative of migration through high endothelial venules.

27. The method of paragraph 22, wherein said property measured is indicative of adhesion to an endothelial cell or lymphatic tissue.

28. The method of paragraph 22, wherein said property measured is indicative of adhesion to a high endothelial venule endothelial cell (HEVEC).

29. The method according to paragraph 15, wherein determining whether said test compound is capable of, or likely to be capable of, inhibiting leukocyte adhesion or migration through blood vessels comprises measuring the sulfation of an L-selectin ligand.

30. The method of paragraph 29, wherein said L-selectin ligand is a sialomucin type L-selectin counter-receptor.

31. The method according to paragraph 15, wherein determining whether said test compound is capable of, or likely to be capable of, inhibiting leukocyte adhesion or migration through blood vessels comprises measuring L-selectin dependent leukocyte adhesion to cells comprising an L-selectin ligand.

32. The method according to paragraph 15, wherein determining whether said test compound is capable of, or likely to be capable of, inhibiting leukocyte adhesion or migration through blood vessels comprises measuring an interaction between L-selectin and L-selectin ligands.

33. The method of paragraph 32, wherein interaction is measured between a sialomucin-type L-selectin counter receptor and L-selectin.

34. The method according to paragraph 15, wherein determining whether said test compound is capable of, or likely to be capable of, inhibiting leukocyte adhesion or migration through blood vessels comprises measuring adhesion of leukocytes to HEVs or HEVECs.

35. The method of paragraph 34, wherein adhesion is measured by observing the ‘rolling phenotype’ in vivo in mouse lymph node HEVs.

36. The method according to paragraph 15, wherein the method further comprises administering a compound determined to be capable of, or likely to be capable of, inhibiting leukocyte adhesion or migration through blood vessels to an animal model of an inflammatory disorder and assessing the ability of the compound to ameliorate said disorder.

37. The method according to paragraph 15, wherein said compound is a candidate compound for the treatment of an inflammatory disorder.

38. The method according to paragraph 15, wherein said inhibitor is a selective inhibitor of said SUT-2 or SUT-3 polypeptide.

39. The method according to paragraph 15, wherein said SUT-2 polypeptide is human SUT-2 polypeptide.

40. The method according to paragraph 15, wherein said SUT-3 polypeptide is human SUT-3 polypeptide.

41. A method of identifying a candidate compound for the amelioration of an inflammatory condition, said method comprising:

(a) providing a compound capable of inhibiting the activity of a SUT-2 or SUT-3 protein; and

(b) administering said compound to an animal model of an inflammatory disorder, and assessing the ability of said compound to ameliorate said disorder.

42. The method of paragraph 41, wherein a determination that said compound is capable of ameliorating said condition indicates that said compound is a candidate compound for the treatment of an inflammatory condition.

43. The method of paragraph 41, wherein said animal model is an animal model of chronic inflammation.

44. The method of paragraph 41, wherein said animal model is an animal model of rheumatoid arthritis.

45. The method of paragraph 41, wherein said animal model is an animal model of inflammatory bowel diseases.

46. The method of paragraph 41, wherein said animal model is an animal model of autoimmune disorder.

47. The method of paragraph 41, wherein said animal model is an animal model of insulin dependent diabetes mellitus.

48. The method of paragraph 41, wherein said animal model is an animal model of graft rejection.

49. The method of paragraph 41, wherein ameliorating said disorder comprises ameliorating a symptom of said disorder.

50. The method according to paragraph 41, wherein said inhibitor is a selective inhibitor of said SUT-2 or SUT-3 polypeptide.

51. The method according to paragraph 41, wherein said SUT-2 polypeptide is human SUT-2 polypeptide.

52. The method according to paragraph 41, wherein said SUT-3 polypeptide is human SUT-3 polypeptide.

53. A method of identifying a candidate SUT-2 or SUT-3 inhibitor, said method comprising:

(a) providing an insect cell transfected with a baculovirus expression vector comprising a nucleic acid encoding a SUT-2 or SUT-3 polypeptide;

(b) contacting said cell with a test compound; and

(c) determining whether said compound selectively inhibits sulfate uptake activity;

wherein a determination that said compound selectively inhibits sulfate uptake activity indicates that said compound is a candidate inhibitor of said SUT-2 or SUT-3 polypeptide.

54. The method of paragraph 53, wherein said polypeptide is a SUT-3 polypeptide.

55. The method of paragraph 53, wherein said polypeptide is a SUT-2 polypeptide.

56. The method according to paragraph 53, wherein the method further comprises administering a compound determined to selectively inhibit sulfate uptake activity to an animal model of an inflammatory disorder and assessing the ability of the compound to ameliorate said disorder.

57. The method according to paragraph 53, wherein said compound is a candidate compound for the treatment of an inflammatory disorder.

58. The method according to paragraph 53, wherein a determination that said compound selectively inhibits sulfate uptake activity indicates that said compound is a candidate compound for the treatment of an inflammatory disorder.

59. The method according to paragraph 53, wherein said SUT-2 polypeptide is human SUT-2 polypeptide.

60. The method according to paragraph 53, wherein said SUT-3 polypeptide is human SUT-3 polypeptide.

61. A method of identifying a candidate SUT-2 inhibitor, said method comprising:

(a) providing a cell comprising a nucleic acid encoding a SUT-2 polypeptide;

(b) contacting said cell with a test compound; and

(c) determining whether said compound selectively inhibits sulfate uptake activity;

wherein a determination that said compound selectively inhibits sulfate uptake activity indicates that said compound is a candidate inhibitor of said SUT-2 polypeptide.

62. The method of paragraph 61, wherein said cell comprises an expression vector comprising a nucleic acid encoding a SUT-2 polypeptide.

63. The method of paragraph 61, wherein said cell is a Xenopus oocyte.

64. The method according to paragraph 61, wherein the method further comprises administering a compound determined to selectively inhibit sulfate uptake activity to an animal model of an inflammatory disorder and assessing the ability of the compound to ameliorate said disorder.

65. The method according to paragraph 61, wherein said compound is a candidate compound for the treatment of an inflammatory disorder.

66. The method according to paragraph 61, wherein a determination that said compound selectively inhibits sulfate uptake activity indicates that said compound is a candidate compound for the treatment of an inflammatory disorder.

67. The method according to paragraph 61, wherein said SUT-2 polypeptide is human SUT-2 polypeptide.

68. A method of identifying a candidate SUT-2 or SUT-3 inhibitor, said method comprising:

(a) providing a Xenopus oocyte comprising a nucleic acid encoding a SUT-2 or SUT-3 polypeptide;

(b) contacting said oocyte with a test compound; and

(c) determining whether said compound selectively inhibits sulfate uptake activity;

wherein a determination that said compound selectively inhibits sulfate uptake activity indicates that said compound is a candidate inhibitor of said SUT-2 or SUT-3 polypeptide.

69. The method of paragraph 68, wherein said polypeptide is a SUT-2 polypeptide.

70. The method of paragraph 68, wherein said polypeptide is a SUT-3 polypeptide.

71. The method according to paragraph 68, wherein the method further comprises administering a compound determined to selectively inhibit sulfate uptake activity to an animal model of an inflammatory disorder and assessing the ability of the compound to ameliorate said disorder.

72. The method according to paragraph 68, wherein said compound is a candidate compound for the treatment of an inflammatory disorder.

73. The method according to paragraph 68, wherein said inhibitor is a selective inhibitor of said SUT-2 or SUT-3 polypeptide.

74. The method according to paragraph 68, wherein said SUT-2 polypeptide is human SUT-2 polypeptide.

75. The method according to paragraph 68, wherein said SUT-3 polypeptide is human SUT-3 polypeptide.

76. An isolated nucleic acid selected from the group consisting of:

(i) a nucleic acid molecule encoding a polypeptide comprising an amino acid sequence selected from the group of sequences consisting of SEQ ID NOs: 5, 6 10 and 27;

(ii) a nucleic acid molecule comprising a nucleic acid sequence selected from the group of sequences consisting of SEQ ID NO: 2, 3 or 9, or a sequence complementary thereto;

(iii) a nucleic acid encoding a polypeptide comprising an amino acid sequence having at least about 95% identity to a sequence selected from the group consisting of the polypeptides of SEQ ID NOs: 5, 6, 10 and 27, and the polypeptides encoded by the nucleic acid of SEQ ID NOs: 2, 3 or 9; or

(iv) a nucleic acid encoding a fragment of said polypeptide which possesses sulfate transport activity, said fragment comprising at least 6 contiguous amino acid residues of amino acid positions 607 to 650 of SEQ ID NO: 5.

(v) a nucleic acid molecule the complementary strand of which hybridizes under stringent conditions to a nucleic acid as defined in (i) to (iv); and

(vi) a nucleic acid the sequence of which is degenerate as a result of the genetic code to a sequence of a nucleic acid as defined in (i) to (v).

77. The nucleic acid of paragraph 76, wherein said polypeptide comprises an amino acid sequence selected from the group consisting of the sequences shown as SEQ ID NOs: 5, 6, 10 and 27 and the polypeptides encoded by the nucleic acid of SEQ ID NOs: 2, 3 or 9.

78. The nucleic acid of paragraphs 76, wherein said nucleic acid is operably linked to a promoter.

79. An expression cassette comprising the nucleic acid of paragraph 76.

80. A host cell comprising the expression cassette of paragraph 79.

81. A method of making a SUT-3 polypeptide, said method comprising

(a) providing a population of host cells comprising a nucleic acid encoding a SUT-3 protein; and

(b) culturing said population of host cells under conditions conducive to the expression of said recombinant nucleic acid;

whereby said polypeptide is produced within said population of host cells.

82. The method of paragraph 81, further comprising purifying said polypeptide from said population of cells.

83. An isolated nucleic acid, said nucleic acid comprising a nucleotide sequence having at least about 98% identity over at least about 100 nucleotides to a sequence selected from the group consisting of the sequences shown as SEQ ID NO: 2, 3 or 9, and sequences complementary thereto.

84. The nucleic acid of paragraph 83, wherein said nucleic acid hybridizes under stringent conditions to a nucleic acid comprising SEQ ID NO: 2, 3 or 9, or the sequence complementary thereto.

85. The nucleic acid of paragraph 83, wherein identity is determined using an algorithm selected from the group consisting of NBLAST with the parameters score=100 and wordlength=12, Gapped BLAST with the default parameters of NBLAST, and BLAST with the default parameters of NBLAST.

86. A polypeptide encoded by the nucleic acid of paragraph 76 wherein said polypeptide has sulfate transport or anion exchange activity.

87. An isolated polypeptide or fragment thereof possessing sulfate transport activity, said polypeptide comprising an amino acid sequence having at least about 95% amino acid sequence identity to a sequence selected from the group consisting of SEQ ID NOs: 5,6, 10and27.

88. The polypeptide of paragraph 87, wherein said polypeptide is selectively bound by an antibody raised against an antigenic polypeptide, or antigenic fragment thereof, said antigenic polypeptide comprising a polypeptide selected from the group consisting of SEQ ID NOs: 5, 6, 10 and 27.

89. The polypeptide of paragraph 87, wherein said polypeptide comprises a polypeptide selected from the group consisting of SEQ ID NOs: 5, 6, 10 and 27.

90. An antibody that selectively binds to the polypeptide of paragraph 86.

91. A method of determining whether a SUT-3 is expressed within a biological sample, said method comprising the steps of:

(a) contacting said biological sample with:

(i) a polynucleotide that hybridizes under stringent conditions to a nucleic acid of paragraph 76; or

(ii) a detectable polypeptide that selectively binds to the polypeptide of paragraph 86 or paragraph 87; and

(b) detecting the presence or absence of hybridization between said polynucleotide and an RNA species within said sample, or the presence or absence of binding of said detectable polypeptide to a polypeptide within said sample;

wherein a detection of said hybridization or of said binding indicates that said SUT-3 is expressed within said sample.

92. The method of paragraph 91, wherein said polynucleotide is a primer, and wherein said hybridization is detected by detecting the presence of an amplification product comprising said primer sequence.

93. The method of paragraph 91, wherein said detectable polypeptide is an antibody.

94. A method of determining whether a mammal has an elevated or reduced level of SUT-3 expression, said method comprising the steps of:

(a) providing a biological sample from said mammal; and

(b) comparing the amount of a SUT-3 polypeptide of paragraph 86 or paragraph 87 or of a SUT-3 RNA species encoding a polypeptide of paragraph 86 or 87 within said biological sample with a level detected in or expected from a control sample;

wherein an increased amount of said SUT-3 polypeptide or said SUT-3 RNA species within said biological sample compared to said level detected in or expected from said control sample indicates that said mammal has an elevated level of SUT-3 expression, and wherein a decreased amount of said SUT-3 polypeptide or said SUT-3 RNA species within said biological sample compared to said level detected in or expected from said control sample indicates that said mammal has a reduced level of SUT-3 expression.

95. A method of identifying a candidate inhibitor of a SUT-3 polypeptide, said method comprising:

(a) contacting a SUT-3 polypeptide according to paragraph 86 or paragraph 87; and

(b) determining whether said compound selectively binds to said polypeptide;

wherein a determination that said compound selectively binds to said polypeptide indicates that said compound is a candidate inhibitor of said polypeptide.

96. A method of identifying a candidate SUT-3 inhibitor, said method comprising:

(a) contacting a SUT-3 polypeptide of paragraph 86 or paragraph 87 with a test compound; and

(b) determining whether said compound selectively inhibits the activity of said polypeptide;

wherein a determination that said compound selectively inhibits the activity of said polypeptide indicates that said compound is a candidate inhibitor of said polypeptide.

97. A method of identifying a candidate SUT-3 inhibitor, said method comprising:

(a) providing a cell comprising a SUT-3 polypeptide of paragraph 86 or paragraph 87 with a test compound;

(b) contacting said cell with a test compound; and

(c) determining whether said compound selectively inhibits SUT-3 activity;

wherein a determination that said compound selectively inhibits the activity of said polypeptide indicates that said compound is a candidate inhibitor of said polypeptide.

98. The method of paragraph 97, wherein said cell is an Sf9 cell.

99. The method of paragraph 97, wherein said cell is a Xenopus laevis oocyte.

100. The method of paragraph 97, wherein step a) comprises introducing a nucleic acid comprising the nucleotide sequence encoding said SUT-3 polypeptide according to paragraph 76 into said cell.

101. The method of paragraph 97, wherein step a) comprises introducing a baculovirus vector comprising a nucleic acid encoding a SUT-3 polypeptide into said cell.

102. The method of paragraph 97 wherein step a) comprises introducing SUT-3 cRNA into said cell.

103. The method of paragraph 97 wherein step d) comprises detecting sulfate uptake by said cell.

104. A polynucleotide according to paragraph 76 attached to a solid support.

105. An array of polynucleotides comprising at least one polynucleotide according to paragraph 104.

106. An array according to paragraph 104, wherein said array is addressable.

107. A polynucleotide according to paragraph 76 further comprising a label.

108. A method of identifying a candidate activator of SUT-3, said method comprising:

(a) contacting a SUT-3 polypeptide of paragraph 86 or paragraph 87 or a cell comprising a SUT-3 polypeptide of paragraphs 86 or 87 with a test compound; and

(b) determining whether said compound selectively increases the activity of said polypeptide;

wherein a determination that said compound selectively increases the activity of said polypeptide indicates that said compound is a candidate activator of said polypeptide.

109. The nucleic acid of Paragraph 76, wherein polypeptide identity is determined using an algorithm selected from the group consisting of XBLAST with the parameters score=50 and wordlength=3, Gapped BLAST with the default parameters of XBLAST, and BLAST with the default parameters of XBLAST.

110. The polypeptide of Paragraph 87, wherein identity is determined using an algorithm selected from the group consisting of XBLAST with the parameters score=50 and wordlength=3, Gapped BLAST with the default parameters of XBLAST, and BLAST with the default parameters of XBLAST.

111. A method of modulating extravasion of lymphocytes in an individual comprising modulating the activity of the SUT-3 protein or SUT-2 protein in said individual.

112. A method of reducing inflammation in an individual comprising inhibiting the activity of the SUT-3 protein or SUT-2 protein in said individual.

113. A method of increasing extravasion of lymphocytes in an individual comprising increasing the activity of the SUT-3 protein or SUT-2 protein in said individual.

### BRIEF DESCRIPTION OF THE SEQUENCE LISTING

SEQ ID NO: 1 is a cDNA sequence encoding the human SUT-3 protein (short isoform).

SEQ ID NO: 2 is a cDNA sequence encoding the human SUT-3 protein (long isoform).

SEQ ID NO: 3 is a cDNA sequence encoding the mouse SUT-3 protein.

SEQ ID NO: 4 and NO: 5 are the amino acid sequences of the human SUT-3 protein short and long isoforms respectively.

SEQ ID NO: 6 is the amino acid sequence of the mouse SUT-3 protein.

SEQ ID NOs: 7 and 8 are oligonucleotide primers used to test expression of SUT-3 mRNA in HEVECs by RT-PCR.

SEQ ID NO: 9 is a cDNA sequence encoding the mouse SUT-3 protein.

SEQ ID NO: 10 is the amino acid sequence of the mouse SUT-3 protein.

SEQ ID NO: 11 is a cDNA sequence encoding a human SUT-2 protein isoform of 656 amino acids in length.

SEQ ID NO: 12 is a cDNA sequence encoding a human SUT-2 protein isoform of 663 amino acids in length.

SEQ ID NO: 13 is the amino acid sequence of a human SUT-2 protein isoform of 656 amino acids in length.

SEQ ID NO: 14 is the amino acid sequence of a human SUT-2 protein isoform of 663 amino acids in length.

SEQ ID NO: 15 is a consensus amino acid sequence of the sulfate transporter signature (Prosite ref. PS01130).

SEQ ID NOs: 16 and 17 are oligonucleotide primers used to amplify the human SUT-2 nucleic acid.

SEQ ID NOs: 18 and 19 are oligonucleotide primers used to test expression of SUT-2 mRNA in HEVECs by RT-PCR.

SEQ ID NO: 20 is a consensus sulfate transporter signature (Pfam reference PF00916).

SEQ ID NOs: 21 and 22 are oligonucleotide primers used to amplify the human SUT-3 nucleic acid.

SEQ ID NO: 23 is the SUT-3-Nde5′ amplification primer.

SEQ ID NO: 24 is the SUT-3-Eco3 amplification primer.

SEQ ID NO: 25 is the Myc5′ amplification primer.

SEQ ID NO: 26 is the SUT-3-Xba3 amplification primer.

SEQ ID NO: 27 is the amino acid sequence of a mouse SUT-3 protein which could be generated by alternative splicing of SEQ ID NO: 9.

### BRIEF DESCRIPTION OF THE DRAWINGS

FIGS. 1a and 1b show the cDNA sequences encoding the human SUT-3 protein. (both isoforms).

FIG. 2 shows the alignment of the two isoforms of SUT-3 at the protein level.

FIG. 3 illustrates the similarity between the SUT-3 protein and the Pfam sulfate transporter signature (PF00916).

FIG. 4 illustrates the similarity between the SUT-3 protein and the Pfam STAS domain (PF01740).

FIG. 5 illustrates the oligonucleotide primers used to test expression of SUT-3 mRNA in HEVECs by RT-PCR.

FIG. 6 depicts a multiple alignment of the SUT-3 amino acid sequence with the orthologues as follows:

>AAF56989.1 [Drosophila melanogaster]

>NP_{—}013193.1 |Sulfate uptake; Sul2p [Saccharomyces cerevisiae]

>T39116 probable sulfate permease—fission yeast (Schizosaccharomyces pombe)

>NP_{—}196859.1 sulfate transporter [Arabidopsis thaliana]

>SUT-3 long isoform.

FIG. 7 shows the transmembrane domains of the SUT-3 long isoform.

FIG. 8 shows the transmembrane domains of the SUT-3 short isoform.

FIG. 9 shows a list of the human and mouse ESTs for SUT-3 and their tissue expression.

FIG. 10 shows the mouse SUT-3 orthologue as predicted from the genomic clone AC084294 (nucleotide positions 2745 to 25101), including the genomic structure of the SUT-3 gene. Also shown is the alignment of the human SUT-3 amino acid sequence.

FIG. 11 depicts the alignment of the mouse and human SUT-3 orthologues at the protein level.

FIG. 12 shows sulfate uptake by Sf9 insect cells infected at a MOI of 5 with baculoviruses expressing SUT-3 (filled bars) and control cells (infected with baculovirus expressing the GFP or wild type baculovirus, open bars). A, Na⁺-independent sulfate transport. Uptake buffer contained either 500 μM Na₂SO₄ (+Na⁺) or 500 μM K₂SO₄ (−Na⁺). B, SUT-3-mediated sulfate transport is sensitive to the anion exchanger inhibitor DIDS. Uptake of 500 μM Na₂SO₄ was performed in the presence (+DIDS) or absence (−DIDS) of 1 mM DIDS. Data are shown as means±SE for a single experiment performed in triplicates and are representative of at least two similar experiments.

FIG. 13 summarizes the exon/intron junction sequences and sizes of each exon and intron of SUT-3 on the human and mouse genomic sequences, GenBank Accession N^{o}AC123764 and N^{o}AL645911, respectively. The disclosures of each of these GenBank Accession Numbers are incorporated herein by reference in their entireties.

FIG. 14 illustrates the homology between the SUT-2 protein and the Pfam sulfate transporter signature PF00916).

FIG. 15 depicts an multiple alignment of the SUT-2 amino acid sequence with the three known human sulfate transporters, hDTD, hDRA and hSAT-1.

FIG. 16 depicts the relationship between SUT-2 and DTDST.

FIG. 17 depicts an alignment of the amino acid sequences of the two human SUT-2 isoforms of SEQ ID NOs: 13 and 14, from amino acid position 600 to the end of the respective isoforms.

FIG. 18 depicts a possible mechanism for production of different SUT-2 isoforms by alternative splicing and polyadenylation involving the penultimate SUT-2 exon.

FIG. 19 shows the partial genomic structure of the human SUT-2 gene, including the last 16 exons and their 3′ splice acceptor and 5′ splice donor sequences and the lengths of the respective introns and exons.

FIG. 20 illustrates the mouse genomic sequence of Genbank Accession Number AC084294.

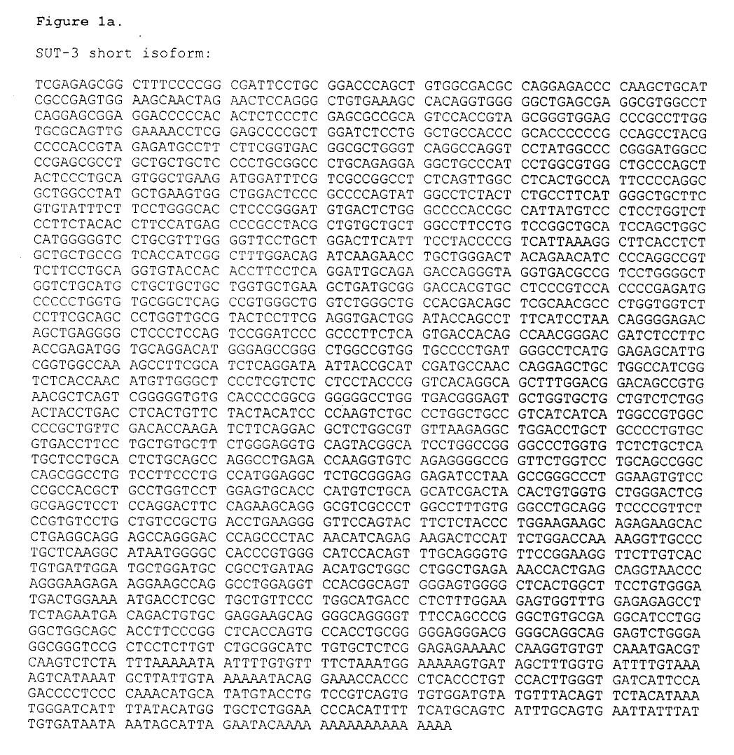

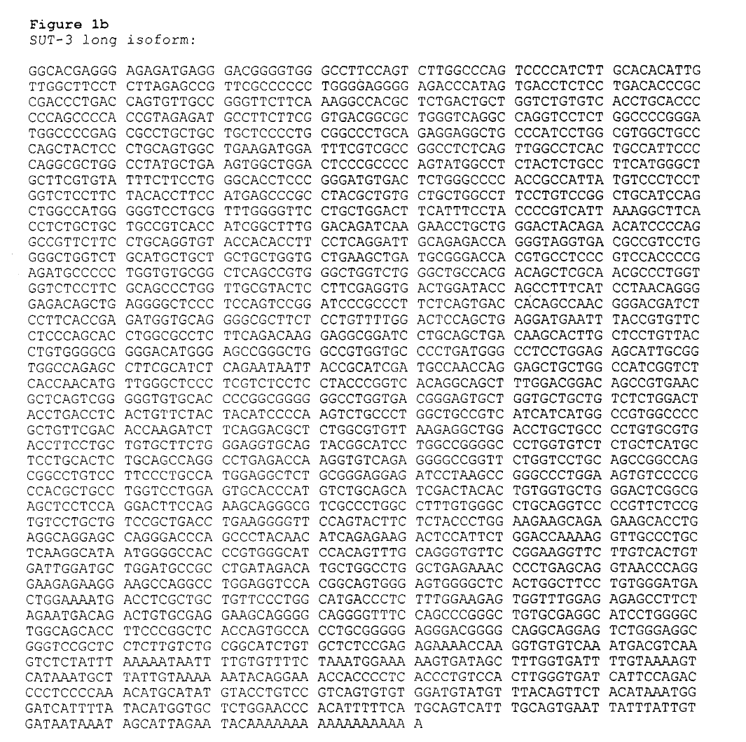

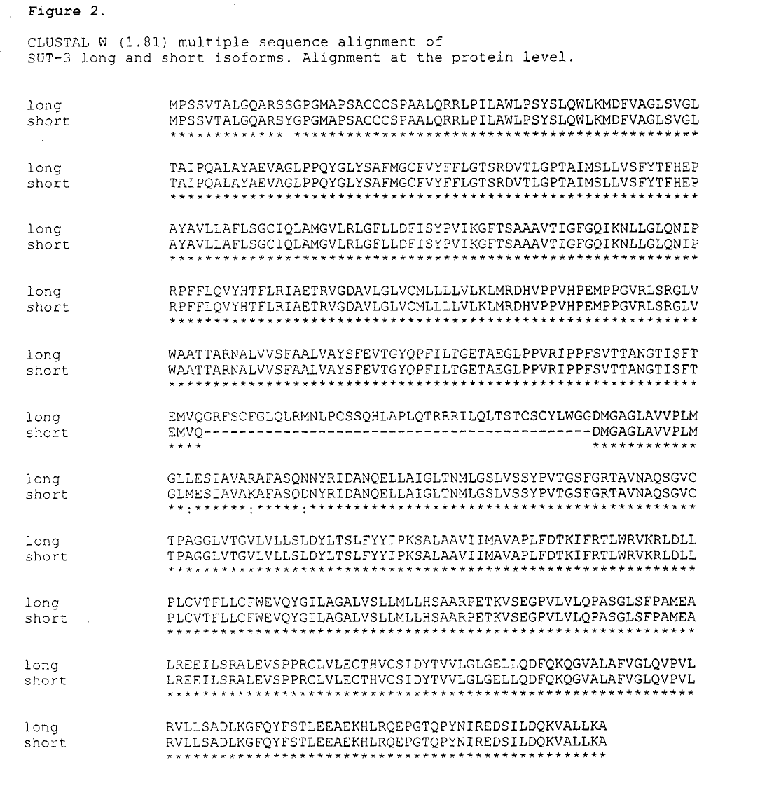

### DETAILED DESCRIPTION OF THE INVENTION

The present invention is based on the discovery of novel molecules, referred to herein as SUT-2 and SUT-3 proteins and SUT-2 and SUT-3 nucleic acid molecules, encoding sulfate/anion exchanging proteins expressed in HEVECs.

SUT-2 and SUT-3 are expressed in HEVs, specialized postcapillary venules found in lymphoid tissues and nonlymphoid tissues during chronic inflammatory diseases that support a high level of lymphocyte extavasation from the blood. HEVs are known to incorporate large amounts of inorganic sulfate, and sulfate residues are essential for the recognition of HEV sialomucins by L-selectin, a cell adhesion molecule that mediates the initial interaction of lymphocytes with HEV. By modulating sulfate incorporation into HEV, SUT-2 and SUT-3 could play a major role in the sulfation of HEV sialomucins and regulation of lymphocyte migration through HEV in organized lymphoid tissues and through HEV-like vessels at the sites of chronic inflammation.

Currently, two distinct classes of mammalian sulfate transporters have been cloned and characterized: Na+ coupled sulfate transporters and Na+ independent sulfate transporters. Rat SAT-1 and human DTDST and DRA are members of the superfamily of anion exchangers that function as Na+ independent transporters and are sensitive to the anion exchange inhibitor 4,4′-diisothiocyano-2,2′-disulphonic acid stilbene (DIDS). Rat NaSi-1 and human SUT-1 are members of the superfamily of Na+ coupled transporters that function as DIDS resistant Na+ dependent sulfate transporters. Both DTDST and DRA have been implicated in the pathogenesis of human disease involving their sulfate/anion exchange functions, where mutations in the DTDST gene result in profound undersulfation of cartilage proteoglycans and thus lead to severe developmental defects of the skeleton, and DRA is mutated in congenital chloride diarrhea (CLD).

Characterization of sulfate/anion exchangers in mammals has resulted in the identification of sulfate transporter signatures (PROSITE PSO1130 and Pfam PF00916). As expected by their transport function, all transporters in this family are highly hydrophobic. All known family members also share a similar core structure of 12 transmembrane domain helices, with highly charged cytoplasmic domains at both the N- and C-termini. Another transporter protein sharing significant amino acid identity with the sulfate/anion exchanger proteins is pendrin (45% amino acid identity with DRA, 32% with DTD and 29% with SAT-1); however, pendrin does not transport sulfate, functioning instead as a transporter of chloride and iodide. Sulfate transporter signatures have also been found in sulfate transporters in yeast, bacteria, fungi and plants that are structurally related to mammalian sulfate transporters.

Cloning and Characterization of SUT-3

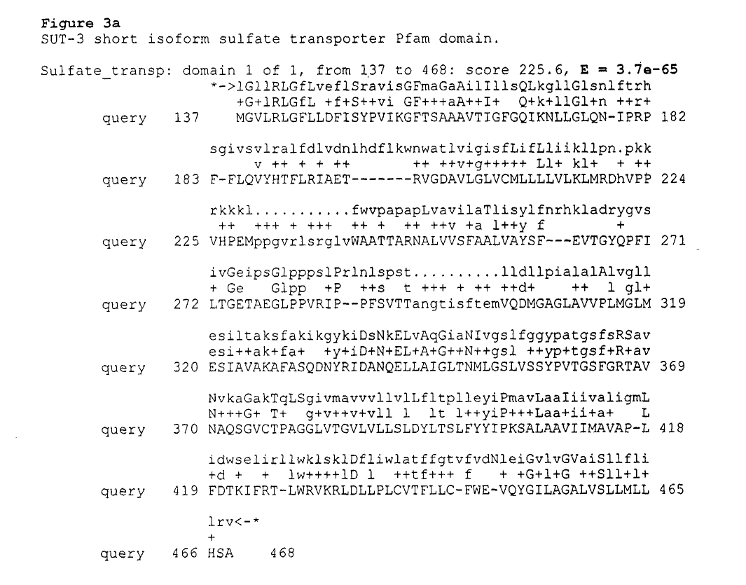

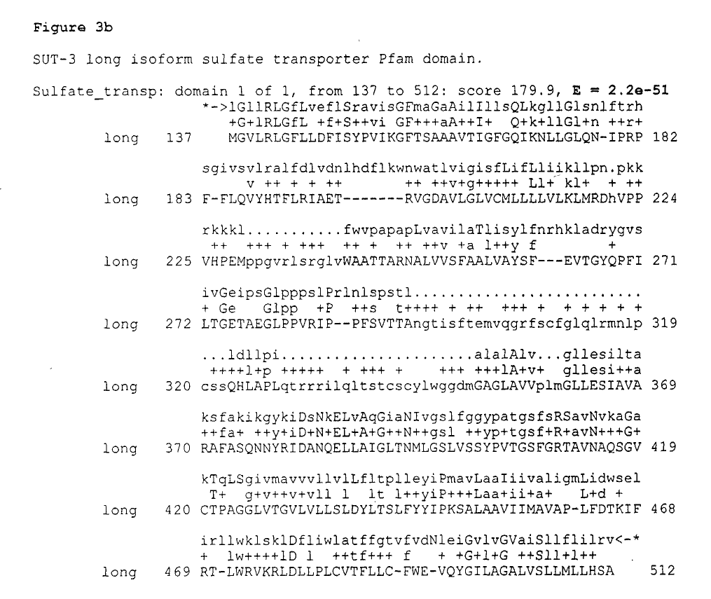

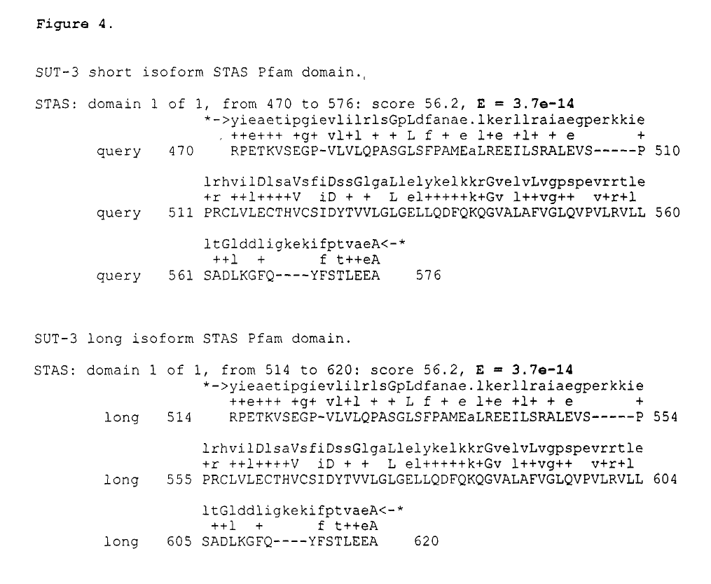

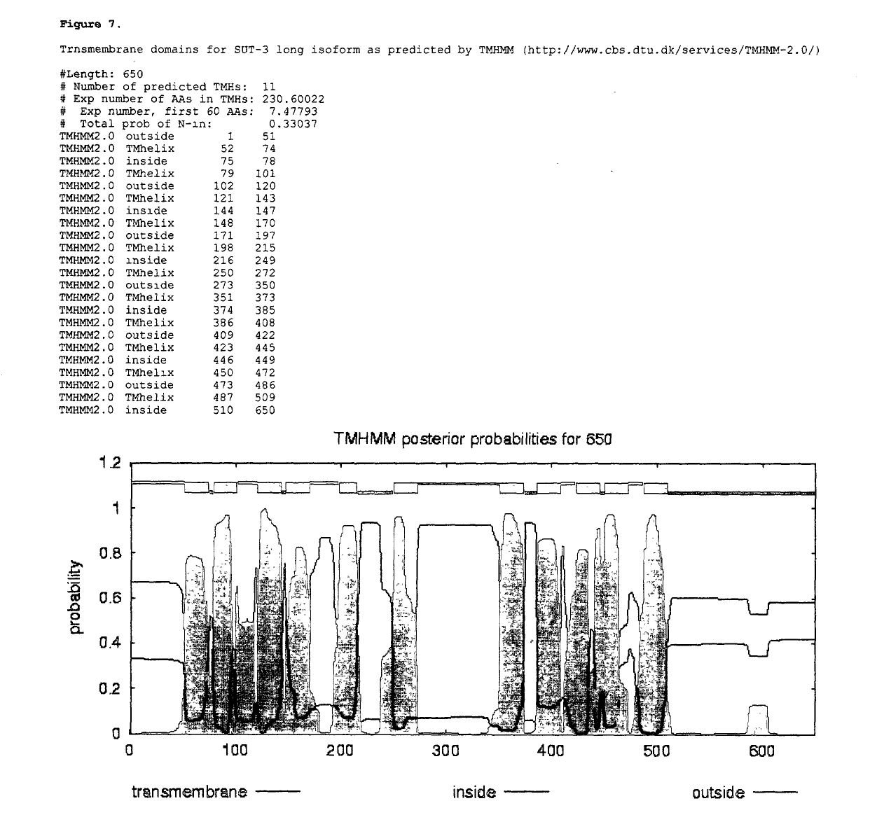

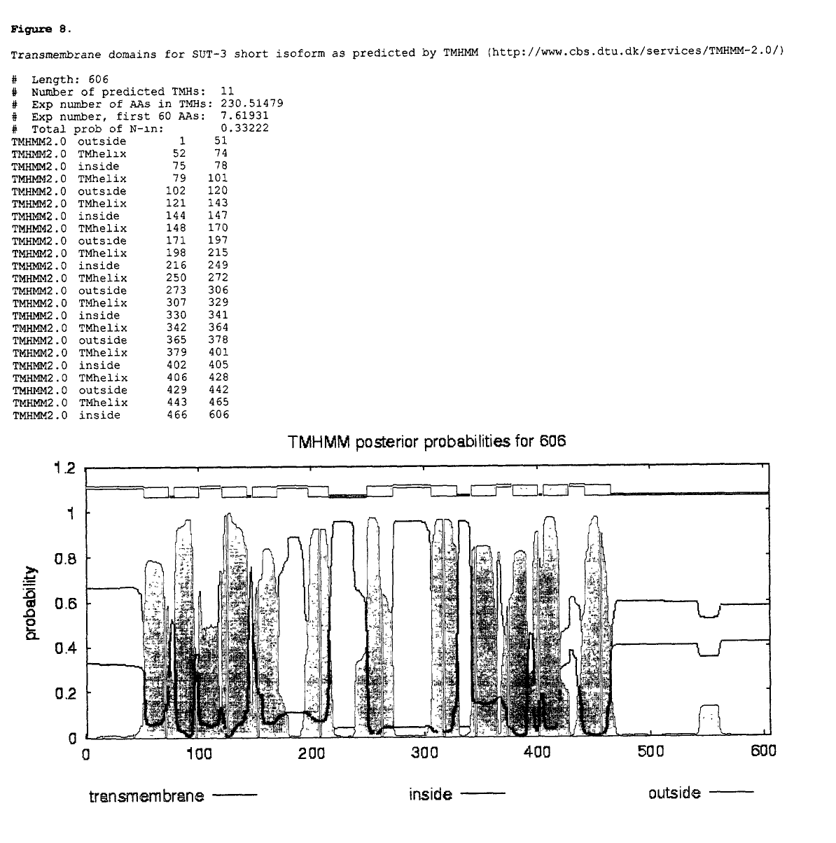

SUT-3 is a novel member of the SLC26 family of sulfate/anion exchanger proteins, the human SUT-3 protein comprising the 606 and 650 amino acid sequences of SEQ ID NO: 4 and 5 respectively, the two human isoforms. Also provided is the mouse SUT-3 coding sequence, a 607 amino acid sequence shown in SEQ ID NOs: 6 and 10. Alternatively, another possible mouse SUT-3 protein which could be generated by alternative splicing has the sequence of SEQ ID NO: 27. The polypeptide of SEQ ID NO: 27 or portions thereof may be used in any of the embodiments described herein. SUT-3 comprises 11 transmembrane domains as shown in FIGS. 7 and 8 for the two human isoforms respectively, and comprises a sequence similar to a sulfate transporter signature (Pfam PF00916) in its N-terminal region at amino acid positions 137-468 of SEQ ID NO: 4 and residues 137-512 of SEQ ID NO: 5 as shown in FIG. 3. In FIG. 4 the alignment with the STAS domain is shown. SUT-3 contains an STAS domain at residues 470-576 of SEQ ID NO 4: and 514-620 of SEQ ID NO: 5. The mouse SUT-3 of SEQ ID NOs: 6 and 10 comprise a sequence similar to a sulfate transporter signature (Pfam PF00916) in their N-terminal regions at amino acid positions 137 to 468.

The SUT-3 cDNA was cloned and characterized as the result of a cloning effort based on yeast high-affinity sulfate transporter mRNA sequences SUL-1 and SUL-2 (Cherest H, et al, Genetics. 1997, (3):627-35). An important element in the cloning of the SUT-3 cDNA from HEVECs was the development of protocols for obtaining HEVECs RNA, since HEVECs are not capable of maintaining their phenotype outside of their native environment for more than a few hours. Total RNA was obtained from HEVECs freshly purified from human tonsils. Highly purified HEVECs were obtained by a combination of mechanical and enzymatic procedures, immunomagnetic depletion and positive selection. Tonsils were minced finely with scissors on a steel screen, digested with collagenase/dispase enzyme mix and unwanted contaminating cells were then depleted using immunomagnetic depletion. HEVECs were then selected by immunomagnetic positive selection with magnetic beads conjugated to the HEV-specific antibody MECA-79. From these HEVEC that were 98% MECA-79-positive, 1 μg of total RNA was used to generate full length cDNAs for SUT-3 cDNA cloning and RT-PCR analysis.

TMHMM and Toppred2 membrane topology prediction programs suggest that the protein possess 11 transmembrane-spanning domains, with the N-terminal extremity at the outside of the cell, in contrast to other members of SLC26 family which have been suggested to possess 12 transmembrane-spanning domains, with both extremities at the inside of the cell (Lohi, H., et al, (2000) Genomics 70(1), 102-12; Vincourt, J. B., et al, (2002) Genomics 79(2), 249-56; Bissig, M., et al, (1994) J Biol Chem 269(4), 3017-21; Hastbacka, J., et al, (1994) Cell 78(6), 1073-87; and Oliver, D., et al, (2001) Science 292(5525), 2340-3).

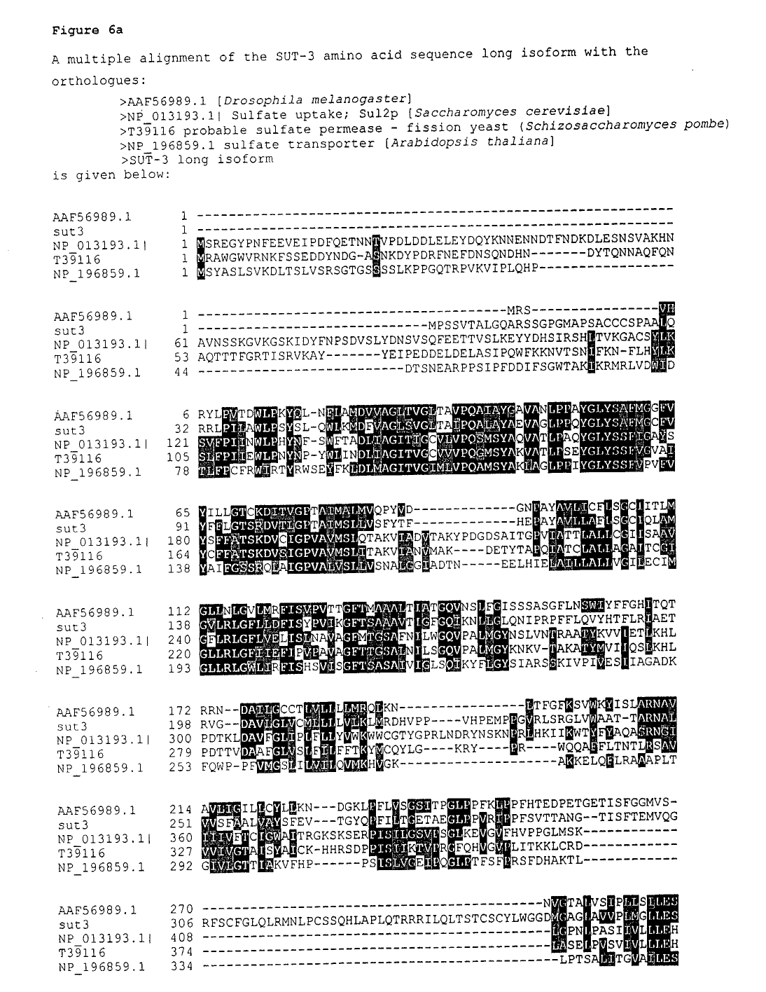

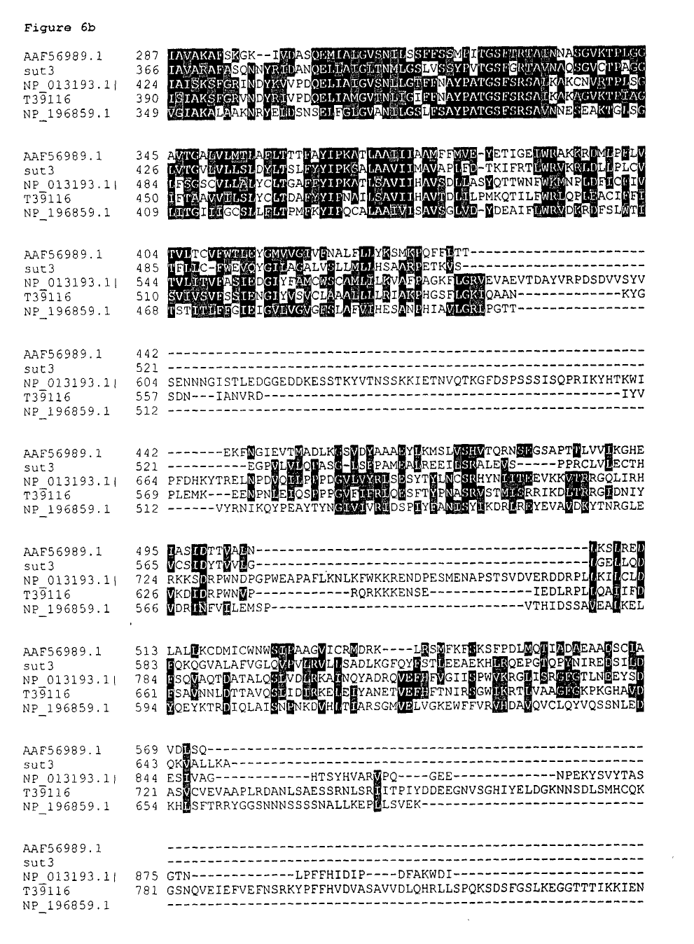

Analysis of protein sequence databases indicated that SUT-3 possesses the Prosite sulfate transporter signature (PS01130) at residues 77-98 of SEQ ID NOs: 4, 5, 6 and 10 as well as the Pfam sulfate transporter domain (Pf00916), suggesting that SUT-3 is a novel member of the SLC26 sulfate/anion exchanger family. Sequence alignments (see FIG. 6) revealed its close homology to seven hypothetical sulfate transporters from Drosophila melanogaster, to Saccharomyces Cerevisiae SUL1 and SUL2 high affinity sulfate transporters (Cherest, H.,et al, (1997) Genetics 145(3), 627-35; and Smith, F. W., et al, (1995) Mol Gen Genet 247(6), 709-15) and to several plant sulfate transporters (Smith, F. W., et al, (1995) Proc Natl Acad Sci U S A 92(20), 9373-7). This homology pattern is a unique characteristic of SUT-3 among human sulfate/anion transporters. SUT-3 exhibits 59%, 52% and 43% homology with the esp gene product from Drosophila Kaufman, R. a. (1996) Molecular Biology of the Cell 7(Suppl.)(120a), yeast and plant high affinity sulfate transporters, respectively.

Further characterization showed that SUT-3 is expressed at the cell membrane. In order to prove that the SUT-3 cDNA contains a physiologically relevant open reading frame and to determine the subcellular localization of the SUT-3 protein, the SUT-3 cDNA was expressed in COS-7 cells. For immunolocalization, a myc-epitope tag was added to the amino terminus of the protein. The myc-tagged SUT-3 cDNA was cloned into a mammalian expression vector downstream the EF-1 alpha promoter, and transiently transfected into COS-7 cells. Immunofluorescence studies on permeabilized cells with anti-myc antibodies revealed strong staining of a perinuclear compartment, which may correspond to the endoplasmic reticulum and Golgi apparatus, and a fainter diffuse pattern including the edges of the cell, characteristic of the plasma membrane. Localization of SUT-3 protein in the Golgi apparatus, endoplasmic reticulum and cell membrane is consistent with the pattern expected for a membrane glycoprotein, with the former two sites indicative of nascent protein. SUT-3 expression was also detected without permeabilization of the transfected cells, suggesting that the myc-tagged amino-terminal part of SUT-3 is on the extracellular side of the cell membrane. In contrast, the myc-tagged S19 ribosomal protein, used as an intracellular control, was only detected after permeabilization of the cells. Thus, SUT-3 is expressed at the cell membrane with the amino-terminus of the protein directed towards the extracellular side, in agreement with the hypothetical model of SUT-3 membrane topology.

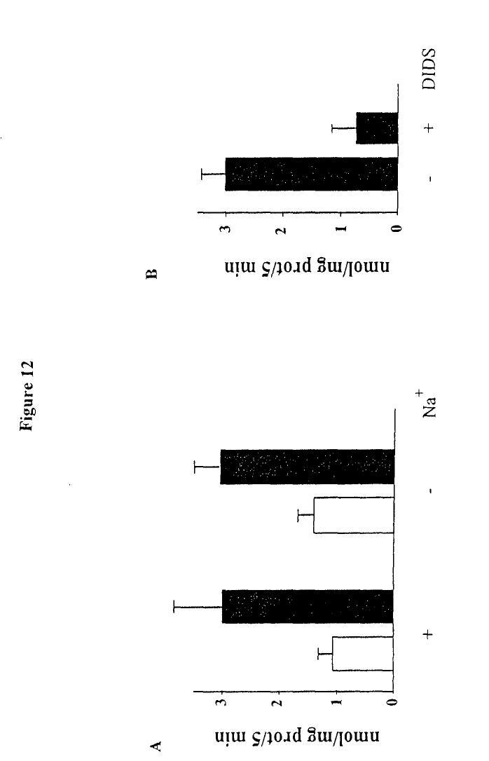

The inventors also determined that SUT-3 functions as a sodium-independent sulfate transporter. The homology of SUT-3 protein to previously reported sulfate transporters, including lower eukaryote sulfate transporters and human sulfate transporters of the SLC26 sulfate/anion exchanger family, suggested a related transport function for human SUT-3. A baculovirus expression system was used to assess SUT-3 sulfate transporter function. A recombinant baculovirus was constructed allowing expression of SUT-3 in Sf9 insect cells under the control of the polyhedrin promoter. It was found that Sf9 cells infected with this SUT-3-recombinant baculovirus incorporate about three times more sulfate than those infected with control baculoviruses in the same conditions (FIG. 12A). The observed sulfate transport activity was independent of the presence of Na+ in the uptake buffer. In contrast, SUT-3-mediated sulfate uptake was efficiently inhibited by the anion exchanger inhibitor DIDS (FIG; 12B). Together, these data show that SUT-3 is a novel Na⁺-independent, DIDS-sensitive, sulfate transporter from the SLC26 sulfate/anion exchanger family.

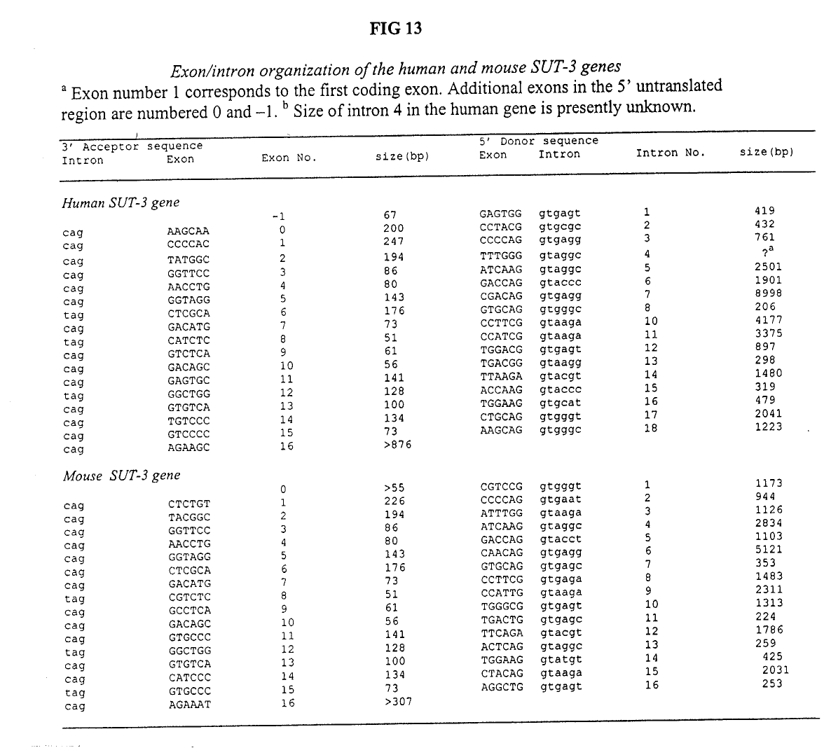

A BLAST search of the draft human genome sequence database at NCBI using SUT-3 cDNA or protein sequences as baits revealed a genomic hit from the Homo Sapiens chromosome 17 working draft sequence (GenBank Accession N^{o}AC123764, the disclosure of which is incorporated herein by reference). Alignment between the SUT-3 cDNA and genomic sequences revealed that there are 18 exons that span more than 25 kb of genomic DNA. FIG. 13 summarizes the exon/intron junction sequences and sizes of each exon and intron. All the exon-intron boundaries follow the GT-AG rule. The exon structure of the SUT-3 gene differed considerably from that of several other members of the SLC26 sulfate/anion exchangers family (SLC26A3, A4, A6, A7, A8, A9) which share 10-15 exons of identical size (Lohi et al., 2002). In contrast, the human SUT-3 gene shares a similar organization with its mouse ortholog identified in the Mus musculus chromosome 11 working draft sequence (GenBank Accession N^{o}AL645911, the disclosure of which is incorporated herein by reference). The size of exons are strictly conserved between the species, with the exception of the 5′- and 3′-flanking regions that are significatively different between the human and mouse SUT-3 genes (FIG. 13).

Two cDNAs which differ from the major human SUT-3 isoform have been identified as potential alternative splicing products. The first one (Genbank Accession N^{o}AL833468) lacks exon 0 but contains exon −1 and an extended exon 1 with 130 additional nucleotides at the 5′end. The second cDNA (Genbank Accession N^{o}BG683172; IMAGE clone 4761567) lacks exons −1 and 0 but contains an extended exon 1 with 202 extra nucleotides at the 5′end as well as an additional exon of 132 nucleotides found between exons 6 and 7 in the genomic sequence of the human SUT-3 gene.

Based on a BAC clone from the Homo Sapiens chromosome 17 working draft sequence (GenBank Acc. AC123764, two UniSTS were identified in the SUT-3 gene. UniSTS:97442 (stSG55037 also known as RH103108), which is found in exon 16 encoding the SUT-3 3′UTR, has previously been mapped at 17q25 near chromosome 7 telomere, and very close to the locus D17S784. UniSTS:176842 (SHGC-149168 from the Stanford Human Genome Center), which is located in intron 5 of the gene has also been mapped on chromosome 17 at 17q25.3. Together, these data indicate that the human SUT-3 gene is located on human chromosome 17 at 17q25, very close to microsatellite marker D17S784. Three distinct hereditary hearing loss diseases loci have been mapped to chromosome 17q25. These include DFNA20 (Morell, R. J., et al, (2000) Genomics 63, 1-6) and DFNA26 (Yang, T. and Smith, R. (2000) Am J Hum Genet 67, 300), two non-syndromic forms of sensorineural hearing loss, and USH1G (Mustapha, M., et al, (2002) Hum Genet 110, 348-350), an Usher type I syndrome, which shows, in addition to a congenital hearing loss, late-onset retinitis pigmentosa. Two members of the SLC26 gene family have previously been shown to be important for the function of the inner ear: SLC26A4/Pendrin, mutations in which cause Pendred syndrome syndrome (Everett, L. A., et al, (1997) Nat Genet 17, 411-422) and non-syndromic deafness type DFNB4 (Li, X. C., et al, (1998) Nat Genet 18, 215-217), and SLC26A5/Prestin, which functions as the motor protein of outer hair cells (Zheng, J., et al,. (2000) Nature 405, 149-155, Oliver, D., et al, (2001) Science 292, 2340-2343). The SUT-3 gene is thus an attractive candidate for the hearing loss diseases DFNA20, DFNA26 and USH1G, particularly DFNA20 and DFNA26 which overlap between D17S806 and D17S914 (Morell, R. J., et al, (2000), Yang, T. and Smith, R. (2000)), a 7 cM interval that includes the marker D17S784 colocalized with SUT-3 at 17q25. The invention thus also provides method for diagnostics and prognostics for deafness or a deafness-related disorder. Methods include detecting a SUT-3 nucleic acid polymorphism or aberrant SUT-3 expression associated with a deafness related disorder.

To determine the tissue distribution of SUT-3 mRNA, Northern blot analysis of 8 different adult human tissues was performed. Transcripts with the expected size of approximately 2.9 kb were detected to various degrees in all tissues tested, with highest levels in placenta, kidney and brain, and low but detectable levels in pancreas, skeletal muscle, liver, lung and heart. The widespread tissue distribution of SUT-3 mRNA in the human body could also be deduced from the presence in the human EST database of ESTs coming from many different tissue sources, including tumor tissues such as neuroblastoma, retinoblastoma and carcinoma.

To analyze expression of SLC26 sulfate transporters in HEVEC from human tonsils, RT-PCR analysis using oligonucleotides specific for each SLC26 family member was performed. With the exception of SLC26A7, all SLC26 family members which have previously been found to exhibit significant sulfate transport activity (SLC26A1, SLC26A2, SLC26A3, SLC26A8 and SUT-3) were included in the analysis. It was found that SUT-3 and SLC26A2 (DTDST) are expressed in tonsillar HEVEC, at levels comparable to those found in kidney. In contrast, SLC26A1, SLC26A3 and SLC26A8 mRNA expression was not detectable in HEVEC. These results indicate that HEVEC coexpress transcripts for two distinct sulfate transporters of the SLC26 sulfate/anion exchanger family, SUT-3 and SLC26A2 (DTDST).

Cloning and Characterization of SUT-2

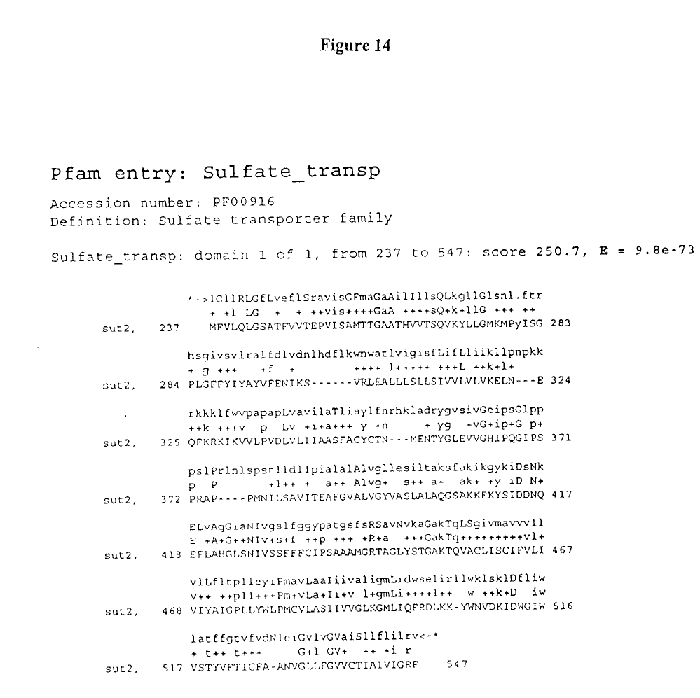

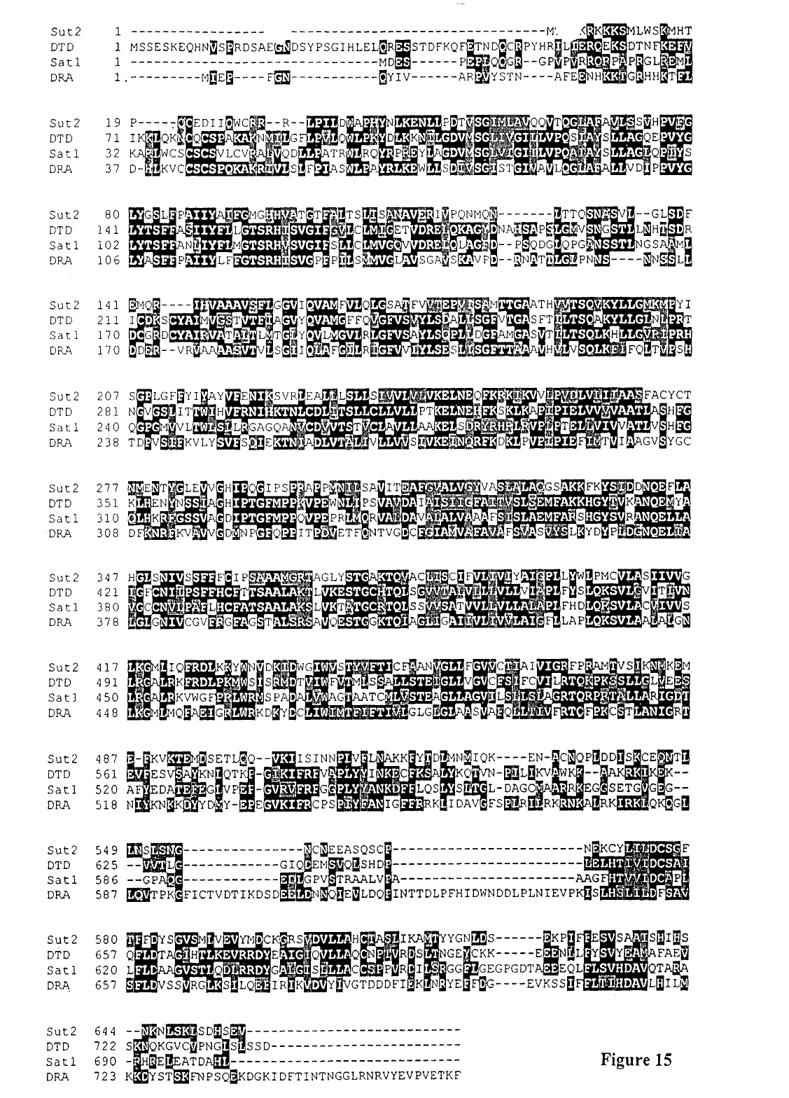

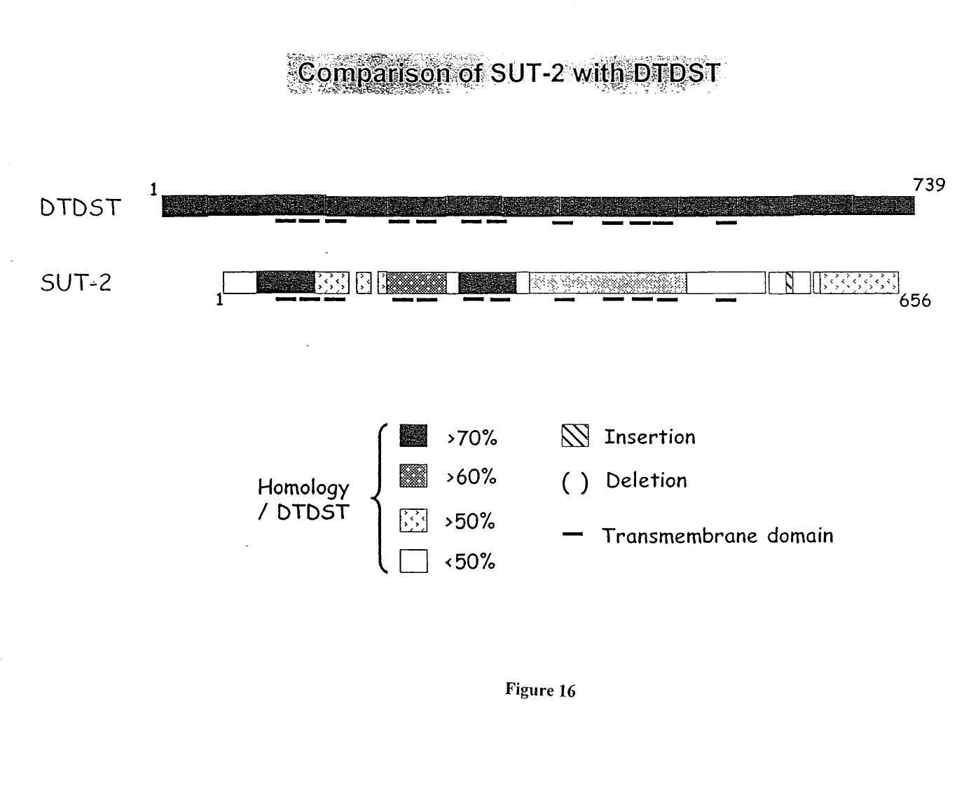

SUT-2 is another novel member of the SLC26 family of sulfate/anion exchanger proteins. The human SUT-2 protein comprises a 656 or 663 amino acid sequence of SEQ ID NOs: 13 or 14 respectively. SUT-2 comprises 12 transmembrane domains and comprises a sequence similar to a sulfate transporter signature (PROSITE PSO1130) in its N-terminal region at amino acid positions 76 to 97 of SEQ ID NOs: 13 or 14. As illustrated in FIG. 14, SUT-2 also exhibits homology to the Pfam sulfate transporter signature PF00916). SUT-2 exhibits 30-40% identity with the sulfate transporters DTD (diastrophic dysplasia), DRA (down regulated in adenoma) and SAT-1 (sulfate anion-transporter-1) (see FIG. 15). FIG. 16 depicts the relationship between SUT-2 and DTDST (diastrophic dysplasia). The SUT2 protein also contains residues and domains which can be involved in interactions with SUT2 target molecules, including an Anti-Sigma factor (STAS Pfam 01740) domain at amino acid positions 571 to 637 of SEQ ID NOs: 13 or 14, as well as a conserved serine residue at amino acid position 585 of SEQ ID NOs: 13 or 14.

The SUT-2 cDNA was cloned and characterized as the result of a cloning effort based on DTDST mRNA sequence. As in the case of SUT-3, an important element in the cloning of the SUT-2 cDNA from HEVECs was the development of protocols for obtaining HEVECs RNA. To determine whether the SUT2 mRNA was expressed in HEVEC, primers derived from the EST were used in RT-PCR analysis with total HEVEC RNA as template. A clear amplification product was obtained indicating that HEVECs express this novel member of the SLC26 family. In order to assemble a full length SUT2 cDNA, 3′ and 5′ RACE were performed with HEVEC total RNA. Sequence assembly resulted in a 2896 bp HEVEC SUT2 cDNA (SEQ ID NO: 11) sequence containing a 240 bp 5′-UTR, an open reading frame (ORF) of 1968 bp encoding a 656-amino-acid protein (SEQ ID NO: 13), and a 675 bp 3′-UTR with a consensus polyadenylation signal (AATAAA) 11 nt upstream of the poly(A) tail.

Comparison of the deduced SUT2 amino acid sequence with sequences in the public databases reveals significant homology with members of the SLC26 family from different species. The SLC26 human members which are most closely related to SUT2 are the sulfate transporters SAT1/SLC26A1, DTDST/SLC26A2 and DRA/SLC26A3 which exhibit about 30% sequence identity with SUT2. A sequence alignment of the amino acid sequence of SUT2 with those of hDTD, hDRA and hSAT-1 is shown in FIG. 15.

SUT2 further contains two functional domains characteristic of the SLC26 family, i.e. the Sulfate Transporter domain (Pfam 00916) and the Sulfate Transporter and Anti-Sigma factor (STAS; Pfam 01740) domain (Aravind, L. and Koonin, E. V. (2000). Curr Biol 10: R53-55). The sulfate transporter domain is localized in the amino-terminal portion of SUT2 from residues 164 to 471 (FIG. 15B) while the STAS domain was identified in the carboxy-terminal region of SUT2 from residue 571 to 637.

Additionally, alignment of SUT2 STAS domain with Bacillus subtilis anti-anti-sigma factor SPOIIAA showed that a key serine residue (Serine 58), the phosphorylation of which regulates protein-protein interactions, is conserved in SUT2 (Serine 585). DRA/SLC26A3 is the only other member of the SLC26 family which also possesses a serine at the equivalent position (S661). Analysis of SUT2 topology using PredictProtein led to the identification of 12 putative transmembrane domains, the amino acid positions described further herein, with both amino- and carboxy-terminal parts predicted to be intracellular. The carboxy-terminal part of SUT2 may therefore asssociate with cellular targets, as it has been shown for sulfate transporter TAT1/SLC26A8, which associates with MgcRacGAP, a protein linked to RhoGTPase and involved in intracellular signalling (Toure, A., et al. (2001) J Biol Chem 276: 20309-20315). In one aspect of the invention, it is envisioned that changes in SUT2 carboxy terminus may modify interactions of SUT2 isoforms with intracellular factors, and that assays may be developed to identify SUT2 target molecules as well as compounds capable of modulating, that is increasing or inhibiting, interactions of SUT2 with SUT2 target molecules.

To determine the tissue distribution of SUT2 mRNA, Northern blot analysis was performed using 12 different adult human tissues. This revealed that SUT2 mRNA exhibits a restricted tissue distribution in the human body with abundant and specific expression in kidney in addition to HEVEC from human tonsils. Additionally, the size of the SUT2 mRNA species detected in kidney was approximately 5.2 kb, 2.3 kb larger than the SUT2 cDNA cloned from HEVEC, suggesting alternative splicing or polyadenylation of the SUT2 pre-mRNA. This tissue-specific expression provides applications for SUT2 and SUT2 modulators in these tissues. Similarly to HEVEC, Na+-independent sulfate transport activity has been shown to play important physiological roles in kidney. In rabbit kidney, Na+-independent sulfate transport activity has been detected on both basolateral (Kuo, S. M. and Aronson, P. S. (1988) J Biol Chem 263: 9710-9717) and apical (Kuo, S. M. and Aronson, P. S. (1996) J Biol Chem 271: 15491-15497) membranes of the proximal tubule. Since rat SAT1/SLC26A1 protein was only detected at the basolateral side (Karniski, L. P., et al. (1998) Am J Physiol 275: F79-87), it has been suggested that a Na+-independent sulfate transporter, other than SAT1/SLC26A1, is expressed at the apical side of rat proximal tubule. However, functional expression cloning of sulfate transporters from rat kidney by injection of mRNA in Xenopus laevis oocytes did not permit its isolation (Markovich, D., et al. (1994) J Biol Chem 269: 3022-3026). SUT2, which is expressed at high levels in kidney, may therefore be the kidney apical Na+-independent sulfate transporter.

To analyze SUT2 mRNA expression in HEVEC from human tonsils, RT-PCR analysis was performed using oligonucleotides specific for DTDST/SLC26A2 as control. SUT2 was found to be expressed in tonsillar HEVEC, at levels comparable to those found in kidney. These results indicate that HEVEC coexpress transcripts for two distinct members of the SLC26 sulfate/anion transporter family, SUT2 and DTDST/SLC26A2.

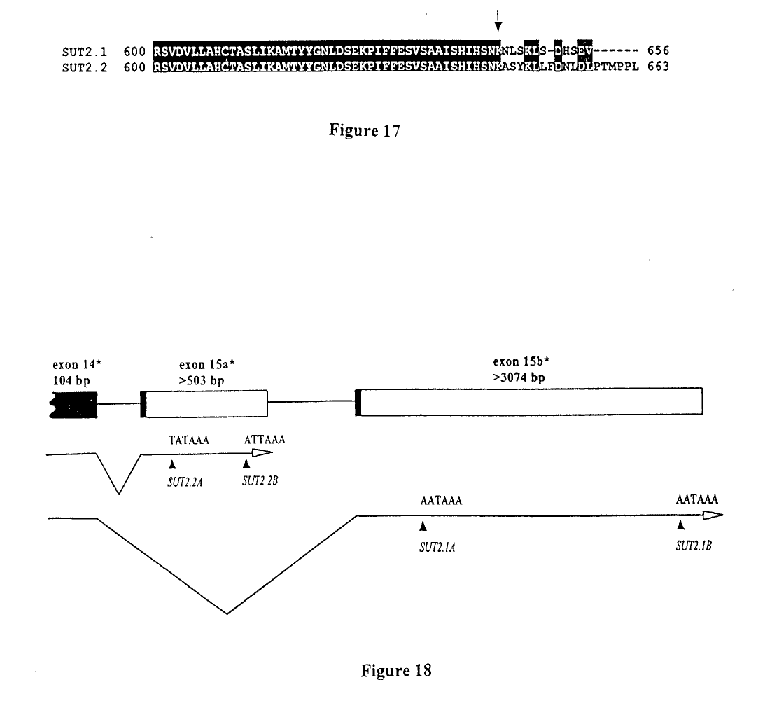

To characterize the nature of the sequences responsible for the difference in size observed between HEVEC SUT2 cDNA and kidney SUT2 mRNA, a human kidney large insert cDNA library was screened by hybridization with the same probe used to hybridize the Northern blot. A high proportion of positive clones over the total number of phages plated was obtained (1 out of 4000), providing further evidence for high levels of SUT2 expression in kidney. A 5.2 kb cDNA insert, found in several positive clones, was entirely sequenced. A 5254 bp sequence including a 25 bp poly(A) tail was assembled. Comparison of the kidney and HEVEC SUT2 cDNA sequences revealed that the first 2836 bp of the kidney sequence were identical to the HEVEC sequence from nucleotide 33 to 2868 of SEQ ID NO: 11. Both sequences differed only by the fact that the 5′UTR of the HEVEC SUT2 cDNA sequence was 32 bp longer than that in the kidney whereas the 3′ UTR (poly(A) tail excluded) of the later was 2400 bp longer than the HEVEC 3′ UTR. Consequently, both sequences share the same ORF of SEQ ID NO: 13. In addition, another SUT2 cDNA (SEQ ID NO: 12) from kidney was found, that encodes a second SUT2 protein isoform (SEQ ID NO: 14) with slight modifications in the carboxy terminal part. This 2345 bp cDNA sequence (SEQ ID NO: 12) is identical to the 5.2 kb kidney SUT2 cDNA at the 5′end, however the two sequences diverge from each other from nucleotide position 2143 to their respective poly(A) tails. This sequence divergence in the two cDNAs give rise to modifications of the SUT2 ORF, with the last 11 residues (e.g. amino acid positions 646 to 656 of SEQ ID NO: 13) in the major 656-amino-acid SUT2 isoform of SEQ ID NO: 13 replaced by a novel 18 amino-acid sequence (e.g. amino acid positions 646 to 663 of SEQ ID NO: 14) in the 663-amino-acid SUT2 isoform of SEQ ID NO: 14 (FIG. 17). However, the SUT2 form of SEQ ID NO: 14 does not appear to be a major SUT2 isoform since among 24 kidney SUT2 phage clones characterized, only one turned out to contain this cDNA species and this 2.3 kb SUT2 mRNA species was not detected in kidney by Northern blot.

To better understand how the different SUT2 cDNAs isoforms are produced, the genomic structure of the SUT2 gene was analyzed. A BLAST search of the draft human genome sequence database at NCBI using SUT2 cDNA sequences as baits revealed a genomic hit (GenBank Accession N^{o}NT023693, the disclosure of which, including the genomic DNA sequence, is incorporated herein by reference) that partially covered all cDNA sequences from the first nucleotide encoding amino acid 160 of SUT2 protein to the 3′ end of the cDNAs. Through further analysis based on this genomic hit originating from the Homo Sapiens chromosome 8 working draft sequence but not precisely mapped, the inventors localized the SUT2 gene to chromosomal region 8q23.

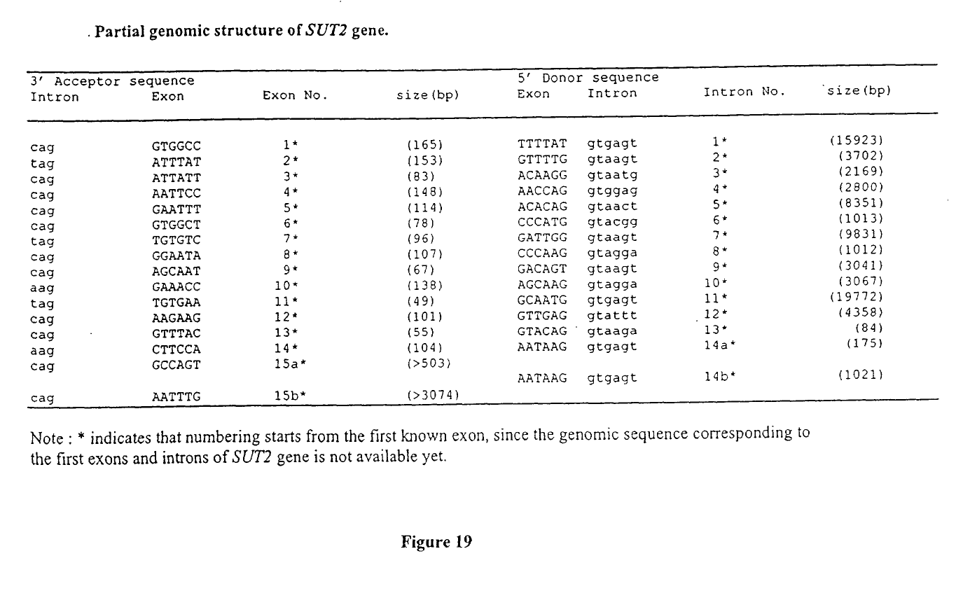

Alignment between the cDNAs and genomic sequences led to the identification of the last 16 exons of the human SUT2 gene. The partial genomic structure of the SUT-2 gene is shown in FIG. 19, including the 3 splice acceptor and 5′ splice donor sequences and the lengths of the SUT2 introns and exons. All the exon-intron boundaries follow the GT-AG rule.

The partial genomic structure of the SUT2 gene offers an explanation about how the different SUT2 cDNAs are produced by alternative splicing and polyadenylation. The penultimate exon, which contains two polyadenylation signals, is spliced out in the 5.2 kb kidney and the 2.9 kb HEVEC SUT2 mRNAs encoding the protein of SEQ ID NO: 13, whereas it is retained in the SUT2 mRNA isoform encoding the protein of SEQ ID NO: 14. The difference in size between SUT2 HEVEC and kidney cDNAs is easily explained by the use of alternative polyadenylation signals in the last exon as shown in FIG. 18.

In order to characterize the function of the SUT-2 protein and develop screening assays that can be used in drug screening, SUT-2 function was tested in assays allowing sulfate transport activity to be examined. Functional assays in cRNA-injected Xenopus laevis oocytes showed that SUT-2 mediates sulfate transport, indicating that SUT-2 has activity as a sulfate transporter.

As described herein, SUT-2 comprises elements of the sulfate tranporter signature, but contains differences in amino acid sequence. In one embodiment, a SUT-2 family member is identified based on the presence of at least a “sulfate transporter signature” in the protein or corresponding nucleic acid molecule. An SUT-2 family member may comprise a “sulfate transporter signature” having an amino acid sequence of at least about 25, 30, 35, 40, 45, 50 or 60 amino acid residues in length, of which at least about 50-80%, preferably at least about 60-70%, more preferably at least about 65% of the amino acid residues are identical or similar amino acids-to the “sulfate transporter signature” consensus domain (SEQ ID NO: 15) as follows:

[PAV]-x-Y-[GS]-L-Y-[STAG](2)-x(4)-[LIVFYA]-[LIVST]-[YI]-x(3)-[GA]-[GST]-S-[KR].

In some embodiments, the SUT-2 family member comprises an amino acid sequence of at least about 25, 30, 35, 40, 45, 50 or 60 amino acid residues in length, of which at least about 50-80%, preferably at least about 60-70%, more preferably at least about 65% of the amino acid residues are identical or similar amino acids-to amino acid residues 76 to 97 of SEQ ID NOs: 13 or 14.

Identity or similarity may be determined using any desired algorithm, including the algorithms and parameters for determining homology which are described herein.

SUT-2 and SUT-3 Features

As described herein, SUT-2 and SUT-3 comprise elements of the sulfate tranporter signature, but contain differences in amino acid sequence. In one embodiment, a SUT-2 or SUT-3 family member is identified based on the presence of at least a “sulfate transporter signature” in the protein or corresponding nucleic acid molecule. A SUT-2 or SUT-3 family member may comprise a “sulfate transporter signature” having an amino acid sequence of at least about 25, 30, 35, 40, 45, 50 or 60 amino acid residues in length, of which at least about 50-80%, preferably at least about 60-70%, more preferably at least about 65% of the amino acid residues are identical or similar amino acids to the “sulfate transporter signature” consensus domain or to residues 76-97 of SEQ ID NOs: 13 or 14 or residues 137-468 of SEQ ID NO: 4, residues 137-512 of SEQ ID NO: 5, residues 137-468 of SEQ ID NO: 6, residues 77-98 of SEQ ID NOs: 4, 5, 6 and 10 or residues 137-468 of SEQ ID NO: 10.

Identity or similarity may be determined using any desired algorithm, including the algorithms and parameters for determining homology which are described herein.

In preferred embodiments, a SUT-2 or SUT-3 family member further comprises an amino acid sequence of at least about 50, 100, 200, 300, 400, 500, 600 or 650 amino acid residues in length, of which amino acid sequence at least about 99%, 98%, 95%, 90%, 50-80%, preferably at least about 60-70%, more preferably at least about 65% of the amino acid residues are identical or similar to the amino acid sequences shown in SEQ ID NOs: 4, 5, 6, 10, 13, 14 or 27. A “similar” amino acid residue is a residue which represents a conservative amino acid change relative to the sequence to which it is being compared.

The sulfate transporter signature domain is further described in PROSITE Document, Accession No. PDOC00870 (http://www.expasy.org/cgibin/nicedoc.pl?PDOC00870) and as PROSITE Accession No. PS01130, the disclosures of which are incorporated herein by reference in their entireties.

In another embodiment of the invention, a SUT-2 protein preferably comprises at least 1, 2, 4, 6, 8, 10, 11 or 12 transmembrane domains. A SUT-3 protein preferably comprises at least 1, 2, 4, 6, 8, 10 or 11 transmembrane domains. As used herein, the term “transmembrane domain” refers to an amino acid sequence having at least about 10, preferably about 13, preferably about 16, more preferably about 19, and even more preferably about 21, 23, 25, 30, 35 or 40 amino acid residues, of which at least about 60-70%, preferably at least about 80% and more preferably at least about 90% of the amino acid residues contain non-polar side chains, for example, alanine, valine, leucine, isoleucine, proline, phenylalanine, tryptophan, and methionine. A transmembrane domain is lipophillic in nature. For example, with respect to SUT-3, 11 transmembrane domains (designated TM1-TM12) and their amino acids positions can be found as shown in FIGS. 7 and 8. With respect to SUT-2, 12 transmembrane domains (designated TM1-TM12) can be found at amino acid positions TM1:54-71; TM2: 77-95; TM3:100-117; TM4: 150-169; TM5:174-193; TM6:228-245; TM7:257-274; TM8:309-326; TM9:349-366; TM10: 384-402; TM11:407-424; and TM12:443-465 of SEQ ID NOs: 13 or 14.

Isolated proteins of the present invention, preferably SUT-3 or SUT-2 proteins, have an amino acid sequence sufficiently homologous to an amino acid sequence presented in SEQ ID NOs: 4, 5, 6, 10, 13, 14 or 27, or are encoded by a nucleotide sequence sufficiently homologous to a sequence presented in SEQ ID NOs: 1, 2, 3, 9, 11 or 12. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least about 30-40% identity, preferably at least about 40-50% identity, more preferably at least about 50-60%, and even more preferably at least about 60-70%, 70-80%, 80%, 90%, 95%, 97%, 98%, 99% or 99.8% identity across the amino acid sequences of the domains and contain at least one and preferably two structural domains or motifs, are defined herein as sufficiently homologous. Furthermore, amino acid or nucleotide sequences which share at least about 30%, preferably at least about 40%, more preferably at least about 60%, 70%, 80%, 90%, 95%, 97%, 98%, 99% or 99.8% identity and share a common functional activity are defined herein as sufficiently homologous.

As used interchangeably herein, a “SUT-3 activity”, “biological activity of SUT-3” or “functional activity of SUT-3”, refers to an activity exerted by a SUT-3 protein, polypeptide or nucleic acid molecule as determined in vivo, or in vitro, according to standard techniques. In one embodiment, a SUT-3 activity is a direct activity, such as an association with a SUT-3-target molecule or most preferably ion exchange or sulfate transport activity. As used herein, a “target molecule” is a molecule with which a SUT-3 protein binds or interacts in nature, such that SUT-3-mediated function is achieved. A SUT-3 target molecule can be a SUT-3 protein or polypeptide of the present invention or a non-SUT-3 molecule. For example, a SUT-3 target molecule can be a non-SUT-3 protein molecule. Alternatively, a SUT-3 activity is an indirect activity, such as an activity mediated by interaction of the SUT-3 protein with a SUT-3 target molecule such that the target molecule modulates a downstream cellular activity (e.g., interaction of a SUT-3 molecule with a SUT-3 target molecule can modulate the activity of that target molecule on an intracellular signalling pathway).

In a preferred embodiment, a SUT-3 activity is selected from the group consisting of (i) anion exchange activity, preferably a sulfate/anion exchange activity; (ii) cellular sulfate uptake, preferably uptake by a Xenopus Oocyte or baculovirus infected-Sf9 insect cells, optionally by a HEVEC. In a preferred embodiment, SUT-3 activity is sulfate ion transport activity. In preferred embodiments, SUT-3 sulfate transport activity is assessed according to any of the methods of Markovitch et al, (1993) PNAS USA 90:8073-8077; Bissig et al, (1994) J. Biol. Chem. 269: 3017-3021; Silberg et al, (1995) J. Biol. Chem. 270: 11897-11902; Girard et al, (1999) PNAS USA 96(22) 12772-12777, the disclosures of which are incorporated herein by reference in their entireties; and Example 2 or Example 5 described herein.

In yet another preferred embodiment, a SUT-3 activity is detected by assessing the following indirect activities: (1) mediating sulfation of L-selectin ligands, preferably sialomucin-type L-selectin counter receptors; (2) mediating L-selectin dependent lymphocyte adhesion to cells expressing a L-selectin ligand, preferably HEVs or HEVECs; (3) mediating interactions between L-selectin ligands, preferably sialomucin-type L-selectin counter receptors and L-selectin; or (4) mediating adhesion of lymphocytes to endothelial cells. Adhesion of lymphocytes to HEVs or HEVECs may in preferred embodiments be assessed by observing the ‘rolling phenotype’ in vivo in mouse lymph node HEVs using intravital microscopy (microscopy on live animals) (von Andrian (1996) Microcirculation (3):287-300; and von Andrian UH, M'Rini C. (1998) Cell Adhes Commun. 6(2-3):85-96), the disclosures of which are incorporated herein by reference in their entireties. SUT-3 activity may be assessed either in vitro or in vivo depending on the assay type and format.

As used interchangeably herein, a “SUT-2 activity”, “biological activity of SUT-2” or “functional activity of SUT-2”, refers to an activity exerted by a SUT-2 protein, polypeptide or nucleic acid molecule as determined in vivo, or in vitro, according to standard techniques. In one embodiment, a SUT-2 activity is a direct activity, such as an association with a SUT-2-target molecule or most preferably ion or chloride exchange, or sulfate transport activity. As used herein, a “target molecule” is a molecule with which a SUT-2 protein binds or interacts in nature, such that SUT-2-mediated function is achieved. A SUT-2 target molecule can be a SUT-2 protein or polypeptide of the present invention or a non-SUT-2 molecule. For example, a SUT-2 target molecule can be a non-SUT-2 protein molecule. Alternatively, a SUT-2 activity is an indirect activity, such as an activity mediated by interaction of the SUT-2 protein with a SUT-2 target molecule such that the target molecule modulates a downstream cellular activity (e.g., interaction of a SUT-2 molecule with a SUT-2 target molecule can modulate the activity of that target molecule on an intracellular signalling pathway).

In a preferred embodiment, a SUT-2 activity is selected from the group consisting of (i) anion exchange activity, preferably a sulfate/anion exchange activity; (ii) cellular sulfate uptake, preferably uptake by a Xenopus Oocyte or baculovirus infected-Sf9 insect cells, optionally by a HEVEC. In a preferred embodiment, SUT-2 activity is sulfate ion transport activity. In preferred embodiments, SUT-2 sulfate transport activity is assessed according to any of the methods of Markovitch et al, (1993) PNAS USA 90:8073-8077; Bissig et al, (1994) J. Biol. Chem. 269: 3017-3021; Silberg et al, (1995) J. Biol. Chem. 270: 11897-11902; Girard et al, (1999) PNAS USA 96(22) 12772-12777, the disclosures of which are incorporated herein by reference in their entireties; and Example 2 or Example 5 described herein.

In yet another preferred embodiment, a SUT-2 activity is detected by assessing the following indirect activities: (1) mediating sulfation of L-selectin ligands, preferably sialomucin-type L-selectin counter receptors; (2) mediating L-selectin dependent lymphocyte adhesion to cells expressing a L-selectin ligand, preferably HEVs or HEVECs; (3) mediating interactions between L-selectin ligands, preferably sialomucin-type L-selectin counter receptors and L-selectin; or (4) mediating adhesion of lymphocytes to endothelial cells. Adhesion of lymphocytes to HEVs or HEVECs may in preferred embodiments be assessed by observing the ‘rolling phenotype’ in vivo in mouse lymph node HEVs using intravital microscopy (microscopy on live animals) (von Andrian (1996) Microcirculation (3):287-300; and von Andrian U H, M'Rini C. (1998) Cell Adhes Commun. 6(2-3):85-96), the disclosures of which are incorporated herein by reference in their entireties. SUT-2 activity may be assessed either in vitro or in vivo depending on the assay type and format.

SUT-2 and SUT-3 Nucleic Acids

As used herein the terminology “SUT-2 nucleic acid” or “SUT-3 nucleic acid” encompasses any of the nucleic acids discussed below.

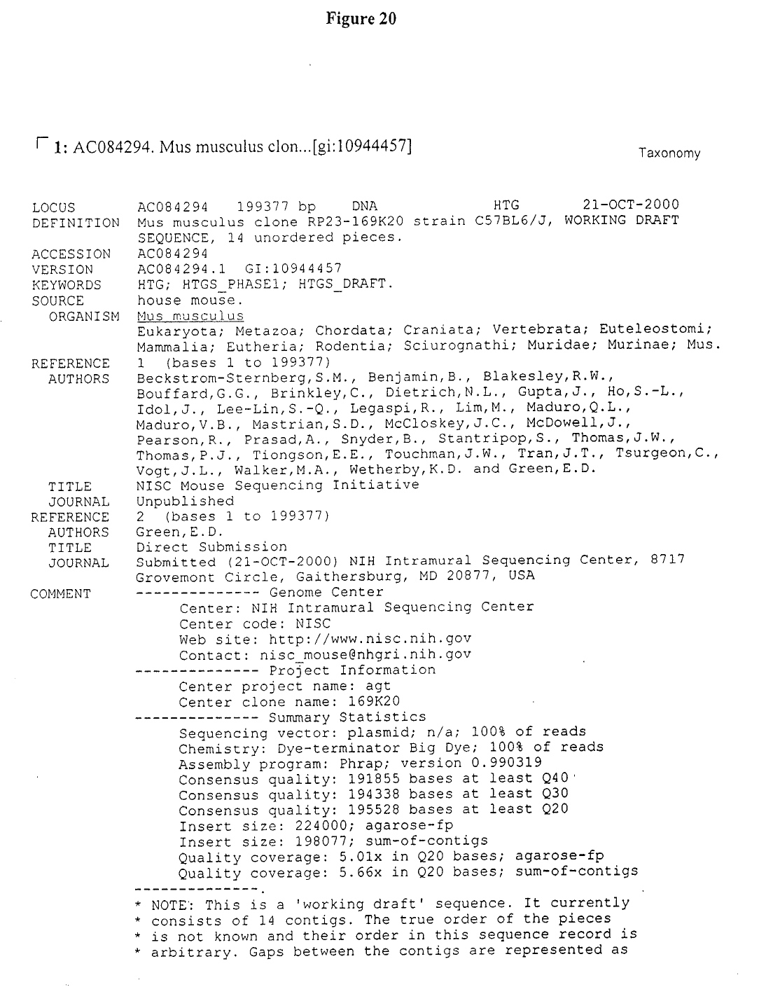

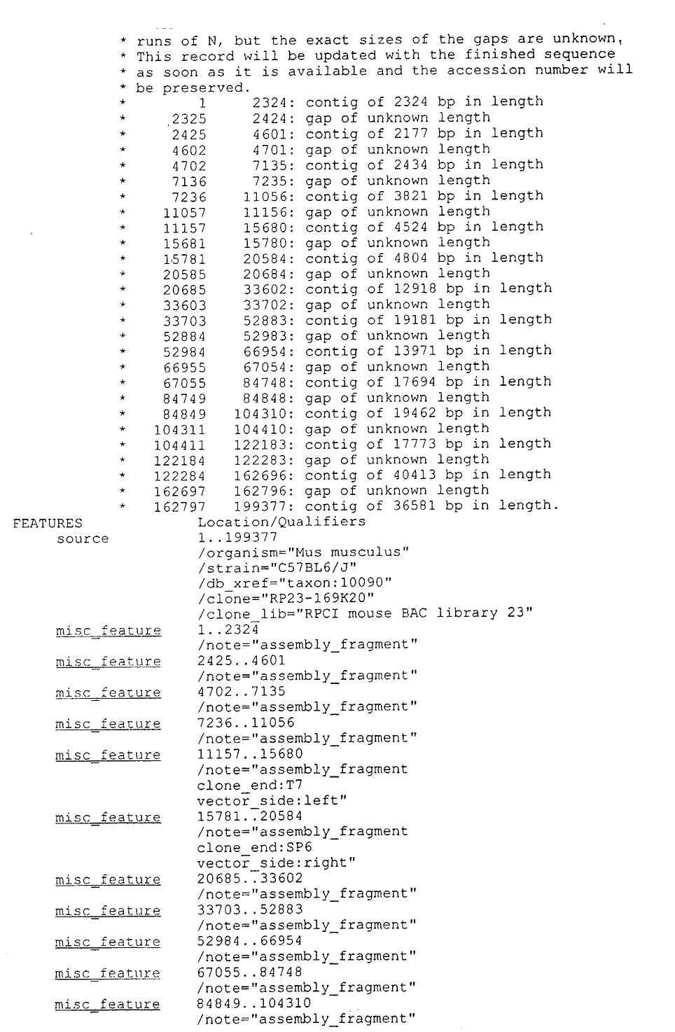

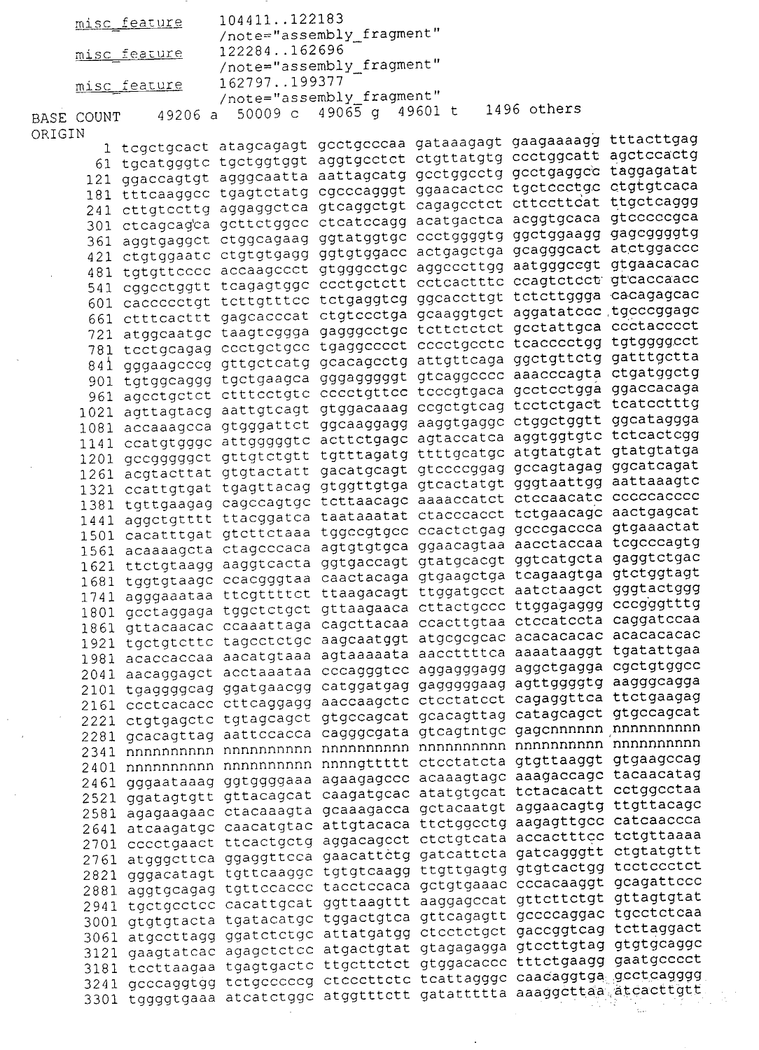

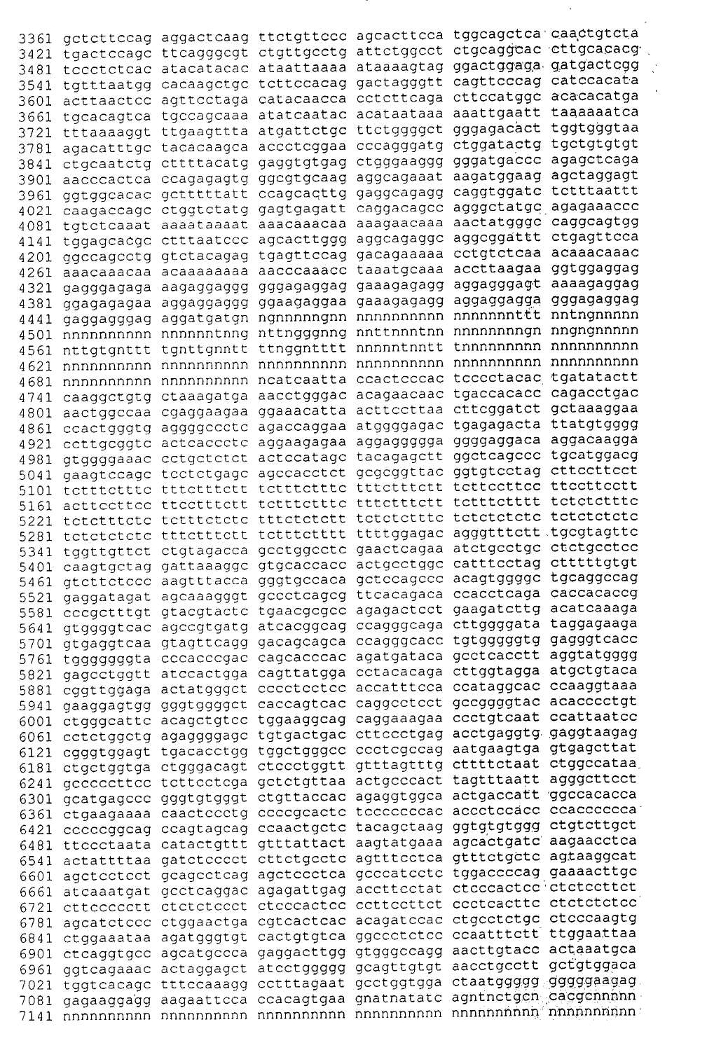

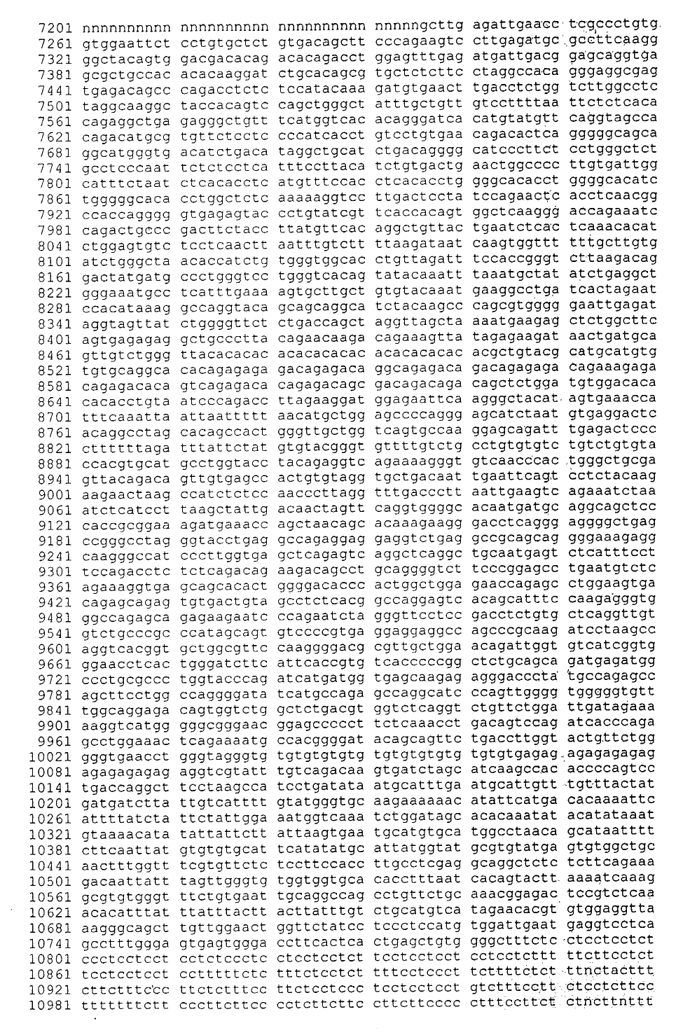

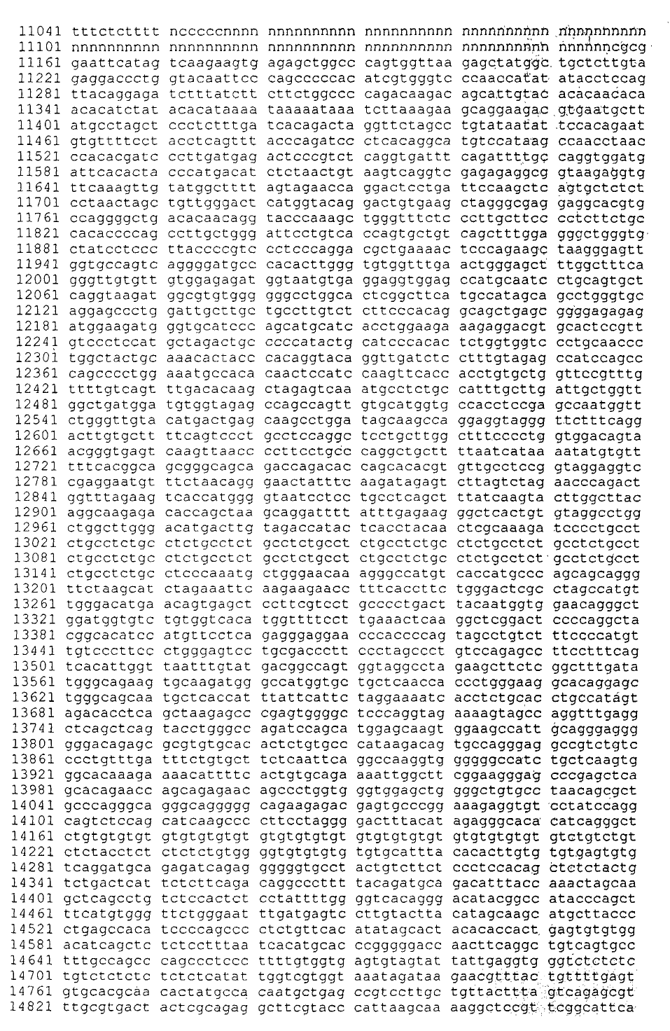

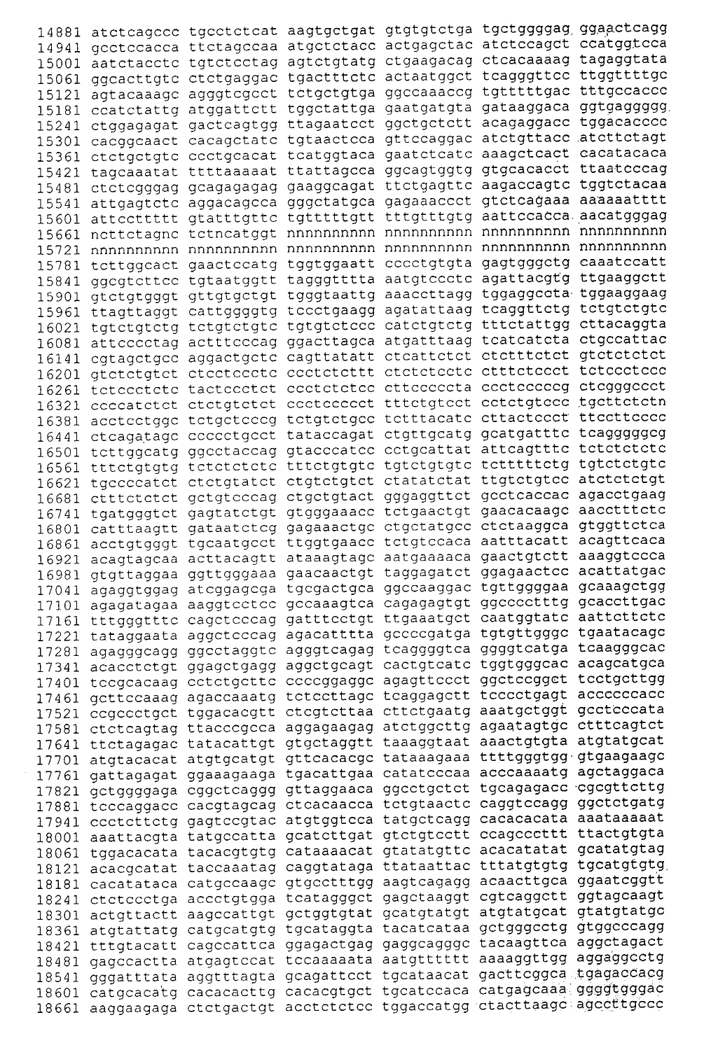

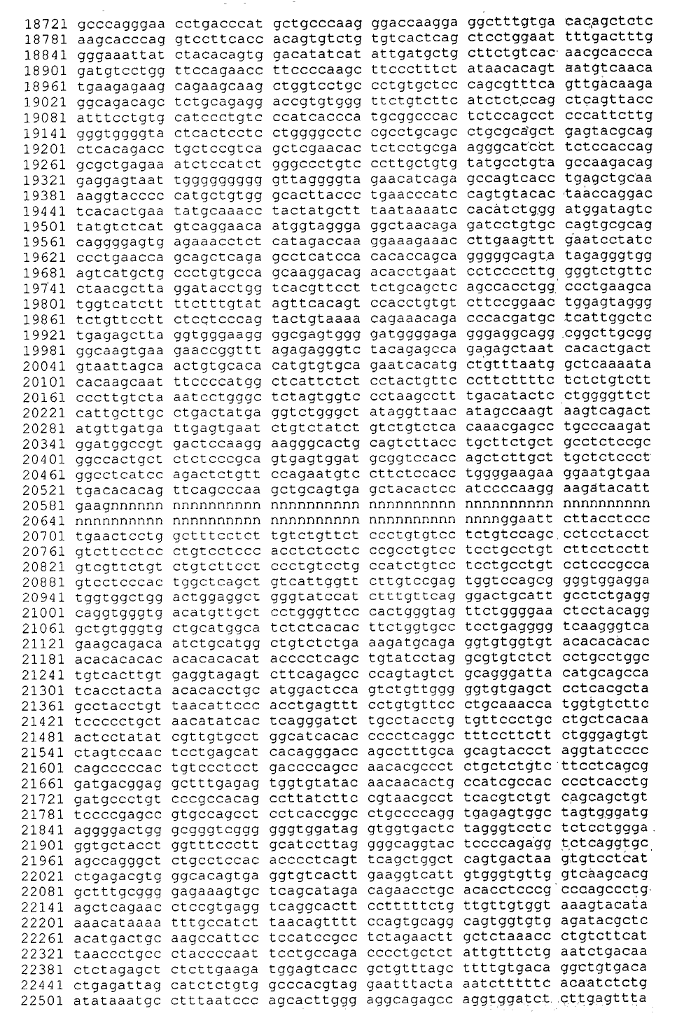

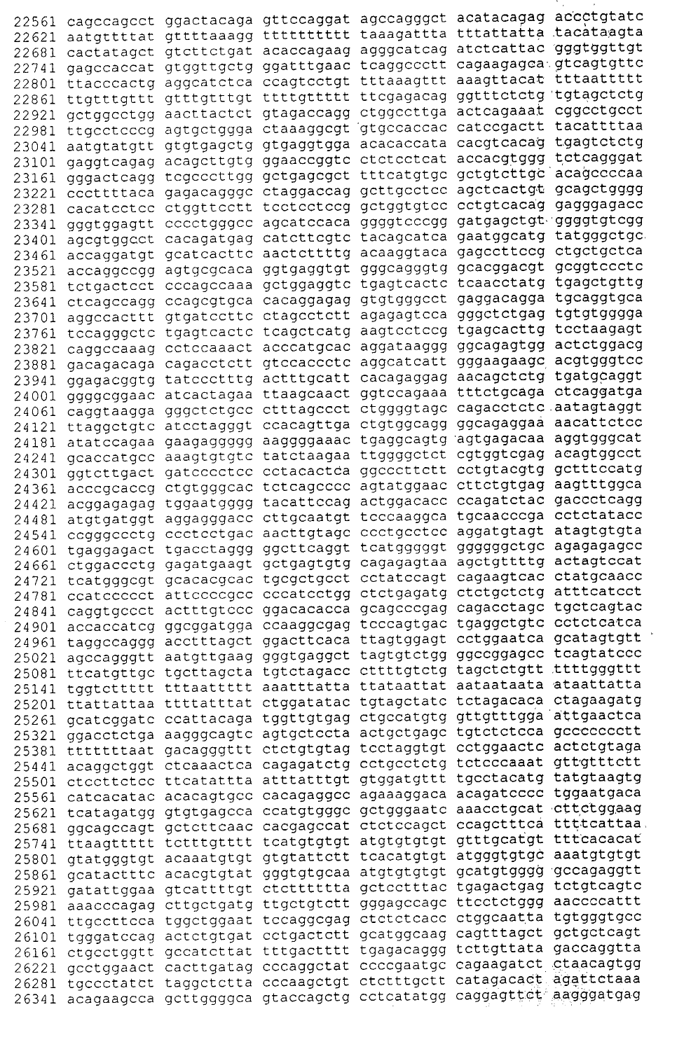

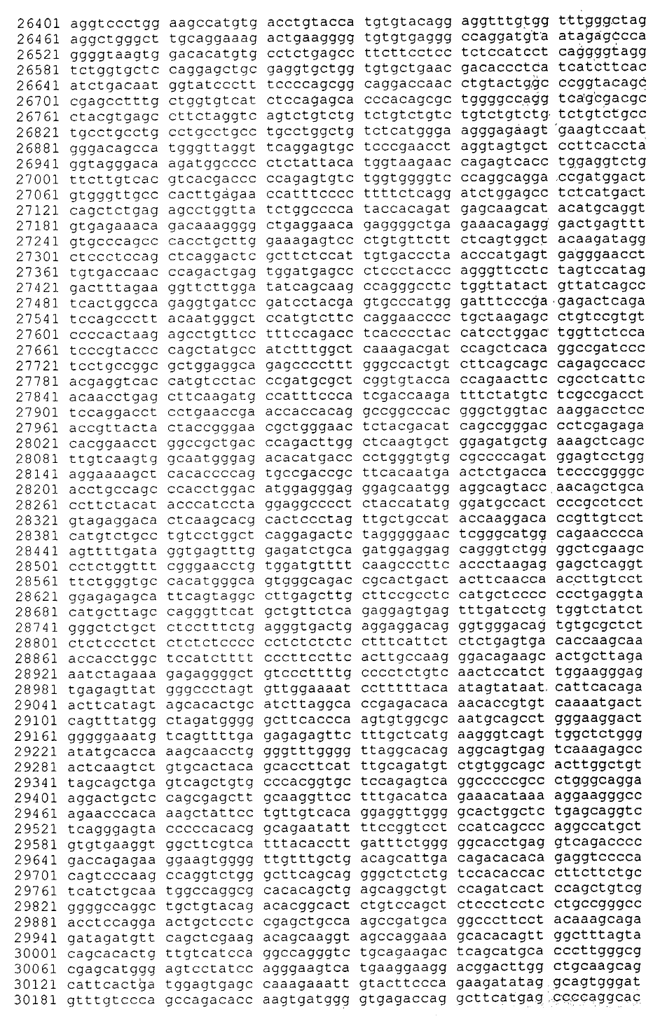

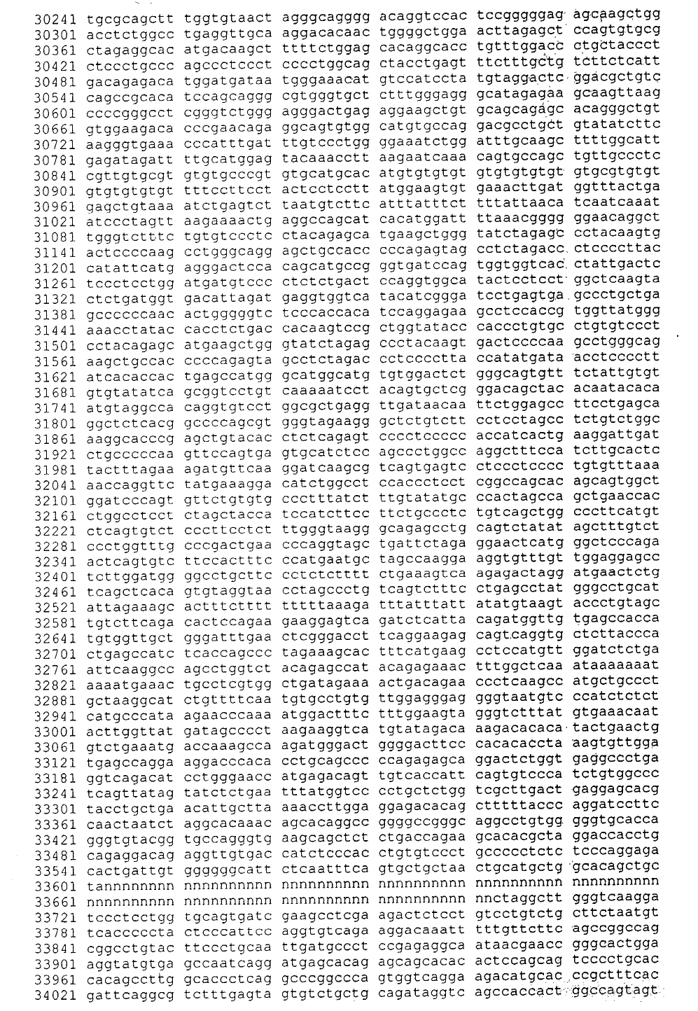

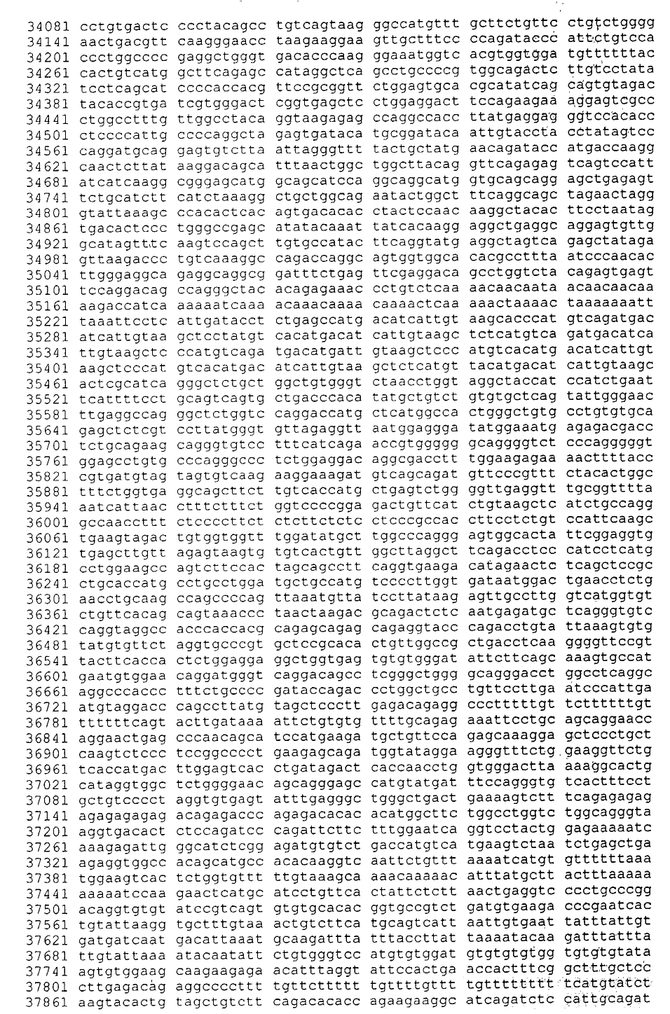

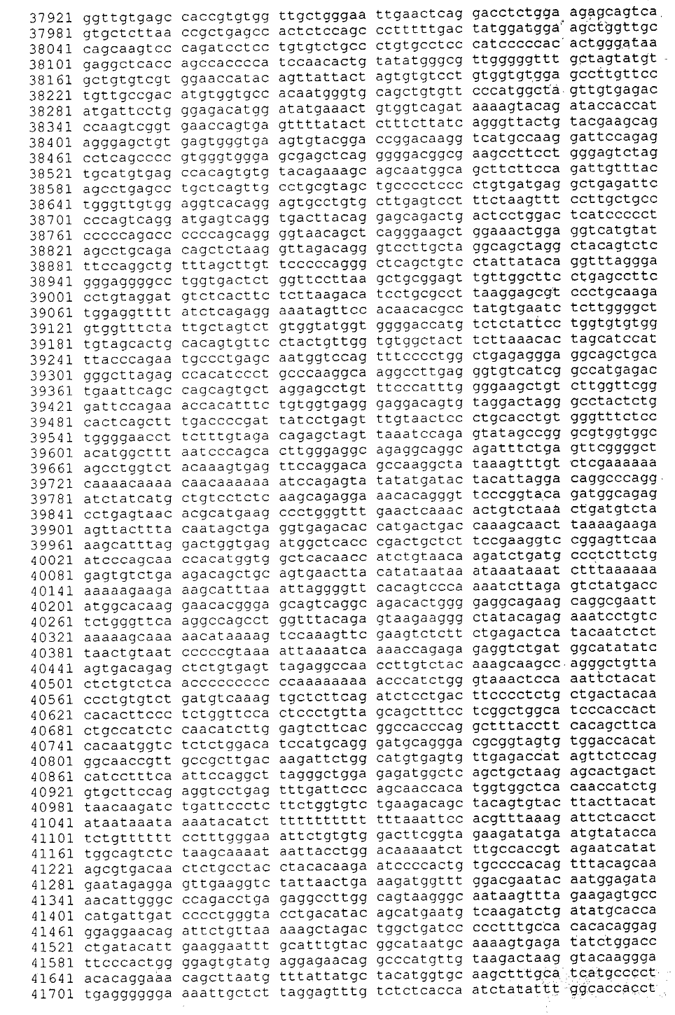

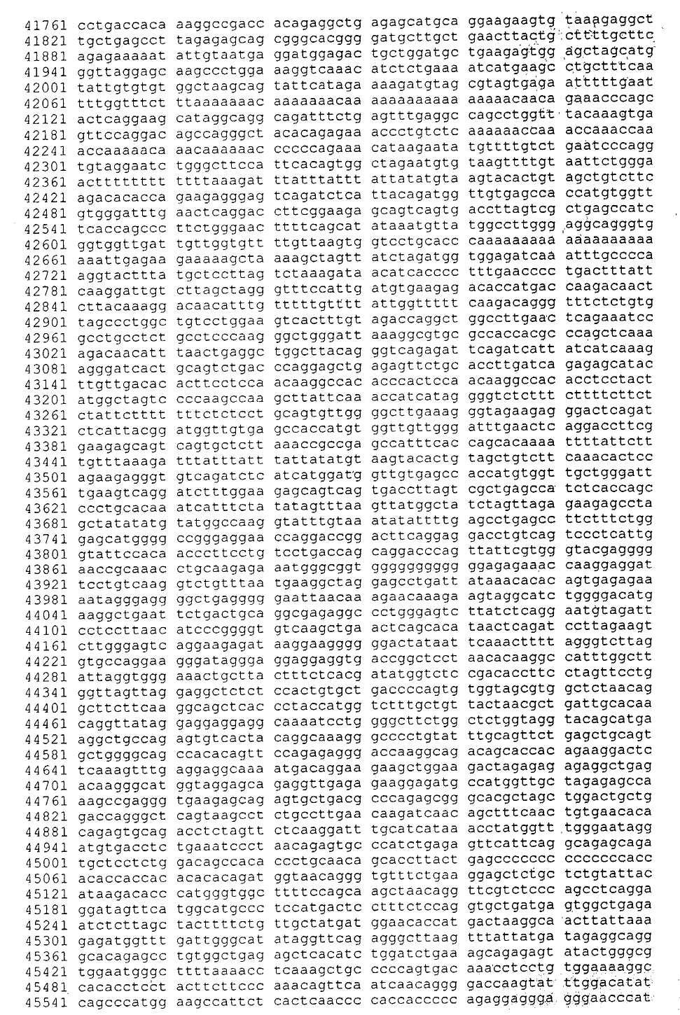

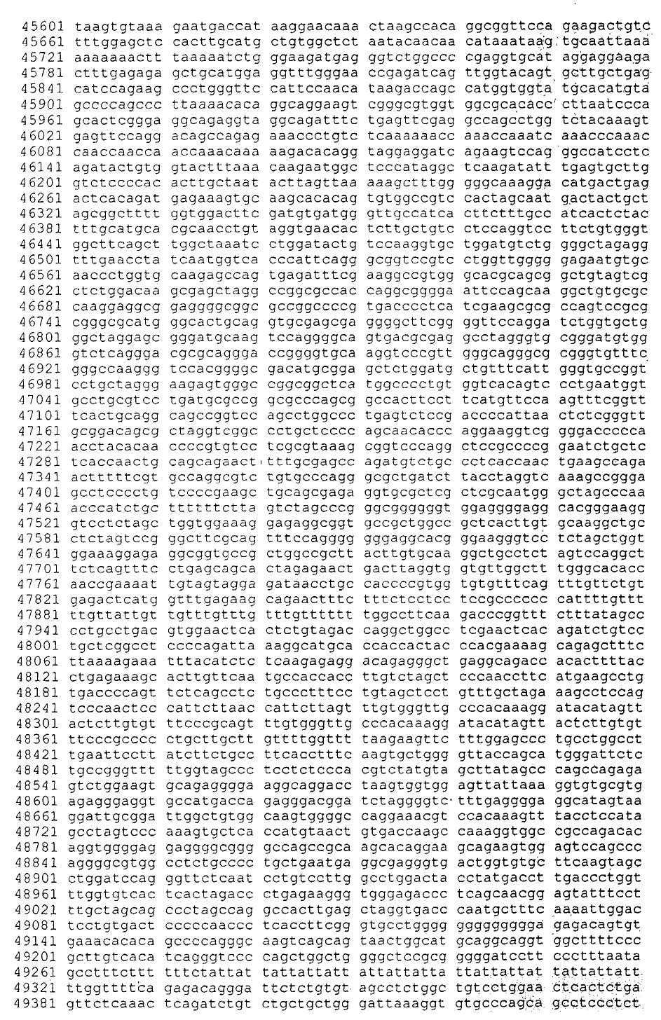

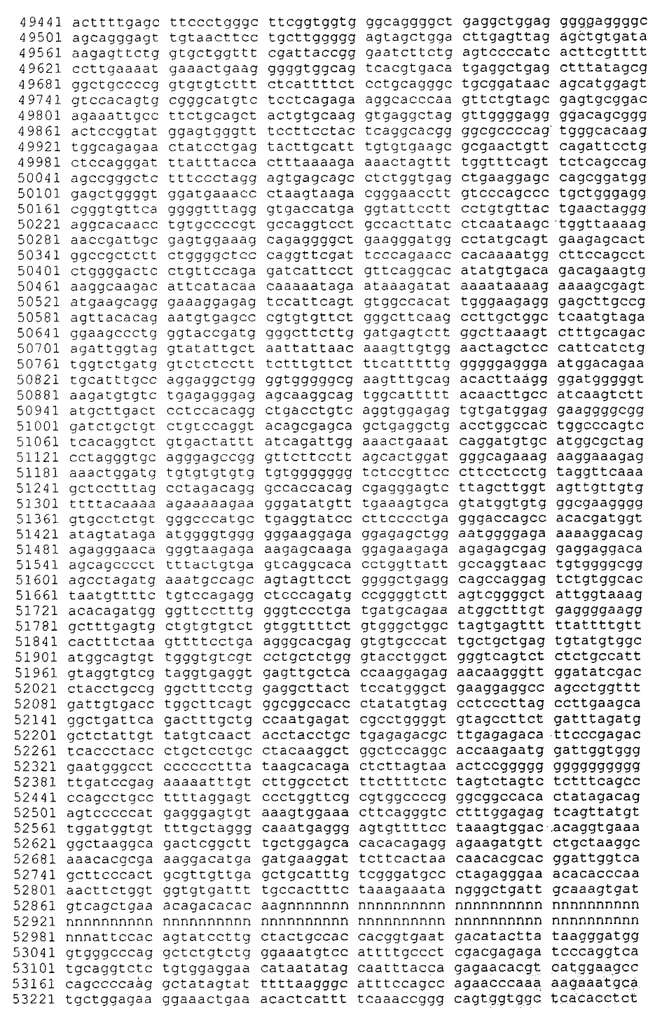

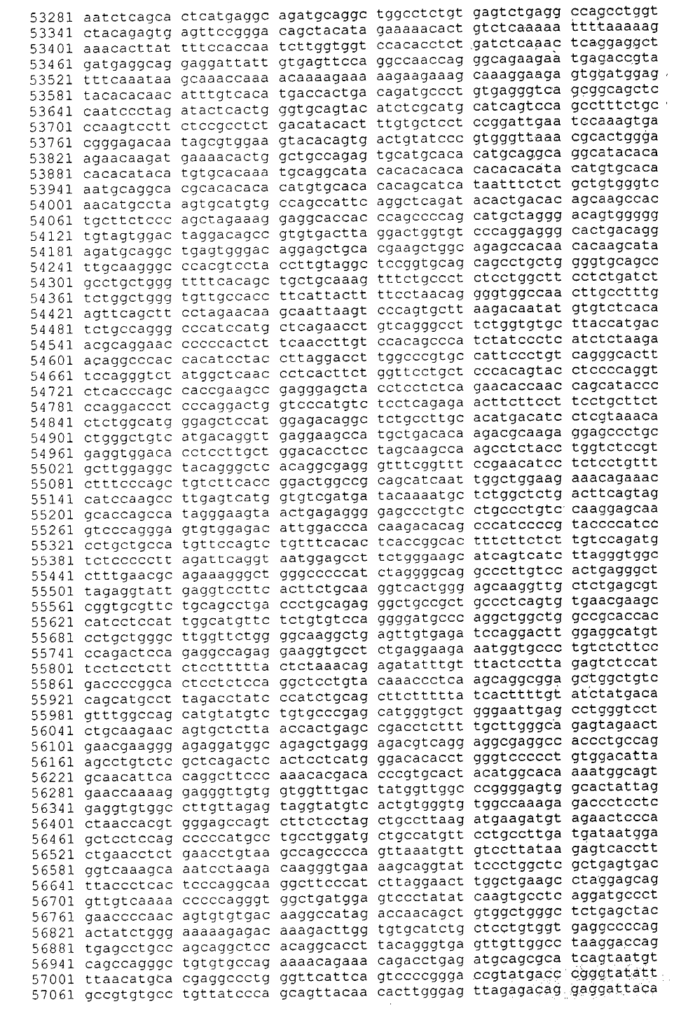

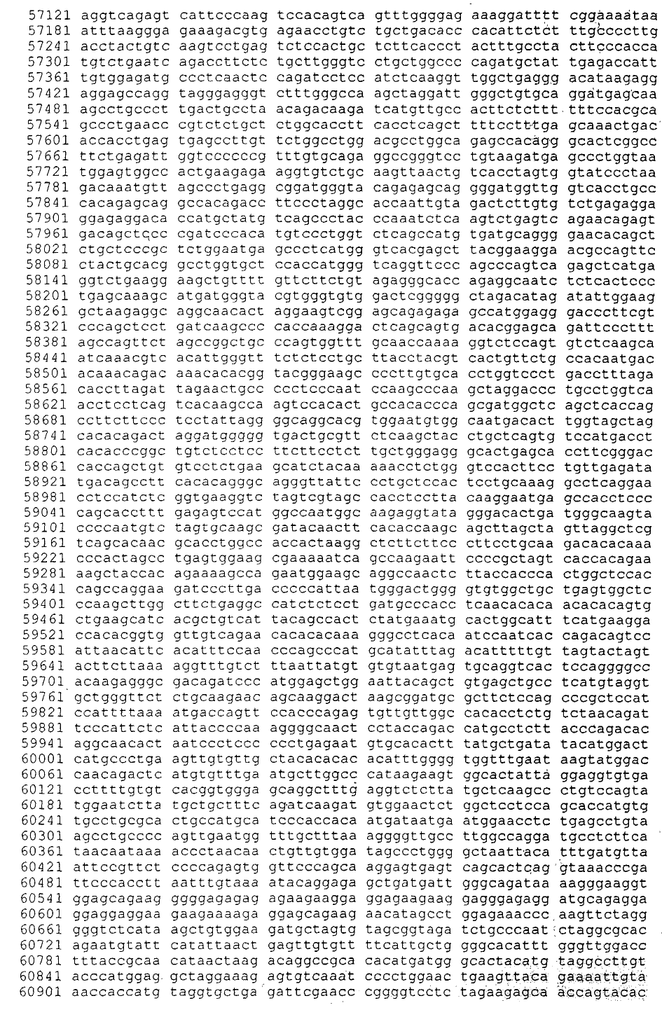

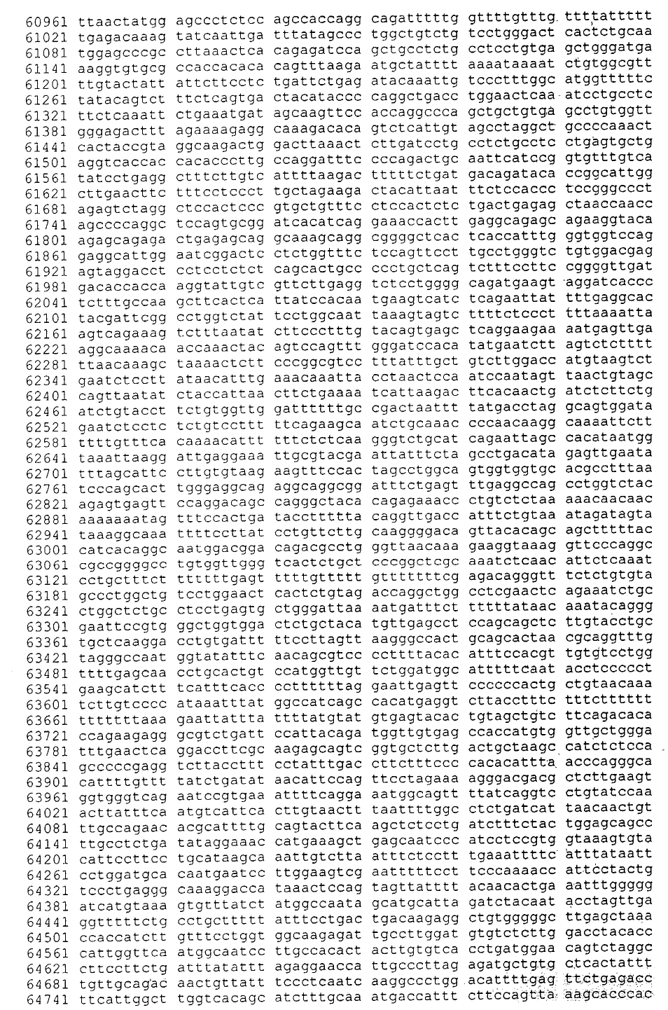

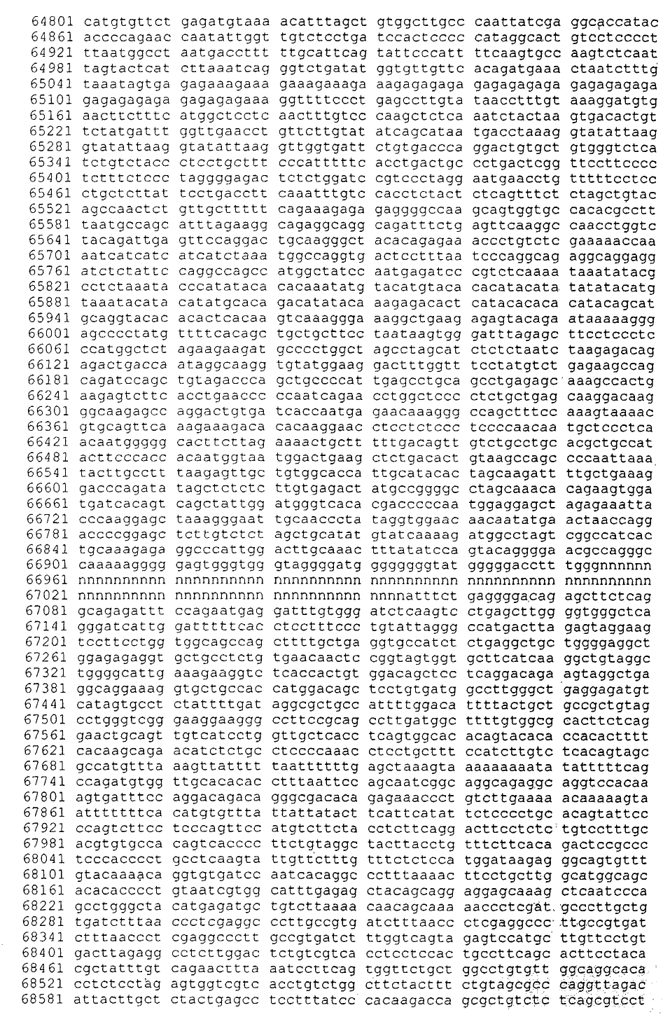

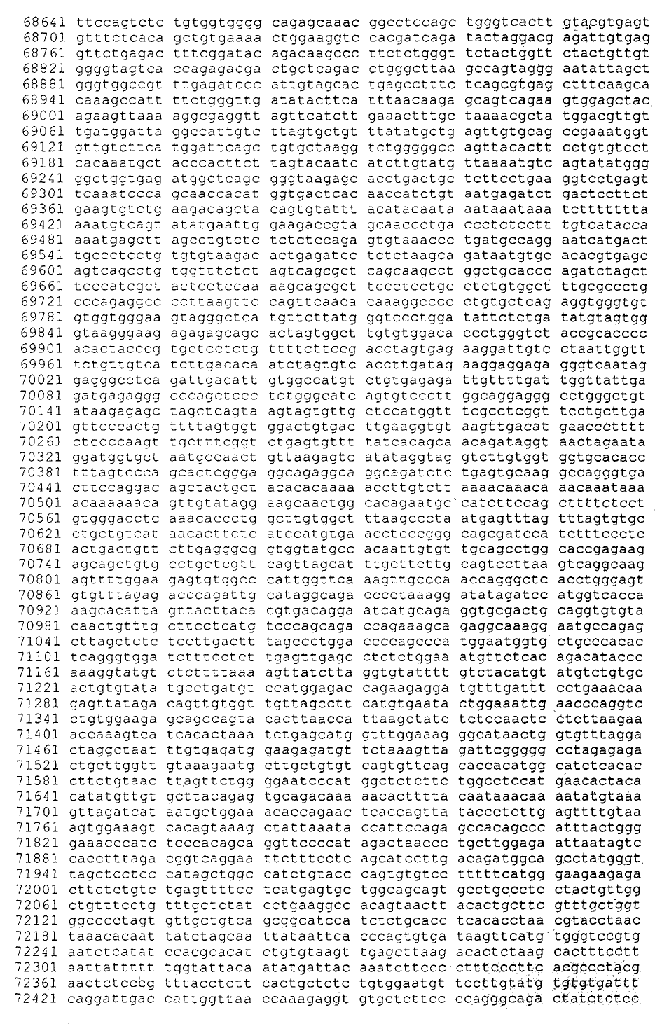

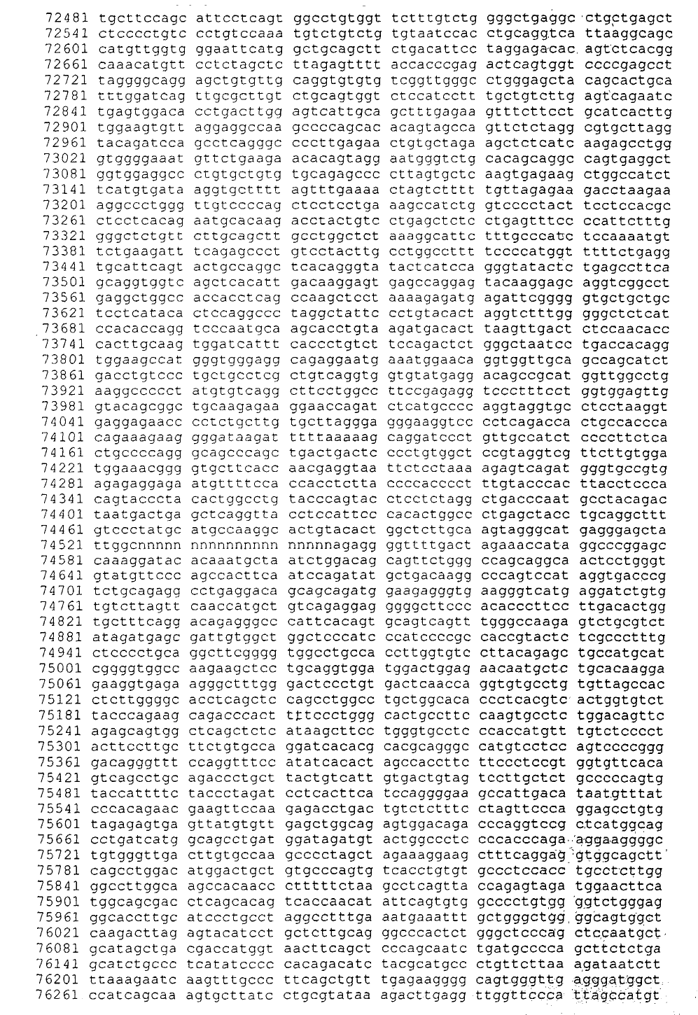

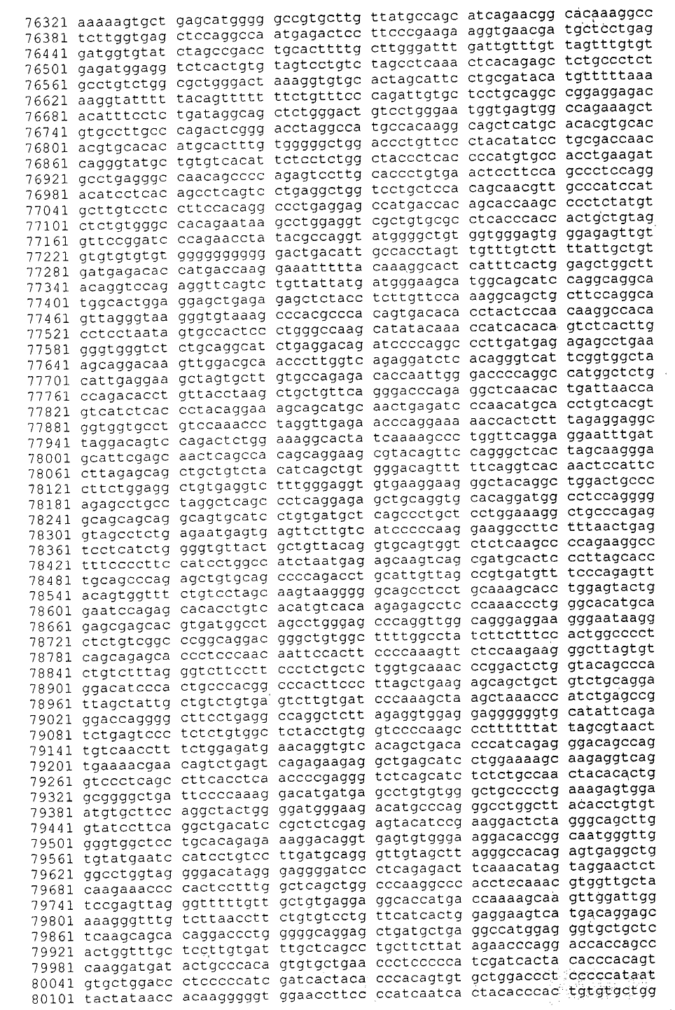

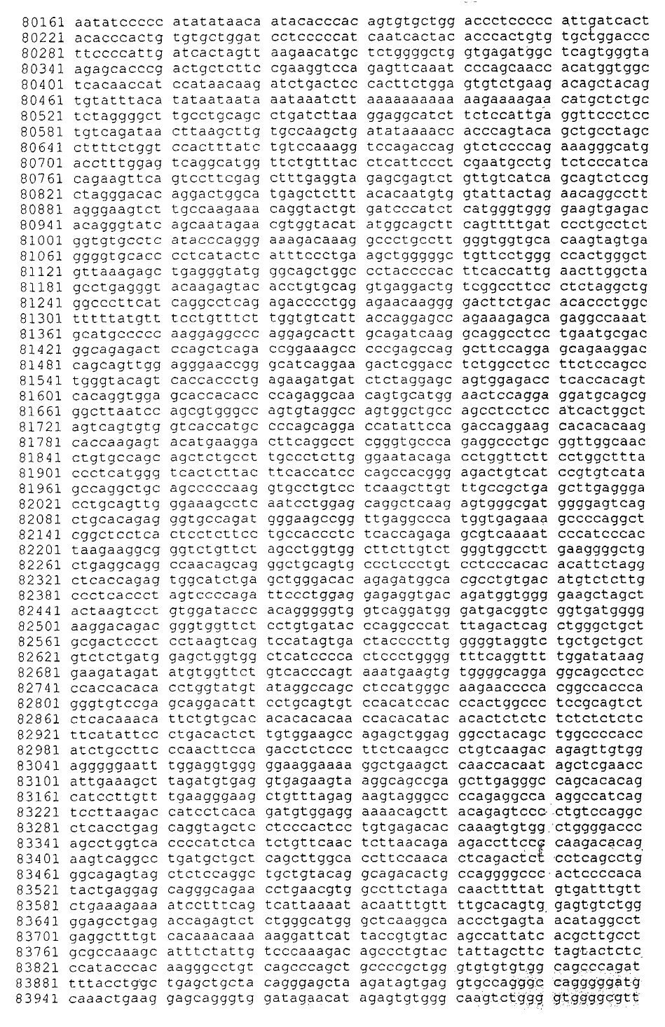

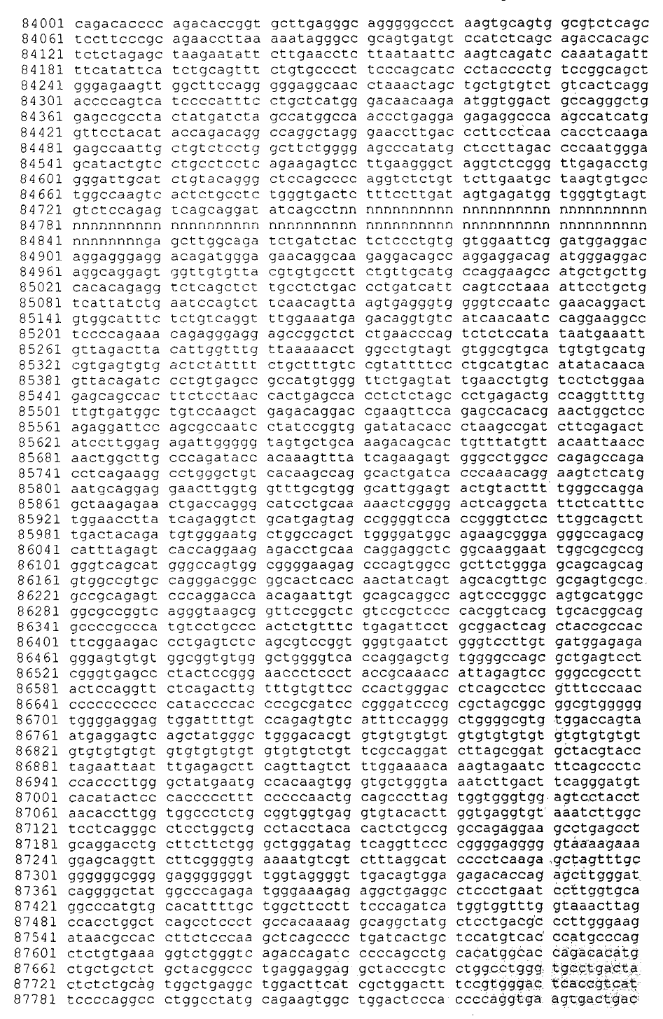

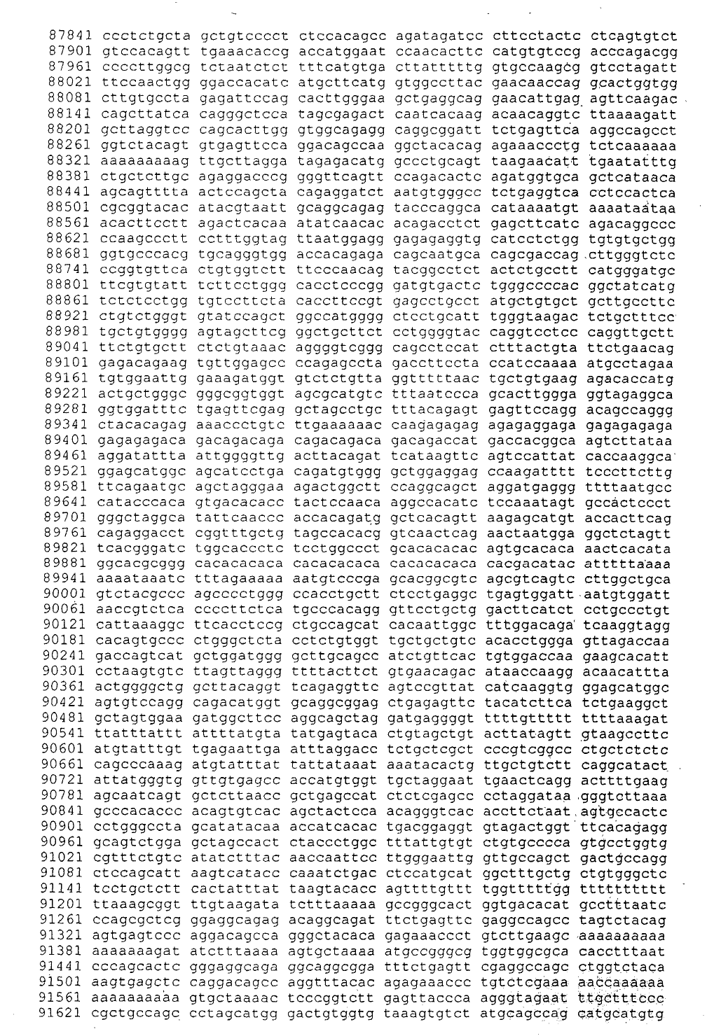

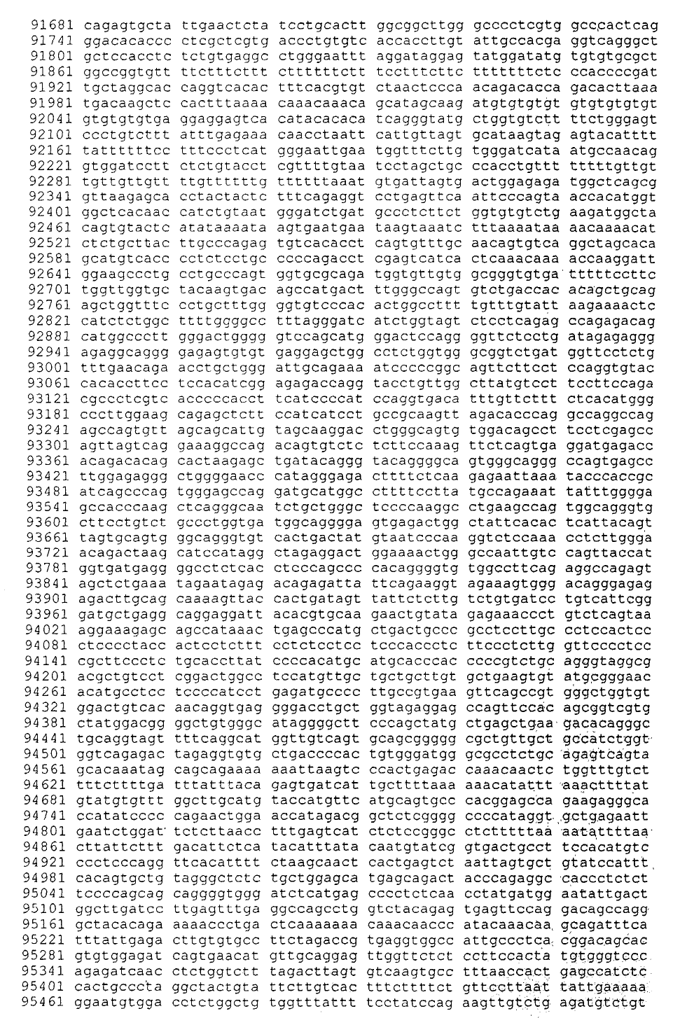

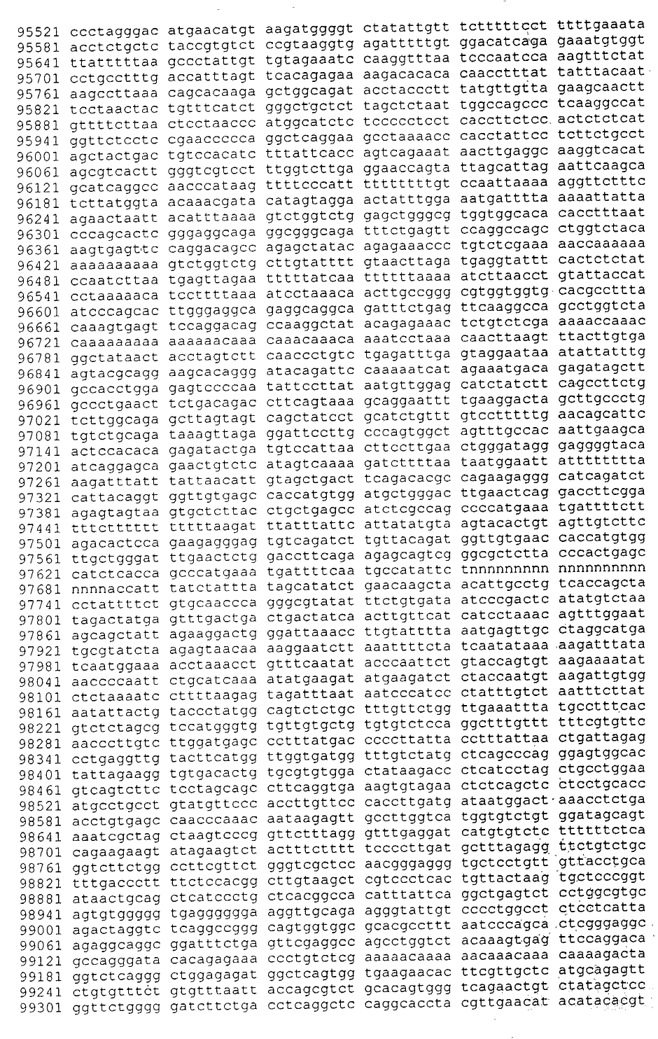

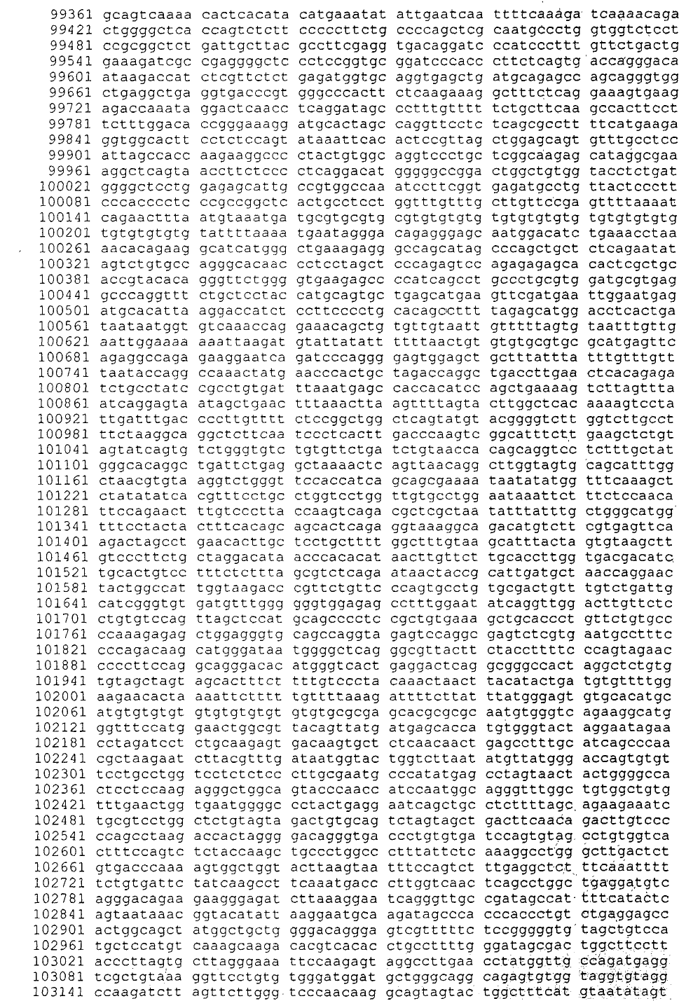

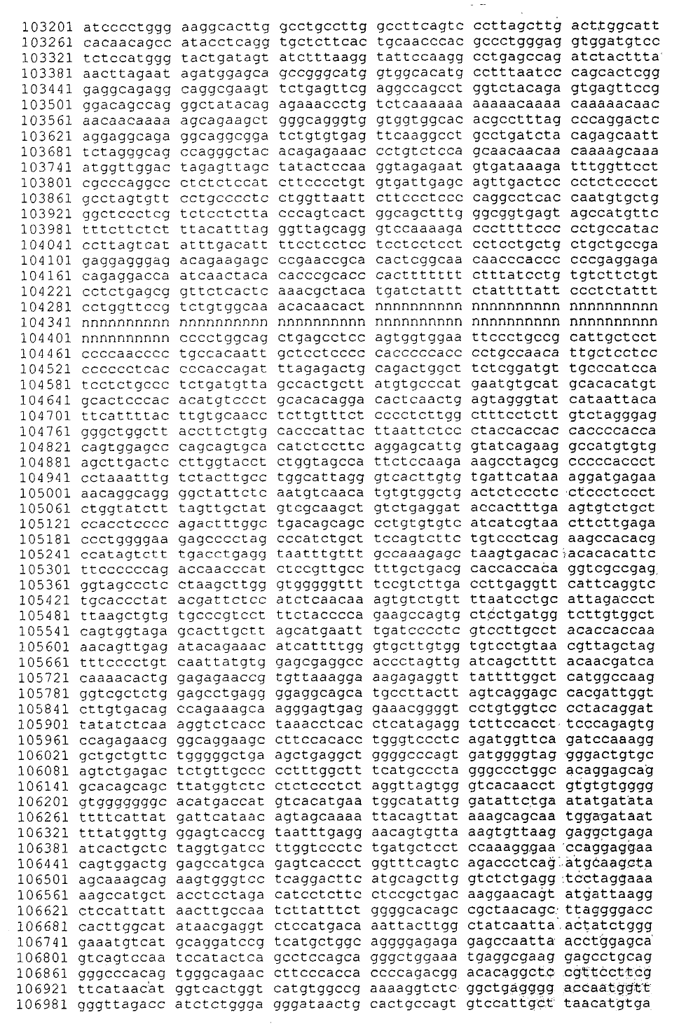

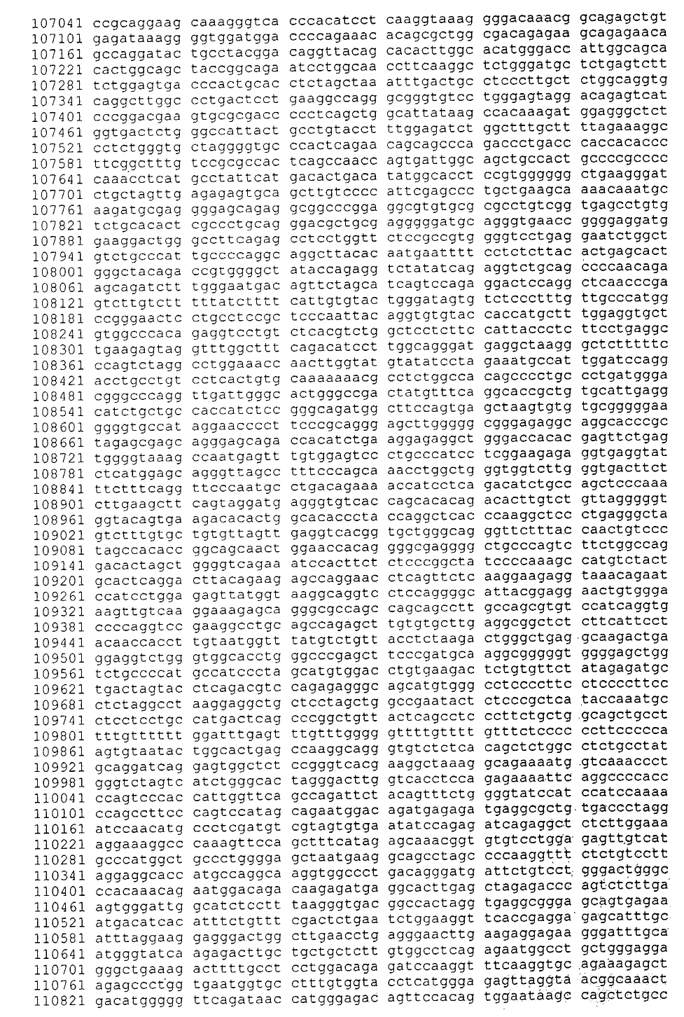

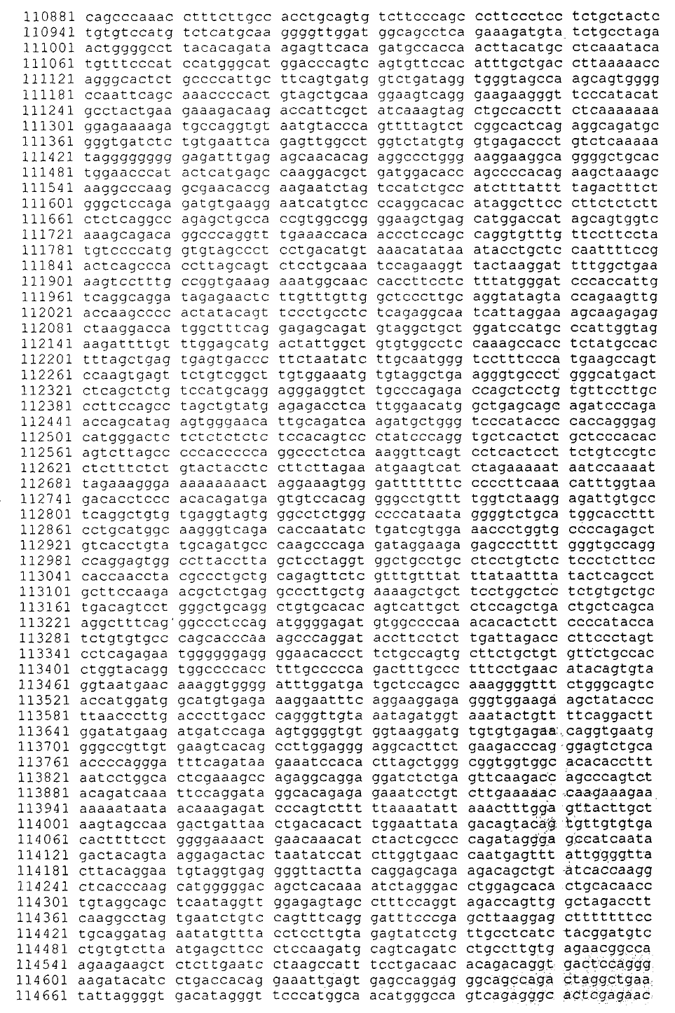

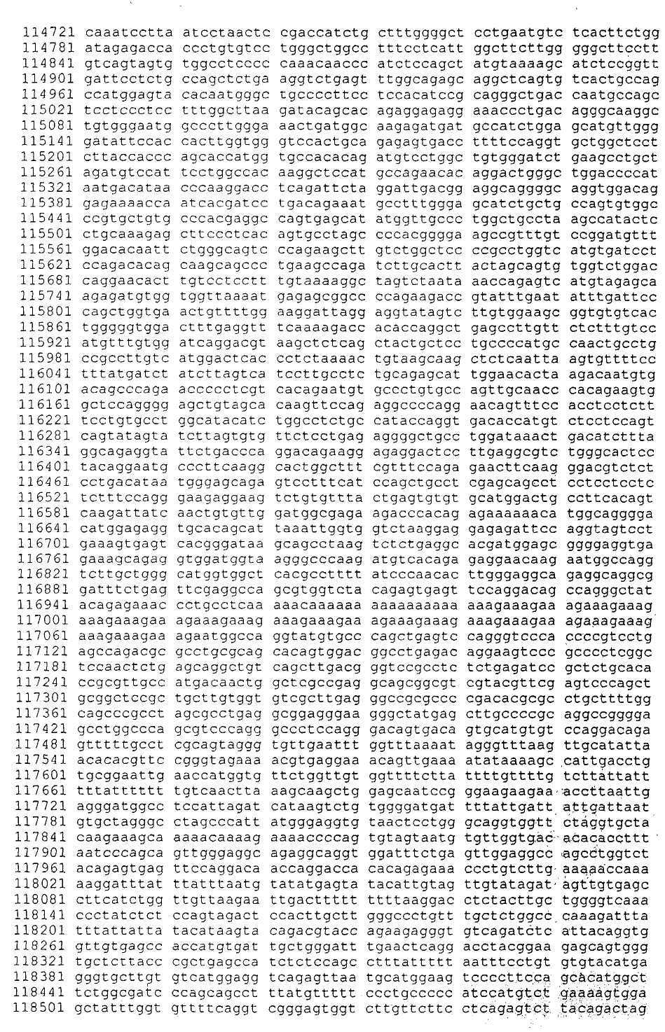

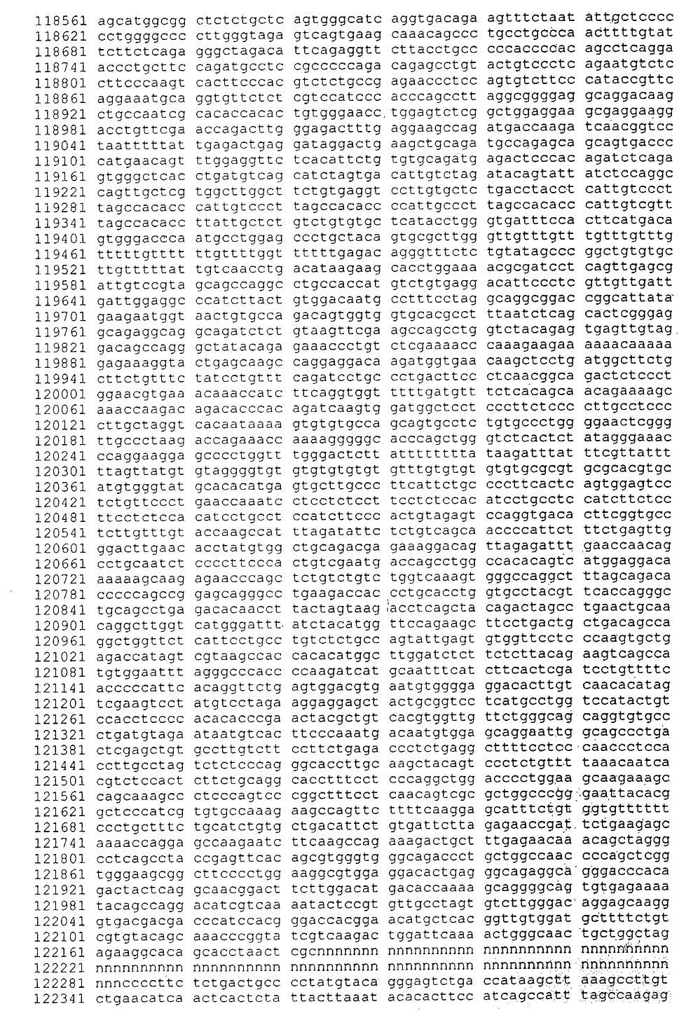

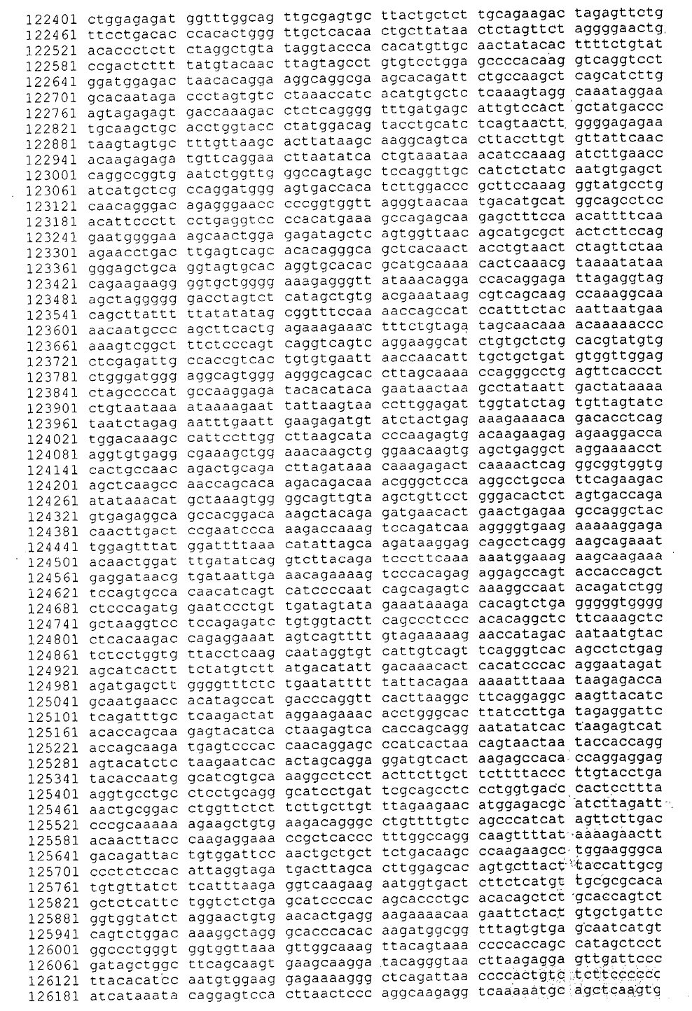

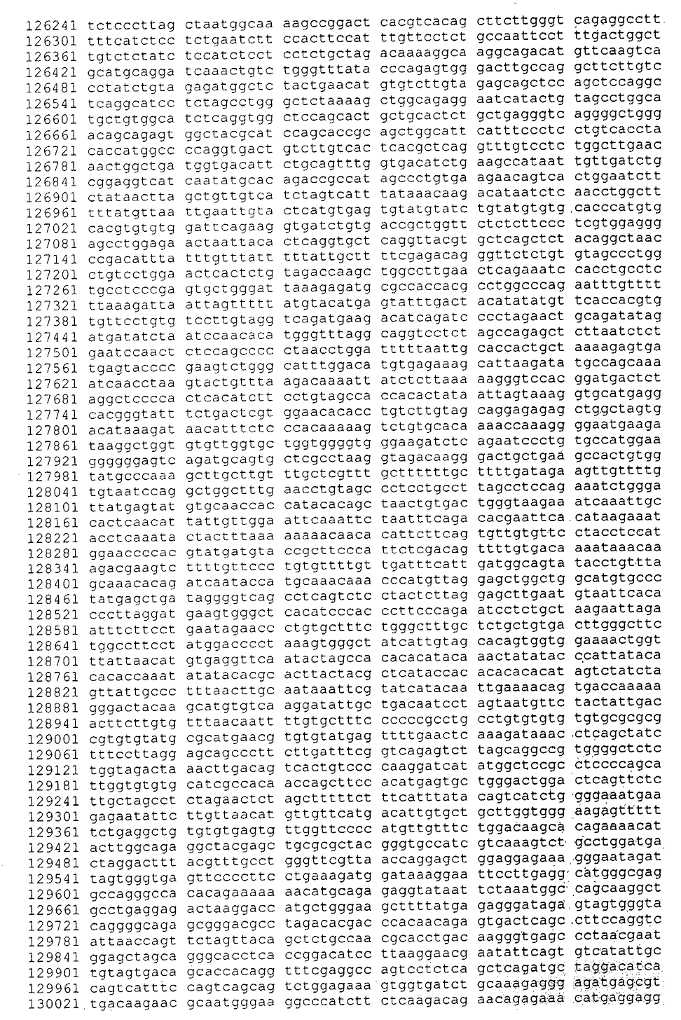

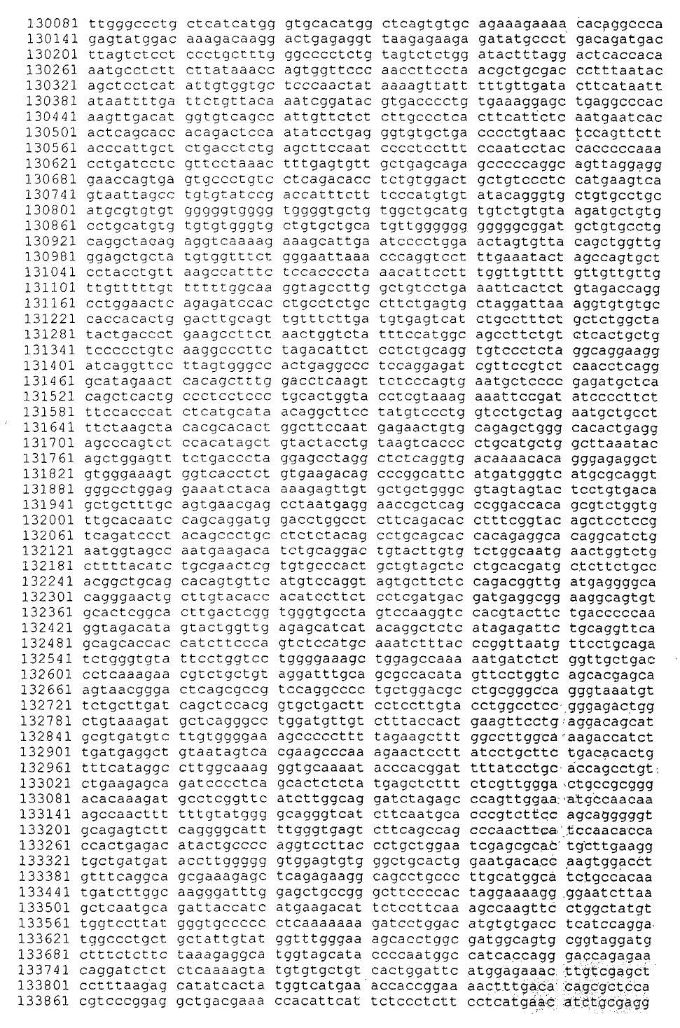

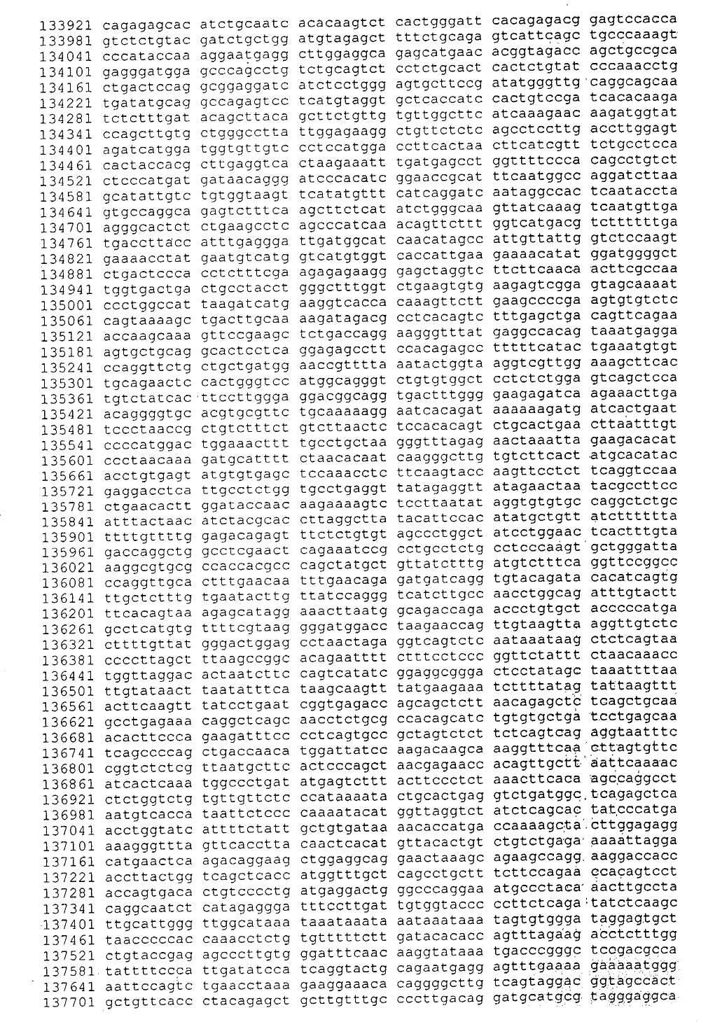

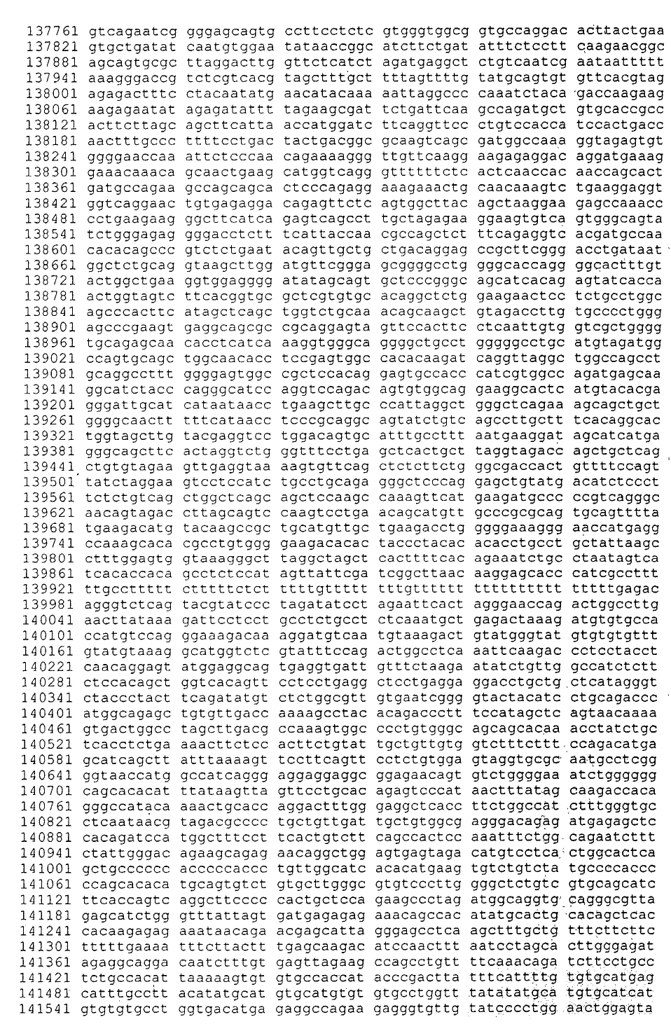

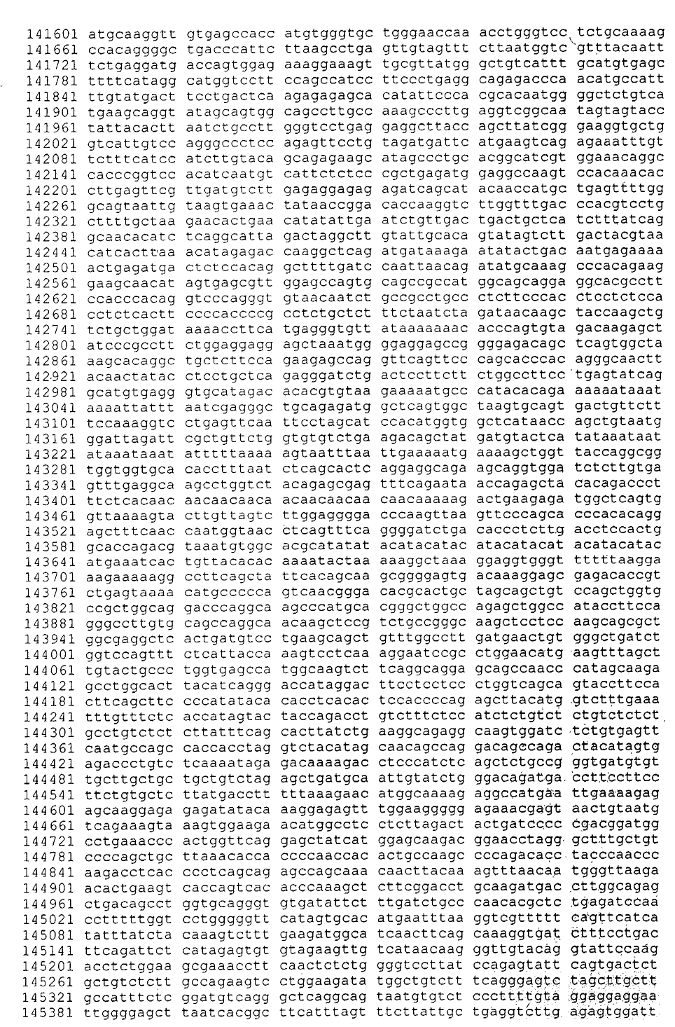

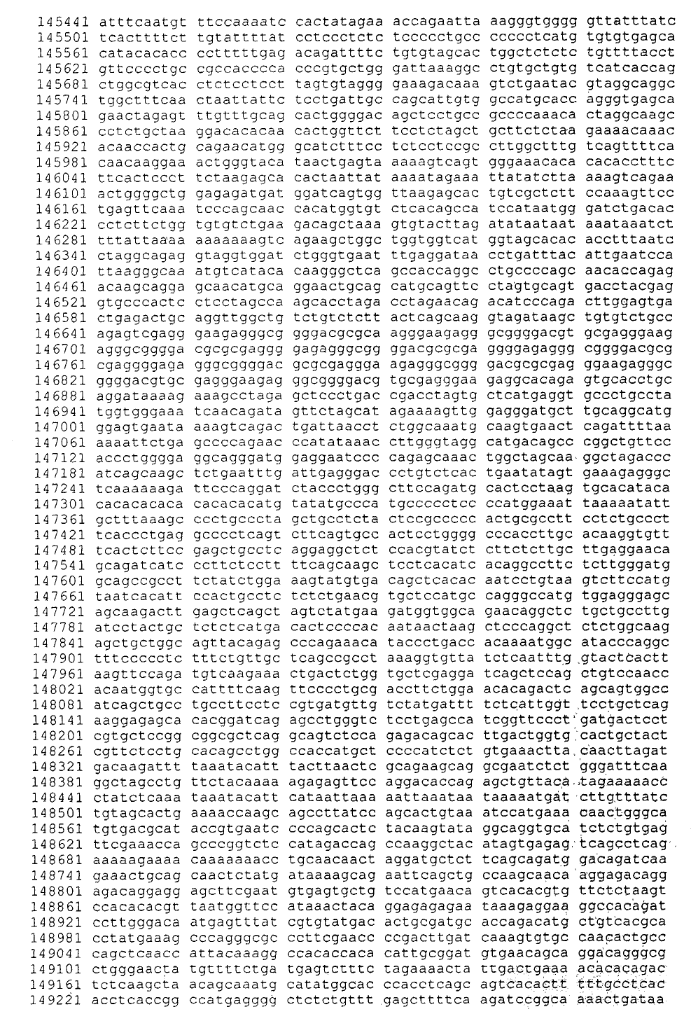

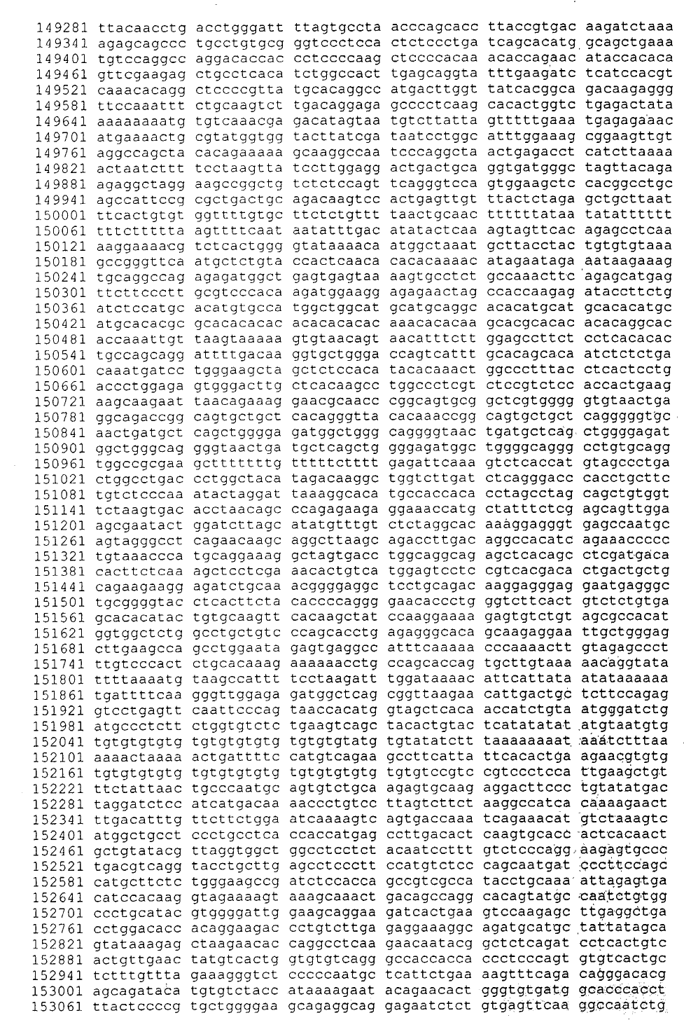

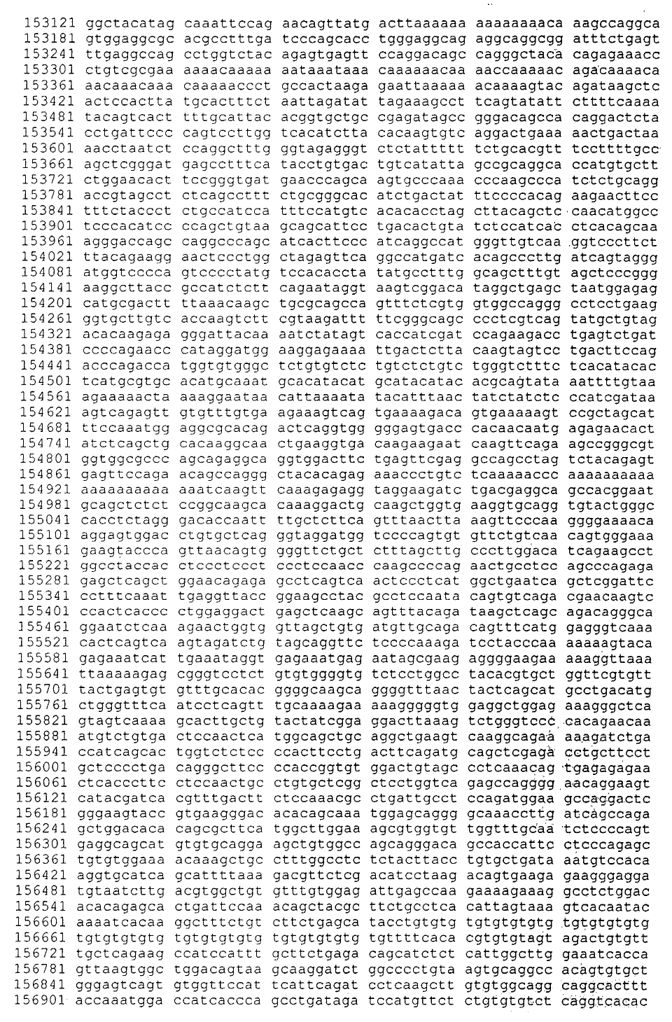

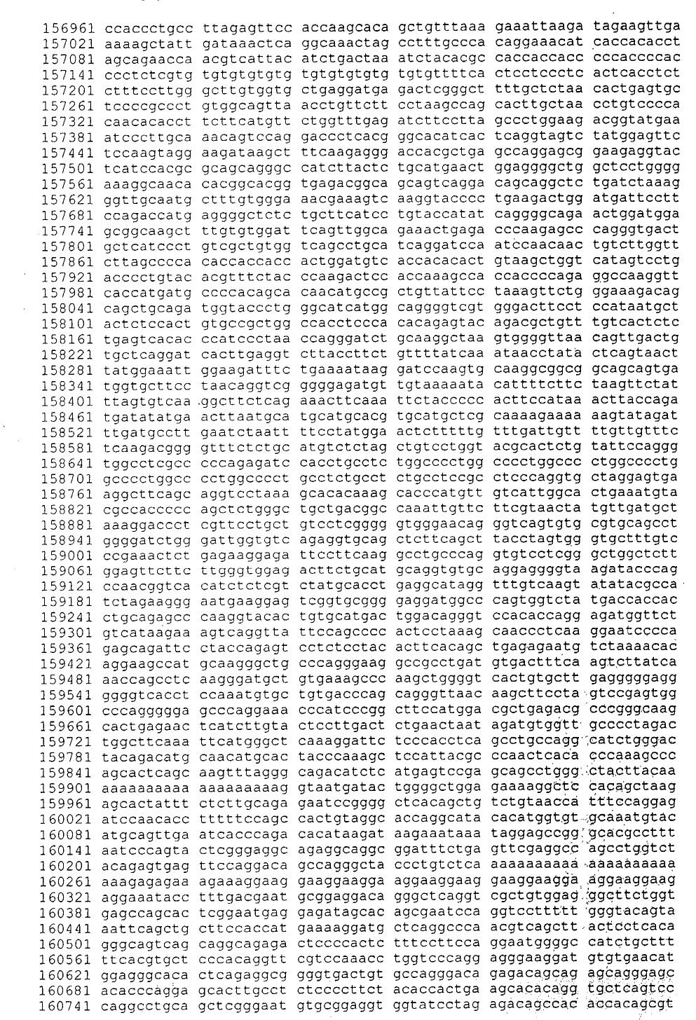

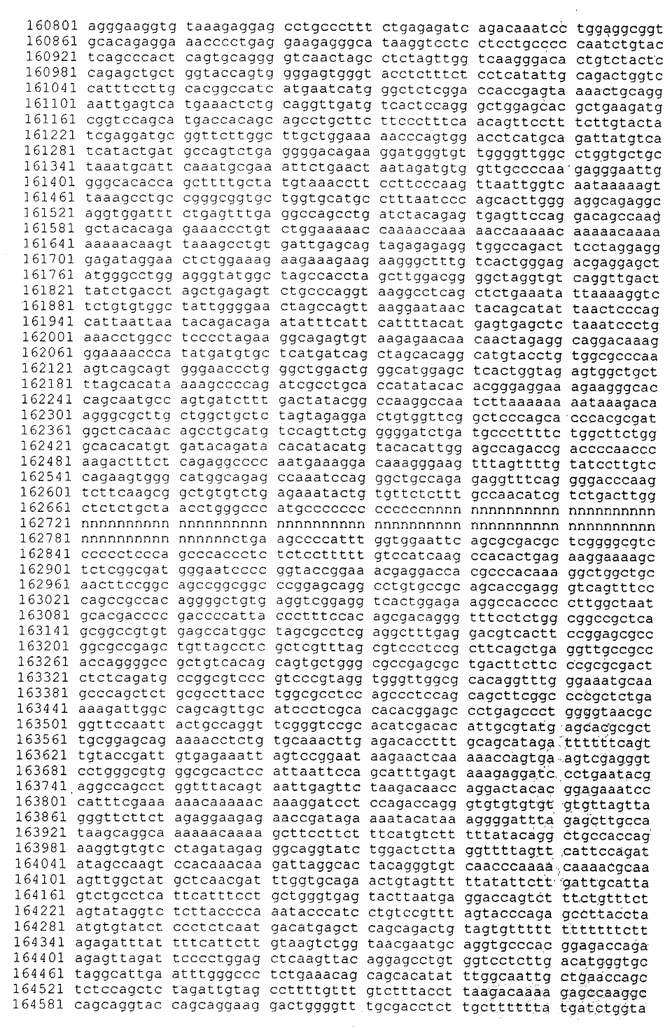

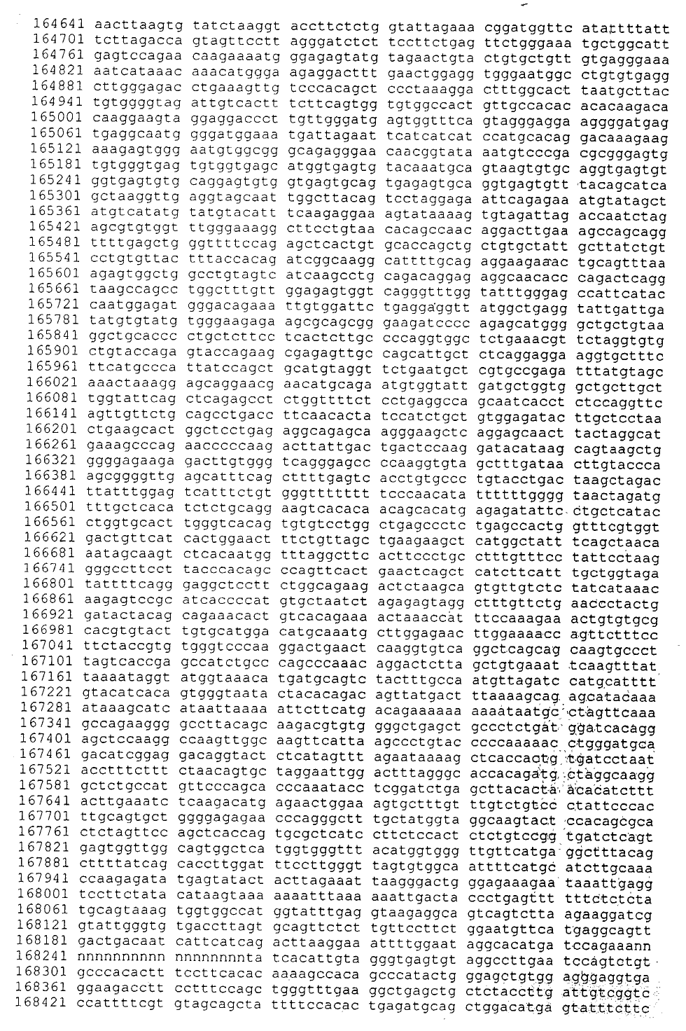

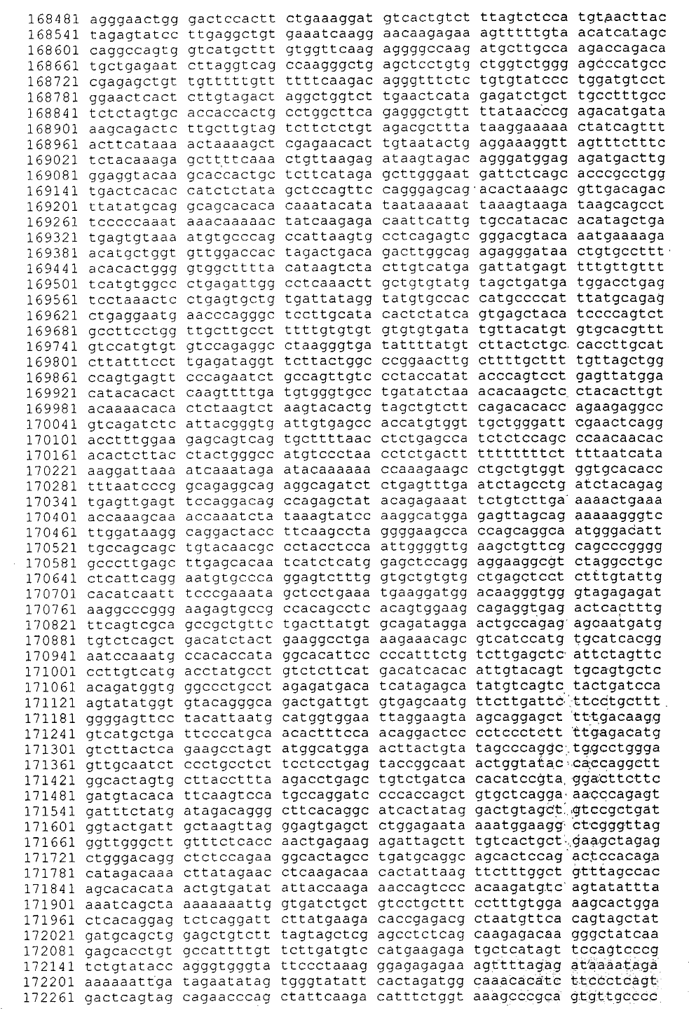

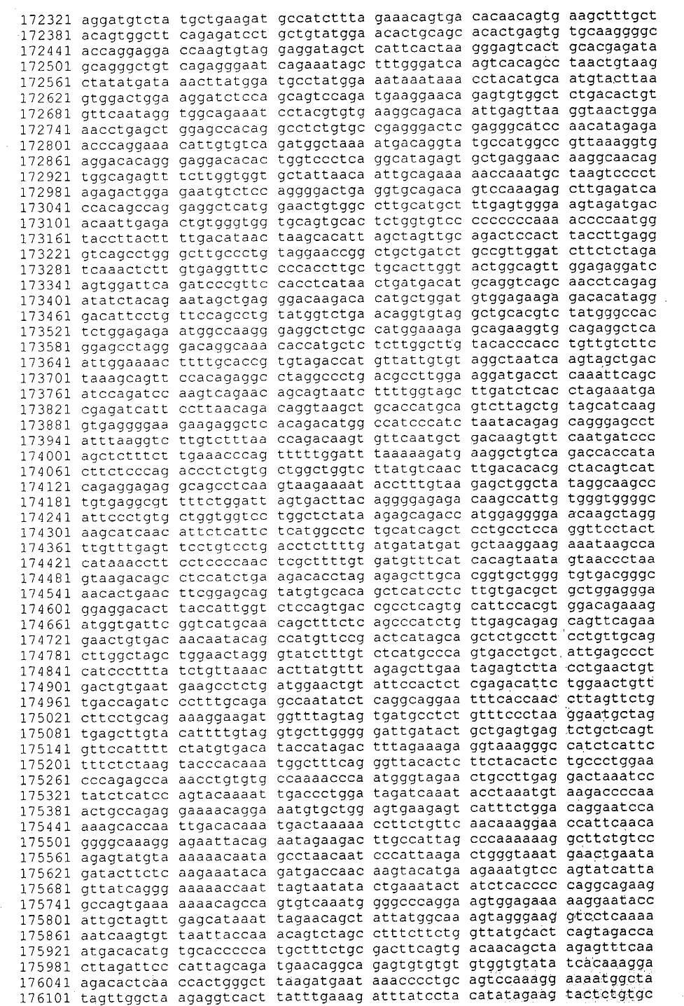

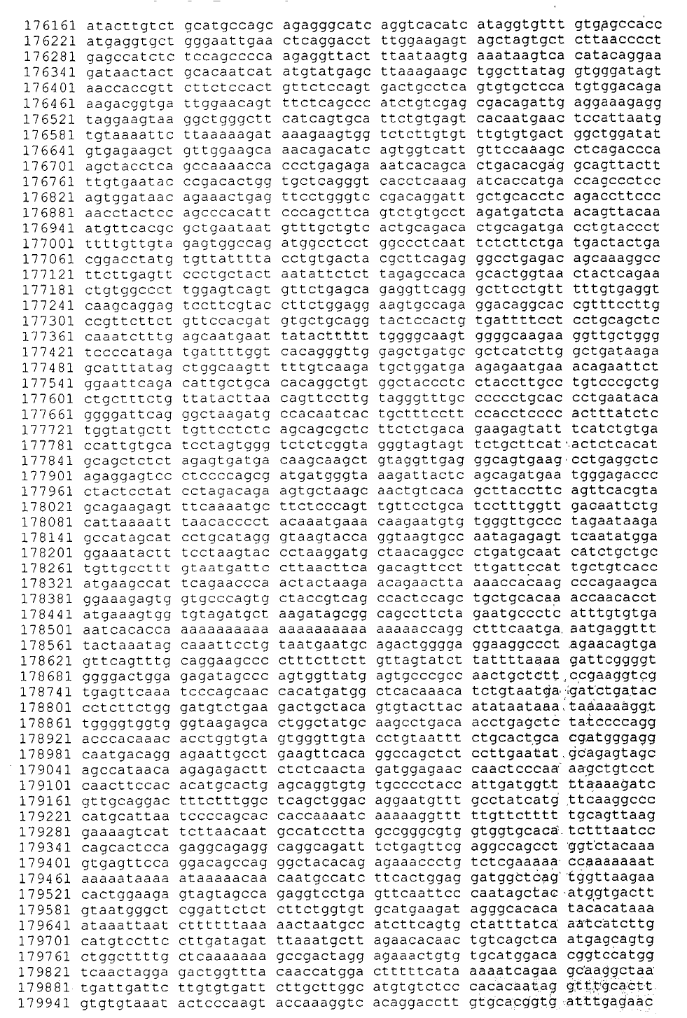

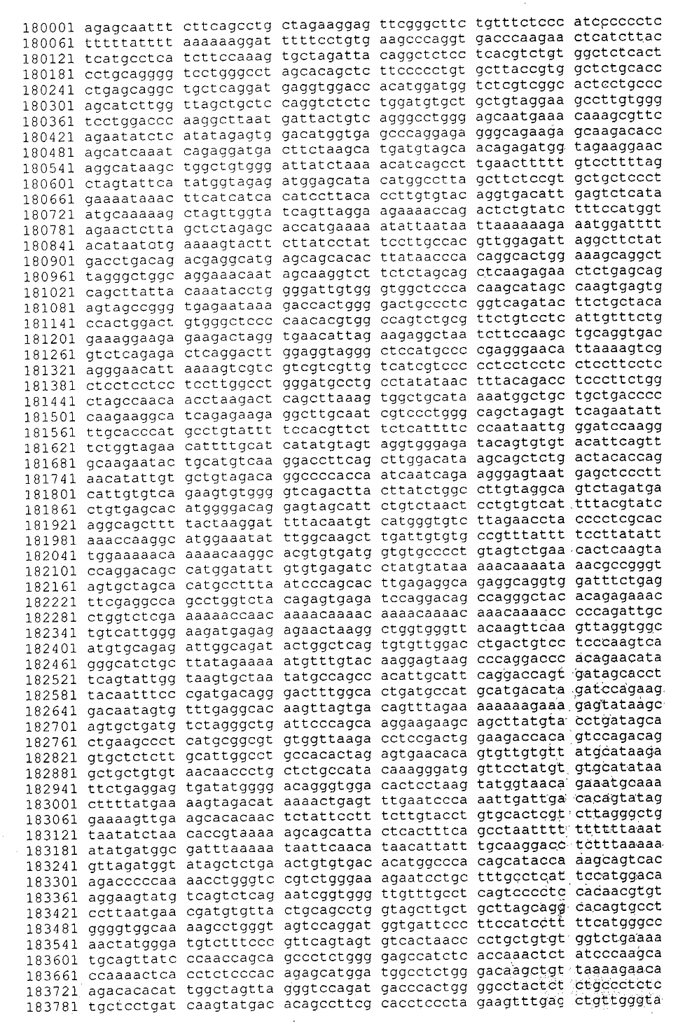

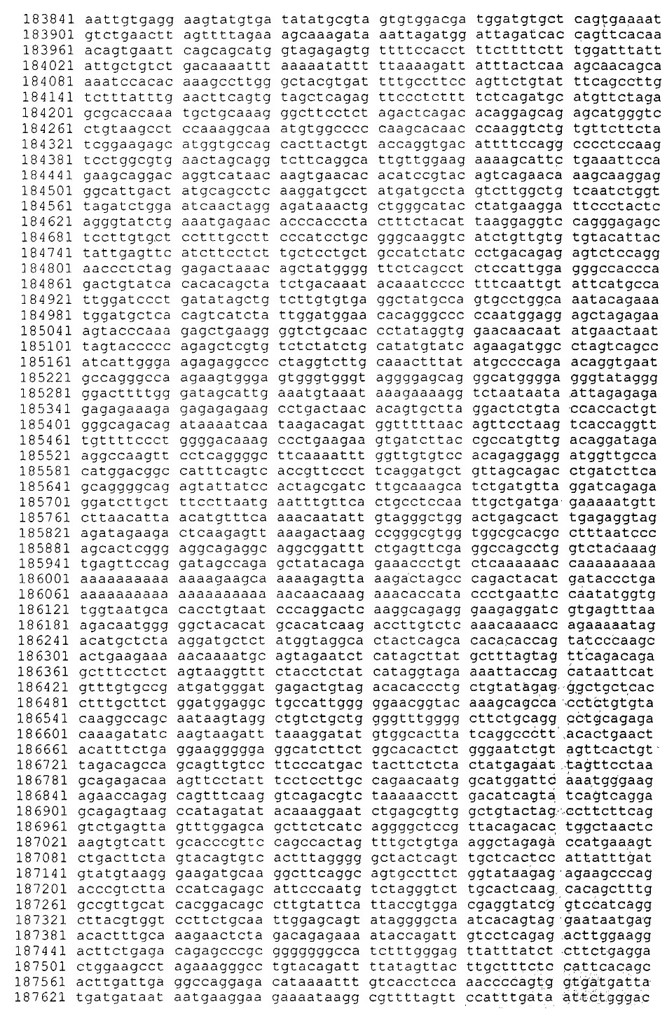

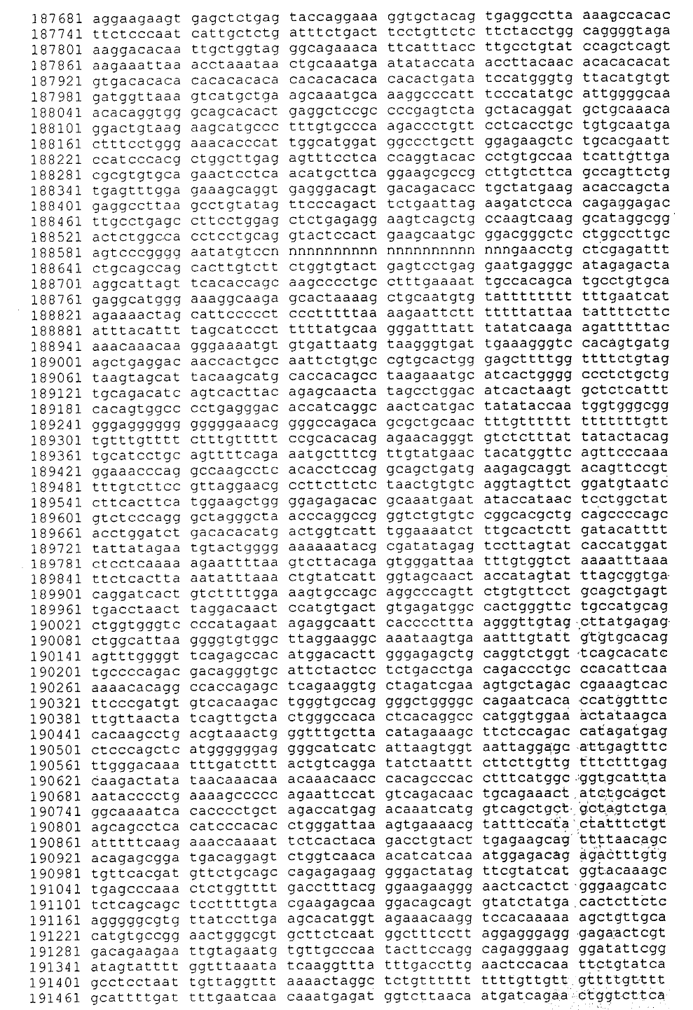

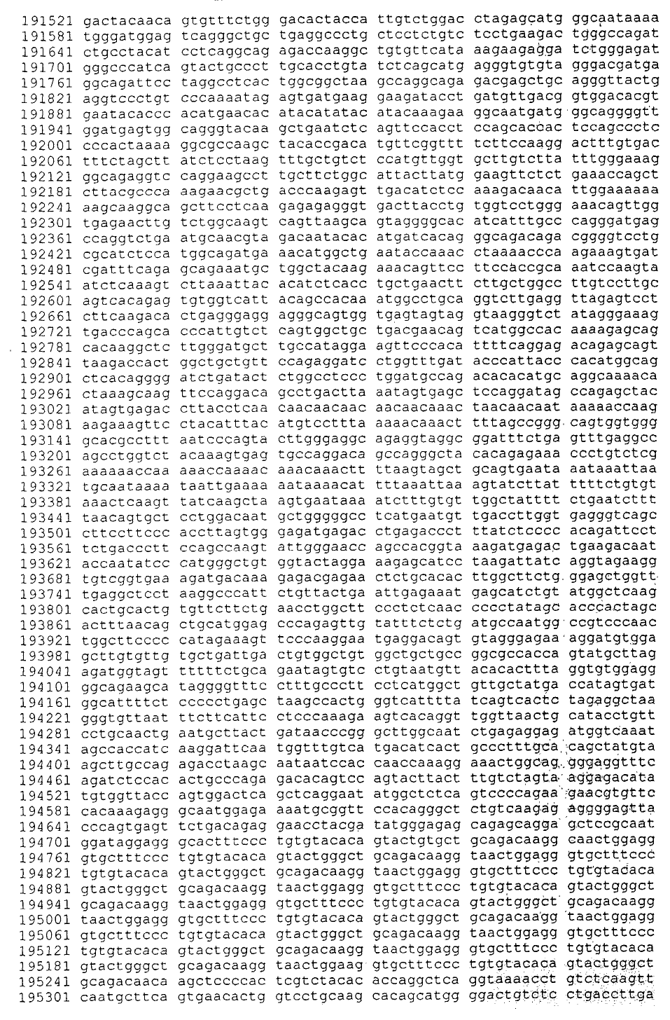

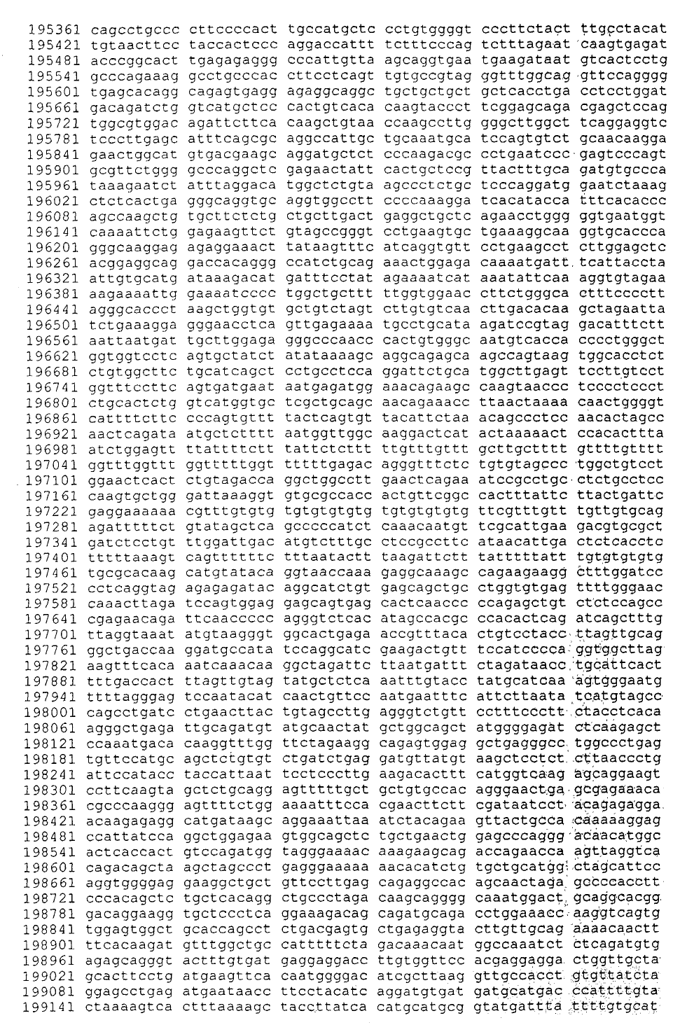

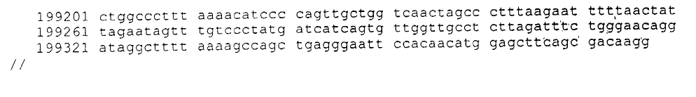

Provided herein are human (SEQ ID NOs: 1 and 2) cDNAs as well as the murine SUT-3 coding sequence (SEQ ID NOs: 3 and 9). Also provided is the genomic structure of the murine SUT-3 gene. The SUT-3 genomic nucleic acid comprises 15 exons. The exon positions of the mouse SUT-3 gene are detailed below in Table A, the positions of which correspond to the positions on the genomic clone having the accession number AC084294, the sequence of which is incorporated herein by reference and shown in FIG. 20.

TABLE A

Position on genomic

| Exon | Beginning | End |
|---|---|---|
| | 2745 | 2978 |
| | 3923 | 4115 |
| | 5242 | 5327 |
| | 8162 | 8241 |
| | 9345 | 9487 |
| | 14609 | 14784 |
| | 15108 | 15210 |
| | 16694 | 16744 |
| | 19056 | 19116 |
| | 20579 | 20634 |
| | 20859 | 21033 |
| | 21930 | 22096 |
| | 22522 | 22655 |
| | 24687 | 24759 |
| | 25013 | 25101 |

The human SUT-3 cDNAs, which are approximately 2914 and 2981 nucleotides in length respectively, encode two proteins which are approximately 606 and 650 amino acid residues in length. The mouse SUT-3 coding sequence, approximately 1821 mucleotides in length, encodes a protein of approximately 607 amino acid residues in length.

Provided also are human SUT-2 cDNAs encoding two SUT2 isoforms, said cDNAs of approximately 2896 nucleotides and 2345 nucleotides in length, respectively encoding protein isoforms of approximately 656 and 663 amino acid residues in length.

One aspect of the invention thus pertains to purified or isolated nucleic acid molecules that encode SUT-2 and SUT-3-proteins or biologically active portions thereof, as well as nucleic acid fragments thereof. Fragments may be used for example as hybridization probes to identify SUT-2 and SUT-3-encoding nucleic acids (e.g., SUT-2 or SUT-3 mRNA) and fragments for use as probes (e.g. for detection of SUT-2 or SUT-3 nucleic acid molecules) or primers (e.g. for sequencing, genotyping, amplification or mutation of SUT-2 or SUT-3 nucleic acid molecules). As used herein, the term “nucleic acids” and “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule. Also, used interchangeably herein are terms “nucleic acids”, “oligonucleotides”, and “polynucleotides”.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated SUT-2 or SUT-3 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequences as given in SEQ ID NOs: 1, 2, 3, 9, 11 or 12 or a portion thereof, can be isolated using standard molecular biology techniques and the sequence information provided herein. Using all or portion of the nucleic acid sequences in SEQ ID NOs: 1, 2, 3, 9, 11 or 12 as a hybridization probe, SUT-2 or SUT-3 nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of the sequences given in SEQ ID NOs: 1, 2, 3, 9, 11 or 12 can be isolated by the polymerase chain reaction (PCR) using synthetic oligonucleotide primers designed based upon the same sequences.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SUT-2 or SUT-3 nucleotide sequences can be prepared by standard synthetic techniques, e.g., using an automated DNA synthesizer.

In a preferred embodiment, an isolated nucleic acid molecule of the invention comprises, consists essentially of, or consists of a nucleotide sequences shown in SEQ ID NOs: 1, 2, 3, 9, 11 or 12, or fragments thereof. The sequences shown in SEQ ID NOs: 1 and 2 correspond to the human SUT-3 cDNAs, and SEQ ID NO: 3 corresponds to the mouse SUT-3 coding sequence. The sequences shown in SEQ ID NOs: 11 and 12 correspond to the human SUT-2 cDNAs. These cDNAs comprise sequences encoding the human SUT-2 and SUT-3 proteins (i.e., “the coding region”), as well as SUT-2 and SUT-3 5′ untranslated sequences and 3′ untranslated sequences. Alternatively, the nucleic acid molecule can comprise, consist essentially of, or consist of only the coding region as given in SEQ ID NOs: 1, 2, 3, 9, 11 or 12.

In a preferred embodiment, an isolated SUT-2 nucleic acid molecule of the invention comprises, consists essentially of, or consists of the nucleotide sequence shown in SEQ ID NOs: 1 or 2, or fragments thereof. The sequences of SEQ ID NOs: 11 or 12 correspond to the human SUT-2 cDNA. The SUT2 cDNAs comprise a sequence encoding the 656 amino acid isoform of human SUT-2 protein (i.e., “the coding region”, from nucleotides 241 to 2211, as well as 5′ untranslated sequences (nucleotides 1-240) and 3′ untranslated sequences (nucleotides 2212 to 2896 of SUT2 as shown in SEQ ID NO: 11). Alternatively, the nucleic acid molecule can comprise, consist essentially of, or consist of only the coding region of SEQ ID NO: 11 (e.g., nucleotides 241 to 2211). Also encompassed is a cDNA comprising sequences encoding the 663 amino acid isoform of human SUT-2 protein (i.e., “the coding region”, from nucleotides 209 to 2200, as well as 5′ untranslated sequences (nucleotides 1-208) and 3′ untranslated sequences (nucleotides 2201 to 2345). Alternatively, the nucleic acid molecule can comprise, consist essentially of, or consist of only the coding region of SEQ ID NO: 12 (e.g., nucleotides 209 to 2200). Referring to SEQ ID NO: 1, the 656-amino acid and 663-amino acid SUT2 isoforms share nucleotide sequence from respective nucleotide positions 1 to 2142, and differ in nucleotide sequence from 2143 to the end of the cDNA.

Also encompassed by the SUT-2 and SUT-3 nucleic acids of the invention are nucleic acid molecules which are complementary to SUT-2 and SUT-3 nucleic acids described herein. Preferably, a complementary nucleic acid is sufficiently complementary to the nucleotide sequence shown in SEQ ID NOs: 1, 2, 3, 9, 11 and 12 such that it can hybridize to the nucleotide sequence shown in SEQ ID NOs: 1, 2, 3, 9, 11 and 12, thereby forming a stable duplex.

Another object of the invention is a purified, isolated, or recombinant nucleic acid encoding a SUT-2 or SUT-3 polypeptide comprising, consisting essentially of, or consisting of the amino acid sequences given in SEQ ID NOs: 4, 5, 6, 10, 13, 14 or 27 or fragments thereof. For example, the purified, isolated or recombinant nucleic acid may comprise a genomic DNA or fragment thereof which encode the polypeptides in SEQ ID NOs: 4, 5, 6, 10, 13, 14 or 27 or a fragment thereof. Preferred polynucleotides of the invention also include purified, isolated, or recombinant SUT-2 and SUT-3 cDNAs consisting of, consisting essentially of, or comprising the sequences shown in SEQ ID NOs: 1, 2, 3, 9, 11 and 12 or fragments thereof. Particularly preferred nucleic acids of the invention include isolated, purified, or recombinant fragments of SUT-2 and SUT-3 nucleic acids comprising a contiguous span of at least 12, 15, 18, 20, 25, 30, 35, 40, 50, 60, 70, 80, 90, 100, 150, 200, 500, 1000 or 2000 nucleotides of the sequences in SEQ ID NOs: 1, 2, 3, 9, 11 and 12 or the complements thereof.

Moreover, the nucleic acid molecule of the invention can comprise only a portion of the nucleic acid sequences in SEQ ID NOs: 1, 2, 3, 9, 11 and 12, for example a fragment which can be used as a probe or primer or a fragment encoding a biologically active portion of a SUT-2 or SUT-3 protein. The nucleotide sequence determined from the cloning of the SUT-2 and SUT-3 genes allows for the generation of probes and primers designed for use in identifying and/or cloning other SUT-2 or SUT-3 family members, as well as SUT-3 SUT-2 or homologues from other species. The probe/primer typically comprises substantially purified oligonucleotide. The oligonucleotide typically comprises a region of nucleotide sequence that hybridizes under stringent conditions to at least about 12, preferably about 25, more preferably about 40, 50, more than 75 consecutive nucleotides of a sequence in SEQ ID NOs: 1, 2, 3, 9, 11 and 12, or a sequence complementary thereto. In an exemplary embodiment, a nucleic acid molecule of the present invention comprises a nucleotide sequence which is at least about 400, 500, 1000, preferably at least about 1000-1250, more preferably at least about 1250-1500, more preferably at least about 1500-1750, and even more preferably at least about 1750-2000 nucleotides in length and hybridizes under stringent hybridization conditions to a nucleic acid molecule in SEQ ID NOs: 1, 2, 3, 9, 11 and 12.

A nucleic acid fragment encoding a “biologically active portion of a SUT-3 protein” can be prepared by isolating a portion of the nucleotide sequence in SEQ ID NOs: 1, 2, 3, 9, 11 and 12 which encodes a polypeptide having a SUT-3 biological activity (the biological activities of the SUT-3 proteins described herein), expressing the encoded portion of the SUT-3 protein (e.g., by recombinant expression in vitro or in vivo) and assessing the activity of the encoded portion of the SUT-3 protein.

Also provided is the genomic structure of the human and murine SUT-3 genes, and the human SUT-2 gene. The exon positions on the respective human and mouse SUT-3 genes are detailed in FIG. 13, the positions of which correspond to the positions on the genomic clone having the GenBank Accession N^{o}AC123764 and N^{o}AL645911 respectively, the sequences of which is incorporated herein by reference. The partial genomic structure of the human SUT-2 gene is shown in FIG. 19, referring to GenBank Accession N^{o}NT023693. Thus, the invention embodies purified, isolated, or recombinant polynucleotides comprising a nucleotide sequence selected from the group consisting of the exons of the mouse SUT-3 gene or the human SUT-2 gene, or a sequence complementary thereto. The invention also deals with purified, isolated, or recombinant nucleic acids comprising a combination of at least two exons of the SUT-3 gene, wherein the polynucleotides are arranged within the nucleic acid, from the 5′-end to the 3′-end of said nucleic acid, in the same order as in genomic clone referenced in FIG. 13.

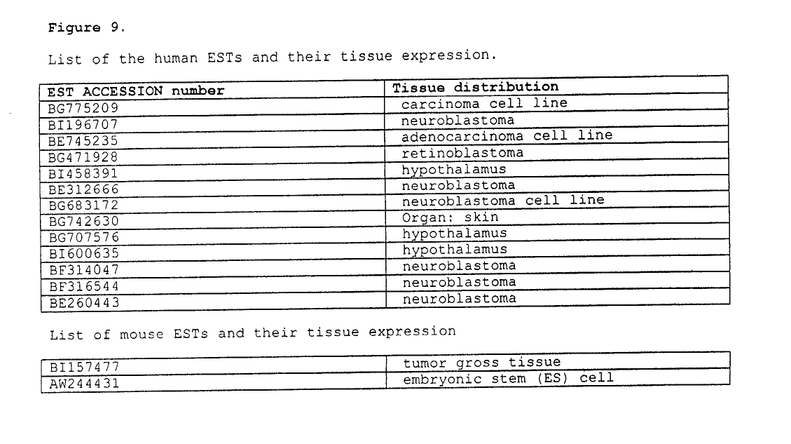

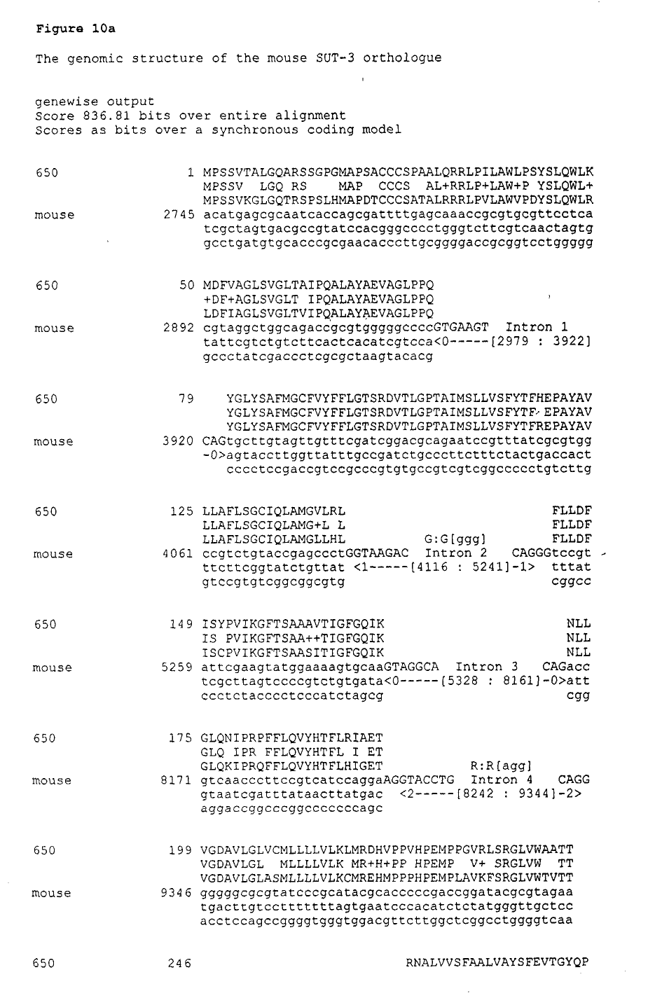

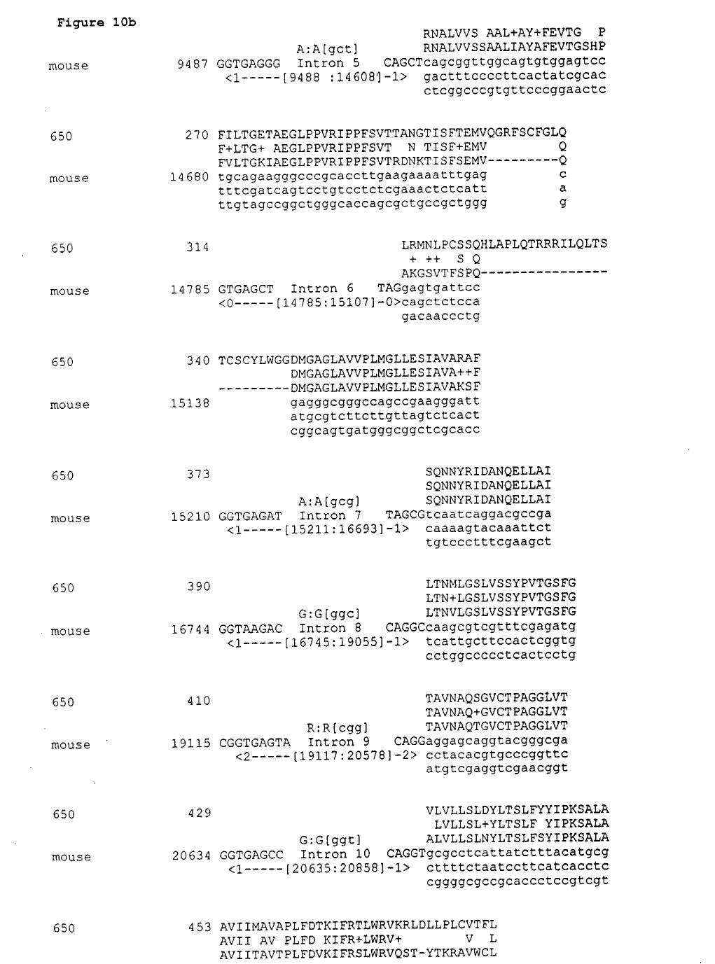

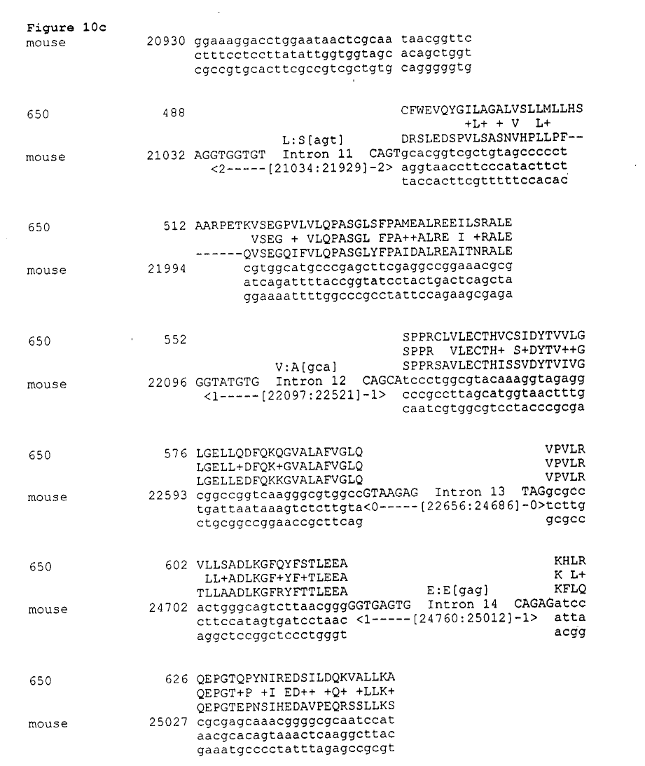

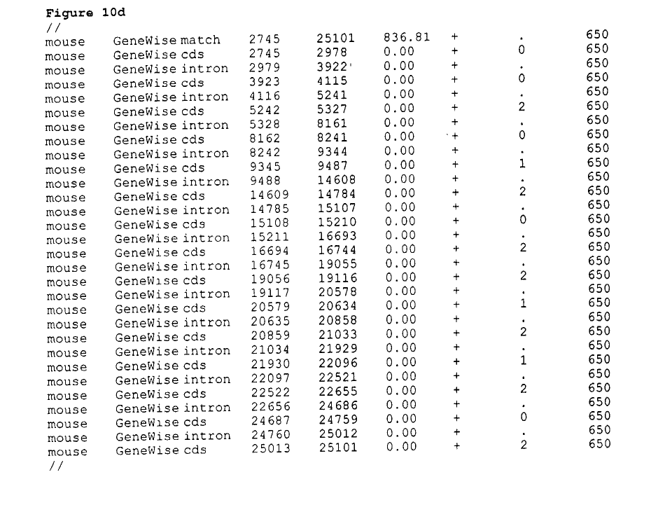

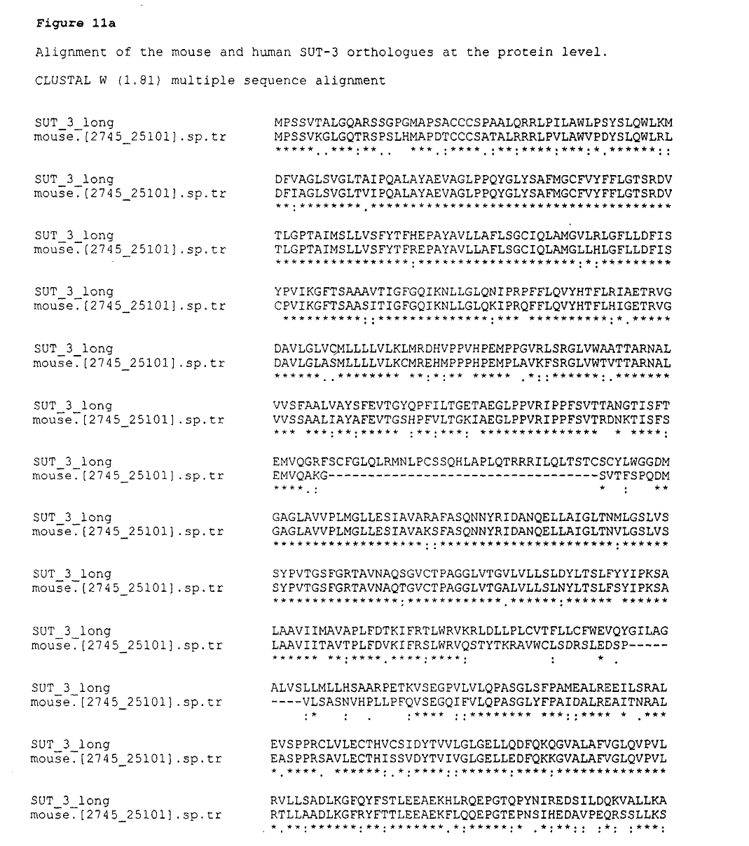

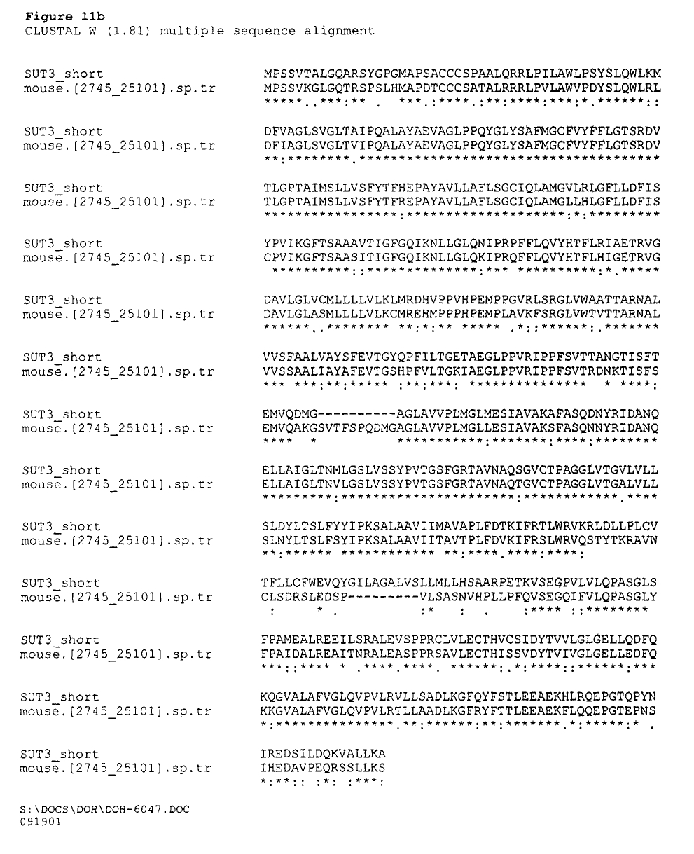

Also provided in FIG. 9 are EST sequences corresponding to SUT-3 sequences demonstrating expression in various tissues in humans and mice. Any of said ESTs sequences may be included or specifically excluded from a nucleic acid sequence of the invention.

The invention further encompasses nucleic acid molecules that differ from the nucleotide sequence shown in SEQ ID NOs: 1, 2, 3, 9, 11 or 12 due to degeneracy of the genetic code and thus encode the same SUT-3 proteins as those encoded by the nucleotide sequence shown in SEQ ID NOs: 1, 2, 3, 9, 11 or 12. In another embodiment, an isolated nucleic acid molecule of the invention comprises a nucleotide sequence encoding a protein comprising an amino acid sequence shown in SEQ ID NOs: 4, 5, 6, 10, 13, 14 or 27 or a fragment thereof.

In addition to the SUT-2 or SUT-3 nucleotide sequences shown in SEQ ID NOs: 1, 2, 3, 9, 11 or 12, it will be appreciated by those skilled in the art that DNA sequence polymorphisms that lead to changes in the amino acid sequences of the SUT-2 or SUT-3 proteins may exist within a population (e.g., the human population). Such genetic polymorphism in the SUT-2 and SUT-3 genes may exist among individuals within a population due to natural allelic variation. As used herein, the terms “gene” and “recombinant gene” refer to nucleic acid molecules comprising an open reading frame encoding a SUT-2 or SUT-3 protein, preferably a mammalian SUT-2 or SUT-3 protein. Such natural allelic variations can typically result in 1-5% variance in the nucleotide sequence of a SUT-2 or SUT-3 gene. Any and all such nucleotide variations and resulting amino acid polymorphisms in SUT-2 or SUT-3 genes that are the result of natural allelic variation and, most preferably, that do not alter the functional activity of a SUT-2 or SUT-3 protein are intended to be within the scope of the invention.

Moreover, nucleic acid molecules encoding other SUT-2 or SUT-3 family members, and thus which have a nucleotide sequence which differs from the SUT-2 or SUT-3 sequences of SEQ ID NOs: 1, 2, 3, 9, 11 or 12 are intended to be within the scope of the invention. For example, a cDNA encoding a SUT-2 or SUT-3 family member can be identified based on the nucleotide sequence of human SUT-2 or SUT-3. Moreover, nucleic acid molecules encoding SUT-2 or SUT-3 proteins from different species, and thus which have a nucleotide sequence which differs from the SUT-2 or SUT-3 sequences of SEQ ID NOs: 1, 2, 3, 9, 11 or 12 are intended to be within the scope of the invention. For example, a mouse SUT-2 or SUT-3 cDNA can be identified based on the nucleotide sequence of a human SUT-2 or SUT-3. Such SUT-2 or SUT-3 family members may be identified by hybridization to a SUT-2 or SUT-3 nucleic acid or fragment thereof, amplification with primers derived from a SUT-2 or SUT-3 nucleic acid or fragment thereof, or bioinformatic comparison with a SUT-2 or SUT-3 nucleic acid or fragment thereof or a SUT-2 or SUT-3 polypeptide or fragment thereof.

Nucleic acid molecules corresponding to natural allelic variants and homologues of the SUT-2 or SUT-3 cDNAs of the invention can be isolated based on their homology to the respective SUT-2 or SUT-3 nucleic acids disclosed herein using the cDNAs disclosed herein, or a portion thereof, as a hybridization probe according to standard hybridization techniques under stringent hybridization conditions.

As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85%, 90%, 95% or 98% homologous to each other typically remain hybridized to each other. Stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6 sodium chloride/sodium citrate (SSC) at about 45° C., followed by one or more washes in 0.2 SSC, 0.1% SDS at 50-65° C. Preferably, an isolated nucleic acid molecule of the invention that hybridizes under stringent conditions to the sequences in SEQ ID NOs: 1, 2, 3, 9, 11 or 12 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the SUT-2 or SUT-3 sequences that may exist in the population, the skilled artisan will further appreciate that changes can be introduced by mutation into the nucleotide sequences in SEQ ID NOs: 1, 2, 3, 9, 11 or 12 thereby leading to changes in the amino acid sequence of the encoded SUT-2 or SUT-3 proteins, without altering the functional ability of the SUT-2 or SUT-3 proteins. For example, nucleotide substitutions leading to amino acid substitutions at “non-essential” amino acid residues can be made in the sequences in SEQ ID NOs: 1, 2, 3, 9, 11 or 12. A “non-essential” amino acid residue is a residue that can be altered from the wild-type sequence of SUT-2 or SUT-3 (e.g., the sequences of SEQ ID NOs: 4, 5, 6, 10, 13,14 or 27) without altering the biological activity, whereas an “essential” amino acid residue is required for biological activity. For example, amino acid residues that are conserved among the SUT-2 or SUT-3 proteins of the present invention, are predicted to be less un-amenable to alteration. Furthermore, additional conserved amino acid residues may be amino acids that are conserved between the SUT-2 or SUT-3 proteins of the present invention and other members of the SLC26 family or proteins containing a sulfate transporter signature.

Accordingly, another aspect of the invention pertains to nucleic acid molecules encoding SUT-2 or SUT-3 proteins that contain changes in amino acid residues that are not essential for activity. Such SUT-2 or SUT-3 proteins differ in amino acid sequence from sequences in SEQ ID NOs: 4, 5, 6, 10, 13, 14 or 27 yet retain biological activity. In one embodiment, the isolated nucleic acid molecule comprises a nucleotide sequence encoding a protein, wherein the protein comprises an amino acid sequence at least about 60% homologous to an amino acid sequences of SEQ ID NOs: 4, 5, 6, 10, 13,14 or 27. Preferably, the protein encoded by the nucleic acid molecule is at least about 65-70% homologous to a sequence of SEQ ID NOs: 1, 2, 3, 9, 11 or 12, more preferably sharing at least about 75-80% identity with a sequences in SEQ ID NOs: 1, 2, 3, 9, 11 or 12, even more preferably sharing at least about 85%, 90%, 92%, 95%, 97%, 98%, 99% or 99.8% identity with a sequence of SEQ ID NOs: 1, 2, 3, 9, 11 or 12.

An isolated nucleic acid molecule encoding a SUT-2 or SUT-3 protein homologous to the proteins in SEQ ID NOs: 4, 5, 6, 10, 13, 14 or 27 can be created by introducing one or more nucleotide substitutions, additions or deletions into a nucleotide sequences in SEQ ID NOs: 1, 2, 3, 9, 11 or 12 such that one or more amino acid substitutions, additions or deletions are introduced into the encoded protein. Mutations can be introduced into the sequences in SEQ ID NOs: 1, 2, 3, 9, 11 or 12 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a SUT-2 or SUT-3 protein is preferably replaced with another amino acid residue from the same side chain family. Alternatively, in another embodiment, mutations can be introduced randomly along all or part of a SUT-2 or SUT-3 coding sequence, such as by saturation mutagenesis, and the resultant mutants can be screened for SUT-2 or SUT-3 biological activity to identify mutants that retain activity. Following mutagenesis of a sequence given in SEQ ID NOs: 1, 2, 3, 9, 11 or 12, the encoded protein can be expressed recombinantly and the activity of the protein can be determined.

In a preferred embodiment, a mutant SUT-2 or SUT-3 protein encoded by a SUT-2 or SUT-3 nucleic acid of the invention can be assayed for SUT-activity in any suitable assay, examples of which are provided herein.

Primers and probes of the invention can be prepared by any suitable method, including, for example, cloning and restriction of appropriate sequences and direct chemical synthesis by a method such as the phosphodiester method of Narang et al. (1979), the phosphodiester method of Brown et al. (1979), the diethylphosphoramidite method of Beaucage et al. (1981) and the solid support method described in EP 0 707 592, the disclosures of which are incorporated herein by reference in their entireties.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group.

Any of the polynucleotides of the present invention can be labeled, if desired, by incorporating any label known in the art to be detectable by spectroscopic, photochemical, biochemical, immunochemical, or chemical means. For example, useful labels include radioactive substances (including, 32P, 35S, 3H, 125I), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al. (1988) or Sanchez-Pescador et al. (1988). In addition, the probes according to the present invention may have structural characteristics such that they allow the signal amplification, such structural characteristics being, for example, branched DNA probes as those described by Urdea et al. in 1991 or in the European patent No. EP 0 225 807 (Chiron).

A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in the SUT-2 or SUT-3 gene or mRNA using other techniques.

Any of the nucleic acids, polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The nucleic acids, polynucleotides, primers and probes of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Consequently, the invention also comprises a method for detecting the presence of a nucleic acid comprising a nucleotide sequence selected from a group consisting of a sequences of SEQ ID NOs: 1, 2, 3, 9, 11 or 12, a fragment or a variant thereof and a complementary sequence thereto in a sample, said method comprising the following steps of:

a) bringing into contact a nucleic acid probe or a plurality of nucleic acid probes which can hybridize with a nucleotide sequence included in a nucleic acid selected form the group consisting of a nucleotide sequences of SEQ ID NOs: 1, 2, 3, 9, 11 or 12, a fragment or a variant thereof and a complementary sequence thereto and the sample to be assayed; and

b) detecting the hybrid complex formed between the probe and a nucleic acid in the sample.

The invention further concerns a kit for detecting the presence of a nucleic acid comprising a nucleotide sequence selected from a group consisting of a nucleotide sequences of SEQ ID NOs: 1, 2, 3, 9, 11 or 12, a fragment or a variant thereof and a complementary sequence thereto in a sample, said kit comprising:

a) a nucleic acid probe or a plurality of nucleic acid probes which can hybridize with a nucleotide sequence included in a nucleic acid selected form the group consisting of the nucleotide sequences of SEQ ID NOs: 1, 2, 3, 9, 11 or 12, a fragment or a variant thereof and a complementary sequence thereto; and

b) optionally, the reagents necessary for performing the hybridization reaction.

In a first preferred embodiment of this detection method and kit, said nucleic acid probe or the plurality of nucleic acid probes are labeled with a detectable molecule. In a second preferred embodiment of said method and kit, said nucleic acid probe or the plurality of nucleic acid probes has been immobilized on a substrate.

Any polynucleotide provided herein may be attached in overlapping areas or at random locations on a solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips, and has been generally described in U.S. Pat. No. 5,143,854; PCT publications WO 90/15070 and 92/10092.

Probes based on the SUT-2 or SUT-3 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a SUT-2 or SUT-3 protein, such as by measuring a level of a SUT-2 or SUT-3-encoding nucleic acid in a sample of cells from a subject e.g., detecting SUT-2 or SUT-3 mRNA levels or determining whether a genomic SUT-2 or SUT-3 gene has been mutated or deleted.

SUT-2 and SUT-3 Polypeptides, and Anti-SUT-2 and Anti-SUT-3 Antibodies

As used herein the terminology “SUT-2 protein or SUT-2 polypeptide” or “SUT-3 protein or SUT-3 polypeptide” encompasses any of the proteins or polypetid discussed below.

One aspect of the invention pertains to isolated SUT-2 or SUT-3 proteins, and biologically active portions thereof, as well as polypeptide fragments suitable for use as immunogens to raise anti-SUT-2 or SUT-3 antibodies. In one embodiment, native SUT-2 or SUT-3 proteins can be isolated from cells or tissue sources by an appropriate purification scheme using standard protein purification techniques. In another embodiment, SUT-2 or SUT-3 proteins are produced by recombinant DNA techniques. Alternative to recombinant expression, a SUT-2 or SUT-3 protein or polypeptide can be synthesized chemically using standard peptide synthesis techniques.

SEQ ID NOs: 4 and 5 show the short and long isoform of human SUT-3 respectively. SEQ ID NOs: 6, 10 and 27 shows the mouse SUT-3 amino acid sequence. SEQ ID NOs: 13 and 14, respectively, show the amino acid sequences of two human SUT-2 protein isoforms (656 and 663 amino acids in length).

An “isolated” or “purified” protein or biologically active portion thereof is substantially free of cellular material or other contaminating proteins from the cell or tissue source from which the SUT-2 or SUT-3 protein is derived, or substantially free from chemical precursors or other chemicals when chemically synthesized. The language “substantially free of cellular material” includes preparations of SUT-2 or SUT-3 protein in which the protein is separated from cellular components of the cells from which it is isolated or recombinantly produced. In one embodiment, the language “substantially free of cellular material” includes preparations of SUT-2 or SUT-3 protein having less than about 30% (by dry weight) of non-SUT-2 or SUT-3 protein (also referred to herein as a “contaminating protein”), more preferably less than about 20% of non-SUT-2 or SUT-3 protein, still more preferably less than about 10% of non-SUT-2 or SUT-3 protein, and most preferably less than about 5% non-SUT-2 or SUT-3 protein. When the SUT-2 or SUT-3 protein or biologically active portion thereof is recombinantly produced, it is also preferably substantially free of culture medium, i.e., culture medium represents less than about 20%, more preferably less than about 10%, and most preferably less than about 5% of the volume of the protein preparation. In one embodiment, the language “substantially free of cellular material” does not encompass preparations of SUT-2 or SUT-3 protein in which the SUT-2 or SUT-3 protein is present in an electrophoretic medium with detectable amounts of other cellular proteins. For example, in this embodiment, the language “substantially free of cellular material” does not encompass SUT-2 or SUT-3 protein which is present in a lane of a gel which also contains detectable amounts of proteins other than SUT-2 or SUT-3.

The language “substantially free of chemical precursors or other chemicals” includes preparations of SUT-2 or SUT-3 protein in which the protein is separated from chemical precursors or other chemicals which are involved in the synthesis of the protein. In one embodiment, the language “substantially free of chemical precursors or other chemicals” includes preparations of SUT-2 or SUT-3 protein having less than about 30% (by dry weight) of chemical precursors or non-SUT-2 or SUT-3 chemicals, more preferably less than about 20% chemical precursors or non-SUT-2 or SUT-3 chemicals, still more preferably less than about 10% chemical precursors or non-SUT-2 or SUT-3 chemicals, and most preferably less than about 5% chemical precursors or non-SUT-2 or SUT-3 chemicals.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

Biologically active portions of a SUT-3 protein include peptides comprising amino acid sequences sufficiently homologous to or derived from the amino acid sequence of the SUT-3 protein, e.g., an amino acid sequence shown in SEQ ID NOs: 4, 5, 6, 10 or 27 which includes less amino acids than the full length SUT-3 proteins, and exhibit at least one activity of a SUT-3 protein. For example, in some embodiments, biologically active portions of the SUT-3 protein have sulfate transporter activity. Typically, biologically active portions comprise a domain or motif with at least one activity of the SUT-3 proteins. A biologically active portion of a SUT-3 protein can be a polypeptide which is, for example at least 15, 25, 50, 100, 150, 200, 300, 400, 500, 600, or 650 or more amino acids in length.

In a preferred embodiment, the SUT-3 protein comprises, consists essentially of, or consists of the amino acid sequence shown in SEQ ID NOs: 4, 5, 6, 10 or 27. The invention also concerns the polypeptide encoded by a nucleotide sequences selected from the group consisting of the sequences in SEQ ID NOs: 1, 2, 3 or 9, a complementary sequence thereof or a fragment thereto. The present invention embodies isolated, purified, and recombinant fragments of one SUT-3 polypeptide comprising a contiguous span of at least 6 amino acids, preferably at least 8 to 10 amino acids, more preferably at least 12, 15, 20, 25, 30, 40, 50, 100,200, 300,400, 500, 600 or 650 amino acids of a sequence of SEQ ID NOs: 4, 5, 6, 10 or 27. In other preferred embodiments the contiguous stretch of amino acids comprises the site of a mutation or functional mutation, including a deletion, addition, swap or truncation of the amino acids in the SUT-3 protein sequence.

Biologically active portions of a SUT-2 protein include peptides comprising amino acid sequences sufficiently homologous to or derived from the amino acid sequence of the SUT-2 protein, e.g., the amino acid sequence shown in SEQ ID NOs: 13 or 14, which include less amino acids than the full length SUT-2 proteins, and exhibit at least one activity of a SUT-2 protein. For example, in some embodiments, biologically active portions of the SUT-2 protein have sulfate transporter activity. Typically, biologically active portions comprise a domain or motif with at least one activity of the SUT-2 protein. A biologically active portion of a SUT-2 protein can be a polypeptide which is, for example at least 15, 25, 50, 100, 150, 200, 300, 400, 500, 600, 640, 650, 660 or more amino acids in length.

In a preferred embodiment, the SUT-2 protein comprises, consists essentially of, or consists of the amino acid sequence shown in SEQ ID NOs: 13 or 14. The invention also concerns the polypeptide encoded by a nucleotide sequence selected from the group consisting of SEQ ID NOs: 11 or 12, a complementary sequence thereof or a fragment thereto. The present invention embodies isolated, purified, and recombinant fragments of one SUT-2 polypeptide comprising a contiguous span of at least 6 amino acids, preferably at least 8 to 10 amino acids, more preferably at least 12, 15, 20, 25, 30, 40, 50, 100, 200, 300, 400, 500, 600, 640, 650 or 660 amino acids of SEQ ID NOs: 13 or 14. In other preferred embodiments the contiguous stretch of amino acids comprises the site of a mutation or functional mutation, including a deletion, addition, swap or truncation of the amino acids in the SUT-2 protein sequence.

In yet other preferred embodiments, the SUT-2 polypeptide comprising a contiguous span of at least 6 amino acids, preferably at least 8 to 10 amino acids, more preferably at least 12, 15, 20, 25, 30, 40, 50, 100, 200, 300, 400, 500, 600, 640, 650 or 660 amino acids of SEQ ID NOs: 13 or 14, wherein:

a) said contiguous span of amino acids comprises a SUT2-target interaction domain;

b) said contiguous span of amino acids comprises a STAS domain;

c) said contiguous span of amino acids comprises a contiguous span of at least 6, 8, 10, 12, 15, 20, 30, 40, 50 or 60 amino acid residues of positions 571 to 637 of SEQ ID NOs: 13 or 14;

d) said contiguous span of amino acids comprises a serine at amino acid position 585 of SEQ ID NOs: 13 or 14; or

e) said contiguous span of amino acids is comprised in the carboxy terminal of the SUT2 protein, more preferably comprised between amino acid positions 400, 450, 500, 550 or 600 and the end of the SUT2 protein of SEQ ID NOs: 13 or 14.

In other embodiments, the SUT-2 or SUT-3 protein is substantially homologous to a sequence of SEQ ID NOs: 4, 5, 6, 10, 13, 14 or 27, and retains the functional activity of a protein of SEQ ID NOs: 4, 5, 6, 10, 13, 14 or 27 yet differs in amino acid sequence due to natural allelic variation or mutagenesis, as described in detail in subsection I above. Accordingly, in another embodiment, the SUT-2 or SUT-3 proteins are proteins which comprise an amino acid sequence at least about 60% homologous to an amino acid sequence of SEQ ID NOs: 4, 5, 6, 10, 13, 14 or 27 and retain the functional activity of the SUT-3 proteins of SEQ ID NOs: 4, 5, 6, 10, 13, 14 or 27. Preferably, the proteins are at least about 30%, 40%, 50%, 60%, 70%, 80%, 85%, 90%, 92%, 95%, 97%, 98%, 99.8% homologous to a protein of SEQ ID NOs: 4, 5, 6, 10, 13, 14 or 27.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence (e.g., for example when aligning a second sequence to a SUT-3 amino acid sequences of SEQ ID NOs: 4, 5 or 6 having 606 or 650 or 607 amino acid residues, at least 100, preferably at least 200, more preferably at least 300, even more preferably at least 400, and even more preferably at least 500, 600 or 650 amino acid residues are aligned or when aligning a second sequence to a SUT-3 nucleic acid sequence of SEQ ID NOs: 1, 2, 3 or 9, preferably a human or mouse SUT-3 sequence comprising, consisting essentially of or consisting of 2914 or 2981 or 1821 nucleotides which encode the amino acids of the SUT-3 protein, preferably at least 100, preferably at least 200, more preferably at least 300, even more preferably at least 400, and even more preferably at least 500, 600, at least 700, at least 800, at least 900, at least 1000, at least 1200, at least 1400, at least 1600, at least 1800, at least 2000 or more than 2000 nucleotides are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “identity” is equivalent to amino acid or nucleic acid “homology”). The percent homology between the two sequences is a function of the number of identical positions shared by the sequences (i.e., % homology=# of identical positions/total # of positions 100).

The comparison of sequences and determination of percent homology between two sequences can be accomplished using a mathematical algorithim. A preferred, non-limiting example of a mathematical algorithim utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77, the disclosures of which are incorporated herein by reference in their entireties. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to SUT-2 or SUT-3 nucleic acid molecules of the invention. BLAST protein searches can be performed with the XBLAST program, score=50, wordlength=3 to obtain amino acid sequences homologous to SUT-2 or SUT-3 protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov, the disclosures of which are incorporated herein by reference in their entireties. Another preferred, non-limiting example of a mathematical algorithim utilized for the comparison of sequences is the algorithm of Myers and Miller, CABIOS (1989), the disclosures of which are incorporated herein by reference in their entireties. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides SUT-2 or SUT-3 chimeric or fusion proteins. As used herein, a SUT-2 or SUT-3 “chimeric protein” or “fusion protein” comprises a SUT-2 or SUT-3 polypeptide operatively linked, preferalby fused in frame, to a non-SUT-2 or non-SUT-3 polypeptide. In a preferred embodiment, a SUT-2 or SUT-3 fusion protein comprises at least one biologically active portion of a SUT-2 or SUT-3 protein. In another preferred embodiment, a SUT-2 or SUT-3 fusion protein comprises at least two biologically active portions of a SUT-2 or SUT-3 protein. For example, in one embodiment, the fusion protein is a GST-SUT-2 or GST-SUT-3 fusion protein in which the SUT-2 or SUT-3 sequences are fused to the C-terminus of the GST sequences. Such fusion proteins can facilitate the purification of recombinant SUT-2 or SUT-3. In another embodiment, the fusion protein is a SUT-2 or SUT-3 protein containing a heterologous signal sequence at its N-terminus, such as for example to allow for a desired cellular localization in a certain host cell.

The SUT-2 or SUT-3 fusion proteins of the invention can be incorporated into pharmaceutical compositions and administered to a subject in vivo. Moreover, the SUT-2 or SUT-3-fusion proteins of the invention can be used as immunogens to produce anti-SUT-2 or anti-SUT-3 antibodies in a subject, to purify SUT-2 or SUT-3 ligands and in screening assays to identify molecules which inhibit the interaction of SUT-2 or SUT-3 with respective SUT-2 or SUT-3 target molecule.

The present invention also pertains to variants of the SUT-2 or SUT-3 proteins which function as either SUT-2 or SUT-3 mimetics or as SUT-2 or SUT-3 inhibitors. Variants of the SUT-2 or SUT-3 proteins can be generated by mutagenesis, e.g., discrete point mutation or truncation of a SUT-2 or SUT-3 protein. An agonist of the SUT-2 or SUT-3 proteins can retain substantially the same, or a subset, of the biological activities of the naturally occurring form of a SUT-2 or SUT-3 protein. An antagonist of a SUT-2 or SUT-3 protein can inhibit one or more of the activities of the naturally occurring form of the SUT-2 or SUT-3 protein by, for example, competitively inhibiting the sulfate transport activity of a SUT-2 or SUT-3 protein. Thus, specific biological effects can be elicited by treatment with a variant of limited function. In one embodiment, variants of a SUT-2 or SUT-3 protein which function as either SUT-2 or SUT-3 agonists (mimetics) or as SUT-2 or SUT-3 antagonists can be identified by screening combinatorial libraries of mutants, e.g., truncation mutants, of a SUT-2 or SUT-3 protein for SUT-2 or SUT-3 protein agonist or antagonist activity. In one embodiment, a variegated library of SUT-2 or SUT-3 variants is generated by combinatorial mutagenesis at the nucleic acid level and is encoded by a variegated gene library. A variegated library of SUT-2 or SUT-3 variants can be produced by, for example, enzymatically ligating a mixture of synthetic oligonucleotides into gene sequences such that a degenerate set of potential SUT-2 or SUT-3 sequences is expressible as individual polypeptides, or alternatively, as a set of larger fusion proteins (e.g., for phage display) containing the set of SUT-2 or SUT-3 sequences therein. There are a variety of methods which can be used to produce libraries of potential SUT-2 or SUT-3 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SUT-2 or SUT-3 sequences.

In addition, libraries of fragments of a SUT-2 or SUT-3 protein coding sequence can be used to generate a variegated population of SUT-2 or SUT-3 fragments for screening and subsequent selection of variants of a SUT-2 or SUT-3 protein. In one embodiment, a library of coding sequence fragments can be generated by treating a double stranded PCR fragment of a SUT-2 or SUT-3 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the SUT-2 or SUT-3 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SUT-2 or SUT-3 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected.

In one embodiment, cell based assays can be exploited to analyze a variegated SUT-2 or SUT-3 library. For example, a library of expression vectors can be transfected into a Xenopus laevis cell line according to the Examples, and sulfate transport activity is assessed.

An isolated SUT-2 or SUT-3 protein, or a portion or fragment thereof, can be used as an immunogen to generate antibodies that bind SUT-2 or SUT-3 using standard techniques for polyclonal and monoclonal antibody preparation. A full-length SUT-2 or SUT-3 protein can be used or, alternatively, the invention provides antigenic peptide fragments of SUT-2 or SUT-3 for use as immunogens. Any fragment of the SUT-2 or SUT-3 proteins which contains at least one antigenic determinant may be used to generate antibodies. The antigenic peptide of SUT-2 or SUT-3 comprises at least 8 amino acid residues of the amino acid sequences shown in SEQ ID NOs: 4, 5, 6, 10, 13, 14 or 27 and encompasses an epitope of SUT-2 or SUT-3 such that an antibody raised against the peptide forms a specific immune complex with SUT-2 or SUT-3. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of SUT-2 or SUT-3 that are located on the surface of the protein, e.g., hydrophilic regions.

A SUT-2 or SUT-3 immunogen typically is used to prepare antibodies by immunizing a suitable subject, (e.g., rabbit, goat, mouse or other mammal) with the immunogen. An appropriate immunogenic preparation can contain, for example, recombinantly expressed SUT-2 or SUT-3 protein or a chemically synthesized SUT-2 or SUT-3 polypeptide. The preparation can further include an adjuvant, such as Freund's complete or incomplete adjuvant, or similar immunostimulatory agent. Immunization of a suitable subject with an immunogenic SUT-2 or SUT-3 preparation induces a polyclonal anti-SUT-2 or anti-SUT-3 antibody response.

Accordingly, another aspect of the invention pertains to anti-SUT-2 or anti-SUT-3 antibodies. The term “antibody” as used herein refers to immunoglobulin molecules and immunologically active portions of immunoglobulin molecules, i.e., molecules that contain an antigen binding site which specifically binds (immunoreacts with) an antigen, such as SUT-2 or SUT-3. Examples of immunologically active portions of immunoglobulin molecules include F(ab) and F(ab′)2 fragments which can be generated by treating the antibody with an enzyme such as pepsin. The invention provides polyclonal and monoclonal antibodies that bind SUT-2 or SUT-3. The term “monoclonal antibody” or “monoclonal antibody composition”, as used herein, refers to a population of antibody molecules that contain only one species of an antigen binding site capable of immunoreacting with a particular epitope of SUT-2 or SUT-3. A monoclonal antibody composition thus typically displays a single binding affinity for a particular SUT-2 or SUT-3 protein with which it immunoreacts.

The invention concerns antibody compositions, either polyclonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, 100, or more than 100 amino acids in a sequence of SEQ ID NOs: 4, 5, 6, 10, 13, 14 or 27. The invention also concerns a purified or isolated antibody capable of specifically binding to a mutated SUT-3 proteins or to a fragment or variant thereof comprising an epitope of the mutated SUT-3 proteins.

Polyclonal anti-SUT-2 or anti-SUT-3 antibodies can be prepared as described above by immunizing a suitable subject with a SUT-2 or SUT-3 immunogen. The anti-SUT-2 or anti-SUT-3 antibody titer in the immunized subject can be monitored over time by standard techniques, such as with an enzyme linked immunosorbent assay (ELISA) using immobilized SUT-2 or SUT-3. If desired, the antibody molecules directed against SUT-2 or SUT-3 can be isolated from the mammal (e.g., from the blood) and further purified by well known techniques, such as protein A chromatography to obtain the IgG fraction. At an appropriate time after immunization, e.g., when the anti-SUT-2 or anti-SUT-3 antibody titers are highest, antibody-producing cells can be obtained from the subject and used to prepare monoclonal antibodies by standard techniques, such as those described in the following references, the disclosures of which are incorporated herein by reference in their entireties: the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36), the disclosures of which are incorporated herein by reference in their entireties. Briefly, an immortal cell line (typically a myeloma) is fused to lymphocytes (typically splenocytes) from a mammal immunized with a SUT-2 or SUT-3 immunogen as described above, and the culture supernatants of the resulting hybridoma cells are screened to identify a hybridoma producing a monoclonal antibody that binds SUT-2 or SUT-3.

Any of the many well known protocols used for fusing lymphocytes and immortalized cell lines can be applied for the purpose of generating an anti-SUT-2 or anti-SUT-3 monoclonal antibody (see, e.g., G. Galfre et al. (1977) Nature 266:55052; Gefter et al. Somatic Cell Genet., cited supra; Lerner, Yale J Biol. Med, cited supra; Kenneth, Monoclonal Antibodies, cited supra), the disclosures of which are incorporated herein by reference in their entireties. Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind SUT-2 or SUT-3, e.g., using a standard ELISA assay.

Alternative to preparing monoclonal antibody-secreting hybridomas, a monoclonal anti-SUT-2 or anti-SUT-3 antibody can be identified and isolated by screening a recombinant combinatorial immunoglobulin library (e.g., an antibody phage display library) with SUT-2 or SUT-3 to thereby isolate immunoglobulin library members that bind SUT-2 or SUT-3. Kits for generating and screening phage display libraries are commercially available (e.g., the Pharmacia Recombinant Phage Antibody System, Catalog No. 27-9400-01; and the Stratagene SurfZAP.TM. Phage Display Kit, Catalog No. 240612), the disclosures of which are incorporated herein by reference in their entireties. Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-SUT-2 or anti-SUT-3 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060, the disclosures of which are incorporated herein by reference in their entireties.

An anti-SUT-2 or SUT-3 antibody (e.g., monoclonal antibody) can be used to isolate SUT-2 or SUT-3 by standard techniques, such as affinity chromatography or immunoprecipitation. An anti-SUT-2 or anti-SUT-3 antibody can facilitate the purification of natural SUT-2 or SUT-3 from cells and of recombinantly produced SUT-2 or SUT-3 expressed in host cells. Moreover, an anti-SUT-2 or SUT-3 antibody can be used to detect SUT-2 or SUT-3 protein (e.g., in a cellular lysate or cell supernatant) in order to evaluate the abundance and pattern of expression of the SUT-2 or SUT-3 protein. Anti-SUT-2 or anti-SUT-3 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Recombinant Expression Vectors and Host Cells

Another aspect of the invention pertains to vectors, preferably expression vectors, containing a nucleic acid encoding a SUT-2 or SUT-3 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a SUT-2 or SUT-3 nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990), the disclosure of which is incorporated herein by reference in its entirety. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SUT-2 or SUT-3 proteins, mutant forms of SUT-2 or SUT-3 proteins, fusion proteins, or fragments of any of the preceding proteins, etc.).

The recombinant expression vectors of the invention can be designed for expression of SUT-2 or SUT-3 proteins in prokaryotic or eukaryotic cells. For example, SUT-2 or SUT-3 proteins can be expressed in bacterial cells such as E. coli, insect cells (using baculovirus expression vectors) yeast cells, or mammalian cells. Suitable host cells are discussed further in Goeddel, Gene Expression Technology: Methods in Enzymology 185, Academic Press, San Diego, Calif. (1990), the disclosure of which is incorporated herein by reference in its entirety. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes:1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.), the disclosures of which are incorporated herein by reference in their entireties, which fuse glutathione S-transferase (GST), maltose E binding protein, or protein A, respectively, to the target recombinant protein.

Purified fusion proteins can be utilized in SUT-2 or SUT-3 activity assays, (e.g., direct assays or competitive assays described in detail below), or to generate antibodies specific for SUT-2 or SUT-3 proteins, for example. In a preferred embodiment, a SUT-2 or SUT-3 fusion protein expressed in a retroviral expression vector of the present invention can be utilized to infect bone marrow cells which are subsequently transplanted into irradiated recipients. The pathology of the subject recipient is then examined after sufficient time has passed (e.g six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89), the disclosures of which are incorporated herein by reference in their entireties. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn 1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128, the disclosure of which is incorporated herein by reference in its entirety). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118, the disclosure of which is incorporated herein by reference in its entirety). Such alteration of nucleic acid sequences of the invention can be carried out by standard DNA synthesis techniques.

In another embodiment, the SUT-2 or SUT-3 expression vector is a yeast expression vector. Examples of vectors for expression in yeast S. cerivisae include pYepSec 1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.), the disclosures of which are incorporated herein by reference in their entireties.

Alternatively, SUT-2 or SUT-3 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39), the disclosures of which are incorporated herein by reference in their entireties. In particularly preferred embodiments, SUT-2 or SUT-3 proteins are expressed according to Karniski et al, Am. J. Physiol. (1998) 275: F79-87, the disclosure of which is incorporated herein by reference in its entirety.

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195), the disclosures of which are incorporated herein by reference in their entireties. When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989, the disclosure of which is incorporated herein by reference in its entirety.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277, the disclosure of which is incorporated herein by reference in its entirety), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the alpha-fetoprotein promoter (Campes and Tilghman (1989) Genes Dev. 3:537-546), the disclosures of which are incorporated herein by reference in their entireties.

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to SUT-2 or SUT-3 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986, the disclosure of which is incorporated herein by reference in its entirety.

Another aspect of the invention pertains to host cells into which a recombinant expression vector of the invention has been introduced. The terms “host cell” and “recombinant host cell” are used interchangeably herein. It is understood that such term refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a SUT-2 or SUT-3 protein can be expressed in bacterial cells such as E. coli, insect cells, yeast or mammalian cells (such as Chinese hamster ovary cells (CHO) or COS cells or human cells). Other suitable host cells are known to those skilled in the art, including Xenopus laevis oocytes as further described in the Examples.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989, the disclosure of which is incorporated herein by reference in its entirety), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a SUT-2 or SUT-3 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a SUT-2 or SUT-3 protein. Accordingly, the invention further provides methods for producing a SUT-2 or SUT-3 protein using the host cells of the invention. In one embodiment, the method comprises culturing the host cell of invention (into which a recombinant expression vector encoding a SUT-2 or SUT-3 protein has been introduced) in a suitable medium such that a SUT-2 or SUT-3 protein is produced. In another embodiment, the method further comprises isolating a SUT-2 or SUT-3 protein from the medium or the host cell.

In another embodiment, the invention encompasses providing a cell capable of expressing a SUT-2 or SUT-3 protein, culturing said cell in a suitable medium such that a SUT-2 or SUT-3 protein is produced, and isolating or purifying the SUT-2 or SUT-3 protein from the medium or cell.

The host cells of the invention can also be used to produce nonhuman transgenic animals. For example, in one embodiment, a host cell of the invention is a fertilized oocyte or an embryonic stem cell into which SUT-2 or SUT-3-coding sequences have been introduced. Such host cells can then be used to create non-human transgenic animals in which exogenous SUT-2 or SUT-3 sequences have been introduced into their genome or homologous recombinant animals in which endogenous SUT-2 or SUT-3 sequences have been altered. Such animals are useful for studying the function and/or activity of a SUT-2 or SUT-3 polypeptide or fragment thereof and for identifying and/or evaluating modulators of SUT-2 or SUT-3 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous SUT-2 or SUT-3 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a SUT-2 or SUT-3-encoding nucleic acid into the male pronuclei of a fertilized oocyte, e.g., by microinjection or retroviral infection, and allowing the oocyte to develop in a pseudopregnant female foster animal. The SUT-2 or SUT-3 cDNA sequence or a fragment thereof such as a sequences of SEQ ID NOs: 1, 2, 3, 9, 11 or 12 can be introduced as a transgene into the genome of a non-human animal. Alternatively, a nonhuman homologue of a human SUT-2 or SUT-3 gene, such as a mouse or rat SUT-2 or SUT-3 gene, can be used as a transgene. Intronic sequences and polyadenylation signals can also be included in the transgene to increase the efficiency of expression of the transgene. A tissue-specific regulatory sequence(s) can be operably linked to a SUT-2 or SUT-3 transgene to direct expression of a SUT-2 or SUT-3 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986, the disclosure of which is incorporated herein by reference in its entirety). Similar methods are used for production of other transgenic animals. A transgenic founder animal can be identified based upon the presence of a SUT-2 or SUT-3 transgene in its genome and/or expression of SUT-2 or SUT-3 mRNA in tissues or cells of the animals. A transgenic founder animal can then be used to breed additional animals carrying the transgene. Moreover, transgenic animals carrying a transgene encoding a SUT-2 or SUT-3 protein can further be bred to other transgenic animals carrying other transgenes.

To create an animal in which a desired nucleic acid has been introduced into the genome via homologous recombination, a vector is prepared which contains at least a portion of a SUT-2 or SUT-3 gene into which a deletion, addition or substitution has been introduced to thereby alter, e.g., functionally disrupt, the SUT-2 or SUT-3 gene. The SUT-2 or SUT-3 gene can be a human gene (e.g., the cDNAs of SEQ ID NOs: 1, 2, 3, 9, 11 or 12), but more preferably, is a non-human homologue of a human SUT-2 or SUT-3 gene (e.g., a cDNA isolated by stringent hybridization with a nucleotide sequence of SEQ ID NOs: 1, 2, 3, 9, 11 or 12). For example, a mouse SUT-2 or SUT-3 gene can be used to construct a homologous recombination vector suitable for altering an endogenous SUT-2 or SUT-3 gene in the mouse genome. In a preferred embodiment, the vector is designed such that, upon homologous recombination, the endogenous SUT-2 or SUT-3 gene is functionally disrupted (i.e., no longer encodes a functional protein; also referred to as a “knock out” vector). Alternatively, the vector can be designed such that, upon homologous recombination, the endogenous SUT-2 or SUT-3 gene is mutated or otherwise altered but still encodes functional protein (e.g., the upstream regulatory region can be altered to thereby alter the expression of the endogenous SUT-2 or SUT-3 protein). In the homologous recombination vector, the altered portion of the SUT-2 or SUT-3 gene is flanked at its 5′ and 3′ ends by additional nucleic acid sequence of the SUT-2 or SUT-3 gene to allow for homologous recombination to occur between the exogenous SUT-2 or SUT-3 gene carried by the vector and an endogenous SUT-2 or SUT-3 gene in an embryonic stem cell. The additional flanking SUT-2 or SUT-3 nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503, the disclosure of which is incorporated herein by reference in its entirety, for a description of homologous recombination vectors). The vector is introduced into an embryonic stem cell line (e.g., by electroporation) and cells in which the introduced SUT-2 or SUT-3 gene has homologously recombined with the endogenous SUT-2 or SUT-3 gene are selected (see e.g., Li, E. et al. (1992) Cell 69:915, the disclosure of which is incorporated herein by reference in its entirety). The selected cells are then injected into a blastocyst of an animal (e.g., a mouse) to form aggregation chimeras (see e.g., Bradley, A. in Teratocarcinomas and Embryonic Stem Cells. A Practical Approach, E. J. Robertson, ed. (IRL, Oxford, 1987) pp. 113-152, the disclosure of which is incorporated herein by reference in its entirety). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al., the disclosures of which are incorporated herein by reference in their entireties.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236, the disclosure of which is incorporated herein by reference in its entirety. Another example of a recombinase system is the FLP recombinase system of Saccharomyces cerevisiae (O'Gorman et al. (1991) Science 251:1351-1355, the disclosure of which is incorporated herein by reference in its entirety). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Drug Screening Assays

The invention provides a method (also referred to herein as a “screening assay”) for identifying inhibitors or activators, i.e., candidate or test compounds or agents (e.g., preferably small molecules, but also peptides, peptidomimetics or other drugs) which bind to SUT-2 or SUT-3 proteins, have an inhibitory or activating effect on, for example, SUT-2 or SUT-3 expression or preferably SUT-2 or SUT-3 activity, or have an inhibitory or activating effect on, for example, the activity of an SUT-2 or SUT-3 target molecule. Any of the SUT-2 or SUT-3 proteins, polypeptides or family members or biologically active portions thereof discussed herein may be used in any of the assays described herein. Likewise, any polypeptide comprising the Pfam and/or STAS domains of any of the SUT-2 or SUT-3 proteins or polypeptides or polypeptides comprising an amino acid sequence having at least at least about 50-60%, at least about 60-70%, 70-80%, 80%, 90%, 95%, 97%, 98% at least about 99.8% identity or similarity to the Pfam and/or STAS domains of any of the SUT-2 or SUT-3 proteins or polypeptides described herein may be used in any of the assays.

In some embodiments small molecules can be generated using combinatorial chemistry or can be obtained from a natural products library. Assays may be cell based or non-cell based assays. Drug screening assays may be binding assays or more preferentially functional assays, as further described. The compounds identified using the methods of the present invention may have sufficient potency to be effective therapeutic agents or may be lead compounds which can be further optimized to generate effective therapeutic compounds.

In preferred embodiments, an assay is a cell-based assay in which a cell which expresses a SUT-2 or SUT-3 protein or biologically active portion thereof is contacted with a test compound and the ability of the test compound to inhibit, activate, or increase SUT-2 or SUT-3 activity determined. Determining the ability of the test compound to inhibit, activate, or increase SUT-2 or SUT-3 activity can be accomplished by monitoring the bioactivity of the SUT-2 or SUT-3 protein or biologically active portion thereof. Preferably, sulfate uptake by the cell is monitored. The cell, for example, can be of mammalian origin, bacterial origin or a yeast cell. For example, in some embodiments, the cell can be a mammalian cell, bacterial cell or yeast cell which has been engineered to lack or have reduced endogenous sulfate transport activity or which naturally lacks or has a low level of sulfate transport activity. Preferably, the cell is a Xenopus laevis Oocyte or an insect cell, preferably an sf9 cell as further detailed herein.

Functional Sulfate Transport Assays

In a preferred embodiment, the invention provides cell-based drug screening assays in Xenopus Oocytes or insect (Sf9) cells. See for example Markovich et al, (1993) 90:8073-8077 PNAS USA, the disclosure of which is incorporated herein by reference in its entirety. These assays can be used for drug screening in a similar manner to those used to assess the ability of SUT-2 or SUT-3 to mediate sulfate transport as demonstrated herein. These assays are particularly suited for drug screening as many compounds can be conveniently assayed for the ability to inhibit sulfate uptake by the cells. The assay is further described in Example 2 and Example 5.

Sulfate transport assays can be carried out by analyzing ³⁵S sulfate uptake into Xenopus oocytes after injection of SUT-2 or SUT-3 cRNA and in the presence of a test compound. Decrease of sulfate transport relative to sulfate transport levels in the absence of said compound indicates a candidate SUT-2 or SUT-3 inhibitor. Increased sulfate transport relative to that in the absence of a test compound indicates a candidate SUT-2 or SUT-3 activator. SUT-2 or SUT-3 protein for injection can be prepared by any suitable means, including using an in vitro translation system such as the rabbit reticulocyte lysate system used in the present examples. The handling of Xenopus oocytes and the sulfate transport assay are further described in Markovich et al, (1993) Proc. Natl. Acad. Sci. USA 90, 8073-8077; Bissig, M et al, (1994) J. Biol. Chem. 269, 3017-3021; and Silberg et al, (1995) J. Biol. Chem. 270, 11897-11902, the disclosures of which are incorporated herein by reference in their entireties.

In other asssays, sulfate transport can be assayed in insect cells as in Karniski et al, (1998) Am. J. Physiol. 275: F79-F87. A baculovirus vector comprising a SUT-2 or SUT-3 nucleic acid sequence can be prepared and used to infect Sf9 cells. The infected cells can then be used to determine uptake of ³⁵S sulfate, the labelled sulfate being detected for example by scintillation spectroscopy. In some embodiments, the cell naturally lacks significant endogenous sulfate transport activity. In other embodiments, cell has been engineered to lack significant sulfate transport activity. For example, the genes encoding sulfate transporters in the cells may have been knocked out, deleted or mutated. For example, sulfate transport can be assayed in mammalian cells, bacterial cells, or yeast cells. For example, in some embodiments, the cell can be a mammalian cell, bacterial cell or yeast cell which has been engineered to lack sulfate transport activity or which naturally lacks sulfate transport activity.

Thus, in preferred embodiments, the invention provides a method of identifying a candidate SUT-2 or SUT-3 inhibitor or activator, said method comprising a) providing a cell comprising a SUT-2 or SUT-3 polypeptide or a fragment thereof; b) contacting said cell with a test compound; and c) determining whether said compound selectively inhibits or activates SUT-2 or SUT-3 activity relative to cells which were not contacted with the compound. Preferably the method comprises a) providing a Xenopus laevis oocyte; b) introducing SUT-2 or SUT-3 cRNA into said Xenopus oocyte; c) contacting said Xenopus oocyte with a test compound; and d) detecting sulfate uptake by said Xenopus oocyte. A determination that said compound inhibits sulfate uptake relative to cells which were not contacted with the compound indicates that said compound is a candidate SUT-2 or SUT-3 inhibitor. In another embodiment, the method comprises a) providing an insect cell; b) introducing a baculovirus vector comprising a nucleic acid sequence encoding SUT-2 or SUT-3 or a fragment thereof into said cell; c) contacting said cell with a test compound; and d) detecting sulfate uptake by said cell. Again, a determination that said compound inhibits sulfate uptake relative to cells which were not contacted with the compound indicates that said compound is a candidate SUT-2 or SUT-3 inhibitor. A determination that said compound increases sulfate uptake relative to cells which were not contacted with the compound indicates that said compound is a candidate SUT-2 or SUT-3 activator.

Further Assays and SUT-2 or SUT-3 Inhibitor Compounds

The invention further encompasses compounds capable of inhibiting or activating SUT-2 or SUT-3 activity. Preferably, a SUT-2 or SUT-3 inhibitor or activator is a selective SUT-2 or SUT-3 inhibitor or activator. In other embodiments, an SUT-2 or SUT-3 inhibitor is capable of inhibiting or increasing the activity of or binding to more than one (e.g. at least two, three, four) sulfate transporter proteins. In some embodiments, the compound is not a general anion exchange inhibitor or activator. Assays of the invention may be used to screen any suitable collection of compounds.

In a preferred embodiment, an inhibitor or activator is capable of inhibiting or increasing a SUT-2 or SUT-3 activity selected from the group consisting of (i) anion exchange activity, (e.g. chloride ion exchange activity); (ii) sulfate ion transport activity, more preferably cellular sulfate uptake, preferably uptake by a Xenopus laevis oocyte or an Sf9 cell, optionally by a HEVEC. In other preferred embodiments, a SUT-2 or SUT-3 inhibitor or activator is capable of inhibiting or increasing (1) sulfation of L-selectin ligands, preferably sialomucin-type L-selectin counter receptors; (2) L-selectin dependent lymphocyte adhesion to cells comprising a L-selectin ligand, preferably HEVs or HEVECs; (3) interactions between L-selectin ligands, preferably sialomucin-type L-selectin counter receptors and L-selectin; and/or (4) adhesion of lymphocytes to endothelial cells, preferably to HEVs or HEVECs.

Sulfation of L-selectin ligands, preferably sialomucin type L-selectin counter-receptors, may be measured by performing metabolic labeling of lymph nodes with radioactive sulfate in the presence or absence of a test compound or a known or suspected SUT-2 or SUT-3 inhibitor or activator and analyzing sulfate incorporation into sialomucin type L-selectin counterreceptors such as CD34 by immunoprecipitation as well as westen blotting with the HEVEC-specific sulfate-dependent antibody MECA-79 as described in Imai et al., Nature, 361 :555-557, 1993 and Hemmerich et al., J Exp Med, 180 :2219-2226, 1994, the disclosures of which are incorporated herein by reference in their entireties.

L-selectin dependent lymphocyte adhesion to cells comprising a L-selectin ligand preferably HEVs or HEVECs may be measured by performing an in vitro Stamper-Woodruff assay of lymphocyte adhesion to HEVs from human tonsils or lymph nodes in frozen sections (Stamper H B, Woodruff J J, J Exp Med, 144 :828-833, 1976, the disclosure of which is incorporated herein by reference in its entirety) in the presence or absence of a test compound or a known or suspected SUT-2 or SUT-3 inhibitor or activator. The assay is performed with human lymphocytes or lymphocytic cell lines expressing L-selectin and frozen sections from human tonsils or lymph nodes treated with SUT-2 or SUT-3 inhibitors.

Interactions between L-selectin ligands, preferably sialomucin-type L-selectin counterreceptors and L-selectin may be measured by metabolic labeling of lymph nodes with radioactive sulfate in the presence or absence of a test compound or a known or suspected SUT-2 or SUT-3 inhibitor or activator and analysis of L-selectin binding to sialomucin type L-selectin counterreceptors by immunoprecipitation with an IgG-L-selectin chimera as described in Watson et al., J Cell Biol, 110 :2221-2229, 1990 and Imai et al., Nature, 361:555-557, 1993, the disclosures of which are incorporated herein by reference in their entireties.

Adhesion of lymphocytes to HEVs or HEVECs may in preferred embodiments be assessed by observing the ‘rolling phenotype’ in vivo in mouse lymph node HEVs in the presence or absence of a test compound or a known or suspected SUT-2 or SUT-3 inhibitor or activator using intravital microscopy (microscopy on live animals). (von Andrian (1996) Microcirculation (3):287-300; and von Andrian U H, M'Rini C. (1998) Cell Adhes Commun. 6(2-3):85-96), the disclosures of which are incorporated herein by reference in their entireties. In one embodiment, a rolling assay is performed.

In brief, the different steps of lymphocyte migration through HEVs (tethering, rolling, sticking, transendothelial migration) are analyzed by intravital microscopy in mice treated with function blocking antibodies against SUT-2 or SUT-3 ,with test compounds or with small molecule inhibitors or activators known to be or suspected of being specific for SUT-2 or SUT-3. In some embodiments, lymphocyte migration in the presence of the test compound, antibody or known or suspected SUT-2 or SUT-3 activator or inhibitor is compared to lymphocyte migration in the absence of the test compound, antibody or known or suspected SUT-2 or SUT-3 activator or inhibitor. For observation of lymph nodes HEVs vessels (and adhesion processes occurring in these vessels) by intravital microscopy, a microsurgical exposition of the subiliac (superficial inguinal) lymph node is made on an anaesthetized mouse. A catheterization of the right jugular vein and the left carotid artery used for administration of drugs and cardiovascular monitoring is also completed. Another catheter in the femoral artery contralateral to the prepared LN allows retrograde injection, into the iliac artery which supplies the subiliac LN, of fluorescent cells, dyes, beads or antibodies depending on the experiments. The mice are then transferred to a customized video microscopy setup and the lymph nodes are put under the lenses. To visualize by intravital microscopy either interactions between cells and vessels (tethering, rolling, sticking) or expression of cell adhesion molecules at the surface of endothelial cells, different fluorophors may be used; 150 kD FITC-dextran serves as an inert plasma marker that does not leak through the intact endothelial barrier and allows exact measurements of microvascular dimensions and anatomic features; the nuclear dye rhodamine 6G can be injected through an IV to identify endogenous circulating leukocytes; MAbs to endothelial surface markers may be tagged with fluorophors to detect luminal antigens; finally, purified immune cells can be labeled in vitro using dyes such as BCECF or calcein AM. Once the fluorophors are injected, the fluorescents are recorded on video tape using video-triggered stroboscopic epi-illumination with appropriate filter sets. Video recordings are used for off-line analysis and determination of a number of parameters. Molecules that inhibit or activate the interactions of lymphocytes with HEV endothelial cells in the rolling assays are selected for further characterization.

In one embodiment, the invention provides assays for screening candidate or test compounds which are target molecules of a SUT-2 or SUT-3 protein or polypeptide or biologically active portion thereof. In another embodiment, the invention provides assays for screening candidate or test compounds which bind to or modulate the activity of a SUT-2 or SUT-3 protein or polypeptide or biologically active portion thereof.

The test compounds which may be used in any of the assays described herein can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is used with peptide libraries, while the other four approaches are applicable to peptide, non-peptide oligomer or small molecule libraries of compounds (Lam, K. S. (1997) Anticancer Drug Des. 12:145, the disclosure of which is incorporated herein by reference in its entirety).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233, the disclosures of which are incorporated herein by reference in their entireties.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devin (1990) Science 249:404-406); (Cwirla et al. (1990) Proc. Natl. Acad. Sci. 87:6378-6382); (Felici (1991) J. Mol. Biol. 222:301-310); (Ladner supra.), the disclosures of which are incorporated herein by reference in their entireties.

Determining the ability of the test compound to inhibit or increase SUT-2 or SUT-3 activity can also be accomplished, for example, by coupling the SUT-2 or SUT-3 protein or biologically active portion thereof with a radioisotope or enzymatic label such that binding of the SUT-2 or SUT-3 protein or biologically active portion thereof to its cognate target molecule can be determined by detecting the labeled SUT-2 or SUT-3 protein or biologically active portion thereof in a complex. For example, compounds (e.g., SUT-2 or SUT-3 protein or biologically active portion thereof) can be labeled with 125 I, 35 S, 14 C, or 3 H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product. The labeled molecule is placed in contact with its cognate molecule and the extent of complex formation is measured. For example, the extent of complex formation may be measured by immuno precipitating the complex or by performing gel electrophoresis. The extent of complex formation in the presence and absence of the test compound is compared.

It is also within the scope of this invention to determine the ability of a compound (e.g., SUT-2 or SUT-3 protein or biologically active portion thereof) to interact with its cognate target molecule without the labeling of any of the interactants. Interaction of the SUT-2 or SUT-3 protein or biologically active fragment thereof with the target molecule may be measured in the presence or absence of the test compound to identify compounds which increase or decrease the extent of interaction. For example, a microphysiometer can be used to detect the interaction of a compound with its cognate target molecule without the labeling of either the compound or the target molecule. McConnell, H. M. et al. (1992) Science 257:1906-1912, the disclosure of which is incorporated herein by reference in its entirety. A microphysiometer such as a cytosensor is an analytical instrument that measures the rate at which a cell acidifies its environment using a light-addressable potentiometric sensor (LAPS). Changes in this acidification rate can be used as an indicator of the interaction between compound and receptor.

In a preferred embodiment, the assay comprises contacting a cell which expresses a SUT-2 or SUT-3 protein or biologically active portion thereof, with a target molecule to form an assay mixture, contacting the assay mixture with a test compound, and determining the ability of the test compound to inhibit or increase the activity of the SUT-2 or SUT-3 protein or biologically active portion thereof, wherein determining the ability of the test compound to inhibit or increase the activity of the SUT-2 or SUT-3 protein or biologically active portion thereof, comprises determining the ability of the test compound to inhibit or increase a biological activity of the SUT-2 or SUT-3 expressing cell (e.g., determining the ability of the test compound to inhibit or increase transduction, protein:protein interactions, sulfation of L-selectin ligands (preferably sialomucin-type L-selectin counter receptors), L-selectin dependent lymphocyte adhesion to cells comprising a L-selectin ligand (preferably HEVs or HEVECs), interactions between L-selectin ligands (preferably sialomucin-type L-selectin counter receptors and L-selectin), or adhesion of lymphocytes to endothelial cells (preferably to HEVs or HEVECs)). Compounds which inhibit or increase the activity of the SUT-2 or SUT-3 protein or a biologically active portion thereof may be identified by performing the assay in the presence or absence of a test compound.

In another preferred embodiment, the assay comprises contacting a cell which is responsive to a SUT-2 or SUT-3 protein or biologically active portion thereof, with a SUT-2 or SUT-3 protein or biologically-active portion thereof, to form an assay mixture, contacting the assay mixture with a test compound, and determining the ability of the test compound to modulate the activity of the SUT-2 or SUT-3 protein or biologically active portion thereof, wherein determining the ability of the test compound to modulate the activity of the SUT-2 or SUT-3 protein or biologically active portion thereof comprises determining the ability of the test compound to modulate a biological activity of the SUT-2 or SUT-3-responsive cell (e.g., determining the ability of the test compound to modulate signal transduction ,protein:protein interactions, sulfation of L-selectin ligands (preferably sialomucin-type L-selectin counter receptors), L-selectin dependent lymphocyte adhesion to cells comprising a L-selectin ligand (preferably HEVs or HEVECs), interactions between L-selectin ligands (preferably sialomucin-type L-selectin counter receptors and L-selectin), or adhesion of lymphocytes to endothelial cells (preferably to HEVs or HEVECs)). Compounds which inhibit or increase the activity of the SUT-2 or SUT-3 protein or a biologically active portion thereof may be identified by performing the assay in the presence or absence of a test compound.

In another embodiment, an assay is a cell-based assay comprising contacting a cell expressing a SUT-2 or SUT-3 target molecule (i.e. a molecule with which SUT-2 or SUT-3 interacts) with a test compound and determining the ability of the test compound to modulate (e.g. stimulate or inhibit) the activity of the SUT-2 or SUT-3 target molecule. Determining the ability of the test compound to modulate the activity of a SUT-2 or SUT-3 target molecule can be accomplished, for example, by determining the ability of the SUT-2 or SUT-3 protein to bind to or interact with the SUT-2 or SUT-3 target molecule. Compounds which modulate the activity of the SUT-2 or SUT-3 target molecule may be identified by performing the assay in the presence or absence of a test compound.

Determining the ability of the SUT-2 or SUT-3 protein to bind to or interact with a SUT-2 or SUT-3 target molecule can be accomplished by one of the methods described above for determining direct binding. In a preferred embodiment, determining the ability of the SUT-2 or SUT-3 protein to bind to or interact with a SUT-2 or SUT-3 target molecule can be accomplished by determining the activity of the target molecule. For example, the activity of the target molecule can be determined by contacting the target molecule with the SUT-2 or SUT-3 protein or a fragment thereof and measuring induction of a cellular second messenger of the target (i.e. intracellular Ca2+, diacylglycerol, IP3, etc.), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response, for example, signal transduction or protein:protein interactions.

In yet another embodiment, an assay of the present invention is a cell-free assay in which a SUT-2 or SUT-3 protein or biologically active portion thereof is contacted with a test compound and the ability of the test compound to bind to the SUT-2 or SUT-3 protein or biologically active portion thereof is determined. Binding of the test compound to the SUT-2 or SUT-3 protein can be determined either directly or indirectly as described above. In a preferred embodiment, the assay includes contacting the SUT-2 or SUT-3 protein or biologically active portion thereof with a known compound which binds SUT-2 or SUT-3 (e.g., a SUT-2 or SUT-3 target molecule) to form an assay mixture, contacting the assay mixture with a test compound, and determining the ability of the test compound to interact with a SUT-2 or SUT-3 protein, wherein determining the ability of the test compound to interact with a SUT-2 or SUT-3 protein comprises determining the ability of the test compound to preferentially bind to SUT-2 or SUT-3 or biologically active portion thereof as compared to the known compound.

In another embodiment, the assay is a cell-free assay in which a SUT-2 or SUT-3 protein or biologically active portion thereof is contacted with a test compound and the ability of the test compound to modulate (e.g., stimulate or inhibit) the activity of the SUT-2 or SUT-3 protein or biologically active portion thereof is determined. Determining the ability of the test compound to modulate the activity of a SUT-2 or SUT-3 protein can be accomplished, for example, by determining the ability of the SUT-2 or SUT-3 protein to bind to a SUT-2 or SUT-3 target molecule by one of the methods described above for determining direct binding. Determining the ability of the SUT-2 or SUT-3 protein to bind to a SUT-2 or SUT-3 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705, the disclosures of which are incorporated herein by reference in their entireties. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules. Compounds which inhibit or increase the activity of the SUT-2 or SUT-3 protein or a biologically active portion thereof may be identified by performing the assay in the presence or absence of a test compound.

In an alternative embodiment, determining the ability of the test compound to modulate the activity of a SUT-2 or SUT-3 protein or a biologically active portion thereof can be accomplished by determining the ability of the SUT-2 or SUT-3 protein or a biologically active portion thereof to further modulate the activity of a downstream effector (e.g., a growth factor mediated signal transduction pathway component) of a SUT-2 or SUT-3 target molecule. For example, the activity of the effector molecule on an appropriate target can be determined or the binding of the effector to an appropriate target can be determined as previously described. Compounds which inhibit or increase the activity of the SUT-2 or SUT-3 protein or a biologically active portion thereof may be identified by performing the assay in the presence or absence of a test compound.

In yet another embodiment, the cell-free assay involves contacting a SUT-2 or SUT-3 protein or biologically active portion thereof with a known compound which binds the SUT-2 or SUT-3 protein to form an assay mixture, contacting the assay mixture with a test compound, and determining the ability of the test compound to interact with the SUT-2 or SUT-3 protein, wherein determining the ability of the test compound to interact with the SUT-2 or SUT-3 protein comprises determining the ability of the SUT-2 or SUT-3 protein to preferentially bind to or modulate the activity of a SUT-2 or SUT-3 target molecule. The extent of binding in the presence of the test compound may be compared to that in the absence of the test compound.

The cell-free assays of the present invention are amenable to use of both soluble and/or membrane-bound forms of isolated proteins (e.g. SUT-2 or SUT-3 proteins or biologically active portions thereof or molecules to which SUT-2 or SUT-3 targets bind). In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton.RTM. X-100, Triton.RTM. X-114, Thesit.RTM., Isotridecypoly(ethylene glycol ether)n,3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either SUT-2 or SUT-3 or its target molecule to facilitate separation of complexed from uncomplexed forms of one or both of the proteins, as well as to accommodate automation of the assay. Binding of a test compound to a SUT-2 or SUT-3 protein, or interaction of a SUT-2 or SUT-3 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/SUT-2 or SUT-3 fuision proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or SUT-2 or SUT-3 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of SUT-2 or SUT-3 binding or activity determined using standard techniques.

Other techniques for immobilizing proteins on matrices can also be used in the screening assays of the invention. For example, either a SUT-2 or SUT-3 protein or a SUT-2 or SUT-3 target molecule can be immobilized utilizing conjugation of biotin and streptavidin. Biotinylated SUT-2 or SUT-3 protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with SUT-2 or SUT-3 protein or target molecules but which do not interfere with binding of the SUT-2 or SUT-3 protein to its target molecule can be derivatized to the wells of the plate, and unbound target or SUT-2 or SUT-3 protein trapped in the wells by antibody conjugation. Methods for detecting such complexes, in addition to those described above for the GST-immobilized complexes, include immunodetection of complexes using antibodies reactive with the SUT-2 or SUT-3 protein or target molecule, as well as enzyme-linked assays which rely on detecting an enzymatic activity associated with the SUT-2 or SUT-3 protein or target molecule.

In another embodiment, modulators of SUT-2 or SUT-3 expression are identified in a method wherein a cell is contacted with a candidate compound and the expression of SUT-2 or SUT-3 mRNA or protein in the cell is determined. The level of expression of SUT-2 or SUT-3 mRNA or protein in the presence of the candidate compound is compared to the level of expression of SUT-2 or SUT-3 mRNA or protein in the absence of the candidate compound. The candidate compound can then be identified as a modulator of SUT-2 or SUT-3 expression based on this comparison. For example, when expression of SUT-2 or SUT-3 mRNA or protein is greater (statistically significantly greater) in the presence of the candidate compound than in its absence, the candidate compound is identified as a stimulator of SUT-2 or SUT-3 mRNA or protein expression. Alternatively, when expression of SUT-2 or SUT-3 mRNA or protein is less (statistically significantly less) in the presence of the candidate compound than in its absence, the candidate compound is identified as an inhibitor of SUT-2 or SUT-3 mRNA or protein expression. The level of SUT-2 or SUT-3 mRNA or protein expression in the cells can be determined by methods described herein for detecting SUT-2 or SUT-3 mRNA or protein.

In yet another aspect of the invention, the SUT-2 or SUT-3 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300, the disclosures of which are incorporated herein by reference in their entireties), to identify other proteins, which bind to or interact with SUT-2 or SUT-3 (“SUT-2 or SUT-3-binding proteins” or “SUT-2-bp or SUT-3-bp”) and are involved in SUT-2 or SUT-3 activity. Such SUT-2 or SUT-3-binding proteins are also likely to be involved in the propagation of signals by the SUT-2 or SUT-3 proteins or SUT-2 or SUT-3 targets as, for example, downstream elements of a SUT-2 or SUT-3-mediated signaling pathway. Alternatively, such SUT-2 or SUT-3-binding proteins are likely to be SUT-2 or SUT-3 inhibitors.

The two-hybrid system is based on the modular nature of most transcription factors, which consist of separable DNA-binding and activation domains. Briefly, the assay utilizes two different DNA constructs. In one construct, the gene that codes for a SUT-2 or SUT-3 protein or a fragment thereof is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a SUT-2 or SUT-3-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the SUT-2 or SUT-3 protein.

This invention further pertains to novel agents identified by the above-described screening assays and to processes for producing such agents by use of these assays. Accordingly, in one embodiment, the present invention includes a compound or agent obtainable by a method comprising the steps of any one of the aformentioned screening assays (e.g., cell-based assays or cell-free assays). For example, in one embodiment, the invention includes a compound or agent obtainable by a method comprising contacting a cell which expresses a SUT-2 or SUT-3 target molecule with a test compound and the determining the ability of the test compound to bind to, or modulate the activity of, the SUT-2 or SUT-3 target molecule. In another embodiment, the invention includes a compound or agent obtainable by a method comprising contacting a cell which expresses a SUT-2 or SUT-3 target molecule with a SUT-2 or SUT-3 protein or biologically-active portion thereof, to form an assay mixture, contacting the assay mixture with a test compound, and determining the ability of the test compound to interact with, or modulate the activity of, the SUT-2 or SUT-3 target molecule. In another embodiment, the invention includes a compound or agent obtainable by a method comprising contacting a SUT-2 or SUT-3 protein or biologically active portion thereof with a test compound and determining the ability of the test compound to bind to, or modulate (e.g., stimulate or inhibit) the activity of, the SUT-2 or SUT-3 protein or biologically active portion thereof. In yet another embodiment, the present invention included a compound or agent obtainable by a method comprising contacting a SUT-2 or SUT-3 protein or biologically active portion thereof with a known compound which binds the SUT-2 or SUT-3 protein to form an assay mixture, contacting the assay mixture with a test compound, and determining the ability of the test compound to interact with, or modulate the activity of the SUT-2 or SUT-3 protein.

Accordingly, it is within the scope of this invention to further use an agent identified as described herein in an appropriate animal model. For example, an agent identified as described herein (e.g., a SUT-2 or SUT-3 modulating agent, an antisense SUT-2 or SUT-3 nucleic acid molecule, a SUT-2 or SUT-3-specific antibody, or a SUT-2 or SUT-3-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention also pertains to uses of novel agents identified by the above-described screening assays for diagnoses, prognoses, and treatments as described herein. Accordingly, it is within the scope of the present invention to use such agents in the design, formulation, synthesis, manufacture, and/or production of a drug or pharmaceutical composition for use in diagnosis, prognosis, or treatment, as described herein. For example, in one embodiment, the present invention includes a method of synthesizing or producing a drug or pharmaceutical composition by reference to the structure and/or properties of a compound obtainable by one of the above-described screening assays. For example, a drug or pharmaceutical composition can be synthesized based on the structure and/or properties of a compound obtained by a method in which a cell which expresses a SUT-2 or SUT-3 target molecule is contacted with a test compound and the ability of the test compound to bind to, or modulate the activity of, the SUT-2 or SUT-3 target molecule is determined. In another exemplary embodiment, the present invention includes a method of synthesizing or producing a drug or pharmaceutical composition based on the structure and/or properties of a compound obtainable by a method in which a SUT-2 or SUT-3 protein or biologically active portion thereof is contacted with a test compound and the ability of the test compound to bind to, or modulate (e.g., stimulate or inhibit) the activity of, the SUT-2 or SUT-3 protein or biologically active portion thereof is determined.

Methods of Treatment

SUT-2 or SUT-3 inhibitors identified according to the methods in the section titled “Drug Screening Assays” can be further tested for their ability to ameliorate or prevent inflammation, preferably chronic inflammation and autoimmune disorders in a suitable animal model of disease. Example include the nonobese diabetic (NOD) mouse model of human insulin-dependent diabetes mellitus or thymic hyperplasia in the AKR/J mouse, a model of aberrant lymphocyte migration (Michie et al, (1995) Am. J. Pathol. 147: 412-421, the disclosure of which is incorporated herein by reference in its entirety) further described herein. Compounds can also be tested as described in the section titled “Screening Assays”, for example testing for adhesion of lymphocytes by observing the ‘rolling phenotype’ in vivo in mouse lymph node HEVs using intravital microscopy (microscopy on live animals) (von Andrian, (1996), supra; von Andrian and M'Rini, (1998) supra, the disclosures of which are incorporated herein by reference in their entireties).

A large body of evidence gathered from experiments carried out with L-selectin blocking strategies suggests that SUT-2 or SUT-3 inhibitors may be effective in the treatment of inflammation and autoimmune disorders. Functional inactivation of L-selectin by blocking antibodies (Gallatin et al, (1983) Nature 304: 30-34; Hamann et al, (1994) J. Immunol. 152: 3283-3293, the disclosures of which are incorporated herein by reference in their entireties) or by gene knockout (Arbones et al, (1994) Immunity 1: 247-260, the disclosure of which is incorporated herein by reference in its entirety) results in a 99% decrease of lymphocyte migration to peripheral lymph nodes (PLNs) and a 50% reduction of lymphocyte emigration in PP HEVs. This latter result is consistent with the low but significant MECA-79 reactivity with PP HEVs (Bargatze et al, (1990) J. Cell Biochem. 42: 219-227, the disclosure of which is incorporated herein by reference in its entirety) and the expression in PP HEVs of a MECA-79+subset of MAdCAM-1 molecules able to support L-selectin mediated lymphocyte rolling in vitro (Berg et al, (1993) Nature 366: 695-698, the disclosure of which is incorporated herein by reference in its entirety). Moreover, in sheep, pig and rabbit, staining of PP HEVs with MECA-79 is as intense as the staining of PP HEVs, suggesting that, in some species, the requirement for L-selectin during lymphocyte emigration in PP HEVs could be even more critical (Mackay et al, (1992) Eur. J. Immunol. 22:887-895, the disclosure of which is incorporated herein by reference in its entirety).

The inhibitors of sulfate incorporation into HEVs, or inhibitors of L-selectin-ligand interaction identified by the SUT-2 or SUT-3 assays of the present invention are useful in treating a number of selectin-mediated disease responses. Since the selectins have several functions related to leukocyte adherence, inflammation, and coagulation, compounds that interfere with binding of L-selectin to its ligands (e.g. sialomucin-type L-selectin counter receptors on HEVs) can be used to modulate the pathological consequences of these events. The inhibitors therefore may be administered locally or systemically to control tissue damage associated with such injuries. Moreover, because of the specificity of such inhibitors for sites of inflammation, these compositions will be more effective and less likely to cause complications when compared to traditional anti-inflammatory agents.

The modes of treatment contemplated in this invention include inhibiting leukocyte adhesion or migration, comprising administering a SUT-2 or SUT-3 inhibitor so as to inhibit binding between a lymphocyte, neutrophil or monocyte and an endothelial cell or lymphatic tissue, particularly an HEVEC.

SUT-2 or SUT-3 inhibitors capable as acting as antagonists of selectin mediated adhesion to HEVs may be used in the treatment of inflammatory disorders, particularly chronic inflammatory disorders. A review of disorders is provided in Girard and Springer, (1995) Immunology Today 16(9): 449-457, the disclosure of which is incorporated herein by reference in its entirety. An inflammatory response can cause damage to the host if unchecked, because leukocytes release many toxic molecules that can damage normal tissues including proteolytic enzymes and free radicals. Vessels with HEV characteristics appear in human tissue in association with long-standing chronic inflammation. Such vessels exhibit plump endothelial cells, take up and incorporate high levels of ³⁵SO₄, contain many luminal and intramural lymphocytes (presumably in the process of extravasating) andmediate in vitrolymphocyte adhesion (Freemont (1998) J. Pathol. 155: 225-230, the disclosure of which is incorporated herein by reference in its entirety).

Rheumatoid arthritis is characterized by symmetric, polyarticular inflammation of synovial-lined joints, and may involve extraarticular tissues, such as the pericardium, lung, and blood vessels. Adhesion molecules appear to play an important role (Postigo et al., Autoimmunity 16:69, 1993, the disclosure of which is incorporated herein by reference in its entirety). Soluble selectins are present in the synovial fluid and blood of affected patients, correlating with elevated ESR and synovial PMN count (Carson C W et al. J. Rheumatol. 21:605, 1994, the disclosure of which is incorporated herein by reference in its entirety). Conventional antirheumatic therapy may modify synovial inflammation by altering leukocyte adhesion. Corticosteroids, gold compounds, and colchicine downregulate endothelial expression of selectins (Corkill et al., J. Rheumatol. 18:1453, 1991; Molad et al., Arthritis Rheum. 35:S35, 1992, the disclosures of which are incorporated herein by reference in their entireties).

In rheumatoid arthritis, it has been observed that the level of sulfate incorporation as well as the ‘plumpness’ (or ‘tallness’) of the edothelium in areas of lymphocyte infiltration in the synovial membrane are closely related to the concentration of the lymphocytes in the perivascular infiltrates (Freemont, (1987) Ann. Rheum. Dis. 46: 924-928, the disclosure of which is incorporated herein by reference in its entirety). Similarly, expression of MECA-79 and HECA-452 on these vessels is most pronounced in association with extensive lymphoid infiltrates (Michie et al, (1993) Am. J. Pathol. 143: 1688-1698; van Dinther-Janssen et al, (1990) J. Rheumatol. 17:11-17, the disclosures of which are incorporated herein by reference in their entireties). Therefore, the development of bona fide HEVs in the synovial membrane of patients with rheumatoid arthritis is likely to facilitate large-scale influx of lymphocytes, leading to amplification and maintenance of chronic inflammation.

The development of HEVs after prolonged inflammatory stimulus is not restricted to the diseased synovium, but can also occur in other tissues, particularly the gut and thyroid. During chronic inflammation of the gut in inflammatory bowel diseases (Crohn's disease and ulcerative colitis), or the thyroid in autoimmune thyroiditis (Graves' disease and Hashimoto's thyroiditis), areas of dense lymophocytic infiltration contain vessels with plump endothelium expressing MECA-79 and HECA-452 (Michie et al, supra; Duijvestijn et al., (1988) Am. J. Pathol. 130: 147-155; Kabel et al., J. (1989) Clin. Endocrinol. Metab. 68: 744-751; and Salmi et al. (1994) Gastroenterology 106: 595-605, the disclosures of which are incorporated herein by reference in their entireties). These observations suggest that HEVs could play an important role in the pathogenesis of these diseases by mediating abnormal lymphocyte recruitment to the gut or the thyroid. MECA-79+ venules with plump endothelium have also been detected in other sites of chronic inflammation, including many cutaneous inflammatory lesions (Michie et al, supra). The presence of MECA-79+ HEV-like vessels in many different chronic inflammatory diseases indicates that L-selectin is likely to play a major role in lymphocyte emigration at chronic inflammation sites. For a review of the role of adhesion molecules in these diseases, the reader is referred to Murray (Semin. Arthritis Rheum. 25:215, 1996, the disclosure of which is incorporated herein by reference in its entirety).

SUT-2 or SUT-3 inhibitors may also be useful in the treatment of disorders characterized by extralymphoid sites of chronic inflammation. In one example, SUT-2 or SUT-3 inhibitors may be useful for the treatment or prevention of diabetes mellitus. In the nonobese diabetic (NOD) mouse model of human insulin-dependent diabetes mellitus (IDDM), vessels with HEV characteristics (e.g. plump endothelial cells, numerous lymphocytes in the vessel walls) are observed during inflammation of the pancreas. Expression of MECA-79 and MECA-367 (MAdCAM-1) in induced on these HEV-like vessels (Hanninen et al., (1993) J. Clin. Invest. 92: 2509-2515; Faveeuw et al., (1994) J. Immunol. 152: 5969-5978, the disclosures of which are incorporated herein by reference in their entireties) during the development of insulitis, whereby lymphocytes infiltrate the pancreatic islets. Staining with MECA-79 in consistent with the induction of functional L-selectin ligands, CD34, MAdCAM-1 and GlyCAM-1 (Baumhueter et al, (1994) Blood 84: 2554-2565, the disclosure of which is incorporated herein by reference in its entirety). The induction of GlyCAM-1 in the inflamed pancreas of NOD mice is particularly striking since GlyCAM-1 expression in mice had previously been shown to be restricted to PLN and mesenteric lymph node (MLN) HEVs (Lasky et al., (1992) Cell 69: 927-938, the disclosure of which is incorporated herein by reference in its entirety). Together these results indicate that HEV-like vessels induced by chronic inflammation in extralymphoid sites appear to be phenotypicall similar to HEVs from lymphoid tissues. The induction of MECA-79 and MAdCAM-1 on the endothelium correlates with the expression of their counter-receptors L-selectin and alpha4-beta7 on cells infiltrating the islets (Hanninen et al., supra, the disclosure of which is incorporated herein by reference in its entirety). In vivo studies have revealed that these two receptor-counter receptor pairs, alpha4-beta7-MAdCAM-1 and L-selectin-MECA-79, play a major role in the recruitment of lymphocytes from blood into the inflamed pancreas (Yang et al., (1993) PNAS USA 90: 10494-10498, the disclosure of which is incorporated herein by reference in its entirety). Treatment of NOD mice with function-blocking monoclonal antibodies specific for L-selectin and alpha-4 integrins resulted in the inhibition of insulitis and the prevention of autoimmune diabetes.

In other examples, a SUT-2 or SUT-3 inhibitor may be used for the treatment or prevention of graft rejection. L-selectin dependent lymphocyte extravasation is a hallmark of acute heart allograft rejection in rates. Evidence further demonstrates a complete correlation between the level of expression of the sulfated sialyl Lewis-x decorated L-selectin ligands and the histological severity of heart allograft rejection (Toppila et al., (1999) Am. J. Pathol. 155:1013-1020, the disclosure of which is incorporated herein by reference in its entirety), suggesting that inhibitors capable of blocking sulfation of L-selectin ligands may be capable of preventing lymphocyte extravasation into human heart allografts at the onset and during acute rejection eposodes. In particular, Toppila et al showed that non-rejecting heart endothelium did not express, or expressed only weakly, sulfated and or sialyl Lewis-x decorations of L-selectin ligands, while said epitopes were readily detectable on endothelium of capillaries and venules at the onset and dring acute rejection eposodes.

Thus, the invention includes in preferred embodiments methods of inhibiting leukocyte adhesion or migration/extravasation, comprising administering a SUT-2 or SUT-3 inhibitor so as to inhibit binding between a lymphocyte, neutrophil or monocyte and an endothelial cell or lymphatic tissue, particularly an HEV cell. Also encompassed are methods of inhibiting lymphocyte adhesion, migration, or activation, comprising administering an L-selectin inhibitor to the lymphocyte.

Activators of SUT-2 or SUT-3 activity may be used to treat conditions in which it is desired to obtain increased lymphocyte infiltration. For example, SUT-2 or SUT-3 activators may be used to enhance the infiltration of lymphocytes into solid tumors, such as endothelial tumors.

An “individual” treated by the methods of this invention is a vertebrate, particularly a mammal (including model animals of human disease, farm animals, sport animals, and pets), and typically a human.

“Treatment” refers to clinical intervention in an attempt to alter the natural course of the individual being treated, and may be performed either for prophylaxis or during the course of clinical pathology. Desirable effects include preventing occurrence or recurrence of disease, alleviation of symptoms, diminishment of any direct or indirect pathological consequences of the disease, such as hyperresponsiveness, inflammation, or necrosis, lowering the rate of disease progression, amelioration or palliation of the disease state, and remission or improved prognosis. The “pathology” associated with a disease condition is anything that compromises the well-being, normal physiology, or quality of life of the affected individual.

Treatment is performed by administering an effective amount of a SUT-2 or SUT-3 inhibitor or activator. An “effective amount” is an amount sufficient to effect a beneficial or desired clinical result, and can be administered in one or more doses.

The criteria for assessing response to therapeutic modalities employing the lipid compositions of this invention are dictated by the specific condition, measured according to standard medical procedures appropriate for the condition.

Pharmaceutical Compositions

Compounds capable of inhibiting SUT-2 or SUT-3 activity, preferably small molecules but also including peptides, SUT-2 or SUT-3 nucleic acid molecules, SUT-2 or SUT-3 proteins, and anti-SUT-2 or anti-SUT-3 antibodies (also referred to herein as “active compounds”) of the invention can be incorporated into pharmaceutical compositions suitable for administration. Such compositions typically comprise a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Where the active compound is a protein, peptide or anti-SUT-2 or SUT-3 antibody, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Most preferably, active compound is delivered to a subject by intravenous injection.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, the disclosure of which is incorporated herein by reference in its entirety.

It is especially advantageous to formulate oral or preferably parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Diagnostic and Prognostic Uses

The nucleic acid molecules, proteins, protein homologues, and antibodies described herein can be used in one or more of the following methods: diagnostic assays, prognostic assays, monitoring clinical trials, and pharmacogenetics; and in drug screening and methods of treatment (e.g., therapeutic and prophylactic) as further described herein. Activities of the SUT-2 or SUT-3 protein are also as further described herein.

The isolated nucleic acid molecules of the invention can be used, for example, to express SUT-2 or SUT-3 protein (e.g., via a recombinant expression vector in a host cell or in gene therapy applications), to detect SUT-2 or SUT-3 mRNA (e.g., in a biological sample) or a genetic alteration in a SUT-2 or SUT-3 gene, and to modulate SUT-2 or SUT-3 activity, as described further below. The SUT-2 or SUT-3 proteins can be used to treat disorders characterized by insufficient or excessive production of a SUT-2 or SUT-3 or SUT-2 or SUT-3 target molecules. In addition, the SUT-2 or SUT-3 proteins can be used to screen for naturally occurring SUT-2 or SUT-3 target molecules, to screen for drugs or compounds which modulate, preferably inhibit SUT-2 or SUT-3 activity, as well as to treat disorders characterized by insufficient or excessive production of SUT-2 or SUT-3 protein or production of SUT-2 or SUT-3 protein forms which have decreased or aberrant activity compared to SUT-2 or SUT-3 wild type protein. Moreover, the anti-SUT-2 or SUT-3 antibodies of the invention can be used to detect and isolate SUT-2 or SUT-3 proteins, regulate the bioavailability of SUT-2 or SUT-3 proteins, and modulate SUT-2 or SUT-3 activity.

Accordingly one embodiment of the present invention involves a method of use (e.g., a diagnostic assay, prognostic assay, or a prophylactic/therapeutic method of treatment) wherein a molecule of the present invention (e.g., a SUT-2 or SUT-3 protein, SUT-2 or SUT-3 nucleic acid, or most preferably a SUT-2 or SUT-3 inhibitor or activator) is used, for example, to diagnose, prognose and/or treat a disease and/or condition in which any of the aforementioned SUT-2 or SUT-3 activities is indicated. In another embodiment, the present invention involves a method of use (e.g., a diagnostic assay, prognostic assay, or a prophylactic/therapeutic method of treatment) wherein a molecule of the present invention (e.g., a SUT-2 or SUT-3 protein, SUT-2 or SUT-3 nucleic acid, or a SUT-2 or SUT-3 inhibitor or activator) is used, for example, for the diagnosis, prognosis, and/or treatment of subjects, preferably a human subject, in which any of the aforementioned activities is pathologically perturbed. In a preferred embodiment, the methods of use (e.g., diagnostic assays, prognostic assays, or prophylactic/therapeutic methods of treatment) involve administering to a subject, preferably a human subject, a molecule of the present invention (e.g., a SUT-2 or SUT-3 protein, SUT-2 or SUT-3 nucleic acid, or a SUT-2 or SUT-3 inhibitor or activator) for the diagnosis, prognosis, and/or therapeutic treatment. In another embodiment, the methods of use (e.g., diagnostic assays, prognostic assays, or prophylactic/therapeutic methods of treatment) involve administering to a human subject a molecule of the present invention (e.g., a SUT-2 or SUT-3 protein, SUT-2 or SUT-3 nucleic acid, or a SUT-2 or SUT-3 inhibitor or activator).

For example, the invention encompasses a method of determining whether a SUT-2 or SUT-3 is expressed within a biological sample comprising: a) contacting said biological sample with: ii) a polynucleotide that hybridizes under stringent conditions to a SUT-2 or SUT-3 nucleic acid; or iii) a detectable polypeptide that selectively binds to a SUT-2 or SUT-3 polypeptide; and b) detecting the presence or absence of hybridization between said polynucleotide and an RNA species within said sample, or the presence or absence of binding of said detectable polypeptide to a polypeptide within said sample. A detection of said hybridization or of said binding indicates that said SUT-2 or SUT-3 is expressed within said sample. Preferably, the polynucleotide is a primer, and wherein said hybridization is detected by detecting the presence of an amplification product comprising said primer sequence, or the detectable polypeptide is an antibody.

Also envisioned is a method of determining whether a mammal, preferably human, has an elevated or reduced level of SUT-2 or SUT-3 expression, comprising: a) providing a biological sample from said mammal; and b) comparing the amount of a SUT-2 or SUT-3 polypeptide or of a SUT-2 or SUT-3 RNA species encoding a SUT-2 or SUT-3 polypeptide within said biological sample with a level detected in or expected from a control sample. An increased amount of said SUT-2 or SUT-3 polypeptide or said SUT-2 or SUT-3 RNA species within said biological sample compared to said level detected in or expected from said control sample indicates that said mammal has an elevated level of SUT-2 or SUT-3 expression, and wherein a decreased amount of said SUT-2 or SUT-3 polypeptide or said SUT-2 or SUT-3 RNA species within said biological sample compared to said level detected in or expected from said control sample indicates that said mammal has a reduced level of SUT-2 or SUT-3 expression.

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining SUT-2 or SUT-3 protein and/or nucleic acid expression as well as SUT-2 or SUT-3 activity, in the context of a biological sample (e.g., blood, serum, cells, tissue) to thereby determine whether an individual is afflicted with a disease or disorder, or is at risk of developing a disorder, associated with aberrant SUT-2 or SUT-3 expression or activity. The invention also provides for prognostic (or predictive) assays for determining whether an individual is at risk of developing a disorder associated with SUT-2 or SUT-3 protein, nucleic acid expression or activity. For example, mutations in a SUT-2 or SUT-3 gene can be assayed in a biological sample. Such assays can be used for prognostic or predictive purpose to thereby phophylactically treat an individual prior to the onset of a disorder characterized by or associated with SUT-2 or SUT-3 protein, nucleic acid expression or activity.

Diagnostic Assays

An exemplary method for detecting the presence or absence of SUT-2 or SUT-3 protein or nucleic acid in a biological sample involves obtaining a biological sample from a test subject and contacting the biological sample with a compound or an agent capable of detecting SUT-2 or SUT-3 protein or nucleic acid (e.g., mRNA, genomic DNA) that encodes SUT-2 or SUT-3 protein such that the presence of SUT-2 or SUT-3 protein or nucleic acid is detected in the biological sample. A preferred agent for detecting SUT-2 or SUT-3 mRNA or genomic DNA is a labeled nucleic acid probe capable of hybridizing to SUT-2 or SUT-3 mRNA or genomic DNA. The nucleic acid probe can be, for example, a full-length SUT-2 or SUT-3 nucleic acid, such as a nucleic acid of sequences of SEQ ID NOs: 1, 2, 3, 9, 11 or 12 such as a nucleic acid of at least 15, 30, 50, 100, 250, 400, 500 or 1000 nucleotides in length and sufficient to specifically hybridize under stringent conditions to SUT-2 or SUT-3 mRNA or genomic DNA. Other suitable probes for use in the diagnostic assays of the invention are described herein.

A preferred agent for detecting SUT-2 or SUT-3 protein is an antibody capable of binding to SUT-2 or SUT-3 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect SUT-2 or SUT-3 mRNA, protein, or genomic DNA in a biological sample in vitro as well as in vivo. For example, in vitro techniques for detection of SUT-2 or SUT-3 mRNA include Northern hybridizations and in situ hybridizations. In vitro techniques for detection of SUT-2 or SUT-3 protein include enzyme linked immunosorbent assays (ELISAs), Western blots, immunoprecipitations and immunofluorescence. In vitro techniques for detection of SUT-2 or SUT-3 genomic DNA include Southern hybridizations. Furthermore, in vivo techniques for detection of SUT-2 or SUT-3 protein include introducing into a subject a labeled anti-SUT-2 or anti-SUT-3 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject. In another embodiment, the methods further involve obtaining a control biological sample from a control subject, contacting the control sample with a compound or agent capable of detecting SUT-2 or SUT-3 protein, mRNA, or genomic DNA, such that the presence of SUT-2 or SUT-3 protein, mRNA or genomic DNA is detected in the biological sample, and comparing the presence of SUT-2 or SUT-3 protein, mRNA or genomic DNA in the control sample with the presence of SUT-2 or SUT-3 protein, mRNA or genomic DNA in the test sample. The invention also encompasses kits for detecting the presence of SUT-2 or SUT-3 in a biological sample. For example, the kit can comprise a labeled compound or agent capable of detecting SUT-2 or SUT-3 protein or mRNA in a biological sample; means for determining the amount of SUT-2 or SUT-3 in the sample; and means for comparing the amount of SUT-2 or SUT-3 in the sample with a standard. The compound or agent can be packaged in a suitable container. The kit can further comprise instructions for using the kit to detect SUT-2 or SUT-3 protein or nucleic acid.

In certain embodiments, detection involves the use of a probe/primer in a polymerase chain reaction (PCR) (see, e.g., U.S. Pat. Nos. 4,683,195 and 4,683,202, the disclosures of which are incorporated herein by reference in their entireties), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364, the disclosures of which are incorporated herein by reference in their entireties), the latter of which can be particularly useful for detecting point mutations in the SUT-2 or SUT-3-gene (see Abravaya et al. (1995) Nucleic Acids Res. 23:675-682, the disclosure of which is incorporated herein by reference in its entirety). This method can include the steps of collecting a sample of cells from a patient, isolating nucleic acid (e.g., genomic, mRNA or both) from the cells of the sample, contacting the nucleic acid sample with one or more primers which specifically hybridize to a SUT-2 or SUT-3 gene under conditions such that hybridization and amplification of the SUT-2 or SUT-3-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Genotyping assays for diagnostics generally require the previous amplification of the DNA region carrying the biallelic marker of interest. However, ultrasensitive detection methods which do not require amplification are also available. Methods well-known to those skilled in the art that can be used to detect biallelic polymorphisms include methods such as, conventional dot blot analyzes, single strand conformational polymorphism analysis (SSCP) described by Orita et al. PNAS 86 : 2766-2770 (1989), the disclosure of which is incorporated herein by reference in its entirety, denaturing gradient gel electrophoresis (DGGE), heteroduplex analysis, mismatch cleavage detection, and other conventional techniques as described in Sheffield et al. (1991), White et al. (1992), and Grompe et al. (1989 and 1993) (Sheffield, V. C. et al, Proc. Natl. Acad. Sci. U.S.A 49:699-706 (1991); White, M. B. et al., Genomics 12:301-306 (1992); Grompe, M. et al., Proc. Natl. Acad. Sci. U.S.A 86:5855-5892 (1989); and Grompe, M. Nature Genetics 5:111-117 (1993), the disclosures of which are incorporated herein by reference in their entireties). Another method for determining the identity of the nucleotide present at a particular polymorphic site employs a specialized exonuclease-resistant nucleotide derivative as described in U.S. Pat. No. 4,656,127, the disclosure of which is incorporated herein by reference in its entirety. Further methods are described as follows.

The nucleotide present at a polymorphic site can be determined by sequencing methods. In a preferred embodiment, DNA samples are subjected to PCR amplification before sequencing as described above. DNA sequencing methods are described in “Sequencing Of Amplified Genomic DNA And Identification Of Single Nucleotide Polymorphisms”. Preferably, the amplified DNA is subjected to automated dideoxy terminator sequencing reactions using a dye-primer cycle sequencing protocol. Sequence analysis allows the identification of the base present at the biallelic marker site.

In microsequencing methods, the nucleotide at a polymorphic site in a target DNA is detected by a single nucleotide primer extension reaction. This method involves appropriate microsequencing primers which, hybridize just upstream of the polymorphic base of interest in the target nucleic acid. A polymerase is used to specifically extend the 3′ end of the primer with one single ddNTP (chain terminator) complementary to the nucleotide at the polymorphic site. Next the identity of the incorporated nucleotide is determined in any suitable way. Typically, microsequencing reactions are carried out using fluorescent ddNTPs and the extended microsequencing primers are analyzed by electrophoresis on ABI 377 sequencing machines to determine the identity of the incorporated nucleotide as described in EP 412 883, the disclosure of which is incorporated herein by reference in its entirety. Alternatively capillary electrophoresis can be used in order to process a higher number of assays simultaneously. Different approaches can be used for the labeling and detection of ddNTPs. A homogeneous phase detection method based on fluorescence resonance energy transfer has been described by Chen and Kwok (1997) and Chen et al. (1997) Chen and Kwok (Nucleic Acids Research 25:347-353 1997) and Chen et al. (Proc. Natl. Acad. Sci. USA 94/20 10756-10761,1997), the disclosures of which are incorporated herein by reference in their entireties) In this method, amplified genomic DNA fragments containing polymorphic sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All these steps can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff and Smirnov, 1997, Genome Research, 7:378-388, 1997, the disclosure of which is incorporated herein by reference in its entirety). In another example, Pastinen et al., (Genome Research 7:606-614, 1997), the disclosure of which is incorporated herein by reference in its entirety) describe a method for multiplex detection of single nucleotide polymorphism in which the solid phase minisequencing principle is applied to an oligonucleotide array format. High-density arrays of DNA probes attached to a solid support (DNA chips) are further described below.

Other assays include mismatch detection assays, based on the specificity of polymerases and ligases. Polymerization reactions places particularly stringent requirements on correct base pairing of the 3′ end of the amplification primer and the joining of two oligonucleotides hybridized to a target DNA sequence is quite sensitive to mismatches close to the ligation site, especially at the 3′ end.

A preferred method of determining the identity of the nucleotide present at an allele involves nucleic acid hybridization. Any hybridization assay may be used including Southern hybridization, Northern hybridization, dot blot hybridization and solid-phase hybridization (see Sambrook et al., Molecular Cloning—A Laboratory Manual, Second Edition, Cold Spring Harbor Press, N.Y., 1989), the disclosure of which is incorporated herein by reference in its entirety). Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a biallelic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., 1989). The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Further, standard heterogeneous assay formats are suitable for detecting the hybrids using the labels present on the primers and probes. (see Landegren U. et al., Genome Research, 8:769-776,1998, the disclosure of which is incorporated herein by reference in its entirety).

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (e.g., the chip) at selected positions. Chips of various formats for use in detecting biallelic polymorphisms can be produced on a customized basis by Affymetrix (GeneChip), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP 785280, the disclosure of which is incorporated herein by reference in its entirety, describes a tiling strategy for the detection of single nucleotide polymorphisms. Briefly, arrays may generally be “tiled” for a large number of specific polymorphisms, further described in PCT application No. WO 95/11995, the disclosure of which is incorporated herein by reference in its entirety. Upon completion of hybridization with the target sequence and washing of the array, the array is scanned to determine the position on the array to which the target sequence hybrides. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186, the disclosures of which are incorporated herein by reference in their entireties. Solid supports and polynucleotides of the present invention attached to solid supports are further described in “Oligonucleotide Probes And Primers”.

Having generally described this invention, a further understanding can be obtained by reference to certain specific examples which are provided herein for purposes of illustration only, and are not intended to be limiting unless otherwise specified.

### EXAMPLES

### Example 1

### SUT-3 In Vitro Transcription

The SUT-3 ORF was cloned by PCR, with the 3′ RACE ready first strand cDNAs from the partial cloning of the 3′ end of SUT-3 cDNA as matrices, and the Pfu (PROMEGA) as a polymerase. Primers were ATG-SUT-3 (5′-ACCGTAGAGATGCCTTCTTCGGTGACG) (SEQ ID NO: 21) and STOP-SUT-3 (5′-CATGTCTATCAGCGGCATCCAGCATCC-3′) (SEQ ID NO: 22). All concentrations were according to supplier's instructions. The reaction was performed with 40 cycles (94° C., 1 min; 68° C., 1 min; 72° C., 4 min).

The product was digested with XbaI, purified, and cloned between HincII and XbaI sites of a modified pSP64p(A) vector (PROMEGA), containing a unique SalI restriction site instead of the EcoRI linearization site. The DTDST ORF (Hastbacka, (1994) supra, the disclosure of which is incorporated herein by reference in its entirety) was cloned in the same vector for use as a control. Constructions were sequenced by GENOME EXPRESS. In vitro transcription was carried out in 20 μl, with 1 μg of SalI-linearized modified pSP64p(A) SUT-3 or pSP64p(A) DTDST, using the SP6 mMessage mMachine (AMBION) Kit according to supplyer's protocol. Nucleotides were then removed throught a Mini Quick Spin RNA Column (Roche Biochemicals), proteins were phenol extracted, and cRNAs were ethanol precipitated and resuspended in 30 μl of water.

### Example 2

### SUT-3 Injection into Xenopus Oocytes and Sulfate Transport Assay

The handling of Xenopus oocytes was carried out as previously described (Silberg et al, (1995) J. Biol. Chem. 270: 11897-11902; and Markovich et al, (1993) PNAS USA 90: 8073-8077, the disclosures of which are incorporated herein by reference in their entireties). Oocytes were injected with 27.6 nl of water with or without SUT-3 capped cRNA (about 1 ng). After 3 days, uptake of 35S-sulfate (Amersham) was measured in different buffers in the presence or absence of inhibitors. The basic wash solution was 100 mM NaHCO3, 2 mM K HCO3, 10 mM Hepes/Tris, pH 7.5. When needed, HCO3 or Na⁺ were replaced with Cl− or K+, respectively, with respect to the osmolarity of the solution. Oocytes were washed twice in the wash solution, and then incubated for 1 hr at 25° C. in the wash solution supplemented with 35S-sulfate (40 Ci/ml) and 1 mM MgSO4. Inhibitors were used at 5 mM, excepted DIDS that was used at 1 mM. After the indicated time period, the uptake was stopped by washing the oocytes 3 times in ice cold wash solution. Single oocytes were then lysed in 0.5 ml of 10% SDS, mixed to 5 ml of FluoranSafe 2 (Polylabo) and the incorporated radioactivity was estimated in a Tri_Carb 2100 liquid scintillation counter (Packard).

### Example 3

### SUT-3 Northern Blot

To determine the tissue distribution of SUT-3 mRNA, Northern blot analysis of 12 different adult human tissues (brain, heart, skeletal muscle, colon, thymus, spleen, kidney, liver, small intestine, placenta, lung and leukocytes) were performed. Multiple Human Tissue Northern Blots (CLONTECH) were hydridized according to manufacturer's instructions. The probe was a PCR product corresponding to the SUT3 ORF, 32P-labelled with the Prime-a-Gene Labeling System (PROMEGA).

A 3.0-kb mRNA band was detected specifically in kidney, brain, skeletal muscle and small intestine. The restricted tissue distribution of SUT-3 mRNA could also be deduced from the presence in dbEST database of very few SUT-3 ESTs coming from respectively, neuroblastoma (6 ESTs), hypothalamus (3 ESTs), retinoblastoma (1 EST), skin (1 EST), carcinoma and adenocarcinoma cell lines (2 ESTs). The presence of SUT-3 mRNAs or ESTs in very few tissues suggests that SUT-3 has a restricted tissue distribution in the human body.

### Example 4

### SUT-3 mRNA Expression in Human Tonsillar HEVEC

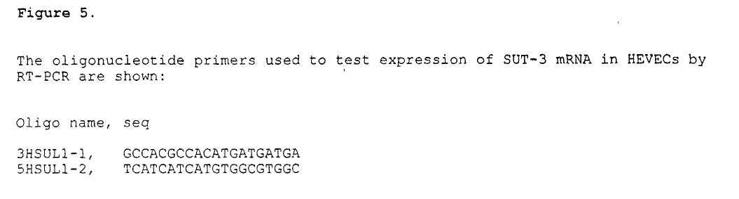

Expression of SUT-3 mRNA in human tonsillar HEVECs was tested by RT-PCR, using KlenTaq Polymerase (2 units) in 25 μl reaction buffer containing 200 μM dNTP, 10 pmol of each primer and 2.5 μl of diluted HEVEC cDNA, prepared as described in (Girard et al, (1999) PNAS USA 96: 12772-12777). Oligonucleotides primers 3HSUL1-1 (5′GCCACGCCACATGATGATGA-3′) (FIG. 5 and SEQ ID NO: 7) and 5HSUL1-2 (5′-TCATCATCATGTGGCGTGGC-3′) (FIG. 5 and SEQ ID NO: 8) were used for PCR reactions (40 cycles: 94° C., 30′; 68° C., 2′). Kidney tissue sample and amplification of DTD were used as controls. SUT-3 is expressed in tonsillar HEVEC cultured for 2 days (HEVEC-2D) or 8 days (HEVEC-8D), at levels comparable with those found in kidney.

### Example 5

### Baculovirus Construction, Sf9 Cell Culture and SUT-3 Transport Assays

The full length SLC26A11 ORF was PCR amplified from IMAGE clone N^{o} 4793646, with 10 pmol of primers ATG-SUT-3 (5′-ACCGTAGAGATGCCTTCTTCGGTGACG) (SEQ ID NO: 21) and STOP-SUT-3 (5′-CATGTCTATCAGCGGCATCCAGCATCC-3′)(SEQ ID NO: 22), using 3 units of Pfu (Promega) in its reaction buffer with 400 μM dNTP. PCR cycles were 30×(94° C., 1′, 60° C., 1′, 72° C., 4′). The PCR product was then phosphorylated at its 5′ extremities using 2 units of T4 Polynucleotide Kinase (PROMEGA). 2 μg of pVL1393 vector (BD Biosciences) were digested with 10 units of EcoRI and NotI endonucleases, extremities were blunt-ended with 10 units of T4 DNA polymerase (PROMEGA) and 5′ extremities were dephosphated with 1 unit of Calf Intestine Phosphatase (Boehringer). 100 ng of purified insert and vector were then ligated together using 1 unit of T4 DNA Ligase (Biolabs). All enzymes were used according to supplier's instructions. After restriction analysis, DNA sequencing was performed using the Big Dye Sequencing Kit (Perkin Elmer) according to manufacturer's protocol.

For Sf9 cell culture, baculovirus production and protein expression, all material was supplied by BD Biosciences. Two 25 cm² flasks, containing 60% confluent Sf9 cells, were transfected with 500 ng of BD Baculogold DNA and 5 μg of pVL1393-SUT-3 or 5 μg of pAc-HLTGFP, respectively, using the Baculogold transfection Kit. Resulting recombinant viruses produce either SUT-3, or the GFP protein under the control of the polyhedrin promoter. Viruses were amplified by two successive Sf9 culture infections to obtain a high titre virus stock.

For all experiments, 80 cm² flasks containing 60% confluent Sf9 cells were infected at a MOI of 5 and incubated for 3 days at 27° C. for protein expression. Floating cells from each flask were then isolated, washed three times in 12 ml pre-wash buffer (300 mM sucrose, 10 mM Hepes/Tris pH 7.5, 1 mM MgCl₂, 27° C.) and incubated in 1 ml pre-wash buffer supplemented with 5 μCi sulfate (Amersham, 1 Ci/mmol) and 1 mM Na₂SO₄ or K₂SO₄, plus eventually 1 mM DIDS. Incorporation was stopped by four washes in post-wash buffer (100 mM Sucrose, 100 mM NaNO₃, 10 mM Hepes/Tris pH 7.5, 1 mM MgCl₂, 4° C.). Cells were then dissolved in 1 ml 1N NaOH. Protein concentration was evaluated using the BIO-RAD protein Assay Kit according to the manufacturer's protocol and cell-associated radioactivity was determined in a Tri-Carb 2100 liquid scintillation counter (Packard).

FIG. 12 shows sulfate uptake by Sf9 insect cells infected at a MOI of 5 with baculoviruses expressing SUT-3 (filled bars) and control cells (infected with baculovirus expressing the GFP or wild type baculovirus, open bars). A, Na⁺-independent sulfate transport. Uptake buffer contained either 500 μM Na₂SO₄ (+Na⁺) or 500 μM K₂SO₄ (−Na⁺). B, SUT-3-mediated sulfate transport is sensitive to the anion exchanger inhibitor DIDS. Uptake of 500 μM Na₂SO₄ was performed in the presence (+DIDS) or absence (−DIDS) of 1 mM DIDS. Data are shown as means±SE for a single experiment performed in triplicates and are representative of at least two similar experiments.

### Example 6

### Subcellular Localization of the SUT-3 Protein

Epitope Tagged SLC26A1 1 Expression Vector Construction

In order to fuse cMyc epitope tag to the N terminus of SLC26A11, SLC26A11 coding sequence was PCR amplified using both SUT-3-Nde5′ (5′GGGAATTCCATATGCCTTCTTCGGTGACGGCG3′) (SEQ ID NO: 23) and SUT-3-Eco3′ (5′CCGGAATTCTTATGCCTTGAGCAGGGCAAC3′) (SEQ ID NO: 24) from 100 ng of IMAGE clone N^{o} 4793646, and the resulting DNA fragment cloned in pGBKT7 vector (Clontech) at NdeI and EcoRI sites. The resulting construction was used as a template in a PCR reaction in which the cMyc-SUT-3 fusion coding sequence was amplified using both oligonucleotides Myc5′ (5-′GCTCTAGAGCCACCATGGAGGAGCAGAAG-3′) (SEQ ID NO: 25) and SUT-3-Xba3′ (5′-GCTCTAGATTATGCCTTGAGCAGGGCAAC-3′) (SEQ ID NO: 26). The amplified DNA fragment was cloned at the XbaI site of pEF BOS vector (Mizushima, S., and Nagata, S. (1990) Nucleic Acids Res 18(17), 5322), downstream from the promoter of the EF1alpha gene, resulting to construction of the pEF-MycSUT-3 plasmid.

Cell Culture and Transfection Procedures

Cos-7 cell line was cultured in DMEM 4.5 g/l glucose, with glutamax and pyridoxin (Gibco), 10% FBS, gentamycin 2 mg/l. Cells were grown either in 100 mm dishes or in 12 well plates (Nunc) at 37° C. 60% confluent Cos-7 cells were transfected on coverslips in 12-well plates with the pEF-MycSUT-3 plasmid (or pcDNA3.1S19 (Soulet, F., et al. (2001) Biochem Biophys Res Commun 289(2), 591-6) as a control) using the jetPEI (Qbiogene) transfection kit according to supplier's instructions.

Immunostaining and Fluorescence Microscopy

Coverslips were washed three times with PBS, fixed for 10 min in PBS+3% paraformaldehyde, washed 3 times again with PBS and neutralised with PBS+50 mM NH₄Cl. After 3 further washes, cells were incubated (or not) in PBS+0.1% Triton-X100 for permeabilisation and washed 3 times again. Coverslips were then incubated for 30 min in PBS+3% BSA for blocking, incubated in the same buffer with the monoclonal anti-Myc antibody (Clontech) diluted at 1/100 for 1 hour, washed 3 times again, incubated with anti mouse Cy3-conjugated secondary antibodies for 1 hour and washed 3 times again in PBS. Samples were then rapidly ethanol dried, and mounted in Vectashield (Vector Laboratories). Fluorescence was observed with an Eclipse TE300 microscope using a 40×objective equiped with a digital camera DXM1200 (Nikon Corp., Tokyo, Japan).

### Example 7

### SUT-2 In Vitro Transcription

The SUT-2 ORF was cloned by PCR, with the 3′ RACE ready first strand cDNAs from the partial cloning of the 3′ end of SUT-2 cDNA as matrices, and the Pfu (PROMEGA) as a polymerase. Primers were ATG-SUT2 (5′-CCACCATGACAGGAGCAAAGAGGAAAAAGAAAAGC-3′) (SEQ ID NO: 16) and 3+XbaI-SUT2 (5′GCGTCTAGATCAGACTTCACTGTGGTCACTGAGTTTGC-3′) (SEQ ID NO: 17). All concentrations were according to supplier's instructions. The reaction was performed with 40 cycles (94° C., 1 min; 68° C., 1 min; 72° C., 4 min).

The product was digested with XbaI, purified, and cloned between HindII and XbaI sites of a modified pSP64p(A) vector (PROMEGA), containing a unique SalI restriction site instead of the EcoRI linearization site. The DTDST ORF (Hastbacka, (1994) supra, the disclosure of which is incorporated herein by reference in its entirety) was cloned in the same vector for use as a control. Constructions were sequenced by GENOME EXPRESS. In vitro transcription was carried out in 20 l, with 1 g of SalI-linearized modified pSP64p(A) SUT-2 or pSP64p(A) DTDST, using the SP6 mMessage mMachine (AMBION) Kit according to supplyer's protocol. Nucleotides were then removed throught a Mini Quick Spin RNA Column (Roche Biochemicals), proteins were phenol extracted, and cRNAs were ethanol precipitated and resuspended in 30 μl of water.

### Example 8

### SUT-2 Injection into Xenopus Oocytes and Sulfate Transport Assay

The handling of Xenopus oocytes was carried out as previously described (Silberg et al, (1995) J. Biol. Chem. 270: 11897-11902; and Markovich et al, (1993) PNAS USA 90: 8073-8077, the disclosures of which are incorporated herein by reference in their entireties). Oocytes were injected with 27.6 nl of water with or without SUT-2 capped cRNA (about 1 ng). After 3 days, uptake of ³⁵S-sulfate (Amersham) was measured in different buffers in the presence or absence of inhibitors. The basic wash solution was 100 mM NaHCO₃, 2 mM K HCO₃, 10 mM Hepes/Tris, pH 7.5. When needed, HCO₃⁻ or Na⁺ were replaced with Cl⁻ or K⁺, respectively, with respect to the osmolarity of the solution. Oocytes were washed twice in the wash solution, and then incubated for 1 hr at 25° C. in the wash solution supplemented with ³⁵S-sulfate (40 Ci/ml) and 1 mM MgSO₄. Inhibitors were used at 5 mM, excepted DIDS that was used at 1 mM. After the indicated time period, the uptake was stopped by washing the oocytes 3 times in ice cold wash solution. Single oocytes were then lysed in 0.5 ml of 10% SDS, mixed to 5 ml of FluoranSafe 2 (Polylabo) and the incorporated radioactivity was estimated in a Tri_Carb 2100 liquid scintillation counter (Packard).

### Example 9

### SUT-2 Northern Blot

To determine the tissue distribution of SUT-2 mRNA, Northern blot analysis of 12 different adult human tissues (brain, heart, skeletal muscle, colon, thymus, spleen, kidney, liver, small intestine, placenta, lung and leukocytes) were performed. Multiple Human Tissue Northern Blots (CLONTECH) were hydridized according to manufacturer's instructions. The probe was a PCR product corresponding to the SUT2 ORF, ³²P-labelled with the Prime-a-Gene Labeling System (PROMEGA). A strong 6.0-kb mRNA band was detected specifically in kidney. The restricted tissue distribution of SUT-2 mRNA could also be deduced from the presence in dbEST database of very few SUT-2 ESTs coming from respectively, kidney (1 EST), head and neck (1 EST), placenta (1 EST) and fetus (1 EST). The presence of SUT-2 mRNAs or ESTs in very few tissues suggests that SUT-2 has a highly restricted tissue distribution in the human body.

### Example 10

### SUT-2 mRNA Expression in Human Tonsillar HEVEC

Expression of SUT-2 mRNA in human tonsillar HEVECs was tested by RT-PCR, using KlenTaq Polymerase (2 units) in 25 μl reaction buffer containing 200 μM dNTP, 10 pmol of each primer and 2.5 μl of diluted HEVEC cDNA, prepared as described in (Girard et al, (1999) PNAS USA 96: 12772-12777). Oligonucleotides primers 5′26A8d (5′-CTTCTTCATTGCAGTTGCCATTGGATAG-3′) (SEQ ID NO: 18 ) and 3′26A8d (5′-GAAACCCTGCAGCAGGTGAAAATTATCT-3′) (SEQ ID NO: 19) ) were used for PCR reactions (40 cycles: 94° C., 30′; 68° C., 2′). Kidney tissue sample and amplification of DTD were used as controls. SUT-2 is expressed in tonsillar HEVEC cultured for 2 days (HEVEC-2D) or 8 days (HEVEC-8D), at levels comparable with those found in kidney.

Numerous literature and patent references have been cited in the present application. All references cited are incorporated by reference herein in their entireties.

What is claimed is:

1. A method of identifying a candidate compound for the amelioration of an inflammatory condition, said method comprising:
(a) identifying a compound which inhibits the activity of a SUT-2 or SUT-3 protein; and
(b) determining whether said compound is capable of, or likely to be capable of, inhibiting leukocyte adhesion or migration through blood vessels.

2. The method of claim 1, wherein said SUT-2 or SUT-3 protein comprises an amino acid sequence selected from the group consisting of SEQ ID NOs: 4, 5, 6, 10, 13, 14 and 27.

3. The method of claim 1, wherein said SUT-2 or SUT-3 protein comprises an amino acid selected from the group consisting of an amino acid sequence having at least about 60% amino acid identity to one of SEQ ID NOs: 4, 5, 6, 10, 13, 14 or 27, an amino acid sequence having at least about 65% amino acid identity to one of SEQ ID NOs: 4, 5, 6, 10, 13, 14 or 27, an amino acid sequence having at least about 70% amino acid identity to one of SEQ ID NOs: 4, 5, 6, 10, 13, 14 or 27, an amino acid sequence having at least about 75% amino acid identity to one of SEQ ID NOs: 4, 5, 6, 10, 13, 14 or 27, an amino acid sequence having at least about 80% amino acid identity to one of SEQ ID NOs: 4, 5, 6, 10, 13 14 or 27, an amino acid sequence having at least about 85% amino acid identity to one of SEQ ID NOs: 4, 5, 6, 10, 13, 14 or 27, an amino acid sequence having at least about 90% amino acid identity to one of SEQ ID NOs: 4, 5, 6, 10, 13, 14 or 27, an amino acid sequence having at least about 92% amino acid identity to one of SEQ ID NOs: 4, 5, 6, 10, 13 14 or 27, an amino acid sequence having at least about 95% amino acid identity to one of SEQ ID NOs: 4, 5, 6, 10, 13, 14 or 27, an amino acid sequence having at least about 97% amino acid identity to one of SEQ ID NOs: 4, 5, 6, 10, 13, 14 or 27, an amino acid sequence having at least about 98% amino acid identity to one of SEQ ID NOs: 4, 5, 6, 10, 13 14 or 27, an amino acid sequence having at least about 98% amino acid identity to one of SEQ ID NOs: 4, 5, 6, 10, 13, 14 or 27, an amino acid sequence having at least about 99% amino acid identity to one of SEQ ID NOs: 4, 5, 6, 10, 13, 14 or 27, and an amino acid sequence having at least about 99.8% amino acid identity to one of SEQ ID NOs: 4, 5, 6, 10, 13, 14 or 27.

4. The method of claim 1, wherein said SUT-2 or SUT-3 protein comprises an amino acid sequence selected from the group consisting of residues 164-471) of SEQ ID NO: 13, residues 137-512 of SEQ ID NO: 5, residues 137-468 of SEQ ID NOs: 4, 6 and 10, and residues 77-98 of SEQ ID NOs: 4, 5, 6 and 10.

5. The method of claim 1, wherein said SUT-2 or SUT-3 protein comprises an amino acid seequence selected from the group consisting of an amino acid sequence in which at least about 50% -80% of the residues are identical or similar to residues 164-471 of SEQ ID NO: 13, residues 137-512 of SEQ ID NO: 5, residues 137-468 of SEQ ID NOs: 4, 6 and 10, or residues 77-98 of SEQ ID NOs: 4, 5, 6 and 10, an amino acid sequence in which at least about 60-70% are identical or similar to residues 164-471 of SEQ ID NO: 13, residues 137-512 of SEQ ID NO: 5, and residues 137-468 of SEQ ID NOs: 4, 6 and 10, or residues 77-98 of SEQ ID NOs: 4, 5, 6 and 10 and an amino acid sequence in which at least about 65% of the amino acid residues are identical or similar amino acids to residues 164-471 of SEQ ID NO: 13, residues 137-512 of SEQ ID NO: 5, residues 137-468 of SEQ ID NOs: 4, 6 and 10 or residues 77-98 of SEQ ID NOs: 4, 5, 6 and 10.

6. The method of claim 1, wherein said SUT-2 or SUT-3 protein comprises an amino acid sequence selected from the group consisting of residues 481-656 of SEQ ID NOs: 13, residues 514-620 of SEQ ID NO: 5, and residues 470-576 of SEQ ID NOs: 4, 6 and 10.

7. The method of claim 1, wherein said SUT-2 or SUT-3 protein comprises an amino acid seequence selected from the group consisting of an amino acid sequence in which at least about 50% -80% of the residues are identical or similar to residues 481-656 of SEQ ID NOs: 13, residues 514-620 of SEQ ID NO: 5, and residues 470-576 of SEQ ID NOs: 4, 6 and 10, an amino acid sequence in which at least about 60-70% are identical or similar to residues 481-656 of SEQ ID NOs: 13, residues 514-620 of SEQ ID NO: 5, and residues 470-576 of SEQ ID NOs: 4, 6 and 10, and an amino acid sequence in which at least about 65% of the amino acid residues are identical or similar amino acids to residues 481-656 of SEQ ID NOs: 13, residues 514-620 of SEQ ID NO: 5, and residues 470-576 of SEQ ID NOs: 4, 6 and 10.

8. The method of claim 1, wherein said compound inhibits a SUT-2 or SUT-3 activity selected from the group consisting of anion exchange activity, sulfate ion transport activity, and sulfation of L-selectin ligands.

9. The method according to claim 1, wherein the method further comprises administering a compound determined to be capable of, or likely to be capable of, inhibiting leukocyte adhesion or migration through blood vessels to an animal model of an inflammatory disorder and assessing the ability of the compound to ameliorate said disorder.

10. The method according to claim 1, wherein said compound is a candidate compound for the treatment of an inflammatory disorder.

11. The method according to claim 1, wherein a determination that said compound is capable of, or likely to be capable of, inhibiting leukocyte adhesion or migration through blood vessels indicates that said compound is a candidate compound for the treatment of an inflammatory disorder.

12. The method according to claim 1, wherein said inhibitor is a selective inhibitor of said SUT-2 or SUT-3 polypeptide.

13. The method according to claim 1, wherein said SUT-2 polypeptide is human SUT-2 polypeptide.

14. The method according to claim 1, wherein said SUT-3 polypeptide is human SUT-3 polypeptide.

15. A method of identifying a candidate compound for the amelioration of an inflammatory condition, said method comprising:
(a) providing a test compound;
(b) determining whether said test compound is capable of inhibiting the activity of a SUT-2 or SUT-3 protein; and
(c) for a compound capable of inhibiting SUT-2 or SUT-3 activity, determining whether said test compound is capable of, or likely to be capable of, inhibiting leukocyte adhesion or migration through blood vessels.

16. The method of claim 15, wherein determining whether said test compound is capable of inhibiting the activity of a SUT-2 or SUT-3 protein comprises determining whether said test compound inhibits sulfate transport activity.

17. The method of claim 15, wherein determining whether said test compound is capable of inhibiting the activity of a SUT-2 or SUT-3 protein comprises:
(i) contacting a SUT-2 or SUT-3 polypeptide or a fragment thereof with a test compound; and
(ii) determining whether said compound selectively inhibits SUT-2 or SUT-3 activity.

18. The method of claim 15, wherein determining whether said test compound is capable of inhibiting the activity of a SUT-2 or SUT-3 protein comprises:
(i) providing a cell comprising a SUT-2 or SUT-3 polypeptide or a fragment comprising at least 6 consecutive amino acids thereof;
(ii) contacting said cell with a test compound; and
(iii) determining whether said compound selectively inhibits SUT-2 or SUT-3 activity.

19. The method of claim 18, wherein said step of determining whether said compound selectively inhibits SUT-2 or SUT-3 activity comprises assessing sulfate uptake activity.

20. The method of claim 18, wherein said cell is an insect cell transfected with a baculovirus vector comprising a nucleic acid sequence encoding a SUT-2 or SUT-3 polypeptide.

21. The method of claim 15, comprising performing a first assay to determine whether said test compound is capable of inhibiting the activity of a SUT-2 or SUT-3 protein, and performing a second assay to determine whether said test compound is capable of, or likely to be capable of, inhibiting leukocyte adhesion or migration through blood vessels, said first assay having a protocol different from said second assay.

22. The method of claim 15, wherein determining whether said test compound is capable of, or likely to be capable of, inhibiting leukocyte adhesion or migration through blood vessels comprises performing an assay designed to measure a property indicative of leukocyte adhesion or migration through blood vessels.

23. The method according to claim 15, wherein a determination that said compound is capable of, or likely to be capable of, inhibiting leukocyte adhesion or migration through blood vessels indicates that said compound is a candidate compound for the treatment of an inflammatory condition.

24. The method according to claim 15, wherein said leukocyte is a lymphocyte, neutrophil or monocyte.

25. The method according to claim 22, wherein the property measured is indicative of migration through endothelial vessels.

26. The method of claim 25, wherein said property measured is indicative of migration through high endothelial venules.

27. The method of claim 22, wherein said property measured is indicative of adhesion to an endothelial cell or lymphatic tissue.

28. The method of claim 22, wherein said property measured is indicative of adhesion to a high endothelial venule endothelial cell (HEVEC).

29. The method according to claim 15, wherein determining whether said test compound is capable of, or likely to be capable of, inhibiting leukocyte adhesion or migration through blood vessels comprises measuring the sulfation of an L-selectin ligand.

30. The method of claim 29, wherein said L-selectin ligand is a sialomucin type L-selectin counter-receptor.

31. The method according to claim 15, wherein determining whether said test compound is capable of, or likely to be capable of, inhibiting leukocyte adhesion or migration through blood vessels comprises measuring L-selectin dependent leukocyte adhesion to cells comprising an L-selectin ligand

32. The method according to claim 15, wherein determining whether said test compound is capable of, or likely to be capable of, inhibiting leukocyte adhesion or migration through blood vessels comprises measuring an interaction between L-selectin and L-selectin ligands.

33. The method of claim 32, wherein interaction is measured between a sialomucin-type L-selectin counter receptor and L-selectin.

34. The method according to claim 15 wherein determining whether said test compound is capable of, or likely to be capable of, inhibiting leukocyte adhesion or migration through blood vessels comprises measuring adhesion of leukocytes to HEVs or HEVECs.

35. The method of claim 34, wherein adhesion is measured by observing the ‘rolling phenotype’ in vivo in mouse lymph node HEVs.

36. The method according to claim 15, wherein the method further comprises administering a compound determined to be capable of, or likely to be capable of, inhibiting leukocyte adhesion or migration through blood vessels to an animal model of an inflammatory disorder and assessing the ability of the compound to ameliorate said disorder.

37. The method according to claim 15, wherein said compound is a candidate compound for the treatment of an inflammatory disorder.

38. The method according to claim 15, wherein said inhibitor is a selective inhibitor of said SUT-2 or SUT-3 polypeptide.

39. The method according to claim 15, wherein said SUT-2 polypeptide is human SUT-2 polypeptide.

40. The method according to claim 15, wherein said SUT-3 polypeptide is human SUT-3 polypeptide.

41. A method of identifying a candidate compound for the amelioration of an inflammatory condition, said method comprising:
(a) providing a compound capable of inhibiting the activity of a SUT-2 or SUT-3 protein; and
(b) administering said compound to an animal model of an inflammatory disorder, and assessing the ability of said compound to ameliorate said disorder.

42. The method of claim 41, wherein a determination that said compound is capable of ameliorating said condition indicates that said compound is a candidate compound for the treatment of an inflammatory condition.

43. The method of claim 41, wherein said animal model is an animal model of chronic inflammation.

44. The method of claim 41, wherein said animal model is an animal model of rheumatoid arthritis.

45. The method of claim 41, wherein said animal model is an animal model of inflammatory bowel diseases.

46. The method of claim 41, wherein said animal model is an animal model of autoimmune disorder.

47. The method of claim 41, wherein said animal model is an animal model of insulin dependent diabetes mellitus.

48. The method of claim 41, wherein said animal model is an animal model of graft rejection.

49. The method of claim 41, wherein ameliorating said disorder comprises ameliorating a symptom of said disorder.

50. The method according to claim 41, wherein said inhibitor is a selective inhibitor of said SUT-2 or SUT-3 polypeptide.

51. The method according to claim 41, wherein said SUT-2 polypeptide is human SUT-2 polypeptide.

52. The method according to claim 41, wherein said SUT-3 polypeptide is human SUT-3 polypeptide.

53. A method of identifying a candidate SUT-2 or SUT-3 inhibitor, said method comprising:
a) providing an insect cell transfected with a baculovirus expression vector comprising a nucleic acid encoding a SUT-2 or SUT-3 polypeptide;
b) contacting said cell with a test compound; and
c) determining whether said compound selectively inhibits sulfate uptake activity;
wherein a determination that said compound selectively inhibits sulfate uptake activity indicates that said compound is a candidate inhibitor of said SUT-2 or SUT-3 polypeptide.

54. The method of claim 53, wherein said polypeptide is a SUT-3 polypeptide.

55. The method of claim 53, wherein said polypeptide is a SUT-2 polypeptide.

56. The method according to claim 53, wherein the method further comprises administering a compound determined to selectively inhibit sulfate uptake activity to an animal model of an inflammatory disorder and assessing the ability of the compound to ameliorate said disorder.

57. The method according to claim 53, wherein said compound is a candidate compound for the treatment of an inflammatory disorder.

58. The method according to claim 53, wherein a determination that said compound selectively inhibits sulfate uptake activity indicates that said compound is a candidate compound for the treatment of an inflammatory disorder.

59. The method according to claim 53, wherein said SUT-2 polypeptide is human SUT-2 polypeptide.

60. The method according to claim 53, wherein said SUT-3 polypeptide is human SUT-3 polypeptide.

61. A method of identifying a candidate SUT-2 inhibitor, said method comprising:
a) providing a cell comprising a nucleic acid encoding a SUT-2 polypeptide;
b) contacting said cell with a test compound; and
c) determining whether said compound selectively inhibits sulfate uptake activity;
wherein a determination that said compound selectively inhibits sulfate uptake activity indicates that said compound is a candidate inhibitor of said SUT-2 polypeptide.

62. The method of claim 61, wherein said cell comprises an expression vector comprising a nucleic acid encoding a SUT-2 polypeptide.

63. The method of claim 61, wherein said cell is a Xenopus oocyte.

64. The method according to claim 61, wherein the method further comprises administering a compound determined to selectively inhibit sulfate uptake activity to an animal model of an inflammatory disorder and assessing the ability of the compound to ameliorate said disorder.

65. The method according to claim 61, wherein said compound is a candidate compound for the treatment of an inflammatory disorder.

66. The method according to claim 61, wherein a determination that said compound selectively inhibits sulfate uptake activity indicates that said compound is a candidate compound for the treatment of an inflammatory disorder.

67. The method according to claim 61, wherein said SUT-2 polypeptide is human SUT-2 polypeptide.

68. A method of identifying a candidate SUT-2 or SUT-3 inhibitor, said method comprising:
a) providing a Xenopus oocyte comprising a nucleic acid encoding a SUT-2 or SUT-3 polypeptide;
b) contacting said oocyte with a test compound; and
c) determining whether said compound selectively inhibits sulfate uptake activity;
wherein a determination that said compound selectively inhibits sulfate uptake activity indicates that said compound is a candidate inhibitor of said SUT-2 or SUT-3 polypeptide.

69. The method of claim 68, wherein said polypeptide is a SUT-2 polypeptide.

70. The method of claim 68, wherein said polypeptide is a SUT-3 polypeptide.

71. The method according to claim 68, wherein the method further comprises administering a compound determined to selectively inhibit sulfate uptake activity to an animal model of an inflammatory disorder and assessing the ability of the compound to ameliorate said disorder.

72. The method according to claim 68, wherein said compound is a candidate compound for the treatment of an inflammatory disorder.

73. The method according to claim 68, wherein said inhibitor is a selective inhibitor of said SUT-2 or SUT-3 polypeptide.

74. The method according to claim 68, wherein said SUT-2 polypeptide is human SUT-2 polypeptide.

75. The method according to claim 68, wherein said SUT-3 polypeptide is human SUT-3 polypeptide.

76. An isolated nucleic acid selected from the group consisting of:
(i) nucleic acid molecule encoding a polypeptide comprising an amino acid sequence selected from the group of sequences consisting of SEQ ID NOs: 5, 6, 10 and 27;
(ii) a nucleic acid molecule comprising a nucleic acid sequence selected from the group of sequences consisting of SEQ ID NOs: 2, 3 or 9, or a sequence complementary thereto;
(iii) a nucleic acid encoding a polypeptide comprising an amino acid sequence having at least about 95% identity to a sequence selected from the group consisting of the polypeptides of SEQ ID NOs: 5, 6, 10 and 27, and the polypeptides encoded by the nucleic acid of SEQ ID NOs: 2, 3 or 9; or
(iv) a nucleic acid encoding a fragment of said polypeptide which possesses sulfate transport activity, said fragment comprising at least 6 contiguous amino acid residues of amino acid positions 607 to 650 of SEQ ID NO: 5.
(v) a nucleic acid molecule the complementary strand of which hybridizes under stringent conditions to a nucleic acid as defined in (i) to (iv); and
(vi) a nucleic acid the sequence of which is degenerate as a result of the genetic code to a sequence of a nucleic acid as defined in (i) to (v).

77. The nucleic acid of claim 76, wherein said polypeptide comprises an amino acid sequence selected from the group consisting of the sequences shown as SEQ ID NOs: 5, 6, 10 and 27 and the polypeptides encoded by the nucleic acid of SEQ ID NOs: 2, 3 or 9.

78. The nucleic acid of claim 76, wherein said nucleic acid is operably linked to a promoter.

79. An expression cassette comprising the nucleic acid of claim 76.

80. A host cell comprising the expression cassette of claim 79.

81. A method of making a SUT-3 polypeptide, said method comprising
a) providing a population of host cells comprising a nucleic acid encoding a SUT-3 protein; and
b) culturing said population of host cells under conditions conducive to the expression of said recombinant nucleic acid;
whereby said polypeptide is produced within said population of host cells.

82. The method of claim 81, further comprising purifying said polypeptide from said population of cells.

83. An isolated nucleic acid, said nucleic acid comprising a nucleotide sequence having at least about 98% identity over at least about 100 nucleotides to a sequence selected from the group consisting of the sequences shown as SEQ ID NO: 2, 3 or 9, and sequences complementary thereto.

84. The nucleic acid of claim 83, wherein said nucleic acid hybridizes under stringent conditions to a nucleic acid comprising SEQ ID NO: 2, 3 or 9, or the sequence complementary thereto.

85. The nucleic acid of claim 83, wherein identity is determined using an algorithm selected from the group consisting of NBLAST with the parameters score=100 and wordlength=12, Gapped BLAST with the default parameters of NBLAST, and BLAST with the default parameters of NBLAST.

86. A polypeptide encoded by the nucleic acid of claim 76 wherein said polypeptide has sulfate transport or anion exchange activity.

87. An isolated polypeptide or fragment thereof possessing sulfate transport activity, said polypeptide comprising an amino acid sequence having at least about 95% amino acid sequence identity to a sequence selected from the group consisting of SEQ ID NOs: 5, 6, 10 and 27.

88. The polypeptide of claim 87, wherein said polypeptide is selectively bound by an antibody raised against an antigenic polypeptide, or antigenic fragment thereof, said antigenic polypeptide comprising a polypeptide selected from the group consisting of SEQ ID NOs: 5, 6, 10 and 27.

89. The polypeptide of claim 87, wherein said polypeptide comprises a polypeptide selected from the group consisting of SEQ ID NOs: 5, 6, 10 and 27.

90. An antibody that selectively binds to the polypeptide of claim 86.

91. A method of determining whether a SUT-3 is expressed within a biological sample, said method comprising the steps of:
a) contacting said biological sample with:
i) a polynucleotide that hybridizes under stringent conditions to a nucleic acid of claim 76; or
ii) a detectable polypeptide that selectively binds to the polypeptide of claim 86 or claim 87; and
b) detecting the presence or absence of hybridization between said polynucleotide and an RNA species within said sample, or the presence or absence of binding of said detectable polypeptide to a polypeptide within said sample;
wherein a detection of said hybridization or of said binding indicates that said SUT-3 is expressed within said sample.

92. The method of claim 91, wherein said polynucleotide is a primer, and wherein said hybridization is detected by detecting the presence of an amplification product comprising said primer sequence.

93. The method of claim 91, wherein said detectable polypeptide is an antibody.

94. A method of determining whether a mammal has an elevated or reduced level of SUT-3 expression, said method comprising the steps of:
a) providing a biological sample from said mammal; and
b) comparing the amount of a SUT-3 polypeptide of claim 86 or claim 87 or of a SUT-3 RNA species encoding a polypeptide of claim 86 or 87 within said biological sample with a level detected in or expected from a control sample;
wherein an increased amount of said SUT-3 polypeptide or said SUT-3 RNA species within said biological sample compared to said level detected in or expected from said control sample indicates that said mammal has an elevated level of SUT-3 expression, and wherein a decreased amount of said SUT-3 polypeptide or said SUT-3 RNA species within said biological sample compared to said level detected in or expected from said control sample indicates that said mammal has a reduced level of SUT-3 expression.

95. A method of identifying a candidate inhibitor of a SUT-3 polypeptide, said method comprising:
a) contacting a SUT-3 polypeptide according to claim 86 or claim 87; and
b) determining whether said compound selectively binds to said polypeptide;
wherein a determination that said compound selectively binds to said polypeptide indicates that said compound is a candidate inhibitor of said polypeptide.

96. A method of identifying a candidate SUT-3 inhibitor, said method comprising:
a) contacting a SUT-3 polypeptide of claim 86 or claim 87 with a test compound; and
b) determining whether said compound selectively inhibits the activity of said polypeptide;
wherein a determination that said compound selectively inhibits the activity of said polypeptide indicates that said compound is a candidate inhibitor of said polypeptide.

97. A method of identifying a candidate SUT-3 inhibitor, said method comprising:
a) providing a cell comprising a SUT-3 polypeptide of claim 86 or claim 87 with a test compound;
b) contacting said cell with a test compound; and
c) determining whether said compound selectively inhibits SUT-3 activity;
wherein a determination that said compound selectively inhibits the activity of said polypeptide indicates that said compound is a candidate inhibitor of said polypeptide.

98. The method of claim 97, wherein said cell is an Sf9 cell.

99. The method of claim 97, wherein said cell is a Xenopus laevis oocyte.

100. The method of claim 97, wherein step a) comprises introducing a nucleic acid comprising the nucleotide sequence encoding said SUT-3 polypeptide according to claim 76 into said cell.

101. The method of claim 97, wherein step a) comprises introducing a baculovirus vector comprising a nucleic acid encoding a SUT-3 polypeptide into said cell.

102. The method of claim 97, wherein step a) comprises introducing SUT-3 cRNA into said cell.

103. The method of claim 97, wherein step d) comprises detecting sulfate uptake by said cell.

104. A polynucleotide according to claim 76 attached to a solid support.

105. An array of polynucleotides comprising at least one polynucleotide according to claim 104.

106. An array according to claim 104, wherein said array is addressable.

107. A polynucleotide according to claim 76 further comprising a label.

108. A method of identifying a candidate activator of SUT-3, said method comprising:
a) contacting a SUT-3 polypeptide of claim 86 or claim 87 or a cell comprising a SUT-3 polypeptide of claims 86 or 87 with a test compound; and
b) determining whether said compound selectively increases the activity of said polypeptide;
wherein a determination that said compound selectively increases the activity of said polypeptide indicates that said compound is a candidate activator of said polypeptide.

109. The nucleic acid of claim 76, wherein polypeptide identity is determined using an algorithm selected from the group consisting of XBLAST with the parameters score=50 and wordlength=3, Gapped BLAST with the default parameters of XBLAST, and BLAST with the default parameters of XBLAST.

110. The polypeptide of claim 87, wherein identity is determined using an algorithm selected from the group consisting of XBLAST with the parameters score=50 and wordlength=3, Gapped BLAST with the default parameters of XBLAST, and BLAST with the default parameters of XBLAST.

111. A method of modulating extravasion of lymphocytes in an individual comprising modulating the activity of the SUT-3 protein or SUT-2 protein in said individual.

112. A method of reducing inflammation in an individual comprising inhibiting the activity of the SUT-3 protein or SUT-2 protein in said individual.

113. A method of increasing extravasion of lymphocytes in an individual comprising increasing the activity of the SUT-3 protein or SUT-2 protein in said individual.


SEQUENCE LISTING (27 sequences)

SEQ ID NO: 1 - DNA, 2914 bp, Homo sapiens
  CDS (294)...(2114)
tcgagagcggctttccccggcgattcctgcggacccagctgtggcgacgccaggagaccccaagctgcatcgccgagtggaagcaactagaactccagggctgtgaaagccacaggtgggggctgagcgaggcgtggcctcaggagcggaggacccccacactctccctcgagcgccgcagtccaccgtagcgggtggagcccgccttggtgcgcagttggaaaacctcggagccccgctggatctcctggctgccacccgcaccccccgccagcctacgccccaccgtagagatgccttcttcggtgacggcgctgggtcaggccaggtcctatggccccgggatggccccgagcgcctgctgctgctcccctgcggccctgcagaggaggctgcccatcctggcgtggctgcccagctactccctgcagtggctgaagatggatttcgtcgccggcctctcagttggcctcactgccattccccaggcgctggcctatgctgaagtggctggactcccgccccagtatggcctctactctgccttcatgggctgcttcgtgtatttcttcctgggcacctcccgggatgtgactctgggccccaccgccattatgtccctcctggtctccttctacaccttccatgagcccgcctacgctgtgctgctggccttcctgtccggctgcatccagctggccatgggggtcctgcgtttggggttcctgctggacttcatttcctaccccgtcattaaaggcttcacctctgctgctgccgtcaccatcggctttggacagatcaagaacctgctgggactacagaacatccccaggccgttcttcctgcaggtgtaccacaccttcctcaggattgcagagaccagggtaggtgacgccgtcctggggctggtctgcatgctgctgctgctggtgctgaagctgatgcgggaccacgtgcctcccgtccaccccgagatgccccctggtgtgcggctcagccgtgggctggtctgggctgccacgacagctcgcaacgccctggtggtctccttcgcagccctggttgcgtactccttcgaggtgactggataccagcctttcatcctaacaggggagacagctgaggggctccctccagtccggatcccgcccttctcagtgaccacagccaacgggacgatctccttcaccgagatggtgcaggacatgggagccgggctggccgtggtgcccctgatgggcctcatggagagcattgcggtggccaaagccttcgcatctcaggataattaccgcatcgatgccaaccaggagctgctggccatcggtctcaccaacatgttgggctccctcgtctcctcctacccggtcacaggcagctttggacggacagccgtgaacgctcagtcgggggtgtgcaccccggcggggggcctggtgacgggagtgctggtgctgctgtctctggactacctgacctcactgttctactacatccccaagtctgccctggctgccgtcatcatcatggccgtggccccgctgttcgacaccaagatcttcaggacgctctggcgtgttaagaggctggacctgctgcccctgtgcgtgaccttcctgctgtgcttctgggaggtgcagtacggcatcctggccggggccctggtgtctctgctcatgctcctgcactctgcagccaggcctgagaccaaggtgtcagaggggccggttctggtcctgcagccggccagcggcctgtccttccctgccatggaggctctgcgggaggagatcctaagccgggccctggaagtgtccccgccacgctgcctggtcctggagtgcacccatgtctgcagcatcgactacactgtggtgctgggactcggcgagctcctccaggacttccagaagcagggcgtcgccctggcctttgtgggcctgcaggtccccgttctccgtgtcctgctgtccgctgacctgaaggggttccagtacttctctaccctggaagaagcagagaagcacctgaggcaggagccagggacccagccctacaacatcagagaagactccattctggaccaaaaggttgccctgctcaaggcataatggggccacccgtgggcatccacagtttgcagggtgttccggaaggttcttgtcactgtgattggatgctggatgccgcctgatagacatgctggcctggctgagaaaccactgagcaggtaacccagggaagagaaggaagccaggcctggaggtccacggcagtgggagtggggctcactggcttcctgtgggatgactggaaaatgacctcgctgctgttccctggcatgaccctctttggaagagtggtttggagagagccttctagaatgacagactgtgcgaggaagcaggggcaggggtttccagcccgggctgtgcgaggcatcctggggctggcagcaccttcccggctcaccagtgccacctgcgggggagggacggggcaggcaggagtctgggaggcgggtccgctcctcttgtctgcggcatctgtgctctcggagagaaaaccaaggtgtgtcaaatgacgtcaagtctctatttaaaaataattttgtgttttctaaatggaaaaagtgatagctttggtgattttgtaaaagtcataaatgcttattgtaaaaaatacaggaaaccacccctcaccctgtccacttgggtgatcattccagacccctccccaaacatgcatatgtacctgtccgtcagtgtgtggatgtatgtttacagttctacataaatgggatcattttatacatggtgctctggaacccacatttttcatgcagtcatttgcagtgaattatttattgtgataataaatagcattagaatacaaaaaaaaaaaaaaaaaa

SEQ ID NO: 2 - DNA, 2981 bp, Homo sapiens
  CDS (229)...(2182)
ggcacgagggagagatgagggacggggtgggccttccagtcttggcccagtccccatcttgcacacattgttggcttcctcttagagccgttcgcccccctggggaggggagacccatagtgacctctcctgacacccgccgaccctgaccagtgttgccgggttcttcaaaggccacgctctgactgctggtctgtgtcacctgcaccccccagccccaccgtagagatgccttcttcggtgacggcgctgggtcaggccaggtcctctggccccgggatggccccgagcgcctgctgctgctcccctgcggccctgcagaggaggctgcccatcctggcgtggctgcccagctactccctgcagtggctgaagatggatttcgtcgccggcctctcagttggcctcactgccattccccaggcgctggcctatgctgaagtggctggactcccgccccagtatggcctctactctgccttcatgggctgcttcgtgtatttcttcctgggcacctcccgggatgtgactctgggccccaccgccattatgtccctcctggtctccttctacaccttccatgagcccgcctacgctgtgctgctggccttcctgtccggctgcatccagctggccatgggggtcctgcgtttggggttcctgctggacttcatttcctaccccgtcattaaaggcttcacctctgctgctgccgtcaccatcggctttggacagatcaagaacctgctgggactacagaacatccccaggccgttcttcctgcaggtgtaccacaccttcctcaggattgcagagaccagggtaggtgacgccgtcctggggctggtctgcatgctgctgctgctggtgctgaagctgatgcgggaccacgtgcctcccgtccaccccgagatgccccctggtgtgcggctcagccgtgggctggtctgggctgccacgacagctcgcaacgccctggtggtctccttcgcagccctggttgcgtactccttcgaggtgactggataccagcctttcatcctaacaggggagacagctgaggggctccctccagtccggatcccgcccttctcagtgaccacagccaacgggacgatctccttcaccgagatggtgcaggggcgcttctcctgttttggactccagctgaggatgaatttaccgtgttcctcccagcacctggcgcctcttcagacaaggaggcggatcctgcagctgacaagcacttgctcctgttacctgtggggcggggacatgggagccgggctggccgtggtgcccctgatgggcctcctggagagcattgcggtggccagagccttcgcatctcagaataattaccgcatcgatgccaaccaggagctgctggccatcggtctcaccaacatgttgggctccctcgtctcctcctacccggtcacaggcagctttggacggacagccgtgaacgctcagtcgggggtgtgcaccccggcggggggcctggtgacgggagtgctggtgctgctgtctctggactacctgacctcactgttctactacatccccaagtctgccctggctgccgtcatcatcatggccgtggccccgctgttcgacaccaagatcttcaggacgctctggcgtgttaagaggctggacctgctgcccctgtgcgtgaccttcctgctgtgcttctgggaggtgcagtacggcatcctggccggggccctggtgtctctgctcatgctcctgcactctgcagccaggcctgagaccaaggtgtcagaggggccggttctggtcctgcagccggccagcggcctgtccttccctgccatggaggctctgcgggaggagatcctaagccgggccctggaagtgtccccgccacgctgcctggtcctggagtgcacccatgtctgcagcatcgactacactgtggtgctgggactcggcgagctcctccaggacttccagaagcagggcgtcgccctggcctttgtgggcctgcaggtccccgttctccgtgtcctgctgtccgctgacctgaaggggttccagtacttctctaccctggaagaagcagagaagcacctgaggcaggagccagggacccagccctacaacatcagagaagactccattctggaccaaaaggttgccctgctcaaggcataatggggccacccgtgggcatccacagtttgcagggtgttccggaaggttcttgtcactgtgattggatgctggatgccgcctgatagacatgctggcctggctgagaaacccctgagcaggtaacccagggaagagaaggaagccaggcctggaggtccacggcagtgggagtggggctcactggcttcctgtgggatgactggaaaatgacctcgctgctgttccctggcatgaccctctttggaagagtggtttggagagagccttctagaatgacagactgtgcgaggaagcaggggcaggggtttccagcccgggctgtgcgaggcatcctggggctggcagcaccttcccggctcaccagtgccacctgcgggggagggacggggcaggcaggagtctgggaggcgggtccgctcctcttgtctgcggcatctgtgctctccgagagaaaaccaaggtgtgtcaaatgacgtcaagtctctatttaaaaataattttgtgttttctaaatggaaaaagtgatagctttggtgattttgtaaaagtcataaatgcttattgtaaaaaatacaggaaaccacccctcaccctgtccacttgggtgatcattccagacccctccccaaacatgcatatgtacctgtccgtcagtgtgtggatgtatgtttacagttctacataaatgggatcattttatacatggtgctctggaacccacatttttcatgcagtcatttgcagtgaattatttattgtgataataaatagcattagaatacaaaaaaaaaaaaaaaaaa

SEQ ID NO: 3 - DNA, 1821 bp, Mus musculus
  CDS (1)...(1821)
atgcccagctctgtgaaaggtctgggtcagaccagatcccccagcctgcacatggcaccagacacatgctgctgctctgctacggccctgaggaggaggctacccgtcctggcctgggtgcctgactactctctgcagtggctgaggctggacttcatcgctggactttccgtgggactcaccgtcattccccaggccctggcctatgcagaagtggctggactcccaccccagtacggcctctactctgccttcatgggatgcttcgtgtatttcttcctgggcacctcccgggatgtgactctgggccccacggctatcatgtctctcctggtgtccttctacaccttccgtgagcctgcctatgctgtgctgcttgccttcctgtctgggtgtatccagctggccatggggctcctgcatttggggttcctgctggacttcatctcctgccctgtcattaaaggcttcacctccgctgccagcatcacaattggctttggacagatcaagaacctgctgggattgcagaaaatcccccggcagttcttcctccaggtgtaccacaccttcctccacatcggagagaccagggtaggcgacgctgtcctcggactggcctccatgttgctgctgcttgtgctgaagtgtatgcgggaacacatgcctcctccccatcctgagatgccccttgccgtgaagttcagccgtgggctggtgtggactgtcacaacagctcgcaatgccctggtggtctcctccgcggctctgattgcttacgccttcgaggtgacaggatcccatccctttgttctgactggaaagatcgccgaggggctccctccggtgcggatcccacccttctcagtgaccagggacaataagaccatctcgttctctgagatggtgcaggcgaaaggctcagtaaccttctcccctcaggacatgggggccggactggctgtggtacctctgatggggctcctggagagcattgccgtggccaaatccttcgcgtctcagaataactaccgcattgatgctaaccaggaactactggccattggcctcaccaatgtgctgggctccctcgtctcctcttacccagtcactggcagctttgggcggacagctgtgaatgcccagacaggggtgtgtaccccggcaggaggcctggtgactggtgccctggtgctgctgtccctgaactacttgacctcactcttctcctatatccccaagtctgccctggctgccgtgatcatcacggctgtgaccccactctttgatgtcaagatcttcaggagtctctggcgcgttcagagtacgtacacaaagcgggcggtgtggtgtctgagtgatcgaagcctcgaagactctcctgtcctgtctgcttctaatgttcaccccctactcccattccaggtgtcagaaggacaaatttttgttcttcagccggccagcggcctgtacttccctgcaattgatgccctccgagaggcaataacgaaccgggcactggaagcatccccaccacgttccgcggttctggagtgcacgcatatcagcagtgtagactacaccgtgatcgtgggactcggtgagctcctggaggacttccagaagaaaggagtcgccctggcctttgttggcctacaggtgcccgtgctccgcacactgttggccgctgacctcaaggggttccgttacttcaccactctggaggaggctgagaaattcctgcagcaggaaccaggaactgagcccaacagcatccatgaagatgctgttccagagcaaaggagctccctgctcaagtct

SEQ ID NO: 4 - protein, 606 aa, Homo sapiens [sequence omitted]

SEQ ID NO: 5 - protein, 650 aa, Homo sapiens
MPSSVTALGQARSSGPGMAPSACCCSPAALQRRLPILAWLPSYSLQWLKMDFVAGLSVGLTAIPQALAYAEVAGLPPQYGLYSAFMGCFVYFFLGTSRDVTLGPTAIMSLLVSFYTFHEPAYAVLLAFLSGCIQLAMGVLRLGFLLDFISYPVIKGFTSAAAVTIGFGQIKNLLGLQNIPRPFFLQVYHTFLRIAETRVGDAVLGLVCMLLLLVLKLMRDHVPPVHPEMPPGVRLSRGLVWAATTARNALVVSFAALVAYSFEVTGYQPFILTGETAEGLPPVRIPPFSVTTANGTISFTEMVQGRFSCFGLQLRMNLPCSSQHLAPLQTRRRILQLTSTCSCYLWGGDMGAGLAVVPLMGLLESIAVARAFASQNNYRIDANQELLAIGLTNMLGSLVSSYPVTGSFGRTAVNAQSGVCTPAGGLVTGVLVLLSLDYLTSLFYYIPKSALAAVIIMAVAPLFDTKIFRTLWRVKRLDLLPLCVTFLLCFWEVQYGILAGALVSLLMLLHSAARPETKVSEGPVLVLQPASGLSFPAMEALREEILSRALEVSPPRCLVLECTHVCSIDYTVVLGLGELLQDFQKQGVALAFVGLQVPVLRVLLSADLKGFQYFSTLEEAEKHLRQEPGTQPYNIREDSILDQKVALLKA

SEQ ID NO: 6 - protein, 607 aa, Mus musculus
MPSSVKGLGQTRSPSLHMAPDTCCCSATALRRRLPVLAWVPDYSLQWLRLDFIAGLSVGLTVIPQALAYAEVAGLPPQYGLYSAFMGCFVYFFLGTSRDVTLGPTAIMSLLVSFYTFREPAYAVLLAFLSGCIQLAMGLLHLGFLLDFISCPVIKGFTSAASITIGFGQIKNLLGLQKIPRQFFLQVYHTFLHIGETRVGDAVLGLASMLLLLVLKCMREHMPPPHPEMPLAVKFSRGLVWTVTTARNALVVSSAALIAYAFEVTGSHPFVLTGKIAEGLPPVRIPPFSVTRDNKTISFSEMVQAKGSVTFSPQDMGAGLAVVPLMGLLESIAVAKSFASQNNYRIDANQELLAIGLTNVLGSLVSSYPVTGSFGRTAVNAQTGVCTPAGGLVTGALVLLSLNYLTSLFSYIPKSALAAVIITAVTPLFDVKIFRSLWRVQSTYTKRAVWCLSDRSLEDSPVLSASNVHPLLPFQVSEGQIFVLQPASGLYFPAIDALREAITNRALEASPPRSAVLECTHISSVDYTVIVGLGELLEDFQKKGVALAFVGLQVPVLRTLLAADLKGFRYFTTLEEAEKFLQQEPGTEPNSIHEDAVPEQRSSLLKS

SEQ ID NO: 7 - DNA, 20 bp, Artificial Sequence
  Oligonucleotide
gccacgccacatgatgatga

SEQ ID NO: 8 - DNA, 20 bp, Artificial Sequence
  Oligonucleotide
tcatcatcatgtggcgtggc

SEQ ID NO: 9 - DNA, 2032 bp, Mus musculus
  CDS (1)...(1815)
  CDS (52)...(1815)
atgcccagctctgtgaaaggtctgggtcagaccagatcccccagcctgcacatggcaccagacacatgctgctgctctgctacggccctgaggaggaggctacccgtcctggcctgggtgcctgactactctctgcagtggctgaggctggacttcatcgctggactttccgtgggactcaccgtcattccccaggccctggcctatgcagaagtggctggactcccaccccagtacggcctctactctgccttcatgggatgcttcgtgtatttcttcctgggcacctcccgggatgtgactctgggccccacggctatcatgtctctcctggtgtccttctacaccttccgtgagcctgcctatgctgtgctgcttgccttcctgtctgggtgtatccagctggccatggggctcctgcatttggggttcctgctggacttcatctcctgccctgtcattaaaggcttcacctccgctgccagcatcacaattggctttggacagatcaagaacctgctgggattgcagaaaatcccccggcagttcttcctccaggtgtaccacaccttcctccacatcggagagaccagggtaggcgacgctgtcctcggactggcctccatgttgctgctgcttgtgctgaagtgtatgcgggaacacatgcctcctccccatcctgagatgccccttgccgtgaagttcagccgtgggctggtgtggactgtcacaacagctcgcaatgccctggtggtctcctccgcggctctgattgcttacgccttcgaggtgacaggatcccatccctttgttctgactggaaagatcgccgaggggctccctccggtgcggatcccacccttctcagtgaccagggacaataagaccatctcgttctctgagatggtgcaggacatgggggccggactggctgtggtacctctgatggggctcctggagagcattgccgtggccaaatccttcgcgtctcagaataactaccgcattgatgctaaccaggaactactggccattggcctcaccaatgtgctgggctccctcgtctcctcttacccagtcactggcagctttgggcggacagctgtgaatgcccagacaggggtgtgtaccccggcaggaggcctggtgactggtgccctggtgctgctgtccctgaactacttgacctcactcttctcctatatccccaagtctgccctggctgccgtgatcatcacggctgtgaccccactctttgatgtcaagatcttcaggagtctctggcgcgttcagaggctggatctgctaccactgtgtgtgacgttcctgctgtccttctgggagatccagtacggtatcctggccggtagcctggtgtctttgctcattctcctgcactcggtagctaggcccaagactcaggtgtcagaaggacaaatttttgttcttcagccggccagcggcctgtacttccctgcaattgatgccctccgagaggcaataacgaaccgggcactggaagcatccccaccacgttccgcggttctggagtgcacgcatatcagcagtgtagactacaccgtgatcgtgggactcggtgagctcctggaggacttccagaagaaaggagtcgccctggcctttgttggcctacaggtgcccgtgctccgcacactgttggccgctgacctcaaggggttccgttacttcaccactctggaggaggctgagaaattcctgcagcaggaaccaggaactgagcccaacagcatccatgaagatgctgttccagagcaaaggagctccctgctcaagtctccctccggcccctgaagagcagatggtataggaagggtttctggaaggttctgtcaccatgacttggagtcacctgatagactcaccaacctggtgggacttaaaaggcactgcataggtggctctggggaacagcagggagccatgtatgatttccagggtgtcactttcctgctgtcccctaggtgtgagtatttgagggctgggctgactg

SEQ ID NO: 10 - protein, 610 aa, Mus musculus
MPSSVKGLGQTRSPSLHMAPDTCCCSATALRRRLPVLAWVPDYSLQWLRLDFIAGLSVGLTVIPQALAYAEVAGLPPQYGLYSAFMGCFVYFFLGTSRDVTLGPTAIMSLLVSFYTFREPAYAVLLAFLSGCIQLAMGLLHLGFLLDFISCPVIKGFTSAASITIGFGQIKNLLGLQKIPRQFFLQVYHTFLHIGETRVGDAVLGLASMLLLLVLKCMREHMPPPHPEMPLAVKFSRGLVWTVTTARNALVVSSAALIAYAFEVTGSHPFVLTGKIAEGLPPVRIPPFSVTRDNKTISFSEMVQDMGAGLAVVPLMGLLESIAVAKSFASQNNYRIDANQELLAIGLTNVLGSLVSSYPVTGSFGRTAVNAQTGVCTPAGGLVTGALVLLSLNYLTSLFSYIPKSALAAVIITAVTPLFDVKIFRSLWRVQRLDLLPLCVTFLLSFWEIQYGILAGSLVSLLILLHSVARPKTQVSEGQIFVLQPASGLYFPAIDALREAITNRALEASPPRSAVLECTHISSVDYTVIVGLGELLEDFQKKGVALAFVGLQVPVLRTLLAADLKGFRYFTTLEEAEKFLQQEPGTEPNSIHEDAVPEQRSSLLKSPSGP

SEQ ID NO: 11 - DNA, 2886 bp, Homo sapiens
  CDS (241)...(2208)
ggaatataaggggaattactggcgagagagctgtagaccaacttaacactgaacccattacttttccaagaccagaaaaaaatattacatgaacaggaactacttctccttcagataagaattcaagctttgacattgtaaaccacagacgaattggagcttggcattgaaaggaggtgttctgcaatgattttttttcttgtttagagaagtttacttctacaagaagaaatctgaaaaatgacaggagcaaagaggaaaaagaaaagcatgctttggagcaagatgcataccccccagtgtgaagacattatacagtggtgtagaaggcgactgcccattttggattgggcaccacattacaatctgaaagaaaacttgcttccagacactgtgtctgggataatgttggcagttcaacaggtgacccaaggattggcctttgctgttctctcatctgtgcacccagtgtttggtttatatgggtctctgtttcctgccataatttatgccatatttggaatgggacatcatgttgccacaggcacctttgccttgacatccttaatatcagccaacgccgtggaacggattgtccctcagaacatgcagaatctcaccacacagagtaacgcaagcgtgctgggcttatccgactttgaaatgcaaaggatccacgttgctgcagcagtttccttcttgggaggtgtgattcaggtggccatgtttgtgctgcaactgggcagtgccacatttgtggtcacagagcctgtgatcagcgcaatgacaactggggctgccacccatgtggtgacttcacaagtcaaatatctcttgggaatgaaaatgccatatatatccggaccacttggattcttttatatttatgcatatgtttttgaaaacatcaagtctgtgcgactggaagcattgcttttatccttgctgagcattgtggtccttgttcttgttaaagagctgaatgaacagtttaaaaggaaaattaaagttgttcttcctgtagatttagttttgattattgctgcatcatttgcttgttattgcaccaatatggaaaacacatatggattagaagtagttggtcatattccacaaggaattccctcacctagagctcccccgatgaacatcctctctgcggtgatcactgaagctttcggagtggcacttgtaggctatgtggcctcactggctcttgctcaaggatctgccaaaaaattcaaatattcaattgatgacaaccaggaatttttggcccatggcctcagcaatatagtttcttcatttttcttctgcataccaagtgctgctgccatgggaaggacggctggcctgtacagcacaggagcgaagacacaggtggcttgtctaatatcttgcattttcgtccttatagtcatctatgcaataggacctttgctttactggctgcccatgtgtgtccttgcaagcattattgttgtgggactgaagggaatgctaatacagttccgagatttaaaaaaatattggaatgtggataaaatcgattggggaatatgggtcagtacatatgtatttacaatatgctttgctgccaatgtgggactgctgtttggtgttgtttgtaccatagctatagtgataggacgcttcccaagagcaatgactgtaagtataaaaaatatgaaagaaatggaatttaaagtgaagacagaaatggacagtgaaaccctgcagcaggtgaaaattatctcaataaacaacccgcttgttttcctgaatgcaaaaaaattttatactgatttaatgaacatgatccaaaaggaaaatgcctgtaatcagccacttgatgatatcagcaagtgtgaacaaaacacattgcttaattccctatccaatggcaactgcaatgaagaagcttcacagtcctgccctaatgagaagtgttatttaatcctggattgcagtggatttaccttttttgactattctggagtctccatgcttgttgaggtttacatggactgtaaaggcaggagtgtggatgtattgttagcccattgtacagcttccttgataaaagcaatgacgtattatggaaacctagactcagagaaaccaattttttttgaatcggtatctgctgcaataagtcatatccattcaaataagaatttgagcaaactcagtgaccacagtgaagtctgagacccttttgtcacagtacagctcttgtctttaccaactgcctgaagaggccatatgctggcattttgcacaactttttggttgtttagatcctacagatgacctctgctacaataagtacgatgtgacttagtaactgcatagcagttggaaagaactgccaacttttttttctcatttttgttagtaagaagattcgcttagttattttatgtaaaaatcagtatgtgtttagttttagtgtactgaagggtaaacatggttttattttattttaccatattattttgtgttgttttatttctattgtgctgtaagttgatgtttaaaattgagaaatacttttgtcataggtaatttggaacatttacaagccatttgtaaaattttaagataatctgtaactaatacataaaaacaacttagcaaatgtgccattttcacacaacttctctctgtataggcctctgaaatatcaataaggctaaatattactttacacagtaagatgtgaaattcacaaaaagtaaaccaaacaaaacgaatgaaaaactggaaataattcgtttccatatctttccatacatccatttctgaagtattcaggaatgttttcataatcgaaagaaacgggtatccaaataaaaccaagttcttaaaaaaaaaaaaaaaaaa

SEQ ID NO: 12 - DNA, 2345 bp, Homo sapiens
  CDS (209)...(2200)
gtagaccaacttaacactgaacccattacttttccaagaccagaaaaaaatattacatgaacaggaactacttctccttcagataagaattcaagctttgacattgtaaaccacagacgaattggagcttggcattgaaaggaggtgttctgcaatgattttttttcttgtttagagaagtttacttctacaagaagaaatctgaaaaatgacaggagcaaagaggaaaaagaaaagcatgctttggagcaagatgcataccccccagtgtgaagacattatacagtggtgtagaaggcgactgcccattttggattgggcaccacattacaatctgaaagaaaacttgcttccagacactgtgtctgggataatgttggcagttcaacaggtgacccaaggattggcctttgctgttctctcatctgtgcacccagtgtttggtttatatgggtctctgtttcctgccataatttatgccatatttggaatgggacatcatgttgccacaggcacctttgccttgacatccttaatatcagccaacgccgtggaacggattgtccctcagaacatgcagaatctcaccacacagagtaacacaagcgtgctgggcttatccgactttgaaatgcaaaggatccacgttgctgcagcagtttccttcttgggaggtgtgattcaggtggccatgtttgtgctgcaactgggcagtgccacatttgtggtcacagagcctgtgatcagcgcaatgacaactggggctgccacccatgtggtgacttcacaagtcaaatatctcttgggaatgaaaatgccatatatatccggaccacttggattcttttatatttatgcatatgtttttgaaaacatcaagtctgtgcgactggaagcattgcttttatccttgctgagcattgtggtccttgttcttgttaaagagctgaatgaacagtttaaaaggaaaattaaagttgttcttcctgtagatttagttttgattattgctgcatcatttgcttgttattgcaccaatatggaaaacacatatggattagaagtagttggtcatattccacaaggaattccctcacctagagctcccccgatgaacatcctctctgcggtgatcactgaagctttcggagtggcacttgtaggctatgtggcctcactggctcttgctcaaggatctgccaaaaaattcaaatattcaattgatgacaaccaggaatttttggcccatggcctcagcaatatagtttcttcatttttcttctgcataccaagtgctgctgccatgggaaggacggctggcctgtacagcacaggagcgaagacacaggtggcttgtctaatatcttgcattttcgtccttatagtcatctatgcaataggacctttgctttactggctgcccatgtgtgtccttgcaagcattattgttgtgggactgaagggaatgctaatacagttccgagatttaaaaaaatattggaatgtggataaaatcgattggggaatatgggtcagtacatatgtatttacaatatgctttgctgccaatgtgggactgctgtttggtgttgtttgtaccatagctatagtgataggacgcttcccaagagcaatgactgtaagtataaaaaatatgaaagaaatggaatttaaagtgaagacagaaatggacagtgaaaccctgcagcaggtgaaaattatctcaataaacaacccgcttgttttcctgaatgcaaaaaaattttatactgatttaatgaacatgatccaaaaggaaaatgcctgtaatcagccacttgatgatatcagcaagtgtgaacaaaacacattgcttaattccctatccaatggcaactgcaatgaagaagcttcacagtcctgccctaatgagaagtgttatttaatcctggattgcagtggatttaccttttttgactattctggagtctccatgcttgttgaggtttacatggactgtaaaggcaggagtgtggatgtattgttagcccattgtacagcttccttgataaaagcaatgacgtattatggaaacctagactcagagaaaccaattttttttgaatcggtatctgctgcaataagtcatatccattcaaataaggccagttataaactgttgtttgataacttggatcttccaacaatgccaccgctctgaggattgggtggttgcctatcatttgcaaactgcttacttgtacaacaaatgcttcttccaggatctactgtcctggggacttgaatccacctttctcaaatataaaaactctaaatatagaaaaaaaaaaaaaaaaaaaaaaaaa

SEQ ID NO: 13 - protein, 656 aa, Homo sapiens [sequence omitted]

SEQ ID NO: 14 - protein, 663 aa, Homo sapiens
MTGAKRKKKSMLWSKMHTPQCEDIIQWCRRRLPILDWAPHYNLKENLLPDTVSGIMLAVQQVTQGLAFAVLSSVHPVFGLYGSLFPAIIYAIFGMGHHVATGTFALTSLISANAVERIVPQNMQNLTTQSNTSVLGLSDFEMQRIHVAAAVSFLGGVIQVAMFVLQLGSATFVVTEPVISAMTTGAATHVVTSQVKYLLGMKMPYISGPLGFFYIYAYVFENIKSVRLEALLLSLLSIVVLVLVKELNEQFKRKIKVVLPVDLVLIIAASFACYCTNMENTYGLEVVGHIPQGIPSPRAPPMNILSAVITEAFGVALVGYVASLALAQGSAKKFKYSIDDNQEFLAHGLSNIVSSFFFCIPSAAAMGRTAGLYSTGAKTQVACLISCIFVLIVIYAIGPLLYWLPMCVLASIIVVGLKGMLIQFRDLKKYWNVDKIDWGIWVSTYVFTICFAANVGLLFGVVCTIAIVIGRFPRAMTVSIKNMKEMEFKVKTEMDSETLQQVKIISINNPLVFLNAKKFYTDLMNMIQKENACNQPLDDISKCEQNTLLNSLSNGNCNEEASQSCPNEKCYLILDCSGFTFFDYSGVSMLVEVYMDCKGRSVDVLLAHCTASLIKAMTYYGNLDSEKPIFFESVSAAISHIHSNKASYKLLFDNLDLPTMPPL

SEQ ID NO: 15 - protein, 22 aa, Artificial Sequence
  Consensus sulfate transporter signature (Prosite Reference PS01130)
XXYXLYXXXXXXXXXXXXXXSX

SEQ ID NO: 16 - DNA, 35 bp, Artificial Sequence
  Oligonucleotide
ccaccatgacaggagcaaagaggaaaaagaaaagc

SEQ ID NO: 17 - DNA, 38 bp, Artificial Sequence
  Oligonucleotide
gcgtctagatcagacttcactgtggtcactgagtttgc

SEQ ID NO: 18 - DNA, 28 bp, Artificial Sequence
  Oligonucleotide
cttcttcattgcagttgccattggatag

SEQ ID NO: 19 - DNA, 28 bp, Artificial Sequence
  Oligonucleotide
gaaaccctgcagcaggtgaaaattatct

SEQ ID NO: 20 - protein, 48 aa, Artificial Sequence
  Consensus sulfate transporter signature (pfam reference PF00916)
GRLGLSGFGAIQLGLLLTGGPADNELAGNIRSNGTLSLPLILDNGGVS

SEQ ID NO: 21 - DNA, 27 bp, Artificial Sequence
  Oligonucleotide
accgtagagatgccttcttcggtgacg

SEQ ID NO: 22 - DNA, 27 bp, Artificial Sequence
  Oligonucleotide
catgtctatcagcggcatccagcatcc

SEQ ID NO: 23 - DNA, 32 bp, SUT-3 amplification primer
gggaattccatatgccttcttcggtgacggcg

SEQ ID NO: 24 - DNA, 30 bp, SUT-3 amplification primer
ccggaattcttatgccttgagcagggcaac

SEQ ID NO: 25 - DNA, 29 bp, SUT-3 amplification primer
gctctagagccaccatggaggagcagaag

SEQ ID NO: 26 - DNA, 29 bp, SUT-3 amplification primer
gctctagattatgccttgagcagggcaac

SEQ ID NO: 27 - protein, 593 aa, Mus musculus
MAPDTCCCSATALRRRLPVLAWVPDYSLQWLRLDFIAGLSVGLTVIPQALAYAEVAGLPPQYGLYSAFMGCFVYFFLGTSRDVTLGPTAIMSLLVSFYTFREPAYAVLLAFLSGCIQLAMGLLHLGFLLDFISCPVIKGFTSAASITIGFGQIKNLLGLQKIPRQFFLQVYHTFLHIGETRVGDAVLGLASMLLLLVLKCMREHMPPPHPEMPLAVKFSRGLVWTVTTARNALVVSSAALIAYAFEVTGSHPFVLTGKIAEGLPPVRIPPFSVTRDNKTISFSEMVQDMGAGLAVVPLMGLLESIAVAKSFASQNNYRIDANQELLAIGLTNVLGSLVSSYPVTGSFGRTAVNAQTGVCTPAGGLVTGALVLLSLNYLTSLFSYIPKSALAAVIITAVTPLFDVKIFRSLWRVQRLDLLPLCVTFLLSFWEIQYGILAGSLVSLLILLHSVARPKTQVSEGQIFVLQPASGLYFPAIDALREAITNRALEASPPRSAVLECTHISSVDYTVIVGLGELLEDFQKKGVALAFVGLQVPVLRTLLAADLKGFRYFTTLEEAEKFLQQEPGTEPNSIHEDAVPEQRSSLLKSPSGP